(19) United States
(12) Patent Application Publication
(10) Pub. No.: US 2026/0224736 A1
(43) Pub. Date: Aug. 6, 2026

(54) HSC-SPECIFIC ANTIBODY CONJUGATED LIPID NANOPARTICLES AND USES THEREOF

(71) Applicant: GENZYME CORPORATION, Cambridge, MA (US)

(72) Inventors: Christopher BORGES, Cambridge, MA (US); Austin Wayne BOESCH, Somerville, MA (US); Daryl Clark DRUMMOND, Lincoln, MA (US); Jacob HOPE, Cambridge, MA (US); Christophe LECHAUVE, Acton, MA (US); Christian MUELLER, Concord, MA (US)

(21) Appl. No.: 19/153,289

(22) PCT Filed: Feb. 2, 2024
(86) PCT No.: PCT/US2024/014265

§ 371 Date: Aug. 1, 2025

Related U.S. Application Data
(60) Provisional application No. 63/443,223, filed on Feb. 3, 2023.
(60) Provisional application No. 63/463,252, filed on May 1, 2023.
(60) Provisional application No. 63/467,282, filed on May 17, 2023.

(51) Int. Cl. A61K 47/69; A61K 9/50; A61K 31/395; A61K 31/7088; A61K 38/19; A61K 38/46; A61K 47/68; A61K 48/00

(52) U.S. Cl. CPC A61K 47/6929; A61K 9/5015; A61K 31/395; A61K 31/7088; A61K 38/193; A61K 38/465; A61K 47/6849; A61K 48/0033; A61K 48/005

(57) ABSTRACT

Provided are ionizable cationic lipids and lipid nanoparticles for the delivery of nucleic acids to cells (e.g., HSC), and methods of making and using such lipids and targeted lipid. nanoparticles.

### CROSS-REFERENCE TO RELATED APPLICATIONS

This application claims the benefit of priority to U.S. Provisional Application No. 63/443,223, filed on Feb. 3, 2023, to U.S. Provisional Application No. 63/463,252, filed on May 1, 2023, and to U.S. Provisional Application No. 63/467,282, filed May 17, 2023, each of which is incorporated by reference herein in its entirety.

### REFERENCE TO AN ELECTRONIC SEQUENCE LISTING

The contents of the electronic sequence listing (183952035040SEQLIST.xml; Size: 57,069 bytes; and Date of Creation: Jan. 30, 2024) is herein incorporated by reference in its entirety.

### FIELD OF THE INVENTION

The invention provides lipid nanoparticles for the delivery of nucleic acids to hematopoietic stem cells, methods of making and using.

### BACKGROUND

There exists a need for safe and effective in vivo methods for targeted gene editing in hematopoietic stem cells (HSCs).

### SUMMARY

Provided herein is a lipid nanoparticle (LNP) for targeted delivery of a nucleic acid into a hematopoietic stem cell (HSC), the LNP comprising a lipid-antibody conjugate comprising the compound of Formula (I): [Lipid]-[optional linker]-[antibody], wherein the antibody binds to CD105 and/or CD117; an ionizable cationic lipid comprising: (i) a compound of Formula (II′):

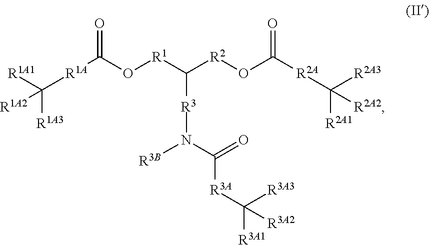

or a salt thereof, wherein: R¹, R², and R³ are each independently a bond or C₁₋₃ alkylene; R^{1A}, R^{2A}, and R^{3A} are each independently a bond or C₁₋₁₀ alkylene; R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}; R^{a1} and R^{a2} are each independently C₁₋₂₀ alkyl or C₁₋₂₀ alkenyl;
R^{3B} is

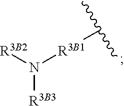

R^{3B1} is C₁₋₆ alkylene; and R^{3B2} and R^{3B3} are each independently H or C₁₋₆ alkyl optionally substituted with one or more substituents each independently selected from the group consisting of —OH and —O—(C₁₋₆ alkyl); or (ii) a compound having the following structure:

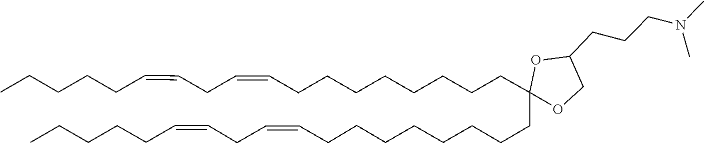 [SMILES: CCCCC/C=C\C/C=C\CCCCCCCCC1(CCCCCCCC/C=C\C/C=C\CCCCC)OCC(CCCN(C)C)O1]

or a salt thereof;
and one or more nucleic acids disposed in the LNP.

In some embodiments, the compound of Formula (II′) is a compound of Formula (II):

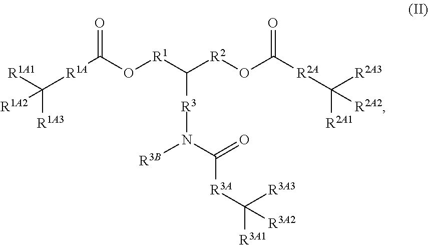

or a salt thereof, wherein: R¹, R², and R³ are each independently a bond or C₁₋₃ alkylene; R^{1A}, R^{2A}, and R^{3A} are each independently a bond or C₁₋₁₀ alkylene; R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}; R^{a1} and R^{a2} are each independently C₁₋₂₀ alkyl or C₁₋₂₀ alkenyl;
R^{3B} is

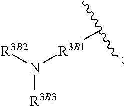

R^{3B1} is C₁₋₆ alkylene; and R^{3B2} and R^{3B3} are each independently H or C₁₋₆ alkyl.

In some embodiments, the LNP comprises two lipid-antibody conjugates comprising the compound of Formula (I), wherein the first lipid-antibody conjugate and the second lipid-antibody conjugate are the same or different.

In some embodiments, the antibody of the LNP that binds to CD105 and/or CD117 comprises an antibody heavy chain variable region (VH) and an antibody light chain variable region (VL). In some embodiments, the antibody binds to CD117, where the VH comprises a CDR-H1 comprising the amino acid sequence of FTFSNYAMS (SEQ ID NO: 1), a CDR-H2 comprising the amino acid sequence of AISGSGGSTYYADSVKG (SEQ ID NO: 2), and a CDR-H3 comprising the amino acid sequence of AKGPPTYHTNYYYMDV (SEQ ID NO: 3), and the VL comprises CDR-L1 comprising the amino acid sequence of RASQGISSWLA (SEQ ID NO: 4), a CDR-L2 comprising the amino acid sequence of AASSLQS (SEQ ID NO: 5), and a CDR-L3 comprising the amino acid sequence of QQTNSFPYT (SEQ ID NO: 6). In some embodiments, the antibody binds to CD117, where the VH comprises a CDR-H1 comprising the amino acid sequence of FTFSDADMD (SEQ ID NO: 10), a CDR-H2 comprising the amino acid sequence of RTRNKAGSYTTEYAASVKG (SEQ ID NO: 11), and a CDR-H3 comprising the amino acid sequence of AREPKYWIDFDL (SEQ ID NO: 12), and the VL comprises CDR-L1 comprising the amino acid sequence of RASQSISSYLN (SEQ ID NO: 13), a CDR-L2 comprising the amino acid sequence of AASSLQS (SEQ ID NO. 14), and a CDR-L3 comprising the amino acid sequence of QQSYIAPYT (SEQ ID NO: 15). In some embodiments, the antibody binds to CD105, where VH comprises a CDR-H1 comprising the amino acid sequence of DAWMD (SEQ ID NO: 19), a CDR-H2 comprising the amino acid sequence of EIRSKASNHATYYAESVKG (SEQ ID NO: 20), and a CDR-H3 comprising the amino acid sequence of WRRFFDS (SEQ ID NO: 21), and VL comprises CDR-L1 comprising the amino acid sequence of RASSSVSYMH (SEQ ID NO: 22), a CDR-L2 comprising the amino acid sequence of ATSNLAS (SEQ ID NO. 23), and a CDR-L3 comprising the amino acid sequence of QQWSSNPLT (SEQ ID NO: 24).

In some embodiments, the antibody of the LNP binds to CD117 and comprises an antibody heavy chain variable region (VH) and an antibody light chain variable region (VL), wherein the VH comprises a CDR-H1 comprising the amino acid sequence of FTFSNYAMS (SEQ ID NO: 1), a CDR-H2 comprising the amino acid sequence of AISGSGGSTYYADSVKG (SEQ ID NO: 2), and a CDR-H3 comprising the amino acid sequence of AKGPPTYHTNYYYMDV (SEQ ID NO: 3), and the VL comprises CDR-L1 comprising the amino acid sequence of RASQGISSWLA (SEQ ID NO: 4), a CDR-L2 comprising the amino acid sequence of AASSLQS (SEQ ID NO: 5), and a CDR-L3 comprising the amino acid sequence of QQTNSFPYT (SEQ ID NO: 6).

In some embodiments, the antibody of the LNP binds to CD117 and comprises an antibody heavy chain variable region (VH) and an antibody light chain variable region (VL), wherein the VH comprises a CDR-H1 comprising the amino acid sequence of FTFSDADMD (SEQ ID NO: 10), a CDR-H2 comprising the amino acid sequence of RTRNKAGSYTTEYAASVKG (SEQ ID NO: 11), and a CDR-H3 comprising the amino acid sequence of AREPKYWIDFDL (SEQ ID NO: 12), and the VL comprises CDR-L1 comprising the amino acid sequence of RASQSISSYLN (SEQ ID NO: 13), a CDR-L2 comprising the amino acid sequence of AASSLQS (SEQ ID NO: 14), and a CDR-L3 comprising the amino acid sequence of QQSYIAPYT (SEQ ID NO: 15).

In some embodiments, the antibody of the LNP binds to CD105 and comprises an antibody heavy chain variable region (VH) and an antibody light chain variable region (VL), wherein VH comprises a CDR-H1 comprising the amino acid sequence of DAWMD (SEQ ID NO: 19), a CDR-H2 comprising the amino acid sequence of EIRSKASNHATYYAESVKG (SEQ ID NO: 20), and a CDR-H3 comprising the amino acid sequence of WRRFFDS (SEQ ID NO: 21), and VL comprises CDR-L1 comprising the amino acid sequence of RASSSVSYMH (SEQ ID NO: 22), a CDR-L2 comprising the amino acid sequence of ATSNLAS (SEQ ID NO: 23), and a CDR-L3 comprising the amino acid sequence of QQWSSNPLT (SEQ ID NO: 24).

In some embodiments, the antibody of the LNP comprises an antibody heavy chain variable region (VH) and an antibody light chain variable region (VL), wherein the antibody binds to CD117, and wherein the VH comprises the amino acid sequence of SEQ ID NO: 7 and the VL comprises the amino acid sequence of SEQ ID NO: 8. In some embodiments, the antibody comprises an antibody heavy chain variable region (VH) and an antibody light chain variable region (VL), wherein the antibody binds to CD117, and wherein the VH comprises the amino acid sequence of SEQ ID NO: 16 and the VL comprises the amino acid sequence of SEQ ID NO: 17. In some embodiments, the antibody comprises an antibody heavy chain variable region (VH) and an antibody light chain variable region (VL), wherein the antibody binds to CD105, and wherein the VH comprises the amino acid sequence of SEQ ID NO: 25 and the VL comprises the amino acid sequence of SEQ ID NO: 26.

In some embodiments, the antibody of the LNP binds to CD117 and comprises an antibody heavy chain variable region (VH) and an antibody light chain variable region (VL), wherein the VH comprises the amino acid sequence of SEQ ID NO: 7 and the VL comprises the amino acid sequence of SEQ ID NO: 8.

In some embodiments, the antibody of the LNP binds to CD117 and comprises an antibody heavy chain variable region (VH) and an antibody light chain variable region (VL), wherein the VH comprises the amino acid sequence of SEQ ID NO: 16 and the VL comprises the amino acid sequence of SEQ ID NO: 17.

In some embodiments, the antibody of the LNP binds to CD105 and comprises an antibody heavy chain variable region (VH) and an antibody light chain variable region (VL), wherein the VH comprises the amino acid sequence of SEQ ID NO: 25 and the VL comprises the amino acid sequence of SEQ ID NO: 26.

In some embodiments, the antibody of the LNP that binds to CD105 and/or CD117 comprises a Fab, F(ab′)2, Fab′-SH, Fv, scFv fragment, or immunoglobulin single variable domain. In some embodiments, the antibody comprises a Fab. In some embodiments, the antibody comprises an Fc domain.

In some embodiments, the antibody comprises a Fab comprising a heavy chain domain and a light chain domain. In some embodiments, the heavy chain and light chain domains of the Fab comprise the amino acid sequences set forth in SEQ ID NOs: 9 and 38, or have at least 85, 90, 95, 96, 97, 98, 99, or 99.5% sequence identity with the amino acid sequences set forth in SEQ ID NOs: 9 and 38. In some embodiments, the heavy chain and light chain domains of the Fab comprise the amino acid sequences set forth in SEQ ID NOs: 18 and 39, or have at least 85, 90, 95, 96, 97, 98, 99, or 99.5% sequence identity with the amino acid sequences set forth in SEQ ID NOs: 18 and 39. In some embodiments, the heavy chain and light chain domains of the Fab comprise the amino acid sequences set forth in SEQ ID NOs: 27 and 40, or have at least 85, 90, 95, 96, 97, 98, 99, or 99.5% sequence identity with the amino acid sequences set forth in SEQ ID NOs: 27 and 40.

In some embodiments, the Fab lacks the native interchain disulfide bond at the C-terminus. In some embodiments, the Fab is engineered to replace one or both cysteines on the native constant light chain and the native constant heavy chain that form the native interchain disulfide with a non-cysteine amino acid, therefor to remove the native interchain disulfide bond in the Fab. In some embodiments, the Fab comprises a heavy chain fragment that comprises C233S substitution, and a light chain fragment that comprises C214S substitution, numbering according to Kabat.

In some embodiments, the Fab has a non-natural interchain disulfide bond. In some embodiments, the Fab has an engineered, buried interchain disulfide bond.

In some embodiments, the Fab comprises F174C substitution in the heavy chain fragment, and S176C substitution in the light chain fragment, numbering according to Kabat. In some embodiments, the Fab comprises a cysteine at the C-terminus of the heavy or light chain fragment. In some embodiments, the Fab further comprises one or more amino acids between the heavy chain fragment of the Fab and the C-terminal cysteine.

In some embodiments, the antibody comprises an immunoglobulin single variable (ISV) domain. In some embodiments, the ISV domain is a Nanobody® ISV domain. In some embodiments, the immunoglobulin single variable domain comprises a cysteine at the C-terminus.

In some embodiments, the antibody comprises two or more VHH domains.

In some embodiments, the immunoglobulin single variable domain comprises a VHH domain and further comprises a spacer comprising one or more amino acids between the VHH domain and the C-terminal cysteine. In some embodiments, the two or more VHH domains are linked by an amino acid spacer. In some embodiments, the antibody comprises a first VHH domain linked to an antibody CH1 domain and a second VHH domain linked to an antibody light chain constant domain.

In some embodiments, the antibody CH1 domain and the antibody light chain constant domain are linked by one or more disulfide bonds. In some embodiments, the CH1 domain comprises F174C and C233S substitutions, and the light chain constant domain comprises S176C and C214S substitutions, numbering according to Kabat.

In some embodiments, the antibody comprises an amino acid spacer or linker with the amino acid sequence of AAA, or with an amino acid sequence set forth in any one of SEQ ID NOs: 45-60.

In some embodiments, the antibody comprises a bispecific antibody. In some embodiments, the antibody of the LNP that binds to CD105 and/or CD117 comprises a bispecific antibody.

In some embodiments, provided herein is a LNP, where the one or more nucleic acids is DNA or RNA. In some embodiments, the RNA is an mRNA.

In some embodiments, the one or more nucleic acids of the LNP comprise an mRNA encoding a site-directed nuclease, a chemical base editor, a prime editor, or an epigenome editor. In some embodiments, the one or more nucleic acids comprise an mRNA encoding a site-directed nuclease. In some embodiments, the site-directed nuclease is a CRISPR-associated (Cas) nuclease, a zinc finger nuclease (ZFN), a transcription activator-like effector nuclease (TALEN), or a megaTAL. In some embodiments, the site-directed nuclease is a Cas nuclease, ZFN, TALEN, or megaTAL comprising an amino acid sequence that confers binding to a target nucleotide sequence.

In some embodiments, the one or more nucleic acids of the LNP comprise an mRNA encoding a CRISPR-associated (Cas) nuclease or a chemical base editor; and a guide RNA (gRNA) comprising a nucleotide sequence that confers binding to a target nucleotide sequence.

In some embodiments, the one or more nucleic acids of the LNP comprise an mRNA encoding a prime editor; and a prime editing guide RNA (pegRNA) comprising a nucleotide sequence that confers binding to a target nucleotide sequence.

In some embodiments, the Cas nuclease is a Type II or a Type V Cas enzyme, or a variant thereof. In some embodiments, the Cas nuclease is a Cas9 enzyme, a Cas12 enzyme, a CasX enzyme, a Cas14 enzyme, or a variant thereof.

In some embodiments, the gRNA or pegRNA of the LNP comprises a sequence having at least 80% identity to at least 15 consecutive nucleotides of the target nucleotide sequence. In some embodiments, the one or more nucleic acids of the LNP further comprise a donor template nucleic acid comprising a sequence having at least 80% identity to at least 15 consecutive nucleotides of the target nucleotide sequence.

In some embodiments, the target nucleotide sequence comprises at least 15 consecutive nucleotides and is located within a coding region of a gene, an intronic region associated with a gene, an exon region associated with a gene, a 5′ untranslated region associated with a gene, or a 3′ untranslated region associated with a gene, wherein the gene is selected from the group consisting of gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FAN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C.

In some embodiments, the target nucleotide sequence is within a regulatory region, optionally an enhancer region or a repressor region, of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF, FANCA, FANCB, FANCC, FANCD1/BRACA2, FANCD2, MPL, CCR5, CXCR4, F5, F2, antithrombin III, and protein C. In some embodiments, the target nucleotide sequence is within the BCL11A erythroid enhancer. In some embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions.

In some embodiments, the ionizable cationic lipid of the LNP comprises a compound of Formula (II′). In some embodiments, the ionizable cationic lipid of the LNP comprises a compound of Formula (II). In some embodiments, R^{3B2} and R^{3B3} are each independently H or C₁₋₆ alkyl, optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each unsubstituted methyl.

In some embodiments

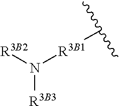

is

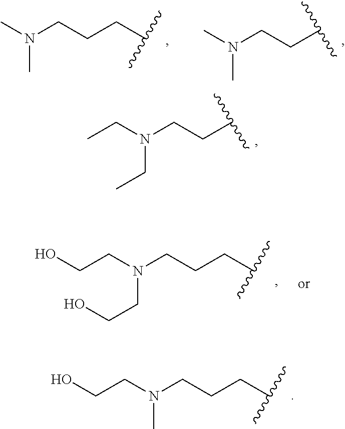

In some embodiments,

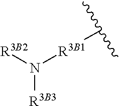

is

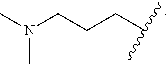 [SMILES: *CCCN(C)C]

In some embodiments, R¹, R², and R³ are each independently a bond or methylene. In some embodiments, R¹ and R² are each methylene and R³ is a bond.

In some embodiments, the ionizable cationic lipid of the LNP is a compound of Formula (IIa):

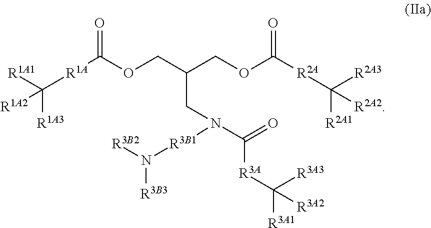

wherein R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{3B1}, R^{3B2}, and R^{3B3} are as defined for Formula (II′), Formula (II), or any variation or embodiment thereof.

In some embodiments, the ionizable cationic lipid of the LNP is a compound of Formula (IIb):

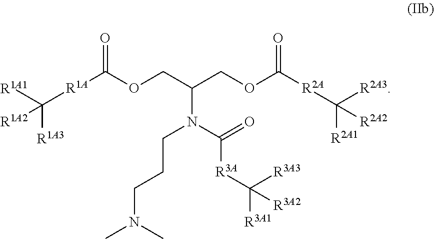

In some embodiments, R^{1A}, R^{2A}, and R^{3A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{3A} is a bond, —CH₂—, —(CH₂)₂—, or —(CH₂)₇—. In some embodiments, R^{1A1}, R^{1A2}, R^{1A3}, R^{2A1}, R^{2A2}, and R^{2A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl). In some embodiments, R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H. In some embodiments, R^{1A1} and R^{2A1} are each

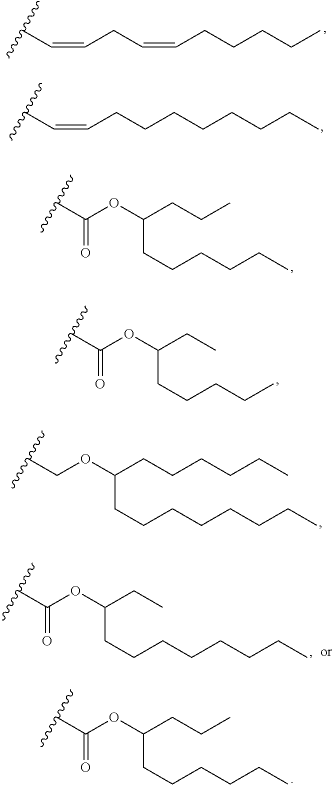

In some embodiments, R^{1A1} and R^{2A1} are each

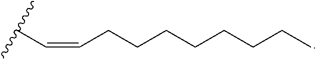 [SMILES: */C=C\CCCCCCCC]

In some embodiments, R^{3A1}, R^{3A2}, and R^{3A3} are each independently H, C₁₋₁₅ alkyl, —(CH₂)₀₋₄C(O)OCH(C₁₋₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

In some embodiments, R^{3A1} and R^{3A2} are each independently C₁₋₁₅ alkyl; and R^{3A3} is H.

In some embodiments, R^{3A1} and R^{3A2} are each independently ethyl, propyl,

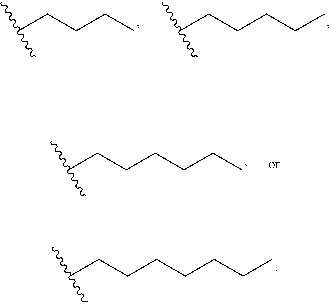

In some embodiments, R^{3A1} is propyl and R^{3A2} is

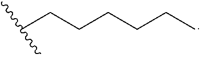 [SMILES: *CCCCCC]

In some embodiments, the ionizable cationic lipid of the LNP is

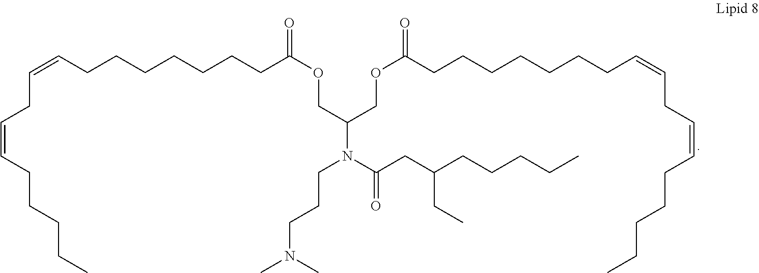 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

In some embodiments, the ionizable cationic lipid of the LNP is

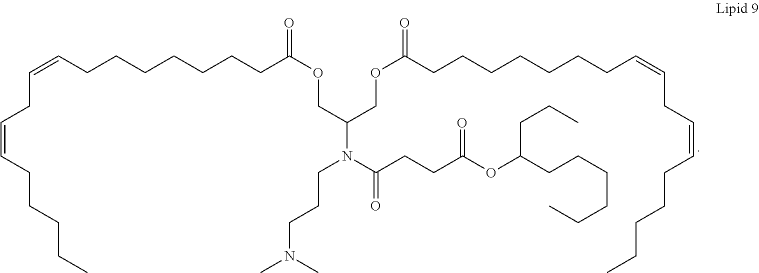 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

In some embodiments, the ionizable cationic lipid of the LNP is

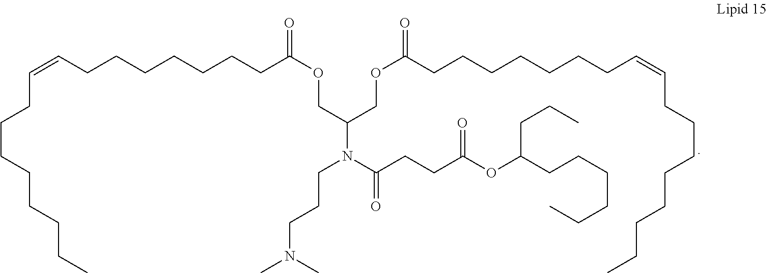 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

In the ionizable cationic lipid comprises the compound having the structure

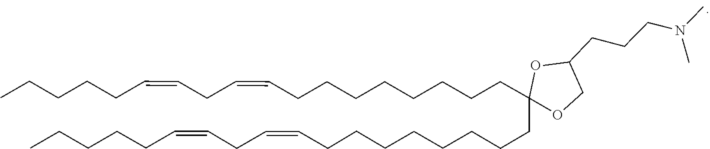 [SMILES: CCCCC/C=C\C/C=C\CCCCCCCCC1(CCCCCCCC/C=C\C/C=C\CCCCC)OCC(CCCN(C)C)O1]

In some aspect, provided herein is an LNP where the antibody is covalently coupled to a lipid in the LNP via a polyethylene glycol (PEG) containing linker. In some embodiments, the antibody that binds to CD105 and/or CD117 is covalently coupled to a lipid in the LNP via a polyethylene glycol (PEG) containing linker. In some embodiments, the lipid covalently coupled to the antibody via a PEG containing linker is distearoylglycerol (DSG), distearoylphosphatidylethanolamine (DSPE), dimyrstoyl-phosphatidylethanolamine (DMPE), distearoyl-glycero-phosphoglycerol (DSPG), dimyristoyl-glycerol (DMG), dipalmitoylphosphatidylethanolamine (DPPE), dipalmitoyl-glycerol (DPG), or ceramide. In some embodiments, the PEG is PEG 2000 or PEG 3400.

In some embodiments, the lipid-antibody conjugate is present in the LNP in a range of 0.001 to 0.5 mole percent. In some embodiments, the lipid-antibody conjugate is present in the LNP in a range of 0.002-0.2 mole percent.

In some embodiments, the ionizable cationic lipid is present in the LNP in a range of 30-70 mole percent. In some embodiments, the ionizable cationic lipid is present in the LNP in a range of 40-60 mole percent.

In some embodiments, the LNP further comprises one or more of a structural lipid, a neutral phospholipid, and a free PEG-lipid. In some embodiments, the structural lipid is a sterol. In some embodiments, the sterol is cholesterol. In some embodiments, the sterol is present in the LNP in a range of 20-70 mole percent. In some embodiments, the sterol is present in the LNP in a range of 30-50 mole percent. In some embodiments, the sterol comprises cholesterol and is present in the LNP at a concentration of about 40 mole percent.

In some embodiments, the neutral phospholipid is selected from the group consisting of phosphatidylcholine, phosphatidylethanolamine, distearoyl-snglycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin. In some embodiments, the neutral phospholipid is present in the LNP in a range of 5-15 mole percent. In some embodiments, the concentration of the neutral phospholipid in the LNP is about 10 mole percent. In some embodiments, the neutral phospholipid is DSPC and the concentration of DSPC in the LNP is about 10 mole percent.

In some embodiments, the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. In some embodiments, the free PEG-lipid comprises PEG-dioleoylgylcerol (PEG-DOG), PEG-dimyristoyl-glycerol (PEG-DMG), PEG-dipalmitoylglycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoylphosphatidylethanolamine (PEG-DPPE), PEG-distearoylglycerol (PEG-DSG), N-palmitoyl-sphingosine-1-{succinyl[methoxy(polyethylene glycol)](PEG-ceramide), PEG-distearoyl-glycero-phosphoglycerol (PEG-DSPG), PEG-dioleoyl-glycerophosphoethanolamine (PEG-DOPE), 2-[(polyethylene glycol)-2000]-N,Nditetradecylacetamide, PEG-distearoyl-phosphatidylethanolamine (PEG-DSPE), or a derivative thereof. In some embodiments, the free PEG-lipid comprises a diacylphosphatidylethanolamine comprising Dipalmitoyl (C16) chain or Distearoyl (C18) chain, and optionally the free PEG-lipid comprises PEG-DPG and PEG-DMG. In some embodiments, the free PEG-lipid is present in the LNP in a range of 1-4 mole percent. In some embodiments, the PEG-lipid is DPG-PEG comprising PEG that has at a molecular weight of 2000 daltons, and the DPG-PEG lipid is present in the LNP at a concentration of about 1.5 mole percent.

In some embodiments, the free PEG-lipid comprises the same or a different lipid as the lipid in the lipid-antibody conjugate.

In some embodiments, the free PEG-lipid comprises a PEG having a molecular weight of at least 2000 daltons. In some embodiments, the PEG has a molecular weight of about 3000 to 5000 daltons.

In some embodiments, the LNP has a mean diameter in the range of 50-200 nm. In some embodiments, the LNP has a mean diameter of about 100 nm. In some embodiments, the LNP has a polydispersity index in a range from 0.05 to 1. In some embodiments, the LNP has a zeta potential of from about +10 mV to about +30 mV at pH 5. In some embodiments, the LNP has a zeta potential of from about −30 mV to about +5 mV at pH 7.4.

In some embodiments, the LNP comprises the ionizable cationic lipid, the lipid-antibody conjugate comprising the compound of the following formula: [Lipid]-[optional linker]-[antibody], wherein the antibody binds to CD105 and/or CD117, a sterol or other structural lipid, a neutral phospholipid, a free Polyethylene glycol (PEG) lipid, and the nucleic acid.

Also provided herein is a method of targeting the delivery of a nucleic acid to a hematopoietic stem cell (HSC), optionally ex vivo or in vivo in a subject, the method comprising administering to the subject the LNP of any one of the preceding embodiments, wherein the LNP comprises the nucleic acid. In some embodiments, the subject is a human.

In some embodiments, the method further comprises administering to the subject an HSC mobilization agent, and wherein the LNP is administered to the subject intravenously. In some embodiments, the HSC mobilization agent is administered to the subject before, during, or before and during administration of the LNP. In some embodiments, the HSC mobilization agent comprises plerixafor, granulocyte colony stimulating factor (G-CSF), granulocyte-macrophage colony stimulating factor (GM-CSF), or any combination thereof. In some embodiments, the HSC mobilization agent comprises plerixafor and G-CSF.

Also provided herein is a method of genetically modifying a hematopoietic stem cell (HSC), optionally ex vivo or in vivo in a subject, the method comprising administering to the subject the LNP of any one of the preceding embodiments, wherein the one or more nucleic acids disposed in the LNP comprise an mRNA encoding a site-directed nuclease, a chemical base editor, a prime editor, or an epigenome editor.

Also provided herein is a method of treating a disease in a subject in need thereof, the method comprising administering to the subject the LNP of any one of the preceding embodiments, wherein the one or more nucleic acids disposed in the LNP comprise a sequence, optionally an mRNA, encoding a site-directed nuclease, a chemical base editor, a prime editor, or an epigenome editor.

In some embodiments, the method further comprises administering to the subject an HSC mobilization agent, and wherein the LNP is administered to the subject intravenously. In some embodiments, the HSC mobilization agent is administered to the subject before, during, or before and during administration of the LNP. In some embodiments, the HSC mobilization agent comprises plerixafor, granulocyte colony stimulating factor (G-CSF), granulocyte-macrophage colony stimulating factor (GM-CSF), or any combination thereof. In some embodiments, the HSC mobilization agent comprises plerixafor and G-CSF.

In some embodiments, the disease is a blood disease. In some embodiments, the disease is a hemoglobinopathy, a primary immune deficiency (PID), a congenital cytopenia, a hemophilia, a thrombophilia, an inborn error of metabolism, a neuropathy, or a viral disease. In some embodiments, the disease is an α-hemoglobinopathy or a β-hemoglobinopathy. In some embodiments, the β-hemoglobinopathy is β-thalassemia or sickle cell disease.

In some embodiments, administration of the LNPs results in one or more of (i) an insertion of an HBB transgene, or a fragment thereof, into at least one HSC of the subject; (ii) increased expression of β-globin in the subject; (iii) an increased amount of α₂β₂ adult hemoglobin (HbA) in the subject; (iv) insertion of an HBG1 transgene, or a fragment thereof, into at least one HSC of the subject; (v) insertion of an HBG2 transgene, or a fragment thereof, into at least one HSC of the subject; (vi) increased expression of γ-globin in the subject; (vii) an increased amount of α₂γ₂ fetal hemoglobin (HbF) in the subject; (viii) disruption of the HBA1 gene, the HBA2 gene, or a combination thereof in at least one HSC of the subject; (ix) decreased expression of α-globin in the subject; and (x) a decreased amount of α₄ α-globin heterotetramers the subject.

In some embodiments, the disease is a PID. In some embodiments, the PID is a severe combined immunodeficiency (SCID), Wiskott-Aldrich syndrome, chronic granulomatous disease, immunodysregulation polyendocrinopathy enteropathay X-linked (IPEX), a hyper IgM syndrome, or X-linked agammaglobulinemia.

In some embodiments, the PID is a SCID. In some embodiments, the SCID is Artemis-SCID (ART-SCID), recombination activating gene SCID (RAG-SCID), X-linked SCID (X-SCID), adenosine deaminase-deficient SCID, interleukin 7 receptor deficiency SCID, or JAK3 SCID. In some embodiments, the SCID is ART-SCID, and wherein administration of the LNP results in insertion of a DCLREIC transgene, or a fragment thereof, into at least one HSC of the subject; increased expression of functional Artemis protein in the subject; or a combination thereof. In some embodiments, the SCID is RAG-SCID, and wherein administration of the LNP results in insertion of a RAG1 transgene or a RAG2 trangene, or a fragment thereof, into at least one HSC of the subject; increased expression of functional RAG1 protein or RAG2 protein in the subject; or a combination thereof. In some embodiments, the SCID is X-SCID, and wherein administration of the LNP results in insertion of an IL2RG transgene, or a fragment thereof, in at least one HSC of the subject; increased expression of functional IL2RG protein in the subject; or a combination thereof.

In some embodiments, the PID is Wiskott-Aldrich syndrome. In some embodiments, the PID is Wiskott-Aldrich syndrome, and wherein administration of the LNP results in insertion of a WAS transgene, or a fragment thereof, into at least one HSC of the subject; increased expression of functional WASP protein expression in the subject; or a combination thereof.

In some embodiments, the PID is chronic granulomatous disease.

In some embodiments, the PID is X-linked chronic granulomatous disease.

In some embodiments, the PID is chronic granulomatous disease, and wherein administration of the LNP results in one or more of (i) insertion of a CYBA transgene, a CYBB transgene, an NCF1 transgene, NCF2 transgene, or an NCF4 transgene, or a fragment thereof, into at least one HSC of the subject; (ii) introduction of a point 676C>T pointe mutation in the CYBB gene of at least one HSC in the subject; (iii) increased expression of functional CYBA protein, CYBB protein, NCF1 protein, NCF2 protein, or NCF4 protein in the subject; and (v) an increased amount of functional NADPH oxidase enzyme complex in the subject.

In some embodiments, the PID is IPEX. In some embodiments, the PID is IPEX, and wherein administration of the LNP results in insertion of an FOXP3 transgene, or a fragment thereof, into at least one HSC of the subject; increased expression of functional FOXP3 protein in the subject; or a combination thereof.

In some embodiments, the PID is hyper IgM syndrome. In some embodiments, the PID is hyper IgM syndrome, and wherein administration of the LNP results in one or more of (i) insertion of a AICDA transgene, a UNG transgene, an CD40 transgene, or a CD40LG transgene, or a fragment thereof, into at least one HSC of the subject; (ii) increased expression of functional AICDA protein, UNG protein, CD40 protein, or CD40LG protein in the subject; (iii) a decreased amount of IgM antibodies in the subject; and (iv) an increased amount of IgG, IgA, or IgE antibodies in the subject.

In some embodiments, the disease is a congenital cytopenia. In some embodiments, the congenital cytopenia is Fanconia anemia, Shwachman-Diamond syndrome, Blackfan-Diamond anemia, dyskeratosis congenita, congenital amegakaryocytic thrombocytopenia, or reticular dysgenesis. In some embodiments, the congenital cytopenia is Fanconia anemia, and wherein administration of the LNP results in insertion of one or more FANC genes, or a fragment thereof, into at least one HSC in the subject; increased expression of one or more functional FANC proteins in the subject; or a combination thereof. In some embodiments, the congenital cytopenia is Fanconia anemia, and wherein administration of the LNP insertion of a FANCA transgene, or a fragment thereof, into at least one HSC of the subject; increased expression of functional FANCA in the subject; or a combination thereof.

In some embodiments, the disease is a hemophilia. In some embodiments, the hemophilia is hemophilia A, hemophilia B, or hemophilia C.

In some embodiments, the disease is a hemophilia, and wherein administration of the LNP results in (i) insertion of a F8 transgene, a F9 transgene, or an F11, or a fragment thereof, into at least one HSC of the subject; (ii) increased expression of functional factor VIII protein, factor IX protein, or factor XI protein in the subject; and (iii) increased blood clotting in the subject.

In some embodiments, the disease is a thrombophilia. In some embodiments, the thrombophilia is amegakaryocytic thrombocytopenia or factor X deficiency.

In some embodiments, the disease is a thrombophilia, and wherein administration of the LNP results in one or more of (i) insertion of a F5 transgene, a F2 transgene, a transgene encoding antithrombin III, a transgene encoding protein C, or a transgene encoding protein S, or a fragment thereof, into at least one HSC of the subject; (ii) increased expression of functional factor V protein, factor II protein, antithrombin III protein, protein C, or protein S in the subject; and (iii) reduced blood clotting in the subject.

In some embodiments, the disease is an inborn error of metabolism. In some embodiments, the inborn error of metabolism is phenylketoneuria, medium-chain acyl-CoA dehydrogenase (MCAD) deficiency, a lysosomal storage disease, a glycogen storage disorder, a peroxisomal disorder, Fabry disease, Gaucher disease, Hurler syndrome, Hunter syndrome, Wolman disease, or pyruvate kinase deficiency. In some embodiments, the peroxisomal disorder is X-linked adrenoleukodystrophy. In some embodiments, the lysosomal storage disease is metachromatic leukodystrophy, mucopolysaccharidosis I, or mucopolysaccharidosis II.

In some embodiments, the disease is a neuropathy. In some embodiments, the neuropathy is Friedrich's ataxia.

In some embodiments, the disease is a viral disease. In some embodiments, the viral disease is HIV/AIDS. In some embodiments, the viral disease is HIV/AIDS, and wherein administration of the LNP prevents infection by HIV, progression of HIV/AIDS, or a combination thereof.

In some embodiments, the one or more nucleic acids of the method of treating disposed in the LNP comprise an mRNA encoding a site-directed nuclease. In some embodiments, the site-directed nuclease is a CRISPR-associated (Cas) nuclease, a zinc finger nuclease (ZFN), a transcription activator-like effector nuclease (TALEN), or a megaTAL. In some embodiments, the site-directed nuclease is a Cas nuclease, ZFN, TALEN, or megaTAL comprising an amino acid sequence that confers binding to a target nucleotide sequence.

In some embodiments, the one or more nucleic acids of the method of treating disposed in the LNP comprise (i) an mRNA encoding a CRISPR-associated (Cas) nuclease or a chemical base editor; and (ii) a guide RNA (gRNA) comprising a nucleotide sequence that confers binding to a target nucleotide sequence. In some embodiments, the one or more nucleic acids disposed in the LNP comprise (i) an mRNA encoding a prime editor; and (ii) a prime editing guide RNA (pegRNA) comprising a nucleotide sequence that confers binding to a target nucleotide sequence. In some embodiments, the Cas nuclease is a Type II or a Type V Cas enzyme, or a variant thereof. In some embodiments, the Cas nuclease is a Cas9 enzyme, a Cas12 enzyme, a CasX enzyme, or a Cas 14 enzyme, or a variant thereof. In some embodiments, the gRNA or pegRNA comprises a sequence having at least 80% identity to at least 15 consecutive nucleotides of the target nucleotide sequence.

In some embodiments, the one or more nucleic acids of the method of treating disposed in the LNP further comprise a donor template nucleic acid comprising a sequence having at least 80% identity to at least 15 consecutive nucleotides of the target nucleotide sequence.

In some embodiments, the target nucleotide sequence of the method of treating comprises at least 15 consecutive nucleotides and is located within a coding region of a gene, an intronic region associated with a gene, an exon region associated with a gene, a 5′ untranslated region associated with a gene, or a 3′ untranslated region associated with a gene, wherein the gene is selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FN, GBA, IDS, IDUA, TCIRG, AICDA, UNG, CD40, CD40LG, FOXP3, IL4, IL10, IL13, IL7R, PRF1, FANCA, FANCB, FANCC, FANCD1, BRACA2, FANCD2, MPL, CCR5, CXCR4, F5, F2, antithrombin III, and protein C.

In some embodiments, the target nucleotide sequence is within a regulatory region, optionally an enhancer region or a repressor region, of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, AML, CCR5, CXCR4, F5, F2, antithrombin III, and protein C.

In some embodiments, the target nucleotide sequence is within the BCL11A erythroid enhancer. In some embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the subject of the methods provided herein is a human.

### BRIEF DESCRIPTION OF THE DRAWINGS

The present application can be understood by reference to the following description taken in conjunction with the accompanying figures.

FIG. 1 depicts an exemplary therapeutic strategy for in vivo CRISPR editing of hematopoietic stem cells (HSCs) with targeted lipid nanoparticles (LNPs).

FIG. 2 depicts proton NMR spectrum of intermediate 13-11.

FIG. 3A depicts proton NMR spectrum of intermediate 13-11a; FIG. 3B depicts proton NMR spectrum of intermediate 13-11b; and FIG. 3C depicts LC-ELSD of intermediate 13-11b.

FIG. 4A depicts proton NMR spectrum of intermediate 13-10; FIG. 4B depicts LC-CAD chromatogram of intermediate 13-10.

FIG. 5A-1 depicts proton NMR spectrum for Lipid 1; FIG. 5A-2 depicts the LC-CAD chromatogram of Lipid 1.

FIG. 5B-1 depicts proton NMR spectrum of Lipid 3; FIG. 5B-2 depicts the LC-CAD chromatogram of Lipid 3.

FIG. 5C-1 depicts proton NMR spectrum of Lipid 4; FIG. 5C-2 depicts the LC-CAD chromatogram L of Lipid 4.

FIG. 5D-I depicts proton NMR spectrum of Lipid 5; FIG. 5D-2 depicts the LC-CAD chromatogram of Lipid 5.

FIG. 5E-1 depicts proton NMR spectrum of Lipid 6; FIG. 5E-2 depicts the LC-CAD chromatogram of Lipid 6.

FIG. 5F-1 depicts proton NMR spectrum of Lipid 7; FIG. 5F-2 depicts the LC-CAD chromatogram of Lipid 7.

FIG. 5G-1 depicts proton NMR spectrum of Lipid 2; FIG. 5G-2 depicts the LC-CAD chromatogram of Lipid 2;

FIG. 5H-1 depicts proton NMR spectrum of Lipid 8; FIG. 5H-2 depicts the LC-CAD chromatogram of Lipid 8.

FIG. 5I-1 depicts proton NMR spectrum of Lipid 9; FIG. 5I-2 depicts the LC-CAD chromatogram of Lipid 9.

FIG. 5J-1 depicts proton NMR spectrum of Lipid 10; FIG. 5J-2 depicts the LC-CAD chromatogram of Lipid 10.

FIG. 5K-1 depicts proton NMR spectrum of Lipid 11; FIG. 5K-2 depicts the LC-CAD chromatogram of Lipid 11.

FIG. 5L-1 depicts proton NMR spectrum of Lipid 12; FIG. 5L-2 depicts the LC-CAD chromatogram of Lipid 12.

FIG. 5M-1 depicts proton NMR spectrum of Lipid 13; FIG. 5M-2 depicts the LC-CAD chromatogram of Lipid 13.

FIG. 5N-1 depicts proton NMR spectrum of Lipid 15; FIG. 5N-2 depicts the LC-CAD chromatogram of Lipid 15.

FIG. 5O-1 depicts proton NMR spectrum of Lipid 16; FIG. 5O-2 depicts the LC-CAD of Lipid 16.

FIG. 5P-1 depicts proton NMR spectrum of Lipid 19; FIG. 5P-2 depicts the LC-CAD chromatogram of Lipid 19.

FIG. 5Q-1 depicts proton NMR spectrum of Lipid 20; FIG. 5Q-2 depicts the LC-CAD chromatogram of Lipid 20.

FIG. 5R-1 depicts proton NMR spectrum of Lipid 31; FIG. 5R-2 depicts the LC-CAD chromatogram of Lipid 31.

FIG. 5S-1 depicts proton NMR spectrum of Lipid 32; FIG. 5S-2 depicts the LC-CAD chromatogram of Lipid 32.

FIG. 5T-1 depicts proton NMR spectrum of Lipid 33; FIG. 5T-2 depicts the LC-CAD chromatogram of Lipid 33.

FIG. 5U-1 depicts proton NMR spectrum of Lipid 34; FIG. 5U-2 depicts the LC-CAD chromatogram of Lipid 34.

FIGS. 6A-6D depict the results of experiment to screen LNP-Fab and LNP-full length antibody conjugates for binding to HSCs. LNP-Fab and LNP-antibody conjugates were formulated with 1,1′-Dioctadecyl-3,3,3′,3′-Tetramethylindocarbocyanine Perchlorate (DiI) and used to treat HSCs in vitro. DiI fluorescence is shown as a measure of LNP targeting, since binding of the LNP-Fab conjugate to the HSCs results in the HSCs having DiI fluorescence. The specific Fab or antibody of each LNP-Fab or LNP-antibody conjugate is shown at the top of each panel. Each column shows DiI fluorescence of HSCs treated with the indicated LNP-Fab or LNP-antibody conjugate prepared at various Fab/antibody densities, as indicated in the detailed layout legend. Unconjugated LNPs and LNPs conjugated with the anti-CD8 mutOKT8 antibody were used as negative controls for HSC targeting, as CD8 is not expressed by HSCs. Hanks Balanced Salt Solution (HBS) was also used as a non-LNP buffer control. FIG. 6A depicts a first experiment to screen HSC targeting of a first set of LNP-Fab conjugates. FIG. 6B depicts a second experiment to screen HSC targeting of the first set of LNP-Fab conjugates. FIG. 6C depicts a first experiment to screen HSC targeting of a second set of LNP-antibody conjugates. FIG. 6D depicts a second experiment to screen HSC targeting of the second set of LNP-antibody conjugates.

FIGS. 7A-7D depict the results of experiment to screen LNP-Fab and LNP-full length antibody conjugates for transfection of HSCs. LNP-Fab and LNP-antibody conjugates were formulated with DiI and an mRNA encoding green fluorescent protein (GFP) and used to treat HSCs in vitro. A combination of DiI fluorescence and GFP fluorescence is shown as a measure of LNP transfection, since binding of the LNP-Fab or LNP-antibody conjugate to the HSCs results in the HSCs having DiI fluorescence, and since transcription of the mRNA into GFP requires the mRNA to enter the cell and escape the endosome. The specific Fab or antibody of each LNP-Fab or LNP-antibody conjugate is shown at the top of each panel. Each column shows double DiI and GFP fluorescence of HSCs treated with the indicated LNP-Fab conjugate or LNP-antibody conjugate prepared at various Fab/antibody densities, as indicated in the detailed layout legend. Unconjugated LNPs and LNPs conjugated with the anti-CD8 mutOKT8 antibody were used as negative controls for HSC targeting, as CD8 is not expressed by HSCs. Hanks Balanced Salt Solution (HBS) was also used as a non-LNP buffer control. FIG. 7A depicts a first experiment to screen HSC transfection of a first set of LNP-Fab conjugates. FIG. 7B depicts a second experiment to screen HSC transfection of the first set of LNP-Fab conjugates. FIG. 7C depicts a first experiment to screen HSC transfection of a second set of LNP-antibody conjugates. FIG. 7D depicts a second experiment to screen HSC transfection of the second set of LNP-antibody conjugates.

FIG. 8 depicts transfection of HSCs by LNPs conjugated with the anti-CD117 Ab1 Fab. DiI fluorescence indicates binding of the (Ab1)-LNP conjugate to the HSCs, and GFP fluorescence indicates successful transfection of the HSCs with GFP mRNA.

FIGS. 9A-9B depict in vitro editing of HSCs by treatment with KC3 LNPs conjugated with the anti-CD117 Ab1 Fab. KC3 LNP formulations conjugated with or without Ab1 were used to encapsulate N1-methyl-pseudo-uridine chemically modified Cas9 mRNA with a gRNA specific for CD45. Primary human HSCs were treated with these LNPs at a dose range of 100 to 800 ng of total RNA for 7 days, after which the HSCs were stained with fluorescent antibodies against CD34 and CD117 to define HSC cell populations and a fluorescent antibody against CD45 to detect CD45 knockout. FIG. 9A depicts CD34 and CD45 fluorescence of HSCs treated with the Ab1-conjugated KC3 LNPs and illustrates a reduction in HSCs expressing CD45 with increasing amounts RNA loading. FIG. 9B depicts the percentage of CD45 knockout in HSCs treated with both Ab1-conjugated (KC3-Ab1) and -unconjugated (KC3) LNPs.

FIGS. 10A-10B depict in vitro editing of HSCs by treatment with Lipid 15 LNPs conjugated with the anti-CD117 Ab1 Fab. Lipid 15 LNP formulations conjugated with or without Ab1 were used to encapsulate N1-methyl-pseudo-uridine chemically modified Cas9 mRNA with a gRNA specific for CD45. Primary human HSCs were treated with these LNPs at a dose range of 100 to 800 ng of total RNA for 7 days, after which the HSCs were stained with fluorescent antibodies against CD34 and CD117 to define HSC cell populations and a fluorescent antibody against CD45 to detect CD45 knockout. FIG. 10A depicts CD34 and CD45 fluorescence of HSCs treated with the Ab1-conjugated Lipid 15 LNPs and illustrates a reduction in HSCs expressing CD45 with increasing amounts RNA loading. FIG. 10B depicts the percentage of CD45 knockout in HSCs treated with both Ab1-conjugated (Ab1 Lipid 15) and unconjugated (Lipid 15) LNPs.

FIG. 11 depicts a plot of percent knockout of CD45 in LNP-treated HSCs as measured by both phenotype (CD45 expression) and genotype (indels detected via sequencing analysis). Each individual point represents a single technical replicate (i.e. a unique well of HSCs). Phenotype KO % is the frequency of cells absent of CD45 protein. Genotype KO % is the percentage of reads that contain out of frame indels at the target cut site.

FIG. 12 depicts structures of various Fab, VHH (Nb), ScFv, Fab-ScFv and Fab-VHH hybrids.

FIG. 13 depicts in vitro transfection of HSCs with LNPs conjugated with Ab1 and encapsulating mCherry mRNA at varying Fab densities.

FIGS. 14A-14C depict in vitro gene editing of HSCs using LNPs conjugated with Ab1 and carrying RNAs for gene editing machinery. Primary HSCs were cultured in vitro and treated with LNP encapsulating CRISPR mRNA and beta-2-microglobulin (B2M) guides. The gene editing effect were measured by flow cytometry targeting the cell surface marker B2M. FIG. 14A shows exemplary flow cytometry results of LNPs conjugated with Ab1 (binding Ab), recognizing long-term hematopoietic stem cells (LT-HSC). FIG. 14B shows exemplary flow cytometry results of LNP conjugated with mutated Ab1 (mutAb1, non-binding Ab^{m}). FIG. 14C shows the average percentage of cells that exhibited a knockout of B2M protein expression in samples treated with LNPs conjugated with Ab1 and non-binding mutAb1.

FIGS. 15A-15F depict in vivo transfection of human long-term HSCs (LT-HSCs) engrafted into NSG™ mice. FIG. 15A depicts exemplary flow cytometry results illustrating the flow cytometry gating strategy to identify LT-HSCs using the CD34 and CD117 markers (CD34 is a ubiquitous HSC marker and CD117 is a marker of long-term HSCs). The transfection efficiency was evaluated by measuring the percentage of mCherry positive cells. Cells were initially selected using antibodies against human and mouse CD45⁺ cells, in order to differentiate the human HSCs from the mixture with mouse HSCs. FIG. 15B depicts an additional exemplary flow cytometry result showing mCherry expression in cell selected using the gating strategy illustrated in FIG. 15A. FIG. 15C shows the average percentage of LT-HSCs mCherry positive from mice treated with Lipid 15 LNPs coated with the Ab1 Fab, non-targeting mutAb1 Fab, or uncoated (naked) LNPs encapsulating mCherry mRNA. FIG. 15D shows the average percentage of LT-HSCs mCherry positive from mice treated with KC3 LNPs coated with the Ab1 Fab or the non-targeting mutAb1 Fab encapsulating mCherry mRNA. FIG. 15E shows the amount of mCherry protein normalized by total protein detected in liver tissues from mice treated with Lipid 15 LNPs coated with the Ab1 Fab, the non-targeting mutAb1 Fab, or uncoated (naked) LNPs encapsulating mCherry mRNA. FIG. 15F shows the amount of mCherry protein normalized by total protein detected in liver tissues from mice treated with KC3 LNPs coated with the Ab1 Fab, the non-targeting mutAb1 Fab, or uncoated (naked) LNPs encapsulating mCherry mRNA. In FIGS. 15B-E, the sample size was 2-9 mice for each treatment condition. Bar charts show data as mean values±SD: **** indicates P<0.0001; * indicates P<0.05; “ns” indicates not significant.

FIG. 16 depicts in vivo transfection of human LT-HSCs engrafted into NSG™ mice, showing the average percentage of LT-HSCs that are mCherry positive from mice treated with Lipid 15 LNPs coated with the Ab1 Fab encapsulating mCherry mRNA, and from untreated mice.

FIG. 17 depicts in vitro transfection of cynomolgus macaque HSCs with LNPs coated with the Ab1 Fab. LT-HSCs were isolated from cynomolgus macaque bone marrow and identified using the CD34 and CD117 markers. The average percentage of LT-HSCs that were mCherry positive is shown for samples treated with Lipid 15 LNPs that were coated with Ab1 Fab, coated with the non-targeting mutAb1 Fab, or were uncoated.

FIG. 18 depicts exemplary flow cytometry results illustrating the flow cytometry gating strategy to identify cynomolgus macaque LT-HSCs using the cynomolgus macaque CD34 and CD117 markers (CD34 is a ubiquitous HSC marker and CD117 is a marker of long-term HSCs). The transfection efficiency was evaluated by measuring the percentage of mCherry positive cells.

FIGS. 19A-19B depict in vivo transfection of LT-HSCs in cynomolgus macaques. Cynomolgus macaques were first treated intramuscularly with diphenhydramine at a dose of 2 mg/kg, and 30 minutes later were treated intravenously with HSC-targeting LNPs at various doses as shown on the x-axis. Whole bone marrow samples were collected 24 h after treatment by aspiration from the iliac crest and analyzed by flow cytometry using mCherry reporter. FIG. 19A shows the average percentage of LT-HSCs that are mCherry positive from cynomolgus macaques treated with Lipid 15 LNPs coated with the Ab1 Fab encapsulating mCherry mRNA. FIG. 19B shows the average percentage of LT-HSCs mCherry positive from cynomolgus macaques treated with KC3 LNPs coated with the Ab1 Fab encapsulating mCherry mRNA. The LNPs were administered intravenously at various doses, as shown on the horizontal axes. Each data point marked represents a single cynomolgus macaque, with one or two cynomolgus macaques tested per LNP and dose combination.

### DETAILED DESCRIPTION

The invention provides lipid nanoparticles (LNP) that specifically target hematopoietic stem cells (HSC) and deliver nucleic acids encapsulated in the LNP into HSC. In some aspects, the LNP for targeted delivery comprises (a) a lipid-antibody-conjugate comprising the compound of formula (I): [Lipid]-[optional linker]-[antibody](I); and (b) an ionizable cationic lipid; and (c) one or more nucleic acids disposed in the LNP. In some embodiments, the antibody specifically binds to CD105 and/or CD117. In some embodiments, the one or more nucleic acids comprise an mRNA encoding a nuclease and an associated guide RNA. Also provided herein are compositions comprising the LNP and methods of making and using the LNP for gene editing in HSC.

The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein should be construed according to the standard rules of chemical valency known in the chemical arts. In addition, when a chemical group is a diradical, for example, it is understood a that the chemical groups can be bonded to their adjacent atoms in the remainder of the structure in one or both orientations, for example, —OC(O)— is interchangeable with —C(O)O— or —OC(S)— is interchangeable with —C(S)O—.

The terms “a” and “an” as used herein mean “one or more” and include the plural unless the context is inappropriate. In some embodiments, “one or more” is 1 or 2. In some embodiments, “one or more” is 1, 2, or 3. In some embodiments, “one or more” is 1, 2, 3, or 4. In some embodiments, “one or more” is 1, 2, 3, 4, or 5. In some embodiments, “one or more” is 1, 2, 3, 4, 5, or more.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, or C₁-C₆alkyl, respectively. In some embodiments, alkyl is optionally substituted. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. In some embodiments, alkylene is optionally substituted. An exemplary alkylene group is —CH₂CH₂—.

The term “haloalkyl” refers to an alkyl group that is substituted with at least one halogen. For example, —CH₂F, —CHF₂, —CF₃, —CH₂CF₃, —CF₂CF₃, and the like.

The term “oxo” is art-recognized and refers to a “═O” substituent. For example, a cyclopentane substituted with an oxo group is cyclopentanone.

The term “morpholinyl” refers to a substituent having the structure of:

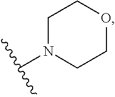 [SMILES: *N1CCOCC1]

which is optionally substituted.

The term “piperidinyl” refers to a substituent having a structure of

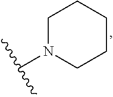 [SMILES: *N1CCCCC1]

which is optionally substituted.

In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds. In some embodiments, “optionally substituted” is equivalent to “unsubstituted or substituted.” In some embodiments, “optionally substituted” indicates that the designated atom or group is optionally substituted with one or more substituents independently selected from optional substituents provided herein. In some embodiments, optional substituent may be selected from the group consisting of: C₁₋₆alkyl, cyano, halogen, —O—C₁₋₆alkyl, C₁₋₆haloalkyl, C₃₋₇cycloalkyl, 3- to 7-membered heterocyclyl, 5- to 6-membered heteroaryl, and phenyl. In some embodiments, optional substituent is alkyl, cyano, halogen, halo, azide, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl, or heteroaryl. In some embodiments, optional substituent is —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R^{s11}, —NR^{s12}C(O)R^{s13}, or —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

The term “haloalkyl” refers to an alkyl group that is substituted with at least one halogen. For example, —CH₂F, —CHF₂, —CF₃, —CH₂CF₃, —CF₂CF₃, and the like.

The term “cycloalkyl” refers to a monovalent saturated cyclic, bicyclic, bridged cyclic (e.g., adamantyl), or spirocyclic hydrocarbon group of 3-12, 3-10, 3-8, 4-8, or 4-6 carbons, referred to herein, e.g., as “C₄₋₈cycloalkyl,” derived from a cycloalkane. In some embodiments, cycloalkyl is optionally substituted. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. In some embodiments, heterocyclyl is optionally substituted. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C3heterocyclyl is aziridinyl. Heterocycles may be, for example, mono-, bi-, or other multi-cyclic ring systems (e.g., fused, spiro, bridged bicyclic). A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl, pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “aryl” is art-recognized and refers to a carbocyclic aromatic group. In some embodiments, aryl is optionally substituted. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6- to 10-membered ring structure. In some embodiments, the aryl group is a C₆-C₁₄ aryl.

The term “heteroaryl” is art-recognized and refers to aromatic groups that include at least one ring heteroatom. In some embodiments, heteroaryl is optionally substituted. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The terms “amine” and “amino” are art-recognized and refer to both unsubstituted and substituted amines, e.g., a moiety represented by the general formula —N(R¹⁰)(R¹¹), wherein R¹⁰ and R¹¹ each independently represent hydrogen, alkyl, cycloalkyl, heterocyclyl, alkenyl, aryl, aralkyl, or (CH₂)ₘ—R¹²; or R¹⁰ and R¹¹, taken together with the N atom to which they are attached complete a heterocycle having from 4 to 8 atoms in the ring structure; R¹² represents an aryl, a cycloalkyl, a cycloalkenyl, a heterocycle or a polycycle; and m is zero or an integer in the range of 1 to 8. In certain embodiments, R¹⁰ and R¹¹ each independently represent hydrogen, alkyl, alkenyl, or —(CH₂)ₘ—R¹².

The terms “alkoxyl” or “alkoxy” are art-recognized and refer to an alkyl group, as defined above, having an oxygen radical attached thereto. In some embodiments, alkoxyl is optionally substituted. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, O-alkynyl, —O—(CH₂)ₘ—R¹², where m and R¹² are described above. The term “haloalkoxyl” refers to an alkoxyl group that is substituted with at least one halogen. For example, —O—CH₂F, —O—CHF₂, —O—CF₃, and the like. In certain embodiments, the haloalkoxyl is an alkoxyl group that is substituted with at least one fluoro group. In certain embodiments, the haloalkoxyl is an alkoxyl group that is substituted with from 1-6, 1-5, 1-4, 2-4, or 3 fluoro groups.

The symbol “ [symbol] ” indicates a point of attachment.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol “ [symbol] ” denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “K” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labeled disclosed compounds (e.g., those labeled with 3H and 14C) are useful in compound and/or substrate tissue distribution assays. Tritiated (i.e., 3H) and carbon-14 (i.e., 14C) isotopes are particularly preferred for their ease of preparation and detectability. Further, substitution with heavier isotopes such as deuterium (i.e., 2H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable excipient” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Remington's The Science and Practice of Pharmacy, 21st Edition, A. R. Gennaro; Lippincott, Williams & Wilkins, Baltimore, MD, 2006.

As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

Abbreviations as used herein include diisopropylethylamine (DIPEA); 4-dimethylaminopyridine (DMAP); tetrabutylammonium iodide (TBAf); 1-ethyl-3-(3-dimethylaminopropyl)carbodiimide (EDC); benzotriazol-1-yl-oxytripyrrolidinophosphonium hexafluorophosphate (PyBOP), 9-Fluorenylmethoxycarbonyl (Fmoc), tetrabutyldimethylsilyl chloride (TBDMSCl), hydrogen fluoride (HF), phenyl (Ph), bis(trimethylsilyl)amine (HMDS), dimethylformamide (DMF); methylene chloride (DCM); tetrahydrofuran (THF); high-performance liquid chromatography (HPLC); mass spectrometry (MS), evaporative light scattering detector (ELSD), electrospray (ES)); nuclear magnetic resonance spectroscopy (NMR).

As used herein, the term “effective amount” refers to the amount of a compound (e.g., a nucleic acid, e.g., an mRNA) sufficient to effect beneficial or desired results. An effective amount can be administered in one or more administrations, applications or dosages and is not intended to be limited to a particular formulation or administration route. The term effective amount can be considered to include therapeutically and/or prophylactically effective amounts of a compound.

The phrase “therapeutically effective amount” as used herein means that amount of a compound (e.g., a nucleic acid, e.g., an mRNA), material, or composition comprising a compound (e.g., a nucleic acid, e.g., an mRNA) which is effective for producing some desired therapeutic effect in at least a sub-population of cells in a mammal, for example, a human, or a subject (e.g., a human subject) at a reasonable benefit/risk ratio applicable to any medical treatment.

The phrase “prophylactically effective amount” as used herein means that amount of a compound (e.g., a nucleic acid, e.g., an mRNA), material, or composition comprising a compound (e.g., a nucleic acid, e.g., an mRNA) which is effective for producing some desired prophylactic effect in at least a sub-population of cells in a mammal, for example, a human, or a subject (e.g., a human subject) by reducing, minimizing or eliminating the risk of developing a condition or the reducing or minimizing severity of a condition at a reasonable benefit/risk ratio applicable to any medical treatment.

As used herein, the terms “treat,” “treating,” and “treatment” include any effect, e.g., lessening, reducing, modulating, ameliorating or eliminating, that results in the improvement of the condition, disease, disorder, and the like, or ameliorating a symptom thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, unless otherwise indicated, the term “antibody” means any antigen-binding molecule or molecular complex comprising at least one complementarity determining region (CDR) that specifically binds to or interacts with a particular antigen. It is understood the term encompasses an intact antibody (e.g., an intact monoclonal antibody), or a fragment thereof, such as an Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment thereof, or Fc fragment that has been modified or engineered. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). The term also encompasses an immunoglobulin single variable domain, such as a Nanobody (e.g., a V_{HH}).

Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}), wherein the V_{H} domain is at the amino-terminus of the polypeptide and the CM domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196: 901-17; Chothia et al., 1989, Nature 342. 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. Identification of predicted CDRs using the amino acid sequence is well known in the field, such as in Martin, A. C. “Protein sequence and structure analysis of antibody variable domains,” In Anibody Engineering, Vol. 2. Kontermann R., Dübel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the V_{H} and C_{H1} domains of one heavy chain, wherein the V_{H}-C_{H1} heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a C_{H1} domain.

A F(ab′) fragment typically includes one light chain and a portion of one heavy chain that contains more of the constant region (between the C_{H1} and C_{H2} domains), such that an interchain disulfide bond can be formed between two heavy chains to form a F(ab′)₂ molecule.

As used here, an “antibody that binds to X” (i.e., X being a particular antigen), or “an anti-X antibody”, is an antibody that specifically recognizes the antigen X.

As used herein, a “buried interchain disulfide bond” or an “interchain buried disulfide bond” refers to a disulfide bond on a polypeptide which is not readily accessible to water soluble reducing agents, or is effectively “buried” in the hydrophobic regions of the polypeptide, such that it is unavailable to both reducing agents and for conjugation to other hydrophilic PEGs. Buried interchain disulfide bonds are further described in WO2017096361A1, which is incorporated by reference in its entirety.

As used herein, specificity of the targeted delivery by an LNP is defined by the ratio between % of hematopoietic stem cells (HSCs) that receive the delivered nucleic acid (e.g., on-target delivery), and % of an undesired or untargeted cell type that is not meant to be the destination of the delivery, but receives the delivered nucleic acid (e.g., off-target delivery). For example, the specificity is higher when more HSCs receive the delivered nucleic acid, and/or when fewer cells of other types receive the delivered nucleic acid. Specificity of the targeted delivery by an LNP can also be defined by the ratio of the amount of nucleic acid being delivered to the HSCs (e.g., on-target delivery) and the amount of nucleic acid being delivered to cells of other types (e.g., off-target delivery). Specificity of the delivery can be determined using any suitable method. As a non-limiting example, expression level of the nucleic acid in HSCs can be measured and compared to that of another cell type that is not meant to be the destination of the delivery.

As used herein, a humanized antibody is an antibody which is wholly or partially of non-human origin and whose protein sequence has been modified to replace certain amino acids, for instance that occur at the corresponding position(s) in the framework regions of the VH and VL domains in a sequence of antibody from a human being, to increase its similarity to antibodies produced naturally in humans, in order to avoid or minimize an immune response in humans. For example, using techniques of genetic engineering, the variable domains of a non-human antibodies of interest may be combined with the constant domains of human antibodies. The constant domains of a humanized antibody are most of the time human CH and CL domains.

As used herein, the term “spacer” or “linker” denotes a peptide that fuses together two or more polypeptides or proteins into a single molecule. The use of spacers to connect two or more (poly)peptides is well known in the art. Further exemplary peptidic spacers are shown in Table C. One often used class of peptidic spacer are known as the “Gly-Ser” or “GS” spacers. These are spacers that essentially consist of glycine (G) and serine (S) residues, and usually comprise one or more repeats of a peptide motif such as the GGGGS (SEQ ID NO:45) motif (for example, having the formula (Gly-Gly-Gly-Gly-Ser)n in which n may be 1, 2, 3, 4, 5, 6, 7 or more). Some often-used examples of such GS spacers are 9GS spacers (GGGGSGGGS, SEQ ID NO: 48), 15GS spacers (n=3) and 35GS spacers (n=7). Reference is for example made to Chen et al. 2013 (Adv. Drug Deliv. Rev. 65(10): 1357-1369) and Klein et al. 2014 (Protein Eng. Des. Sel. 27 (10): 325-330).

As used herein, the term “structural lipid” refers to sterols and also to lipids containing sterol moieties.

It should be understood that the order of steps or order for performing certain actions is immaterial so long as the present invention remain operable. Moreover, two or more steps or actions may be conducted simultaneously.

At various places in the present specification, substituents are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of other examples, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Lipid Nanoparticle Compositions

The invention provides a lipid nanoparticle (LNP) composition comprising an ionizable cationic lipid described herein and/or a lipid-HSC targeting group conjugate (e.g., lipid-antibody conjugate) described herein. In certain embodiments, the LNP may comprise an ionizable, cationic lipid described herein and one or more of a sterol, a neutral phospholipid, a PEG-lipid, and a lipid-immune cell targeting group conjugate. In some embodiments, the LNP comprises a lipid blend comprising the ionizable cationic lipid and one or more of a sterol, a neutral phospholipid, a PEG-lipid, and a lipid-HSC targeting group conjugate (e.g., lipid-antibody conjugate).

### (a) Ionizable Cationic Lipids

In some embodiments, the lipid nanoparticle compositions provided herein comprise an ionizable cationic lipid. When used in a lipid nanoparticle composition, such ionizable cationic lipids may facilitate the delivery of a payload (e.g., a nucleic acid, such as a DNA or RNA, such as an mRNA) disposed therein to cells, e.g., mammalian cells, e.g., hematopoietic stem cells (HSCs). Such ionizable cationic lipids have been designed to enable intracellular delivery of a nucleic acid, e.g., mRNA, to the cytosolic compartment of a target cell type and rapidly degrade into non-toxic components. The complex functionalities of the ionizable cationic lipids are facilitated by the interplay between the chemistry and geometry of the ionizable lipid head group, the hydrophobic “acyl-tail” groups and the linkers connecting the head group and the acyl tail groups. Typically, the pKₐ of the ionizable amine head group is designed to be in the range of 6-8, such as between 6.2-7.4, or between 6.7-7.2, such that it remains strongly cationic under acidic formulation conditions (e.g., pH 4-pH 5.5), neutral or slightly anionic in physiological pH (7.4) and cationic in the early and late endosomal compartments (e.g., pH 5.5-pH 7). The acyl-tail groups play a key role in fusion of the lipid nanoparticle with endosomal membranes and membrane destabilization through structural perturbation. The three-dimensional structure of the acyl-tail (determined by its length, and degree and site of unsaturation) along with the relative sizes of the head group and tail group are thought to play a role in promoting membrane fusion, and hence lipid nanoparticle endosomal escape (a key requirement for cytosolic delivery of a nucleic acid payload). The linker connecting the head group and acyl tail groups is designed to degrade by physiologically prevalent enzymes (e.g., esterases, or proteases) or by acid catalyzed hydrolysis.

In some embodiments, the lipid nanoparticle compositions provided herein comprise an ionizable cationic lipid represented by Formula (II′):

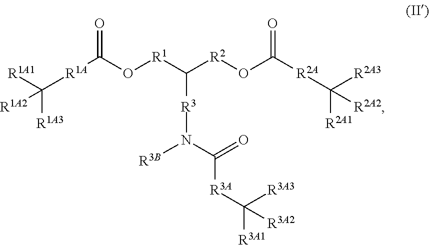

or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is

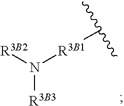

R^{3B1} is C₁₋₆ alkylene; and R^{3B2} and R^{3B3} are each independently H or C₁₋₆ alkyl optionally substituted with one or more substituents each independently selected from the group consisting of —OH and —O—(C₁₋₆ alkyl).

Any of the variables or substituents provided herein is unsubstituted or substituted with one or more substituents. In some embodiments, any of the variables or substituents provided herein is optionally substituted. In some embodiments, any of the variables or substituents provided herein is optionally substituted with one or more substituents independently selected from the group consisting of —OR^{s1}, —NR^{s2}R^{s3}, —C(O)R^{s4}, —C(O)OR^{s5}—, C(O)NR^{s6}R^{s7}, —OC(O)R^{s8}, —OC(O)OR^{s9}, —OC(O)NR^{s10}R^{s11}, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted.

In some embodiments, R¹, R², and R³ are each independently a bond or C₁₋₃ alkylene. In some embodiments, R¹, R², and R³ are each independently a bond or methylene. In some embodiments, R¹ and R² are each methylene and R³ is a bond. In some embodiments, R¹, R², and R³ are each methylene. In some embodiments, R¹, R², and R³ are each independently unsubstituted or substituted.

In some embodiments, R^{1A}, R^{2A}, and R^{3A} are each independently a bond or C₁₋₁₀ alkylene. In some embodiments, R^{1A}, R^{2A}, and R^{1A} are each independently a bond or —(CH₂)₁₋₁₀—. In some embodiments, R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—. In some embodiments, R^{1A} and R^{2A} are each a bond, each —CH₂—, each —(CH₂)₂—, each —(CH₂)₃—, each —(CH₂)₄—, each —(CH₂)₅—, each —(CH₂)₆—, each —(CH₂)₇—, or each —(CH₂)₈—. In some embodiments, R^{1A} and R^{2A} are each independently a bond, —(CH₂)₂—, —(CH₂)₄—, —(CH₂)₆—, —(CH₂)₇—, or —(CH₂)₈—. In some embodiments, R^{1A} and R^{2A} are each a bond, each —(CH₂)₂—, each —(CH₂)₄—, each —(CH₂)₆—, each —(CH₂)₇—, or each —(CH₂)₈—. In some embodiments, R^{3A} is a bond, —CH₂—, —(CH₂)₂—, or —(CH₂)₇—. In some embodiments, R^{1A}, R^{2A}, and R^{1A} are each independently unsubstituted or substituted.

In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2}. In some embodiments, R^{1A1}, R^{1A2}, R^{1A3}, R^{2A1}, R^{2A2}, R^{2A3}, R^{3A1}, R^{3A2}, and R^{3A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl). In some embodiments, R^{1A1}, R^{1A2}, R^{1A3}, R^{2A1}, R^{2A2}, R^{2A3}, R^{3A1}, R^{3A2}, and R^{3A3} are each independently unsubstituted or substituted.

In some embodiments, R^{1A1} and R^{2A1} are each independently —CH═CH—(C₁₁₋₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl), and R^{1A2}, R^{1A3}, R^{2A2}, and R^{2A3} are each H. In some embodiments, R^{1A1} and R^{2A1} are each

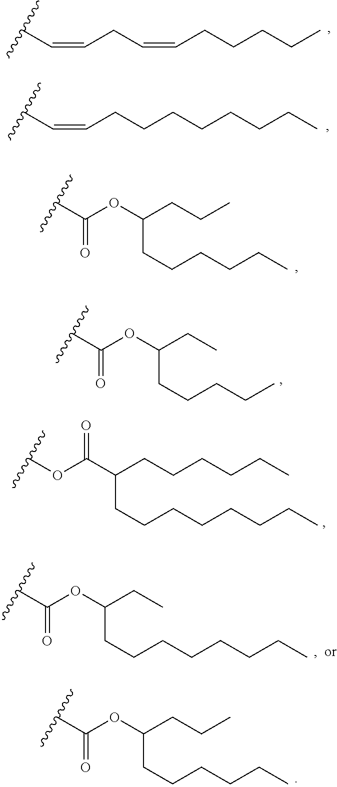

In some embodiments, R^{1A1} and R^{2A1} are each C₁₋₁₅ alkyl; R^{1A2} and R^{2A2} are each C₁₋₁₅ alkyl; and R^{1A3} and R^{2A3} are each H. In some embodiments, R^{1A1} and R^{2A1} are each

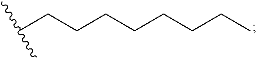 [SMILES: *CCCCCCCC]

and R^{1A2} and R^{2A2} are each

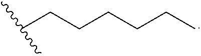 [SMILES: *CCCCCC]

In some embodiments, R^{1A1} and R^{2A1} are each —(CH₂)₀₋₄OC(O)CH₂(C₁₋₁₅ alkyl); R^{2A1} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

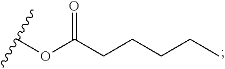 [SMILES: *OC(=O)CCCCC]

and R^{2A1} and R^{2A2} are each

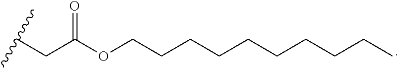 [SMILES: *CC(=O)OCCCCCCCCCC]

In some embodiments, R^{1A1} and R^{2A1} are each —C(O)OCH₂(C₁₋₁₅ alkyl); R^{1A1} and R^{2A2} are each —(CH₂)₀₋₄C(O)OCH₂(C₁₋₁₅ alkyl); and R^{1A3} and R^{2A3} are each H. In some embodiments, R^{1A1} and R^{2A1} are each

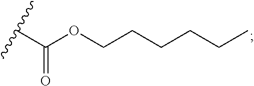 [SMILES: *C(=O)OCCCCCC]

and R^{1A2} and R^{2A2} are each

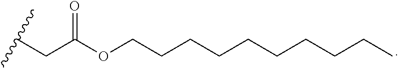 [SMILES: *CC(=O)OCCCCCCCCCC]

In some embodiments, R^{3A1}, R^{3A2}, and R^{3A3} are each independently H, C₁₋₁₅ alkyl, —(CH₂)₀₋₄C(O)OCH(C₁₋₆ alkyl)(C₁₋₁₀ alkyl), —(CH₂)₀₋₄OC(O)CH(C₁₋₁₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

In some embodiments, R^{3A1} and R^{3A2} are each independently C₁₋₁₅ alkyl; and R^{3A3} is H. In some embodiments, R^{3A1} and R^{3A2} are each independently ethyl,

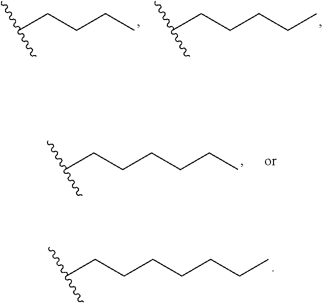

In some embodiments R^{3A1} is C₁₋₁₅ alkyl, and R^{3A2} and R^{3A3} are each H. In some embodiments, R^{3A1} is

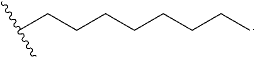 [SMILES: *CCCCCCCC]

In some embodiments, R^{3A1} is —C(O)OCH(C₁₋₅ alkyl)(C₁₋₁₀ alkyl); and R^{3A2} and R^{3A3} are each H. In some embodiments, R^{3A1} is

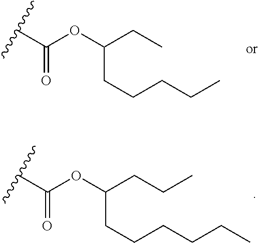 [SMILES: *C(=O)OC(CC)CCCCC ; *C(=O)OC(CCC)CCCCCC]

In some embodiments, R^{3A1} is —(CH₂)₀₋₄OC(O)CH₂(C₁₋₁₀ alkyl); R^{3A2} is —(CH₂)₀₋₄(O)OCH₂(C₁₋₁₀ alkyl); and R^{3A3} is H. In some embodiments, R^{3A1} is

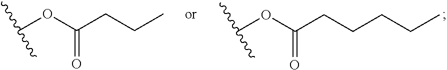

### and R^{3A2} is

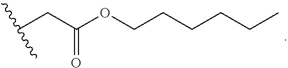 [SMILES: *CC(=O)OCCCCCC]

In some embodiments, R^{3A1} is —(CH₂)₀₋₄C(O)OCH₂(C₁₋₁₀ alkyl); R^{3A2} is —(CH₂)₀₋₄C(O)OCH₂(C₁₋₁₀ alkyl); and R^{3A3} is H. In some embodiments, R^{3A1} is

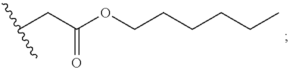 [SMILES: *CC(=O)OCCCCCC]

### and R^{3A2} is

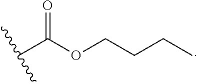 [SMILES: *C(=O)OCCCC]

In some embodiments, R^{3A1}, R^{3A2}, and R^{3A3} are each H.

R^{a1} and R^{a2} are each independently C₁₋₂₀ alkyl or C₁₋₂₀ alkenyl. In some embodiments, R^{a1} and R^{a2} are each independently —(CH₂)₀₋₁₅CH₃ or —CH(C₁₋₁₀ alkyl)(C₁₋₁₅ alkyl). In some embodiments, R^{a1} and R^{a2} are each independently

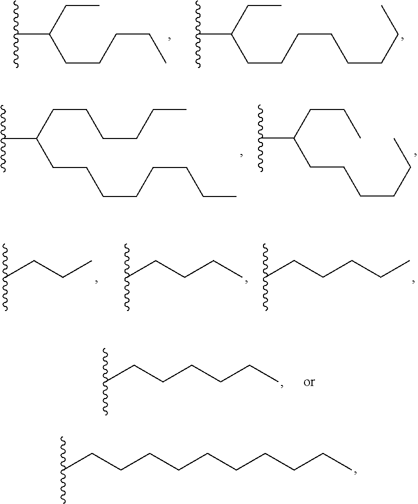

each of which is optionally substituted. In some embodiments, R^{a1} and R^{a2} are each independently unsubstituted or substituted.

In some embodiments, R^{3B} is

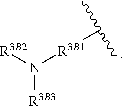

In some embodiments, R^{3B} is H. In some embodiments, R^{3B} is unsubstituted or substituted.

In some embodiments, R^{3B1} is C₁₋₆ alkylene. In some embodiments, R^{3B1} is ethylene or propylene. In some embodiments, R^{3B1} is unsubstituted or substituted. In some embodiments, R^{3B1} is optionally substituted.

In some embodiments, R^{3B2} and Rau are each independently and optionally substituted. In some embodiments, R^{3B2} and R^{3B3} are each independently H or C₁₋₆ alkyl optionally substituted with one or more substituents each independently selected from the group consisting of —OH and —O—(C₁₋₆ alkyl). In some embodiments, R^{3B2} and R^{3B3} are each independently H or C₁₋₆ alkyl optionally substituted with one or more substituents independently selected from the group consisting of —OR^{s1}, —NR^{s2}R^{s3}, —C(O)R^{s4}, —C(O)OR^{s5}, C(O)NR^{s6}R^{s7}, —OC(O)R^{s8}, —OC(O)OR^{s9}, —OC(O)NR^{s10}R^{s11}, —NR^{s12}C(O)R^{s13}, and —NR^{s14}C(O)OR^{s15}, wherein R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, and R^{s15} are each independently H, C₁₋₆ alkyl, C₃₋₁₀ cycloalkyl, C₆₋₁₄ aryl, 5- to 10-membered heteroaryl, or 3- to 10-membered heterocyclyl, each of which is optionally substituted. In some embodiments, R^{3B2} and R^{3B3} are each independently H, methyl, ethyl, propyl, butyl, or pentyl, each of which is optionally substituted with one or more substituents each independently selected from the group consisting of —OH and —O—(C₁₋₆ alkyl). In some embodiments, R^{3B2} and R^{3B3} are each independently methyl or ethyl, each optionally substituted with one or more —OH. In some embodiments, R^{3B2} and R^{3B3} are each methyl or each ethyl, each optionally substituted with one or more —OH. In some embodiments, R^{3B2} and R^{3B3} are each unsubstituted methyl.

In some embodiments,

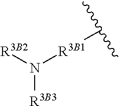

is

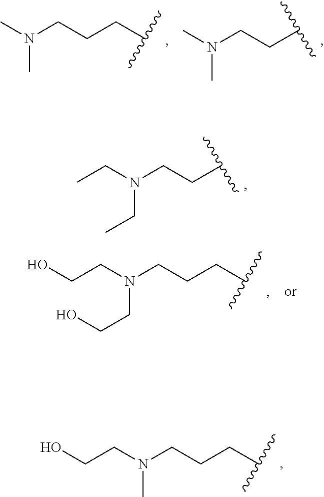

each of which is optionally substituted.

In some embodiments, the lipid nanoparticle compositions provided herein comprise an ionizable cationic lipid represented by Formula (IIa):

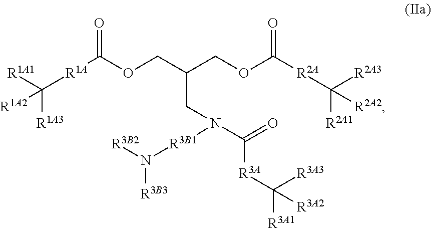

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{3B1}, R^{3B2}, and R^{3B3} are as defined for Formula (II′), Formula (II), or any variation or embodiment thereof.

In some embodiments, the lipid nanoparticle compositions provided herein comprise an ionizable cationic lipid represented by Formula (IIb);

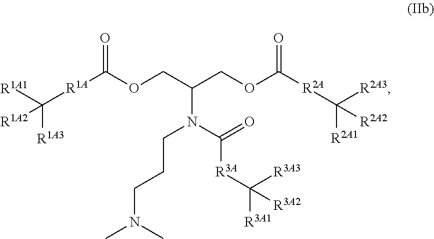

or a salt thereof, wherein R^{1A}, R^{2A}, R^{3A}, R^{1A1}, R^{1A2}, R^{1A3}, R^{2A1}, R^{2A2}, R^{2A3}, R^{3A1}, R^{3A2}, and R^{3A3}, are as defined for Formula (II′), Formula (II), or any variation or embodiment thereof.

In some embodiments, the lipid nanoparticle compositions provided herein comprise an ionizable cationic lipid of formula:

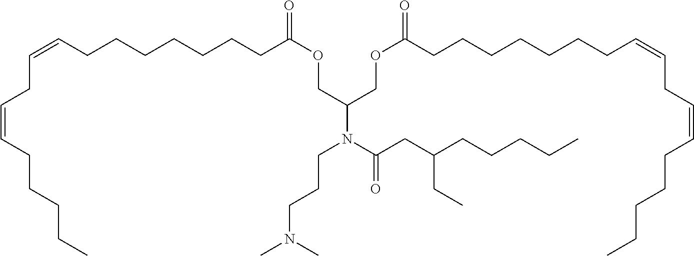 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

or a salt thereof.

In some embodiments, the lipid nanoparticle compositions provided herein comprise an ionizable cationic lipid of formula:

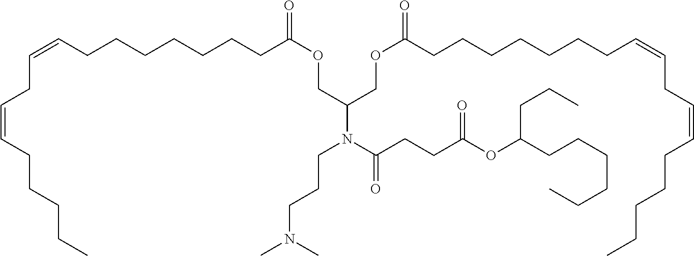 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

or a salt thereof.

In some embodiments, the lipid nanoparticle compositions provided herein comprise an ionizable cationic lipid of formula:

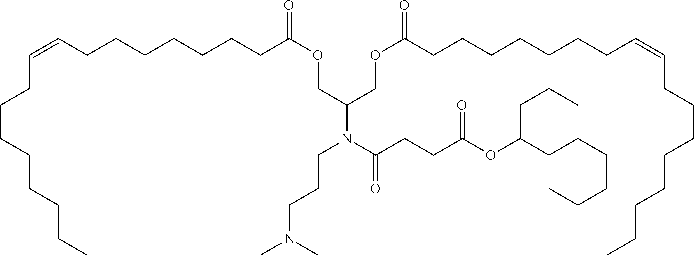 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

or a salt thereof.

In some embodiments, the lipid nanoparticle compositions provided herein comprise an ionizable cationic lipid represented by Formula IIIa or Formula IIIb:

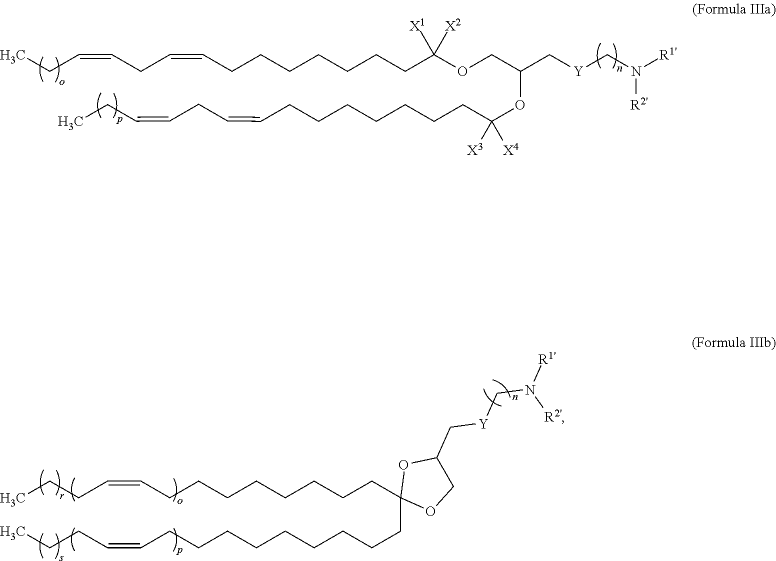

or a salt thereof, wherein:
   R^{1′} and R^{2′} are independently C₁₋₃alkyl, or R^{1′} and R^{2′} are taken together with the nitrogen atom to form an optionally substituted piperidinyl or morpholinyl;
   Y is selected from the group consisting of —O—, —OC(O)—, —OC(S)—, and —CH₂—;
   X¹, X², X³, and X⁴ are hydrogen or X¹ and X² or X³ and X⁴ independently are taken together to form an oxo;
   n is 0 or 3;
   o and p are independently an integer selected from 2-6.

In some embodiments, the lipid nanoparticle compositions provided herein comprise an ionizable cationic lipid represented by Formula IIIa. In some embodiments, the lipid nanoparticle compositions provided herein comprise an ionizable cationic lipid represented by Formula IIIb.

In some embodiments, the compound of formula IIIa is not a compound selected from the group consisting of

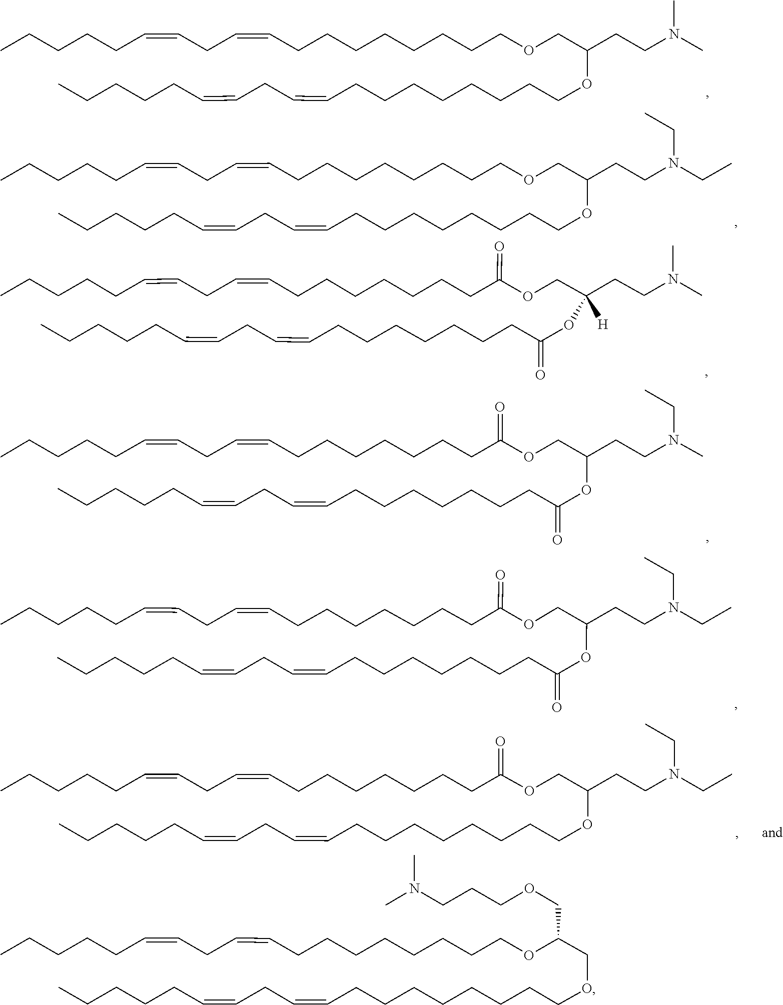

or a salt thereof.

In certain embodiments, o and p may be 2. In certain embodiments, o and p may be 3. In other embodiments, o and p may be 4. In some embodiments, o and p may be 5. In other embodiments, o and p may be 6.

In certain embodiments, X and X² may be taken together to form an oxo and X³ and X⁴ are taken together to form an oxo. In other embodiments, X¹, X², X³, and X⁴ may be hydrogen.

In certain embodiments, Y may be selected from the group consisting of —O—, —OC(O)—, OC(S)— and —CH₂—. For example, in certain embodiments, Y may be —O—. In certain embodiments, Y may be —OC(O)—. In certain embodiments, Y may be —CH₂—. In certain embodiments, Y may be —OC(S)—.

In certain embodiments, R^{1′} and R^{2′} may be independently C₁₋₃alkyl. In other embodiments, R^{1′} and R^{2′} may be —CH₃. In certain embodiments, R^{1′} and R^{2′} are —CH₂CH₃. In certain embodiments, R^{1′} and R^{2′} are C₃ alkyl.

In certain embodiments, n may be 0. In other embodiments, n may be 3.

Also provided herein, in part, is a compound represented by Formula IV:

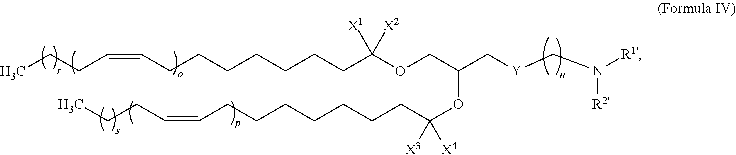

or a salt thereof, wherein:
   R^{1′} and R^{2′} are independently C₁₋₃alkyl, or R^{1′} and R^{2′} are taken together with the nitrogen atom to form an optionally substituted piperidinyl or morpholinyl;
   Y is selected from the group consisting of —O—, —OC(O)—, —OC(S)—, and —CH₂—;
   X¹, X², X³, and X⁴ are hydrogen or X¹ and X² or X³ and X⁴ are taken together to form an oxo;
   n is 0-4;
   o is 1 and r is an integer selected from 3-8 or o is 2 and r is an integer selected from 1-8,
   p is 1 and s is an integer selected from 3-8 or p is 2 and s is an integer selected from 1-8,
   wherein,
   when o and p are both 1, r and s are independently 4, 5, 7, or 8, and
   when o and p are both 2, r and s are independently 1, 2, 4, or 5.

In certain embodiments, X¹ and X² may be taken together to form an oxo and X³ and X⁴ may be taken together to form an oxo. In other embodiments, X¹, X², X³, and X⁴ may be hydrogen.

In certain embodiments, Y may be selected from the group consisting of —O—, —OC(O)—, and —CH₂—. For example, in certain embodiments, Y may be —O—. In certain embodiments, Y may be —OC(O)—. In certain embodiments, Y may be —CH₂—. In certain embodiments, Y may be —OC(S)—.

In certain embodiments, R^{1′} and R^{2′} may be independently C₁₋₃alkyl. In other embodiments, R^{1′} and R^{2′} may be —CH₃. In certain embodiments, R^{1′} and R^{2′} may be —CH₂CH₃. In some embodiments, R^{1′} and R^{2′} may be C₃ alkyl. In certain embodiments, R^{1′} and R^{2′} are taken together with the nitrogen atom to form an optionally substituted piperidinyl.

In certain embodiments, n may be 0. In other embodiments, n may be 3.

In some embodiments, the lipid nanoparticle compositions provided herein comprise an ionizable cationic lipid selected from the group consisting of:

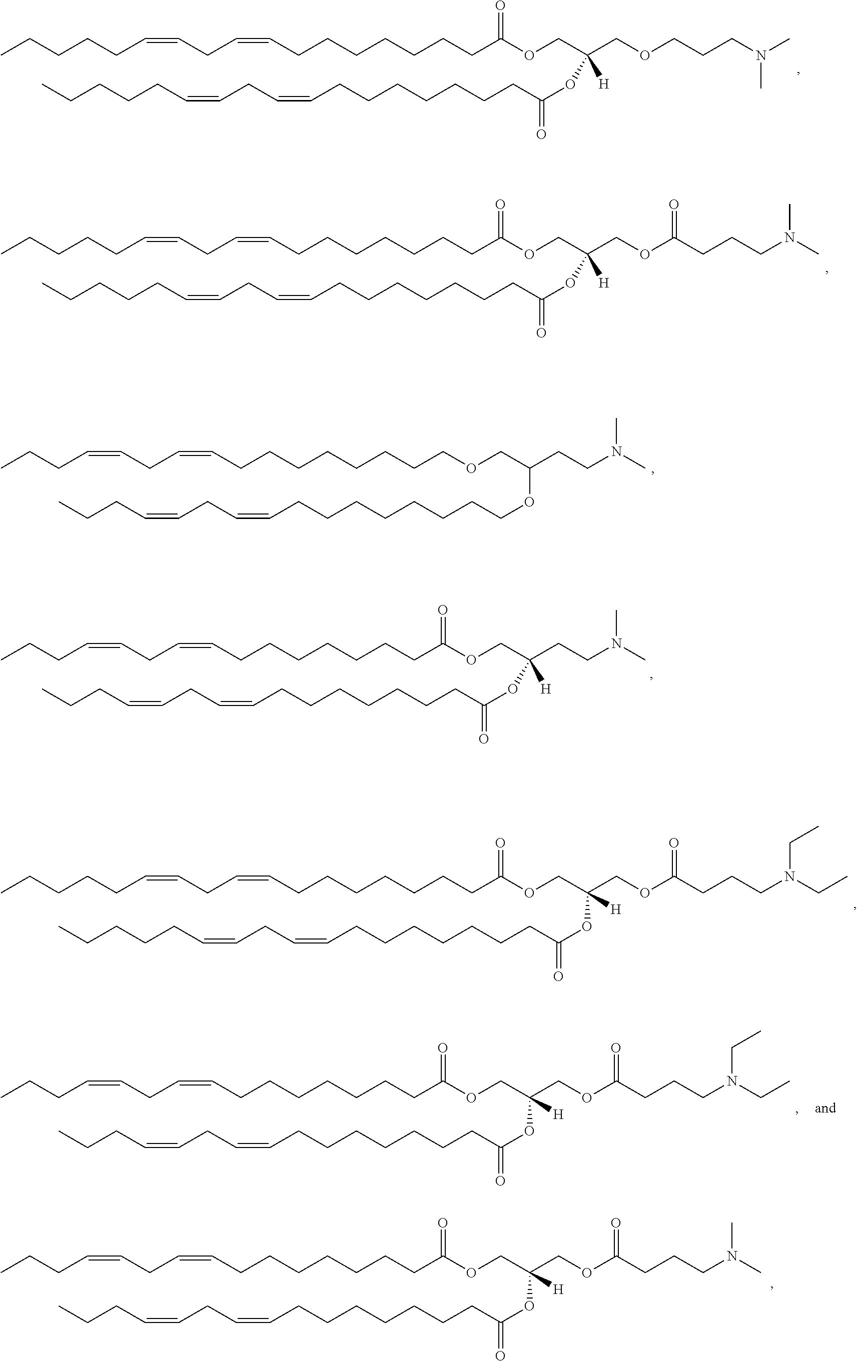

or a salt thereof.

In some embodiments, the lipid nanoparticle compositions provided herein comprise an ionizable cationic lipid of formula:

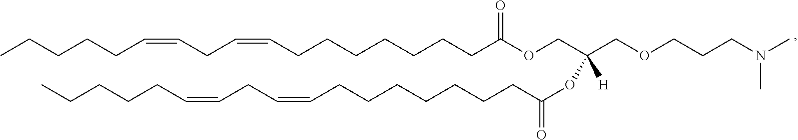 [SMILES: CCCCC/C=C\C/C=C\CCCCCCCC(=O)OC[C@H](COCCCN(C)C)OC(=O)CCCCCCC/C=C\C/C=C\CCCCC]

or a salt thereof.

In some embodiments, the lipid nanoparticle compositions provided herein comprise an ionizable cationic lipid of formula:

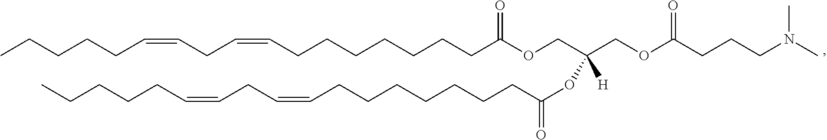 [SMILES: CCCCC/C=C\C/C=C\CCCCCCCC(=O)OC[C@H](COC(=O)CCCN(C)C)OC(=O)CCCCCCC/C=C\C/C=C\CCCCC]

or a salt thereof.

In some embodiments, the lipid nanoparticle compositions provided herein comprise an ionizable cationic lipid of formula:

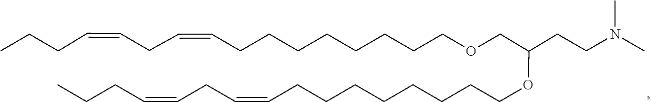 [SMILES: CCC/C=C\C/C=C\CCCCCCCCOCC(CCN(C)C)OCCCCCCCC/C=C\C/C=C\CCC]

or a salt thereof.

In some embodiments, the lipid nanoparticle compositions provided herein comprise an ionizable cationic lipid of formula:

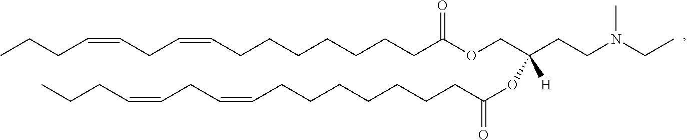 [SMILES: CCC/C=C\C/C=C\CCCCCCCC(=O)OC[C@H](CCN(C)CC)OC(=O)CCCCCCC/C=C\C/C=C\CCC]

or a salt thereof.

In some embodiments, the lipid nanoparticle compositions provided herein comprise an ionizable cationic lipid of formula:

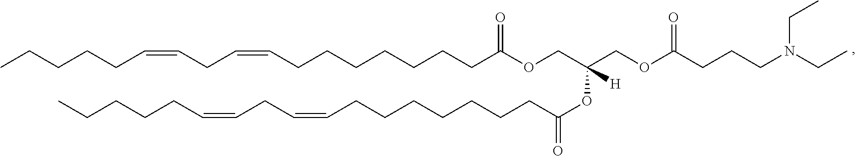 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

or a salt thereof.

In some embodiments, the lipid nanoparticle compositions provided herein comprise an ionizable cationic lipid of formula:

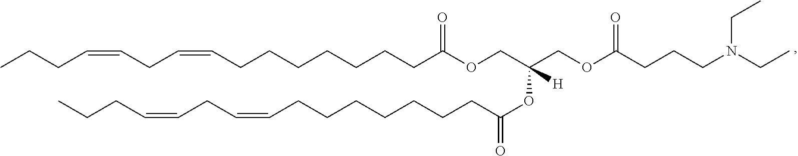 [SMILES: CCC/C=C\C/C=C\CCCCCCCC(=O)OC[C@H](COC(=O)CCCN(CC)CC)OC(=O)CCCCCCC/C=C\C/C=C\CCC]

or a salt thereof.

In some embodiments, the lipid nanoparticle compositions provided herein comprise an ionizable cationic lipid of formula:

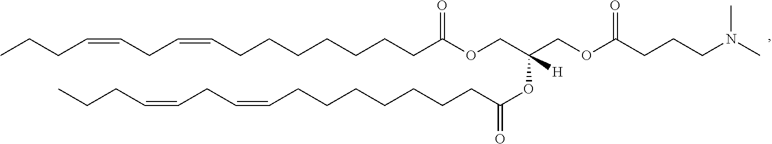 [SMILES: CCC/C=C\C/C=C\CCCCCCCC(=O)OC[C@H](COC(=O)CCCN(C)C)OC(=O)CCCCCCC/C=C\C/C=C\CCC]

or a salt thereof.

In some embodiments, the lipid nanoparticle compositions provided herein comprise an ionizable cationic lipid of Formula V:

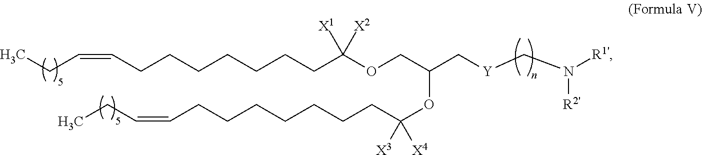

or a salt thereof, wherein:
   R^{1′} and R^{2′} are independently C₁₋₃alkyl, or R^{1′} and R^{2′} are taken together with the nitrogen atom to form an optionally substituted piperidinyl or morpholinyl;
   Y is selected from the group consisting of —O—, —OC(O)—, —OC(S)—, and —CH₂—;
   X¹, X², X³, and X⁴ are hydrogen or X¹ and X² or X³ and X⁴ are taken together to form an oxo; and
   n is an integer selected from 0-4.

In certain embodiments, X¹ and X² may be taken together to form an oxo and X³ and X⁴ may be taken together to form an oxo. In other embodiments, X¹, X², X³, and X⁴ may be hydrogen.

In certain embodiments, Y may be selected from the group consisting of —O—, —OC(O)—, and —CH₂—. For example, in certain embodiments, Y may be —O—. In certain embodiments, Y may be —OC(O)—. In certain embodiments, Y may be —CH₂—. In certain embodiments, Y may be —OC(S)—.

In certain embodiments, R^{1′} and R^{2′} may be independently C₁₋₃alkyl. In other embodiments, R^{1′} and R^{2′} may be —CH₃. In certain embodiments, R^{1′} and R^{2′} may be —CH₂CH₃. In some embodiments, R^{1′} and R^{2′} may be C₃ alkyl. In certain embodiments, R^{1′} and R^{2′} are taken together with the nitrogen atom to form an optionally substituted piperidinyl.

In certain embodiments, n may be 0. In other embodiments, n may be 3.

In some embodiments, the lipid nanoparticle compositions provided herein comprise an ionizable cationic lipid of the formula:

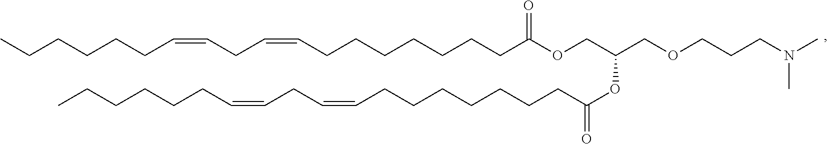 [SMILES: CCCCCC/C=C\C/C=C\CCCCCCCC(=O)OC[C@H](COCCCN(C)C)OC(=O)CCCCCCC/C=C\C/C=C\CCCCCC]

or a salt thereof.

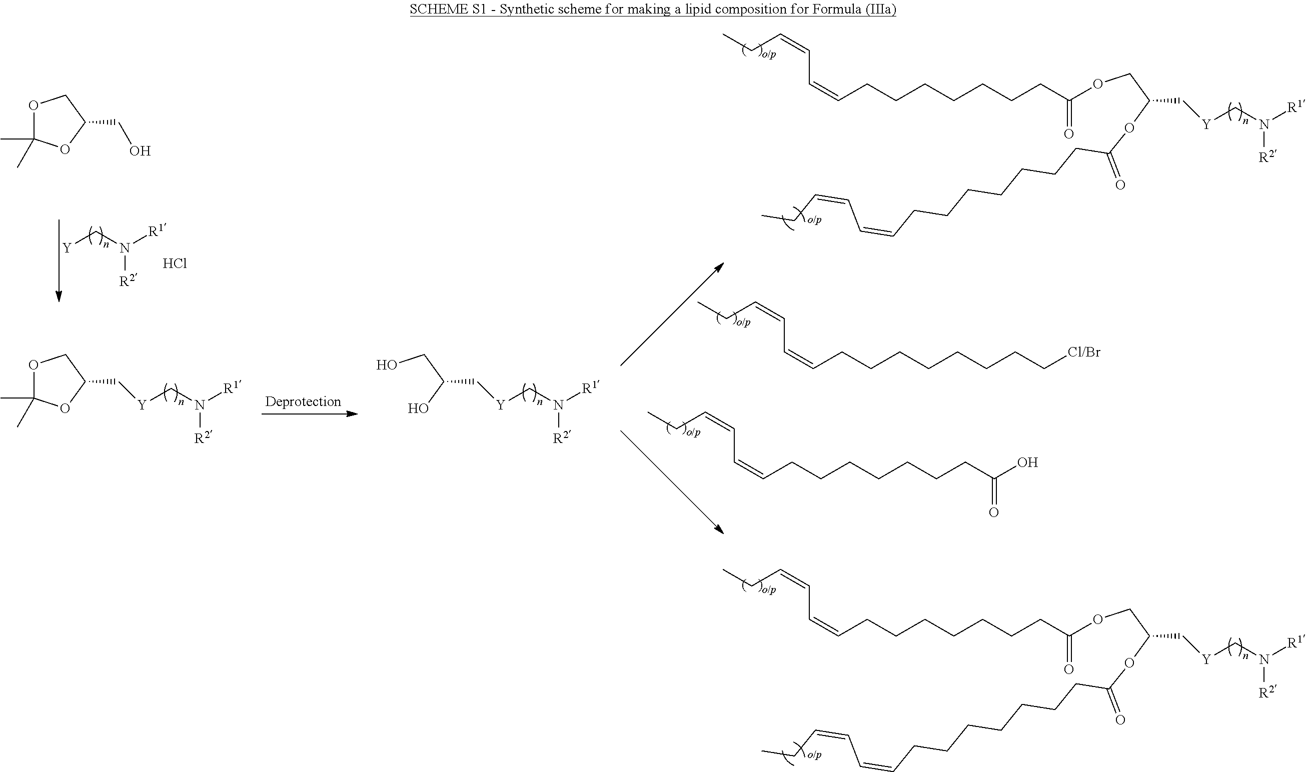

A compound of Formula IIIa may be prepared, e.g., according to Scheme S1. A hydroxy-functional protected propane diol is converted to the corresponding dimethyl amino-function ether (Y=oxo) or ester (Y═O—C(O)). The ether bond formation results from a reaction of the alkyl halide with alcohol in the presence of tertiary butylammonium iodide/NaOH in THF at 80° C. The ester bond formation utilizes treatment of an acid functional dimethylamine with alcohol under carbodiimide activation (DCM, EDC, DIEPA, DMAP). The diol deprotection yields a vicinal diol intermediate that is subsequently converted to the corresponding ether linked or ester linked diacyl lipids by treatment with TBAI/NaOH and bromo-acyl or by carbodiimide mediated carboxylic acid activation for ester bond formation, respectively.

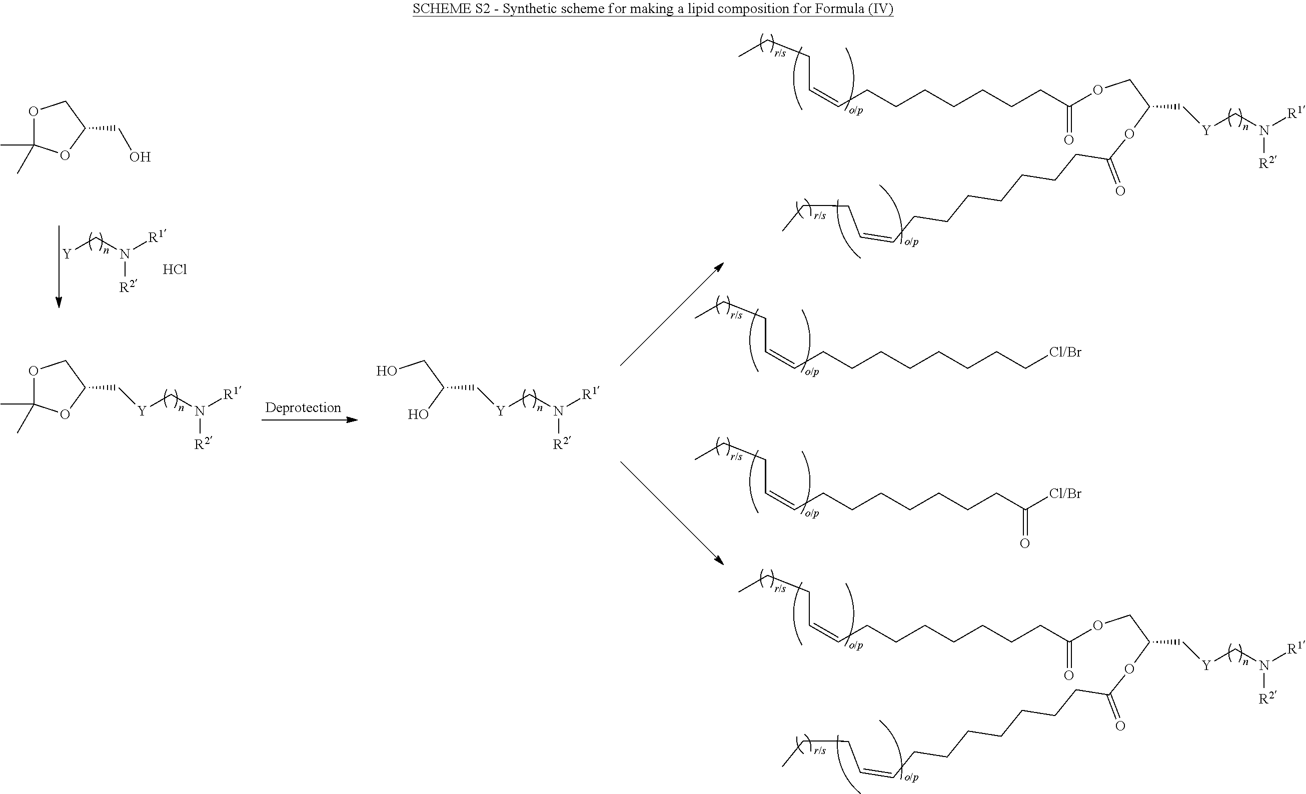

A compound of Formula IV may be prepared, e.g., according to Scheme S2. The synthetic procedure is as outlined above for Scheme S1; however, in Scheme S2, either bis-unsaturated acyl groups or mono-unsaturated acyl groups may be employed to obtain a lipid of Formula IV.

In some embodiments, ionizable cationic lipid used in the LNPs of the present disclosure is selected from the lipids in Table A, or a combination thereof. In some embodiments, the ionizable cationic lipid is:

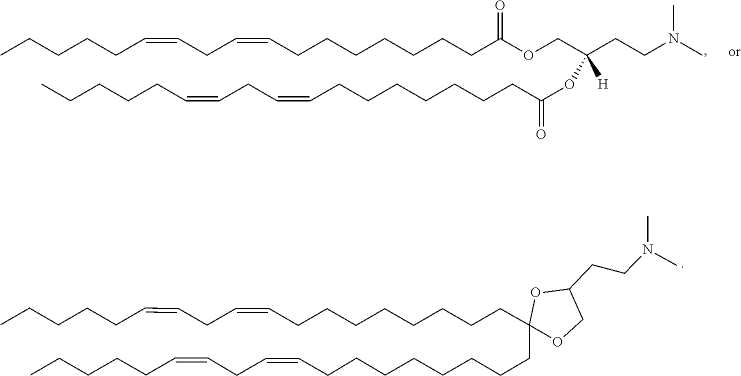

In some embodiments, the ionizable cationic lipid used in the LNPs of the present disclosure is a compound of Formula (KC3):

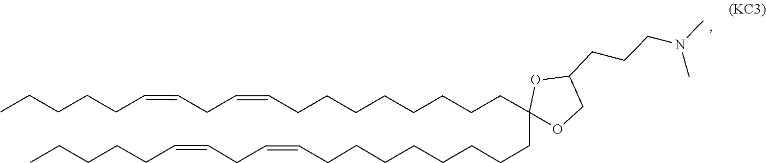 [SMILES: CCCCC/C=C\C/C=C\CCCCCCCCC1(CCCCCCCC/C=C\C/C=C\CCCCC)OCC(CCCN(C)C)O1]

or a salt thereof. References to “KC3” or “lipid KC3” herein refer to a compound of Formula (KC3), or a salt thereof. References to “KC3 LNPs” herein refer to lipid nanoparticles comprising a compound of Formula (KC3), or a salt thereof.

TABLE A
Exemplary ionizable cationic lipids.
(Lipid 1)

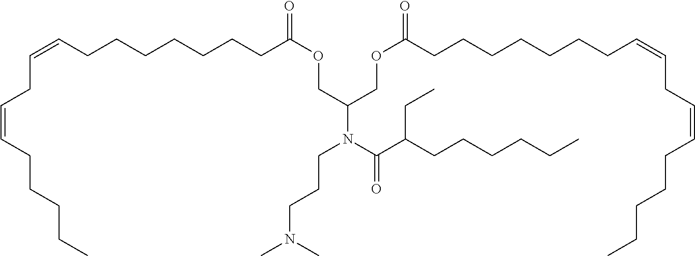 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

Lipid 1
(Lipid 2)

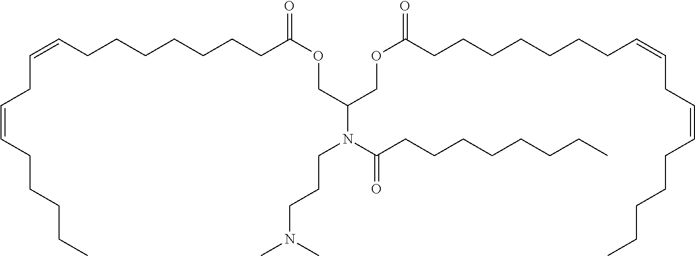 [SMILES: CCCCC/C=C\C/C=C\CCCCCCCC(=O)OCC(COC(=O)CCCCCCC/C=C\C/C=C\CCCCC)N(CCCN(C)C)C(=O)CCCCCCCC]

Lipid 2
(Lipid 3)

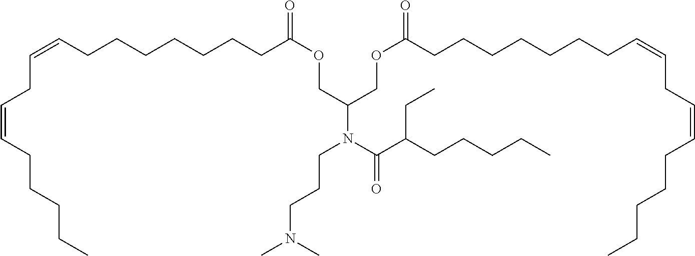 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

Lipid 3
(Lipid 4)

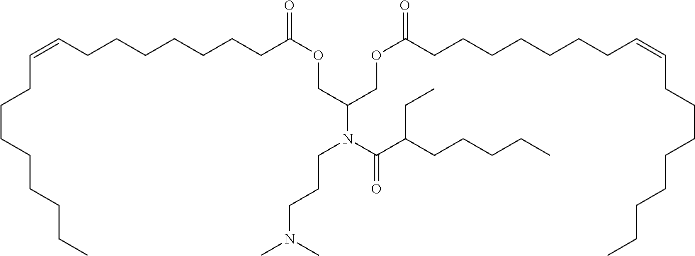 [SMILES: CCCCCCCC/C=C\CCCCCCCC(=O)OCC(COC(=O)CCCCCCC/C=C\CCCCCCCC)N(CCCN(C)C)C(=O)C(CC)CCCCC]

Lipid 4
(Lipid 5)

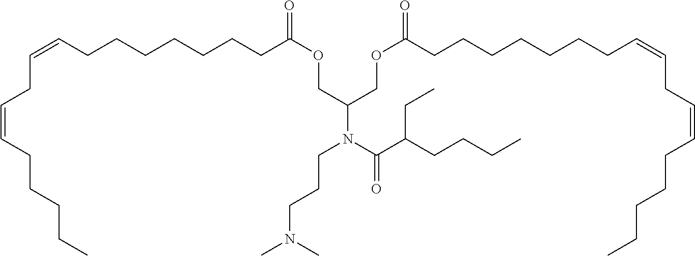 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCC]

Lipid 5
(Lipid 6)

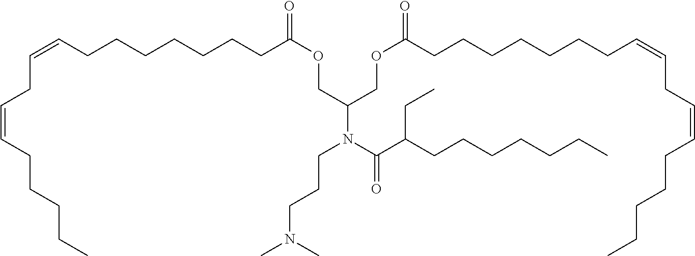 [SMILES: CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

Lipid 6
(Lipid 7)

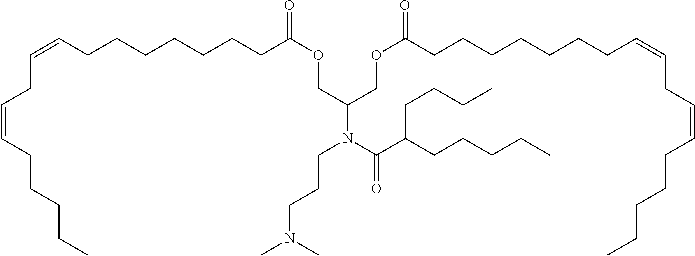 [SMILES: CCCCC/C=C\C/C=C\CCCCCCCC(=O)OCC(COC(=O)CCCCCCC/C=C\C/C=C\CCCCC)N(CCCN(C)C)C(=O)C(CCCC)CCCCC]

Lipid 7
(Lipid 8)

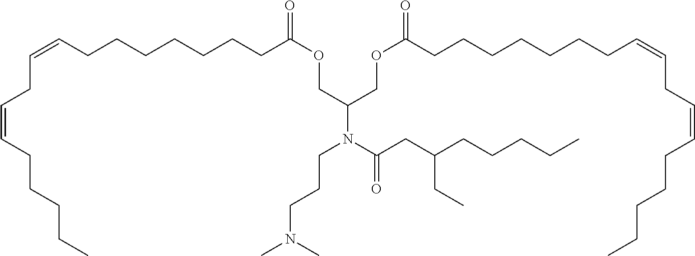 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

Lipid 8
(Lipid 9)

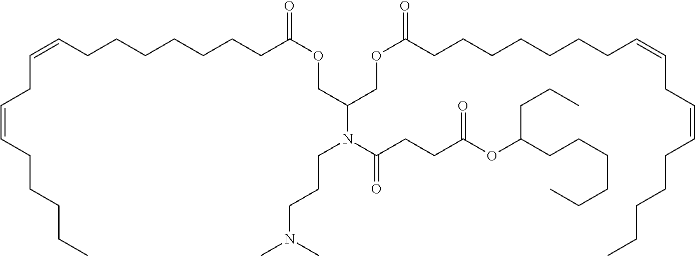 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

Lipid 9
(Lipid 10)

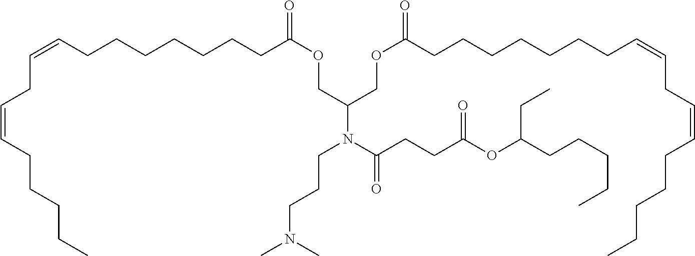 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CC)CCCCC]

Lipid 10
(Lipid 11)

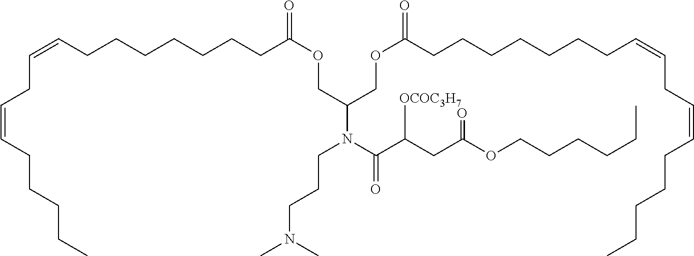

Lipid 11
(Lipid 12)

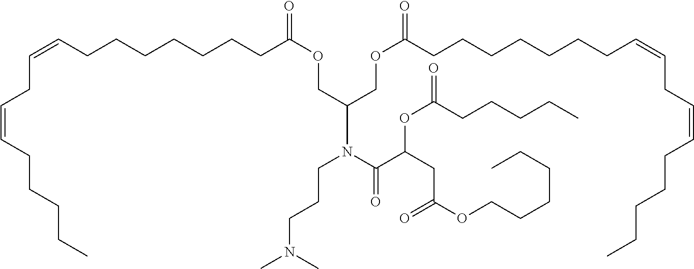 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 12
(Lipid 13)

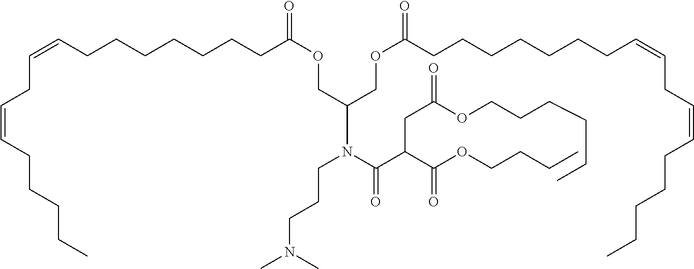 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)C(=O)OCCCC]

Lipid 13
(Lipid 14)

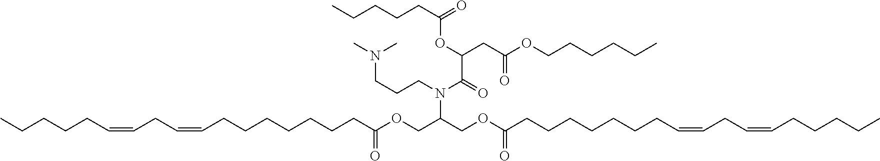 [SMILES: CCCCC/C=C\C/C=C\CCCCCCCC(=O)OCC(COC(=O)CCCCCCC/C=C\C/C=C\CCCCC)N(CCCN(C)C)C(=O)C(CC(=O)OCCCCCC)OC(=O)CCCCC]

Lipid 14
(Lipid 15)

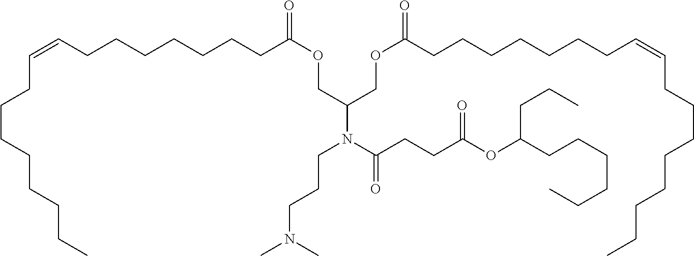 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

Lipid 15
(Lipid 16)

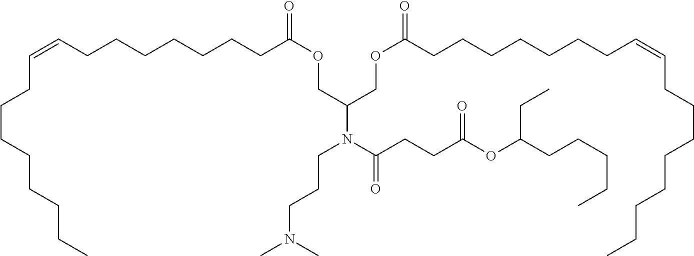 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CC)CCCCC]

Lipid 16
(Lipid 17)

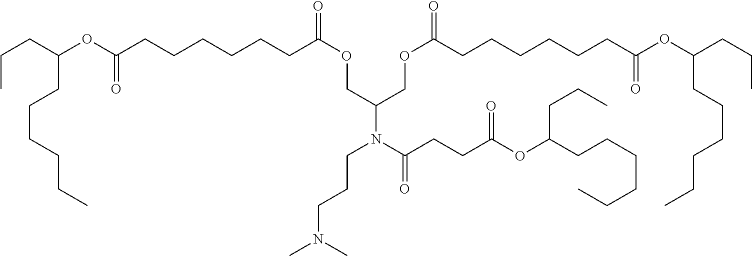 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 17
(Lipid 18)

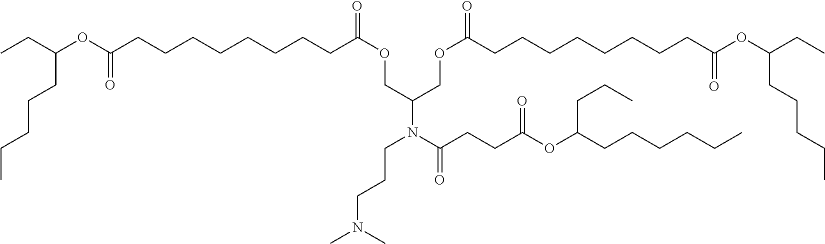 [SMILES: CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

Lipid 18
(Lipid 19)

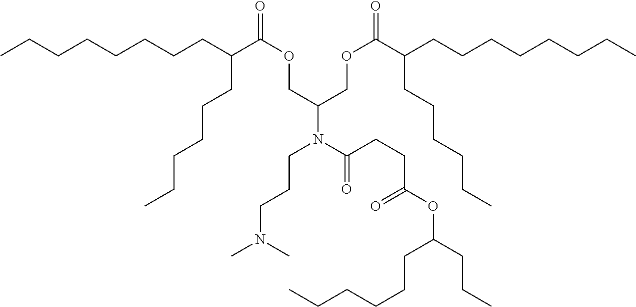 [SMILES: CCCCCCCCC(CCCCCC)C(=O)OCC(COC(=O)C(CCCCCC)CCCCCCCC)N(CCCN(C)C)C(=O)CCC(=O)OC(CCC)CCCCCC]

Lipid 19
(Lipid 20)

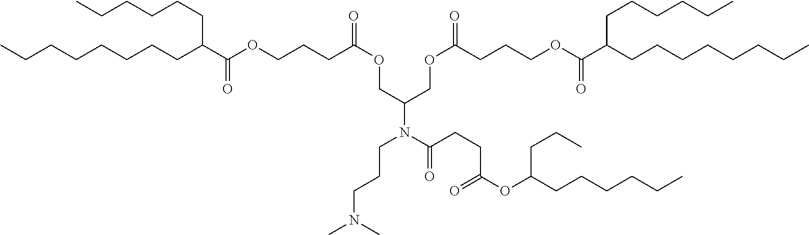 [SMILES: CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

Lipid 20
(Lipid 21)

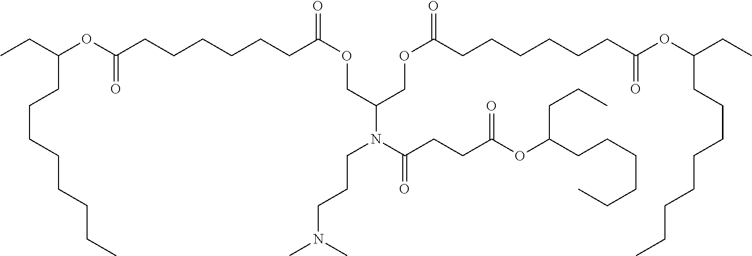 [SMILES: CCCCCCCCC(CC)OC(=O)CCCCCCC(=O)OCC(COC(=O)CCCCCCC(=O)OC(CC)CCCCCCCC)N(CCCN(C)C)C(=O)CCC(=O)OC(CCC)CCCCCC]

Lipid 21
(Lipid 22)

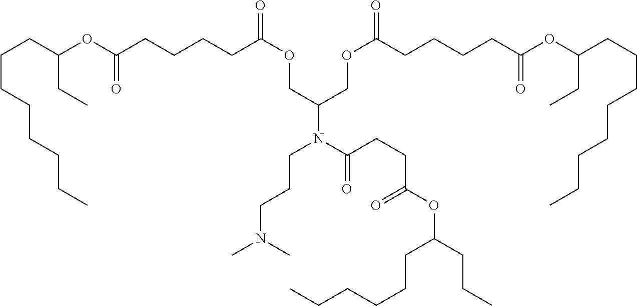 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

Lipid 22
(Lipid 23)

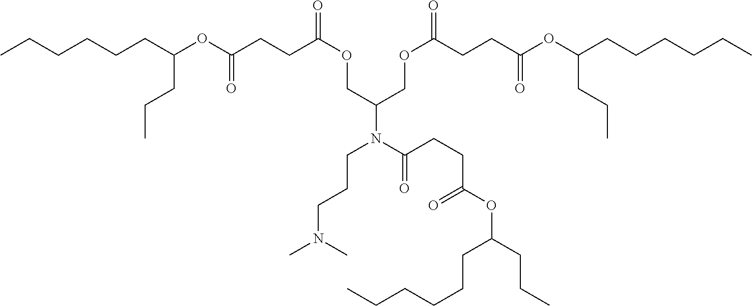 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

Lipid 23
(Lipid 24)

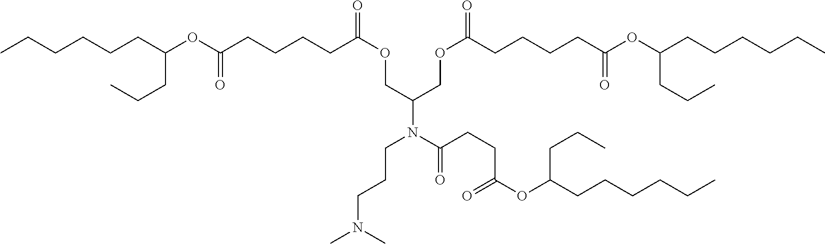 [SMILES: CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

Lipid 24
(Lipid 25)

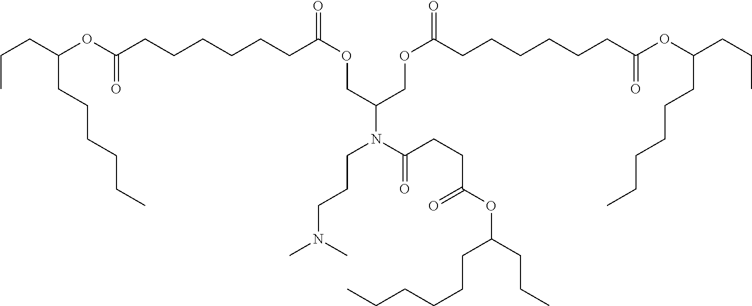 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

Lipid 25
(Lipid 31)

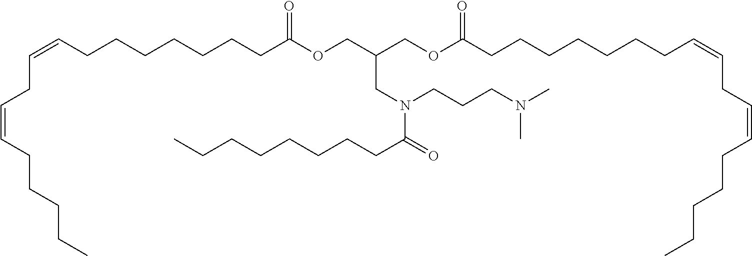 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC]

Lipid 31
(Lipid 32)

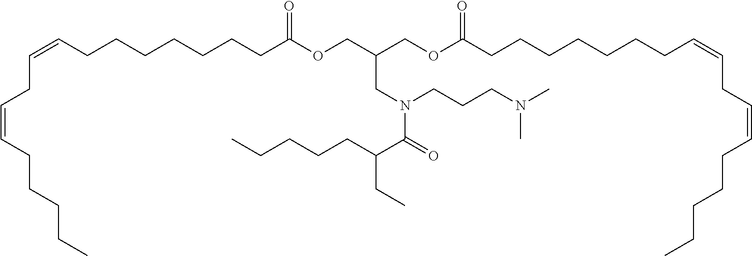 [SMILES: CCCCC/C=C\C/C=C\CCCCCCCC(=O)OCC(COC(=O)CCCCCCC/C=C\C/C=C\CCCCC)CN(CCCN(C)C)C(=O)C(CC)CCCCC]

Lipid 32
(Lipid 33)

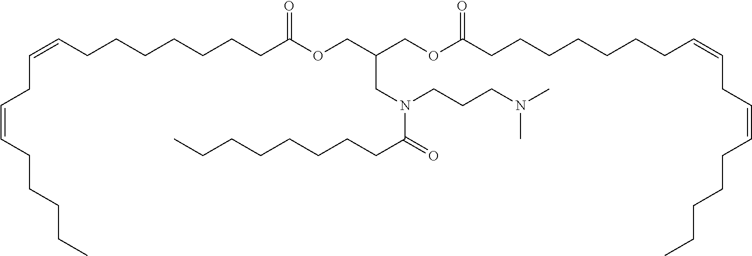 [SMILES: CCCCC/C=C\C/C=C\CCCCCCCC(=O)OCC(COC(=O)CCCCCCC/C=C\C/C=C\CCCCC)CN(CCCN(C)C)C(=O)CCCCCCCC]

Lipid 33
(Lipid 34)

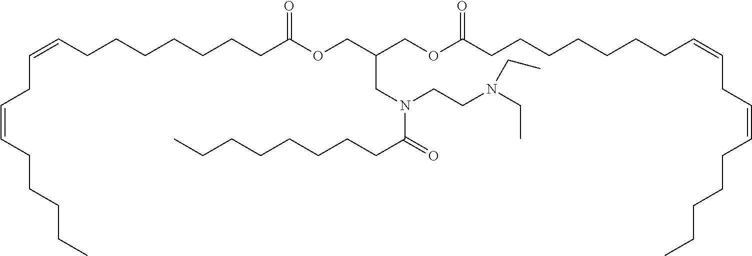 [SMILES: CCCCC/C=C\C/C=C\CCCCCCCC(=O)OCC(COC(=O)CCCCCCC/C=C\C/C=C\CCCCC)CN(CCN(CC)CC)C(=O)CCCCCCCC]

Lipid 34
(Lipid 35)

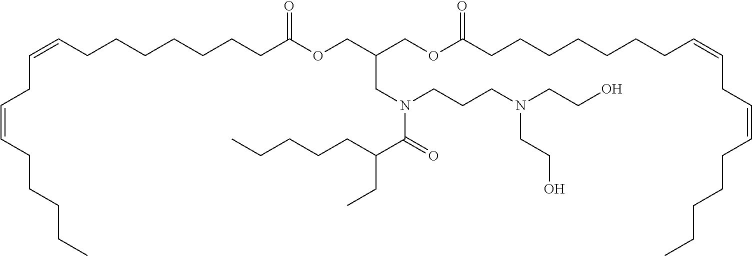 [SMILES: CCCCC/C=C\C/C=C\CCCCCCCC(=O)OCC(COC(=O)CCCCCCC/C=C\C/C=C\CCCCC)CN(CCCN(CCO)CCO)C(=O)C(CC)CCCCC]

Lipid 35
(Lipid 36)

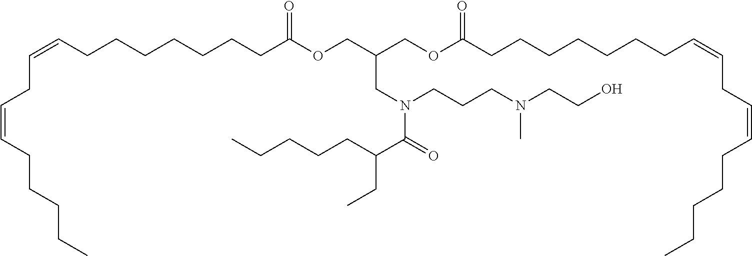 [SMILES: CCCCC/C=C\C/C=C\CCCCCCCC(=O)OCC(COC(=O)CCCCCCC/C=C\C/C=C\CCCCC)CN(CCCN(C)CCO)C(=O)C(CC)CCCCC]

Lipid 36
(Lipid 37)

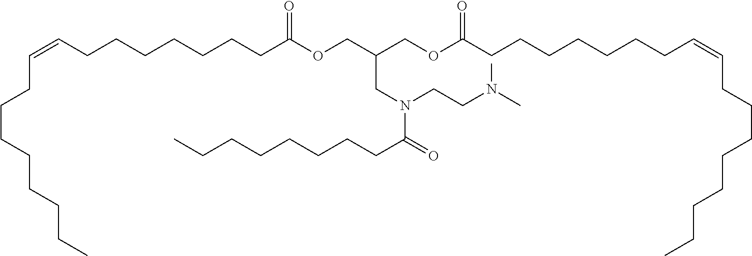 [SMILES: CCCCCCCC/C=C\CCCCCCCC(=O)OCC(COC(=O)CCCCCCC/C=C\CCCCCCCC)CN(CCN(C)C)C(=O)CCCCCCCC]

Lipid 37
(Lipid 38)

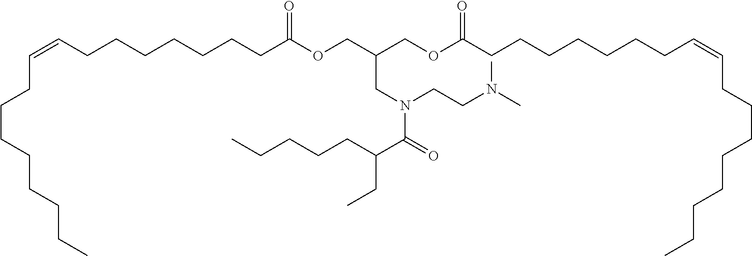 [SMILES: CCCCCCCC/C=C\CCCCCCCC(=O)OCC(COC(=O)CCCCCCC/C=C\CCCCCCCC)CN(CCN(C)C)C(=O)C(CC)CCCCC]

Lipid 38

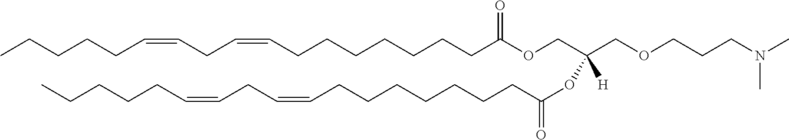 [SMILES: CCCCC/C=C\C/C=C\CCCCCCCC(=O)OC[C@H](COCCCN(C)C)OC(=O)CCCCCCC/C=C\C/C=C\CCCCC]

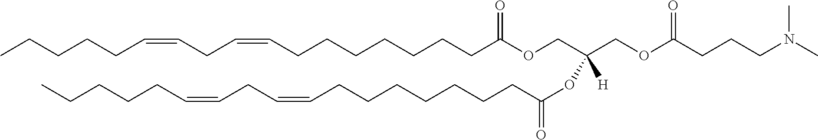 [SMILES: CCCCC/C=C\C/C=C\CCCCCCCC(=O)OC[C@H](COC(=O)CCCN(C)C)OC(=O)CCCCCCC/C=C\C/C=C\CCCCC]

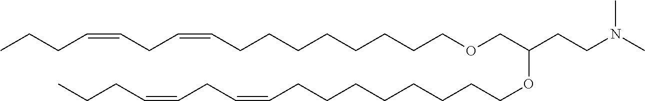 [SMILES: CCC/C=C\C/C=C\CCCCCCCCOCC(CCN(C)C)OCCCCCCCC/C=C\C/C=C\CCC]

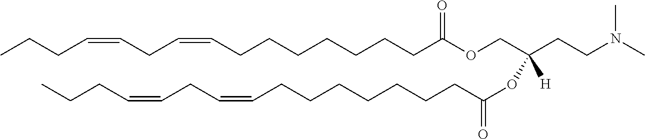 [SMILES: CCC/C=C\C/C=C\CCCCCCCC(=O)OC[C@H](CCN(C)C)OC(=O)CCCCCCC/C=C\C/C=C\CCC]

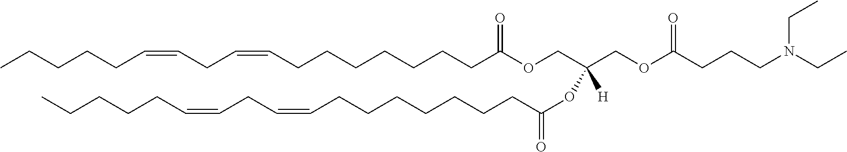 [SMILES: CCCCC/C=C\C/C=C\CCCCCCCC(=O)OC[C@H](COC(=O)CCCN(CC)CC)OC(=O)CCCCCCC/C=C\C/C=C\CCCCC]

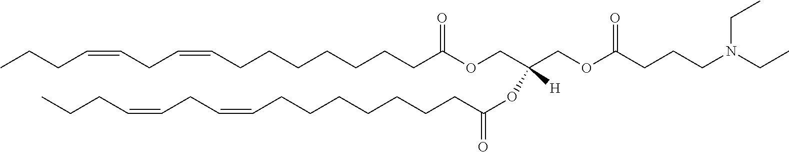 [SMILES: CCC/C=C\C/C=C\CCCCCCCC(=O)OC[C@H](COC(=O)CCCN(CC)CC)OC(=O)CCCCCCC/C=C\C/C=C\CCC]

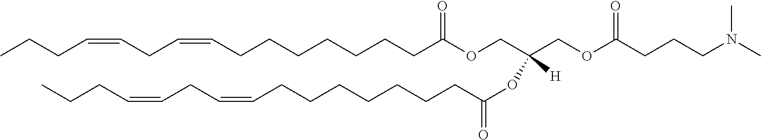 [SMILES: CCC/C=C\C/C=C\CCCCCCCC(=O)OC[C@H](COC(=O)CCCN(C)C)OC(=O)CCCCCCC/C=C\C/C=C\CCC]

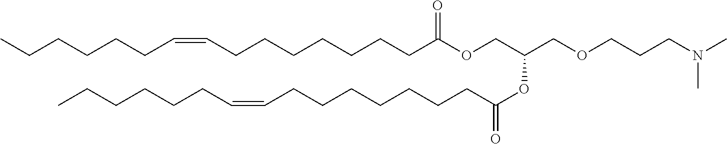 [SMILES: CCCCCC/C=C\CCCCCCCC(=O)OC[C@H](COCCCN(C)C)OC(=O)CCCCCCC/C=C\CCCCCC]

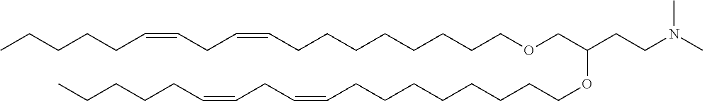 [SMILES: CCCCC/C=C\C/C=C\CCCCCCCCOCC(CCN(C)C)OCCCCCCCC/C=C\C/C=C\CCCCC]

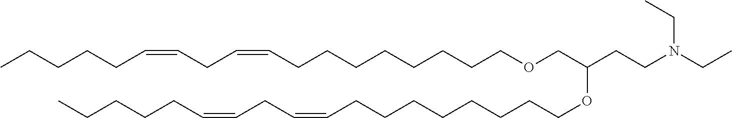 [SMILES: CCCCC/C=C\C/C=C\CCCCCCCCOCC(CCN(CC)CC)OCCCCCCCC/C=C\C/C=C\CCCCC]

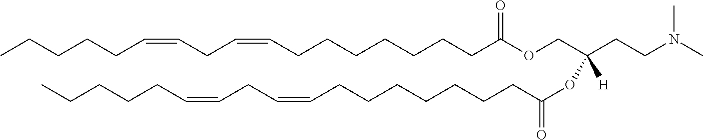 [SMILES: CCCCC/C=C\C/C=C\CCCCCCCC(=O)OC[C@H](CCN(C)C)OC(=O)CCCCCCC/C=C\C/C=C\CCCCC]

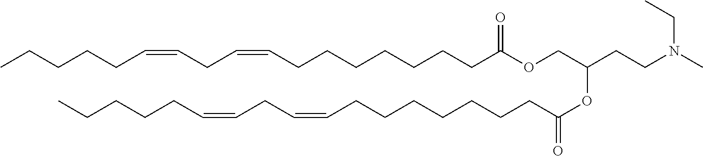 [SMILES: CCCCC/C=C\C/C=C\CCCCCCCC(=O)OCC(CCN(C)CC)OC(=O)CCCCCCC/C=C\C/C=C\CCCCC]

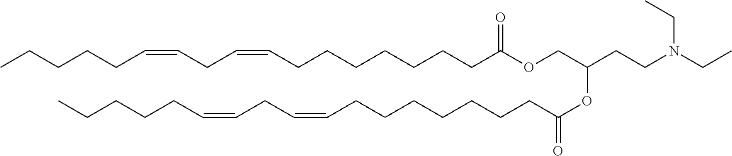 [SMILES: CCCCC/C=C\C/C=C\CCCCCCCC(=O)OCC(CCN(CC)CC)OC(=O)CCCCCCC/C=C\C/C=C\CCCCC]

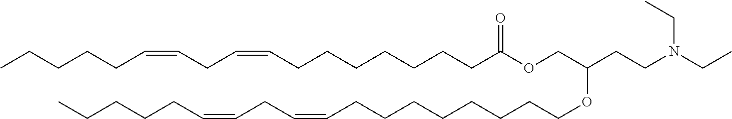 [SMILES: CCCCC/C=C\C/C=C\CCCCCCCCOC(CCN(CC)CC)COC(=O)CCCCCCC/C=C\C/C=C\CCCCC]

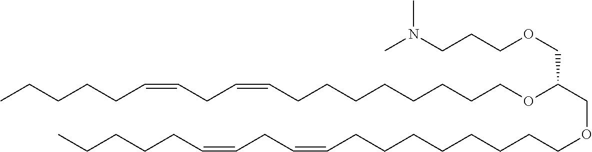 [SMILES: CCCCC/C=C\C/C=C\CCCCCCCCOC[C@@H](COCCCN(C)C)OCCCCCCCC/C=C\C/C=C\CCCCC]

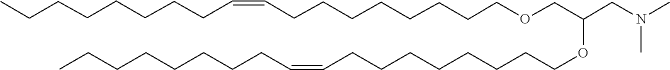 [SMILES: CCCCCCCC/C=C\CCCCCCCCOCC(CN(C)C)OCCCCCCCC/C=C\CCCCCCCC]

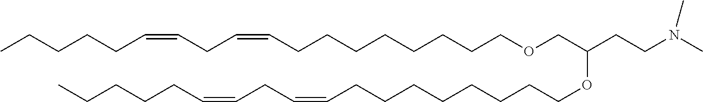 [SMILES: CCCCC/C=C\C/C=C\CCCCCCCCOCC(CCN(C)C)OCCCCCCCC/C=C\C/C=C\CCCCC]

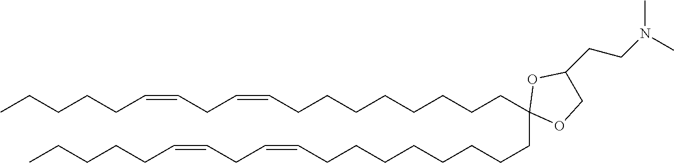 [SMILES: CCCCC/C=C\C/C=C\CCCCCCCCC1(CCCCCCCC/C=C\C/C=C\CCCCC)OCC(CCN(C)C)O1]

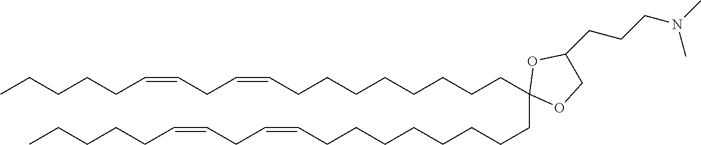 [SMILES: CCCCC/C=C\C/C=C\CCCCCCCCC1(CCCCCCCC/C=C\C/C=C\CCCCC)OCC(CCCN(C)C)O1]

(KC3)

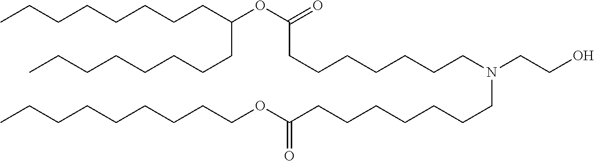 [SMILES: CCCCCCCCCOC(=O)CCCCCCCN(CCO)CCCCCCCC(=O)OC(CCCCCCCC)CCCCCCCC]

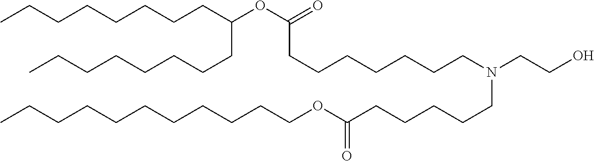 [SMILES: CCCCCCCCCCCOC(=O)CCCCCN(CCO)CCCCCCCC(=O)OC(CCCCCCCC)CCCCCCCC]

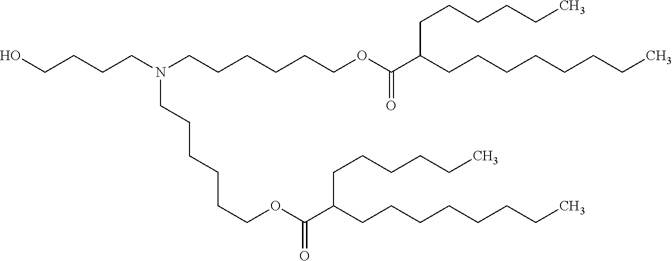 [SMILES: CCCCCCCCC(CCCCCC)C(=O)OCCCCCCN(CCCCO)CCCCCCOC(=O)C(CCCCCC)CCCCCCCC]

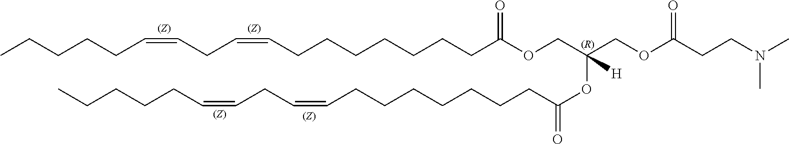

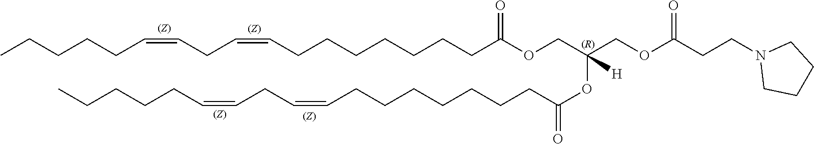

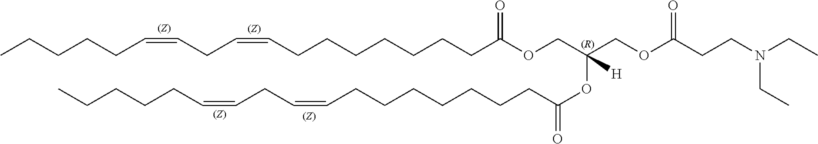

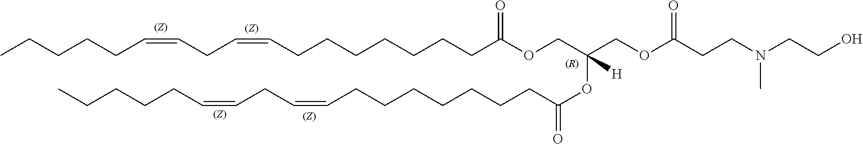

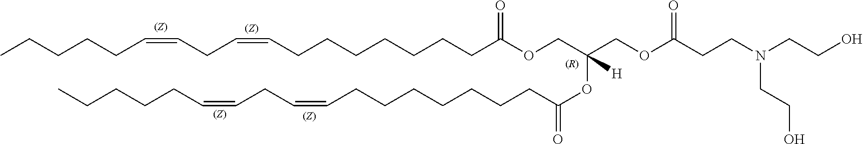

In some embodiments, the ionizable cationic lipid is not Dlin-MC3-DMA.

In certain embodiments, the ionizable cationic lipid described herein may be present in the LNP or the lipid blend in a range of 30-70 mole percent, 30-60 mole percent 30-50 mole percent, 40-70 mole percent, 40-60 mole percent, 40-50 mole percent, 50-70 mole percent, 50-60 mole percent, or of about 30 mole percent, about 35 mole percent, about 40 mole percent, about 45 mole percent, about 50 mole percent, about 55 mole percent, about 60 mole percent, about 65 mole percent, or about 70 mole percent.

### (b) Sterol

In certain embodiments, the LNP or lipid blend may comprise a sterol component which may comprise, for example cholesterol, fecosterol, β-sitosterol, ergosterol, campesterol, stigmasterol, stigmastanol, or brassicasterol. In certain embodiments, the sterol is cholesterol.

The sterol (e.g., cholesterol) may be present in the LNP or the lipid blend in a range of 20-70 mole percent, 20-60 mole percent, 20-50 mole percent, 30-70 mole percent, 30-60 mole percent, 30-50 mole percent, 40-70 mole percent, 40-60 mole percent, 40-50 mole percent, 50-70 mole percent, 50-60 mole percent, or about 20 mole percent, about 25 mole percent, about 30 mole percent, about 35 mole percent, about 40 mole percent, about 45 mole percent, about 50 mole percent, about 55 mole percent, about 60 mole percent or about 65 mole percent.

### (c) Neutral Phospholipid

In some embodiments, the LNP or the lipid blend may comprise one or more neutral phospholipids described herein. In certain embodiments, the one or more neutral phospholipids may comprise, for example, phosphatidylcholine, phosphatidylethanolamine, distearoyl-sn-glycero-3-phosphoethanolamine (DSPE), 1,2-distearoyl-sn-glycero-3-phosphocholine (DSPC), hydrogenated soy phosphatidylcholine (HSPC), 1,2-dioleoyl-sn-glycero-3-phosphoethanolamine (DOPE), or 1,2-dioleoyl-sn-glycero-3-phosphocholine (DOPC), sphingomyelin (SM).

Neutral phospholipids include, for example, distearoyl-phosphatidylethanolamine (DSPE), dimyrstoyl-phosphatidylethanolamine (DMPE), distearoyl-glycero-phosphocholine (DSPC), hydrogenated soy phosphatidylcholine (HSPC), dioleoyl-glycero-phosphoethanolamine (DOPE), dilinoleoyl-glycero-phosphocholine (DLPC), dimyristoyl-glycero-phosphocholine (DMPC), dioleoyl-glycero-phosphocholine (DOPC), dipalmitoyl-glycero-phosphocholine (DPPC), diundecanoyl-glycero-phosphocholine (DUPC), palmitoyl-oleoyl-glycero-phosphocholine (POPC), dioctadecenyl-glycero-phosphocholine, oleoyl-cholesterylhemisuccinoyl-glycero-phosphocholine, hexadecyl-glycero-phosphocholine, dilinolenoyl-glycero-phosphocholine, diarachidonoyl-glycero-3-phosphocholine, didocosahexaenoyl-glycero-phosphocholine, or sphingomyelin.

The neutral phospholipid may be present in the LNP or the lipid blend in a range of 1-10 mole percent, 1-15 mole percent, 1-12 mole percent, 1-10 mole percent, 3-15 mole percent, 3-12 mole percent, 3-10 mole percent, 4-15 mole percent, 4-12 mole percent, 4-10 mole percent, 4-8 mole percent, 5-15 mole percent, 5-12 mole percent, 5-10 mole percent, 6-15 mole percent, 6-12 mole percent, 6-10 more percent, or about 1 mole percent, about 2 mole percent, about 3 mole percent, about 4 mole percent, about 5 mole percent, about 6 mole percent, about 7 mole percent, about 8 mole percent, about 9 mole percent, about 10 mole percent, about 11 mole percent, about 12 mole percent, about 13 mole percent, about 14 mole percent, or about 15 mole percent.

### (d) PEG-Lipid

The LNP or the lipid blend may include one or more polyethylene glycol (PEG) or PEG-modified lipids. Such species may be alternately referred to as PEGylated lipids. A PEG lipid is a lipid modified with polyethylene glycol. As noted above, free PEG-lipids can be included in the LNP or the lipid blend to reduce or eliminate non-specific binding via a targeting group when a lipid-HSC targeting group conjugate (e.g., antibody conjugate) is included in the LNP or lipid blend.

The one or more PEG lipids may comprise, for example, PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-dioleoylgylcerol (PEG-DOG), PEG-dimyristoyl-glycerol (PEG-DMG), PEG-dipalmitoyl-glycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoyl-phosphatidylethanolamine (PEG-DPPE), PEG-distearoylglycerol (PEG-DSG), PEG-diacylglycerol (PEG-DAG, e.g., PEG-DMG, PEG-DPG, and PEG-DSG), PEG-ceramide, PEG-distearoyl-glycero-phosphoglycerol (PEG-DSPG), PEG-dioleoyl-glycero-phosphoethanolamine (PEG-DOPE), 2-[(polyethylene glycol)-2000]—N,N-ditetradecylacetamide, or a PEG-distearoyl-phosphatidylethanolamine (PEG-DSPE) lipid.

In certain embodiments, the LNP or the lipid blend may contain one or more free PEG-lipids that can comprise, for example, PEG-distearoylglycerol (PEG-DSG), PEG-diacylglycerol (PEG-DAG, e.g., PEG-DMG, PEG-DPG, and PEG-DSG), PEG-dimyristoyl-glycerol (PEG-DMG), PEG-distearoyl-phosphatidylethanolamine (PEG-DSPE) and PEG-dimyrstoyl-phosphatidylethanolamine (PEG-DMPE). In some embodiments, the free PEG-lipid comprises a diacylphosphatidylcholines comprising Dipalmitoyl (C16) chain or Distearoyl (C18) chain.

The PEG-lipid may be present in the LNP or in the lipid blend in a range of 1-10 mole percent, 1-8 mole percent, 1-7 mole percent, 1-6 mole percent, 1-5 mole percent, 1-4 mole percent, 1-3 mole percent, 2-8 mole percent, 2-7 mole percent, 2-6 mole percent, 2-5 mole percent, 2-4 mole percent, 2-3 mole percent, or about 1 mole percent, about 2 mole percent, about 3 mole percent, about 4 mole percent, or about 5 mole percent. In some embodiments, the PEG-lipid is a free PEG-lipid.

In some embodiments, the PEG-lipid may be present in the LNP or the lipid blend in the range of 0.01-10 mole percent, 0.01-5 mole percent, 0.01-4 mole percent, 0.01-3 mole percent, 0.01-2 mole percent, 0.01-1 mole percent, 0.1-10 mole percent, 0.1-5 mole percent, 0.1-4 mole percent, 0.1-3 mole percent, 0.1-2 mole percent, 0.1-1 mole percent, 0.5-10 mole percent, 0.5-5 mole percent, 0.5-4 mole percent, 0.5-3 mole percent, 0.5-2 mole percent, 0.5-1 mole percent, 1-2 mole percent, 3-4 mole percent, 4-5 mole percent, 5-6 mole percent, or 1.25-1.75 mole percent. In some embodiments, the PET-lipid may be about 0.5 mole percent, about 1 mole percent, about 1.5 mole percent, about 2 mole percent, about 2.5 mole percent, about 3 mole percent, about 3.5 mole percent, about 4 mole percent, about 4.5 mole percent, about 5 mole percent, or about 5.5 mole percent of the lipid blend. In some embodiments, the PEG-lipid is a free PEG-lipid.

In some embodiments, the lipid anchor length of PEG-lipid is C14 (as in PEG-DMG). In some embodiments, the lipid anchor length of PEG-lipid is C16 (as in DPG). In some embodiments, the lipid anchor length of PEG-lipid is C18 (as in PEG-DSG). In some embodiments, the backbone or head group of PEG-lipid is diacyl glycerol or phosphoethanolamine. In some embodiments, the PEG-lipid is a free PEG-lipid.

A LNP of the present disclosure may comprise one or more free PEG-lipid that is not conjugated to an HSC targeting group (e.g., an antibody that binds to CD105 and/or CD117), and a PEG-lipid that is conjugated to an HSC targeting group (e.g., an antibody that binds to CD105 and/or CD117). In some embodiments, the free PEG-lipid comprises the same or a different lipid as the lipid in the lipid-HSC targeting group conjugate (e.g., lipid-antibody conjugate).

### (e) HSC Targeting Group

As discussed herein, the LNPs may be targeted to a particular cell type, e.g., a hematopoietic stem cell (HSC). This can be accomplished by using one or more of the lipids described herein. Furthermore, targeting can be enhanced by including an HSC targeting group at a solvent accessible surface of an LNP particle. For example, HSC targeting groups may include a member of a specific binding pair, e.g., an antibody-antigen pair, a ligand-receptor pair, etc. In some embodiments, the HSC targeting group is an antibody. In certain embodiments, the antibody binds to an HSC surface antigen, such as CD105 (also known as endoglin) and/or CD117 (also known as c-kit, tyrosine-protein kinase KIT, or mast/stem cell growth factor receptor (SCFR)). Targeting can be implemented, for example, by using lipid-HSC targeting group conjugates (e.g., lipid-antibody conjugates) described herein.

Optionally, the HSC targeting group is an antibody fragment (e.g., an antibody fragment that binds to CD105 and/or CD117) without an Fc component. scFv, Fab, or VHH fragments can also be directly conjugated to activated PEG-lipids to make insertable conjugates. In some embodiments, the HSC targeting group is an antibody fragment-lipid conjugate comprising an scFv, Fab, or VHH fragments. In some embodiments, the antibody fragment of the conjugate is directly conjugated to an activated PEG-lipid.

In some embodiments, PEG-(lipid) is equivalent to (lipid)-PEG.

In certain embodiments, an HSC targeting group may be a surface-bound antibody or surface bound antigen binding fragment thereof, which can permit tuning of cell targeting specificity. This is especially useful since highly specific antibodies can be raised against an epitope of interest for the desired targeting site. In one embodiment, multiple different antibodies can be incorporated into, and presented at the surface of an LNP, where each antibody binds to different epitopes on the same antigen or different epitopes on different antigens. Such approaches can increase the avidity and specificity of targeting interactions to a particular target cell.

In some embodiments, targeting can be implemented, for example, by using lipid-HSC targeting group conjugates (e.g., lipid-antibody conjugates) described herein. Exemplary lipid-HSC targeting group conjugates (e.g., lipid-antibody conjugates) can include compounds of Formula (VI),

[Lipid]-[optional linker]-[HSC targeting group, e.g., antibody that binds an HSC surface antigen, e.g., an antibody that binds to CD105 and/or CD117] (Formula VI).

In certain embodiments, targeting can be implemented, for example, by using lipid-HSC targeting group conjugates (e.g., lipid-antibody conjugates) described herein. Exemplary lipid-HSC targeting group conjugates (e.g., lipid-antibody conjugates) can include compounds of Formula (I),

[Lipid]-[optional linker]-[antibody], (I), wherein the antibody binds to CD105 and/or CD117 (Formula I).

In some embodiments, the HSC targeting group comprises a polypeptide, and the lipid of the conjugate (e.g., a lipid-antibody conjugate) is conjugated to the N-terminus, C-terminus, or anywhere in the middle part of the polypeptide. In some embodiments, the HSC targeting group comprises a polypeptide, and the lipid of the conjugate is conjugated to the N-terminus of the polypeptide. In some embodiments, the lipid is conjugated to the N-terminus of the polypeptide. In some embodiments, the lipid is conjugated to the polypeptide at a position between the N-terminus and the C-terminus. In some embodiments, the HSC targeting group comprises an antibody or an antigen-binding fragment thereof, conjugated to the lipid at the N-terminus, C-terminus, or any position between the N-terminus and C-terminus of the antibody or antigen-binding fragment thereof. In some embodiments, the HSC targeting group comprises an antibody or an antigen-binding fragment thereof, conjugated to the lipid, wherein the antibody or an antigen-binding fragment thereof of the lipid-antibody conjugate binds to CD105 and/or CD117.

Exemplary anti-CD105 antibodies include, for example, TRC105 (US Patent No. US20180311359A1), muRH105 (PCT Application No. WO2012149412A3), 43A3 (Biolegend), 166707 (Novus Biologicals), MEM-229 (Abcam), MJ7/18 (Ge A. Z et al., Cloning and expression of a cDNA encoding mouse endoglin, an endothelial cell TGF-β ligand” Gene 1994:138(1-2):201-206), OTI8A1 (OriGene), EPR19911-220 (Sigma Aldrich), 3A9 (Abcam), MAB1320 (R&D Systems), GTX100508 (GeneTex), SN6 (https://doi.org/10.1002/ijc.11551), MEM-226 (Thermo Fisher), 10862-1-AP (Proteintech), JE60-59 (Thermo Fisher), 103 (Invitrogen), ARC0446 (Invitrogen), PA5-111623 (Invitrogen), PA5-29555 (Invitrogen), PA5-80582 (Invitrogen), PA5-27205 (Invitrogen), PA5-117933 (Invitrogen), PA5-29554 (Invitrogen), 2D5E8 (Proteintech), OTI3H5 (OriGene), OT19E5 (OriGene), 4C11 (Thermo Fisher), 1E5 (Abnova), OTI6G8 (OriGene), QA19A14 (Biolegend), 43A4E1 (Miltenyi Biotec), REA794 (Miltenyi Biotec), D50G1 (Cell Signlaing), AF1097 (Novus Biologicals), 001 (Novus Biologicals), EPR10145-12 (Abcam), EPR10145-10 (Abcam), EPR19911 (Abcam), ENG/3269 (Abcam), P3D1 (Santa Cruz), P4A4 (Santa Cruz), 2Q1707 (Santa Cruz), RM0030-6J9 (Santa Cruz), A-8 (Santa Cruz), 8E11 (Santa Cruz), and antigen-binding fragment thereof. In certain embodiments, the anti-CD105 antibody comprises a heavy chain variable domain (VII) and a light chain variable domain (VL) of an antibody selected from the group consisting of EPR19911-220, GTX100508, PA5-111623, PA5-29555, PA5-80582, PA5-27205, PA5-117933, PA5-29554, AF1097, EPR10145-12, EPR10145-10, EPR19911, and 10862-1-AP. In certain embodiments, the anti-CD105 antibody comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Choth, (1987), J. MOL. BIOL. 196: 901-917), MacCallum (see, MacCallum R M et al., (1996) J. MOL. BIOL. 262: ia C & Lesk A M732-745), or any other CDR determination method known in the art, of the V_{H} and V_{L} sequences of an antibody selected from the group consisting of EPR19911-220, GTX100508, AF1097, PA5-111623, PA5-29555, PA5-80582, PA5-27205, PA5-117933, PA5-29554, EPR10145-12, EPR10145-10, EPR19911, and 10862-1-AP. In some embodiments, the anti-CD105 antibody comprises the heavy chain CDR₁, CDR₂, and CDR₃ and the light chain CDR₁, CDR₂, and CDR3 of any one of the anti-CD105 antibodies described herein, or others anti-CD105 antibodies known in the art.

Exemplary anti-CD117 antibodies include, for example, Ab58 (PCT Publication No. WO2019084067A1), Ab67 (PCT Publication No. WO2019084067A1), Ab55 (PCT Publication No. WO2019084067A1), CK6 (U.S. Pat. No. 8,552,157B2), hSR-1 (U.S. Pat. No. 7,915,391B2), 6LUN1, 104D2 (Biolegend), A3C6E2 (European Patent No. EP0787743), OTI3F9 (OriGene), BA7.3C.9 (ATCC), B—K15 (OriGene), 2B8 (US Patent Publication No. US20160324982A1), ACK2 (Invitrogen), K45 (Blechmen J. et al., J Biol Chem 1993 Feb. 25; 268(6):4399-406), YB5.B8 (Ashman L. K. et al., “Epitope mapping and functional studies with three monoclonal antibodies to the c-kit receptor tyrosine kinase, YB5.B8, 17F11, and SR-1” J Cell Physiol 1994; 158(3):545-554), 1C5 (Thermo Fisher), 34-8800 (Invitrogen), PA5-14694 (Invitrogen), PA5-16458 (Invitrogen), PA5-16770 (Invitrogen), 18696-1-AP (Proteintech), HC34LC14 (Thermo Fisher), ST04-99 (Invitrogen), MA5-44656 (Invitrogen), YR145 (Abcam), EPR25707-134 (Abeam), YR145 (Abeam), D13A2 (Cell Signaling), Ab8l (Cell Signaling), 2C11 (Sigmaaldrich), S18022G (Biolegend), QA18A19 (Biolegend), W18195C (Biolegend), A3C6E2 (Biolegend), AF1356 (R&D Systems), AF332 (R&D Systems), MAB332 (R&D Systems), AF3267 (R&D Systems), E-3 (Santa-Cruz), E-1 (Santa-Cruz), H-10 (Santa-Cruz), 3C11 (Santa-Cruz), 3H1825 (Santa-Cruz), C-14 (Santa-Cruz), 47233 (Novus Biologicals), NBP2-45508 (Novus Biologicals), NBP2-52975 (Novus Biologicals), AF3267 (Novus Biologicals), NBP2-34487 (Novus Biologicals), NBP1-85593 (Novus Biologicals), and antigen-binding fragment thereof. In certain embodiments, the anti-CD117 antibody comprises a heavy chain variable domain (VH) and a light chain variable domain (VL) of an antibody selected from the group consisting of PA5-14694, PA5-16458, PA5-16770, 18696-1-AP, HC34LC14, ST04-99, MA5-44656, EPR25707-134, AF1356, AF332, MAB332, AF3267, NBP2-45508, NBP2-52975, AF3267, NBP2-34487, 34-8800, and NBP1-85593. In certain embodiments, the anti-C117 antibody comprises the heavy chain CDR₁, CDR₂, and CDR₃ and the light chain CDR₁, CDR₂, and CDR₃, determined under Kabat (see, Kabat et al., (1991) Sequences of Proteins of Immunological Interest, NIH Publication No. 91-3242, Bethesda), Chothia (see, e.g., Choth, (1987), J. MOL. BIOL. 196: 901-917), MacCallum (see, MacCallum R M et al., (1996) J. MOL. BIOL. 262: ia C & Lesk A M732-745), or any other CDR determination method known in the art, of the V_{H} and V_{L} sequences of an antibody selected from the group consisting of PA5-14694, PA5-16458, PA5-16770, 18696-1-AP, HC34LC14, ST04-99, MA5-44656, EPR25707-134, AF1356, AF332, MAB332, AF3267, NBP2-45508, NBP2-52975, AF3267, NBP2-34487, 34-8800, and NBP1-85593. In some embodiments, the anti-CD117 antibody comprises the heavy chain CDR₁, CDR₂, and CDR₃ and the light chain CDR₁, CDR₂, and CDR₃ of any one of the anti-CD117 antibodies described herein, or others anti-CD117 antibodies known in the art.

In some embodiments, the HSC targeting group (e.g., the antibody that binds to CD105 and/or CD117) comprises an antibody Fc fragment. The most common immunoglobulin isotype in humans is IgG, which is composed of two identical heavy chain polypeptides and two identical light chain polypeptides. Disulfide bonds link both heavy chain polypeptides to each other. In addition, a disulfide bond also links each light chain polypeptide to a heavy chain polypeptide. Heavy chain polypeptides contain four distinct domains including the variable heavy (VH), constant heavy 1 (CH1), constant heavy 2 (CH2), and constant heavy 3 (CH3) domains. Each light chain contains a variable light (VL) and a variable heavy (VH) domain. The variable domains of the heavy and light chains provide the antibody with antigen binding activity and are responsible for the diversity and specificity of immunoglobulins. Importantly, the heavy chain constant domains, primarily CH2 and CH3, are involved in non-antigen binding functions of antibodies, and constitute the Fc region. The Fc region is capable of binding complement, which may trigger phagocytosis or complement dependent cytotoxicity (CDC). In addition, the Fc region can also bind to Fc receptors, which may trigger phagocytosis or antibody dependent cellular cytotoxicity (ADCC). Moreover, the Fc region is known to improve the maintenance of the antibody during circulation.

In some embodiments, the HSC targeting group (e.g., the antibody that binds to CD105 and/or CD117) comprises an antibody or antigen-binding fragment thereof selected from the group consisting of a Fab, F(ab′)2, Fab′-SH, Fv, and scFv fragment. In some embodiments, the antibody is a human or humanized antibody. In some embodiments, the HSC targeting group comprises a Fab or an immunoglobulin single variable domain, such as a Nanobody.

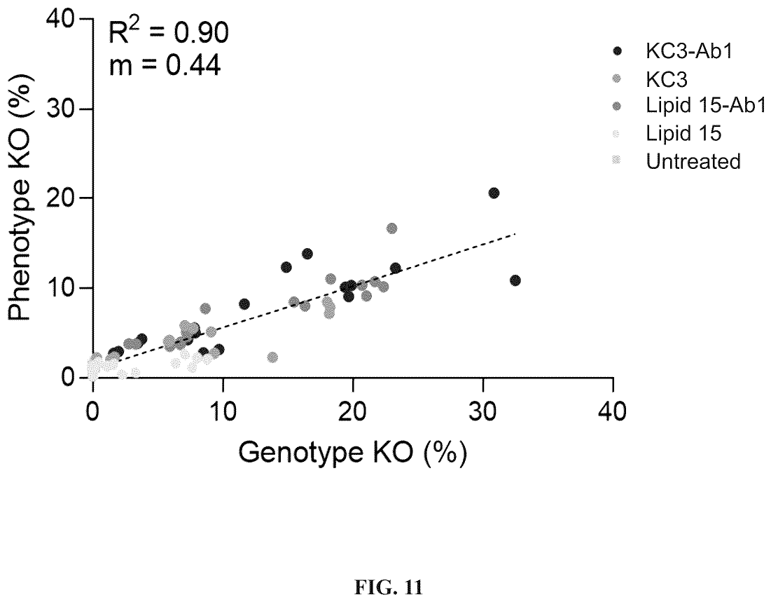

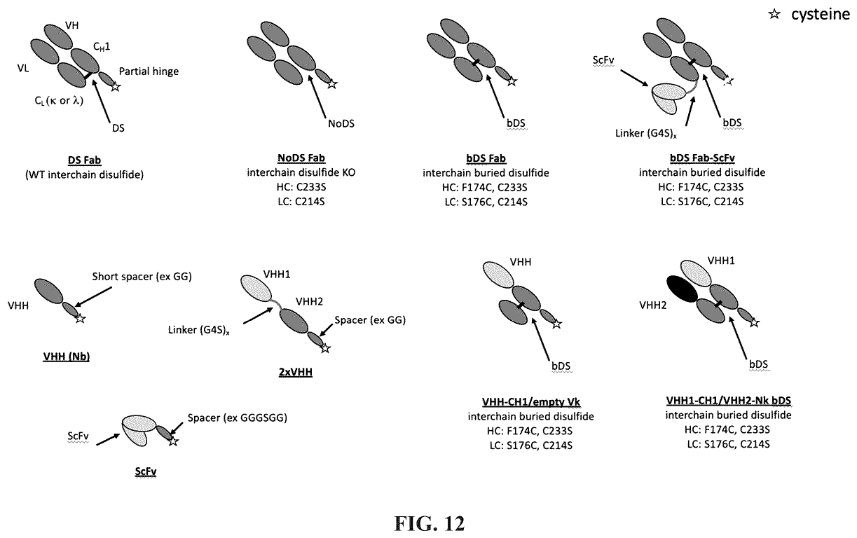

In some embodiments, HSC targeting group comprises a Fab that does not comprise a natural interchain disulfide bond. For example, in some embodiments, the Fab comprises a heavy chain fragment that comprises a C233S substitution, and/or a light chain fragment that comprises a C214S substitution, numbering according to Kabat. In some embodiments, the HSC targeting group comprises a Fab that comprises one or more non-native interchain disulfide bonds. In some embodiments, the interchain disulfide bonds are between two non-native cysteine residues on the light chain fragment and heavy chain fragment, respectively. For example, in some embodiments, the Fab comprises a heavy chain fragment that comprises F174C substitution, and/or a light chain fragment that comprises S176C substitution, numbering according to Kabat. In some embodiments, the Fab comprises a heavy chain fragment that comprises F174C and C233S substitutions, and/or a light chain fragment that comprises S176C and C214S substitutions, numbering according to Kabat. In some embodiments, the HSC targeting group comprises a C-terminal cysteine residue. In some embodiments, the HSC targeting group comprises a Fab that comprises a cysteine at the C-terminus of the heavy or light chain fragment. In some embodiments, the Fab further comprises one or more amino acids between the heavy chain of the Fab and the C-terminal cysteine. For example, in some embodiments, the Fab comprises two or more amino acids derived from an antibody hinge region (e.g., a partial hinge sequence) between the C-terminus of the Fab and the C-terminal cysteine. In some embodiments, the Fab comprises a heavy chain variable domain linked to an antibody CH1 domain and a light chain variable domain linked to an antibody light chain constant domain, wherein the CH1 domain and the light chain constant domain are linked by one or more interchain disulfide bonds, and wherein the HSC targeting group further comprises a single chain variable fragment (scFv) linked to the C-terminus of the light chain constant domain by an amino acid linker. In some embodiments, the Fab antibody is a DS Fab, a NoDS Fab, a bDS Fab, a bDS Fab-ScFv, as demonstrated in FIG. 12.

In some embodiments, the conjugate (e.g., a lipid-antibody conjugate) comprises a Fab, wherein the Fab comprises a heavy chain and a light chain fragment. In some embodiments, the heavy chain fragment comprises a heavy chain variable domain linked to an antibody CH1 domain. In some embodiments, the heavy chain variable domain is an IgG1 VH. In some embodiments, the antibody CH1 domain is an IgG CH1 domain. In some embodiments, the light chain fragment comprises a light chain variable domain linked to an antibody light chain constant domain. In some embodiments, the light chain variable domain is a Kappa VL domain. In some embodiments, the antibody light chain constant domain is a Kappa CL domain. In some embodiments, the CH1 domain and the light chain constant domain are linked by one or more interchain disulfide bonds.

In some embodiments, the HSC targeting group (e.g., the antibody that binds to CD105 and/or CD117) comprises an immunoglobulin single variable domain, such as a Nanobody (e.g., a VHH). In some embodiments, the Nanobody comprises a cysteine at the C-terminus.

An exemplary HSC targeting group (e.g., the antibody that binds to CD105 and/or CD117) may comprise one or more amino sequences as described for the antibodies set forth in Table B. In some embodiments, the HSC targeting group comprises the amino sequences as described for Ab1 as set forth in Table B. In some embodiments, the HSC targeting group comprises the amino sequences as described for Ab2 as set forth in Table B. In some embodiments, the HSC targeting group comprises the amino sequences as described for Ab3 as set forth in Table B.

TABLE B
Exemplary HSC targeting antibodies.

| | | Sequence | SEQ | |
|---|---|---|---|---|
| Antibody | Target | Name | ID NO | Amino Acid Sequence |
| Ab1 | CD117 | CDR-H1 | 1 | FTFSNYAMS |
| | | CDR-H2 | 2 | AISGSGGSTYYADSVKG |
| | | CDR-H3 | 3 | AKGPPTYHTNYYYMDV |
| | | CDR-L1 | 4 | RASQGISSWLA |
| | | CDR-L2 | | AASSLQS |
| | | CDR-L3 | 6 | QQTNSFPYT |
| | | VH | 7 | EVQLLESGGGLVQPGGSLRISCAASGFTFSNY |
| | | | | AMSWVRQAPGKGLEWVSAISGSGGSTYYADSV |
| | | | | KGRFTISRDNSKNTLYLQMNSLRAEDTAVYYC |
| | | | | AKGPPTYHTNYYYMDVWGKGTTVTVSS |
| | | VL | 8 | DIQMTQSPSSVSASVGDRVTITCRASQGISSW |
| | | | | LAWYQQKPGKAPKLLIYAASSLQSGVPSRFSG |
| | | | | SGSGTDETLTISSLQPEDFATYYCQQTNSFPY |
| | | | | TFGGGTKVEIK |
| | | Heavy | 9 | EVQLLESGGGLVQPGGSLRISCAASGFTESNY |
| | | chain | | AMSWVRQAPGKGLEWVSAISGSGGSTYYADSV |
| | | | | KGRFTISRDNSKNTLYLQMNSLRAEDTAVYYC |
| | | | | AKGPPTYHTNYYYMDVWGKGTTVTVSSASTKG |
| | | | | PSVFPLAPSSKSTSGGTAALGCLVKDYFPEPV |
| | | | | TVSWNSGALTSGVHTCPAVLQSSGLYSLSSVV |
| | | | | TVPSSSLGTQTYICNVNHKPSNTKVDKKVEPK |
| | | | | SSDKTHTCGGHHHHHH |
| | | Light | 38 | DIQMTQSPSSVSASVGDRVTITCRASQGISSW |
| | | chain | | LAWYQQKPGKAPKLLIYAASSLQSGVPSRESG |
| | | | | SGSGTDFTLTISSLQPEDFATYYCQQTNSFPY |
| | | | | TFGGGTKVEIKRTVAAPSVFIFPPSDEQLKSG |
| | | | | TASVVCLLNNEYPREAKVQWKVDNALQSGNSQ |
| | | | | ESVTEQDSKDSTYSLCSTLTLSKADYEKHKVY |
| | | | | ACEVTHQGLSSPVTKSENRGES |
| Ab2 | CD117 | CDR-H1 | 10 | FTFSDADMD |
| | | CDR-H2 | 11 | RTRNKAGSYTTEYAASVKG |
| | | CDR-H3 | 12 | AREPKYWIDFDL |
| | | CDR-L1 | 13 | RASQSISSYLN |
| | | CDR-L2 | 14 | AASSLQS |
| | | CDR-L3 | 15 | QQSYIAPYT |
| | | VH | 16 | EVQLVESGGGLVQPGGSLRLSCAASGFTFSDA |
| | | | | DMDWVRQAPGKGLEWVGRTRNKAGSYTTEYAA |
| | | | | SVKGRFTISRDDSKNSLYLQMNSLKTEDTAVY |
| | | | | YCAREPKYWIDFDLWGRGTLVTVSS |
| | | VL | 17 | DIQMTQSPSSLSASVGDRVTITCRASQSISSY |
| | | | | LNWYQQKPGKAPKLLIYAASSLQSGVPSRESG |
| | | | | SGSGTDFTLTISSLQPEDFATYYCQQSYIAPY |
| | | | | TEGGGTKVEIK |
| | | Heavy | 18 | EVQLVESGGGLVQPGGSLRLSCAASGFTESDA |
| | | chain | | DMDWVRQAPGKGLEWVGRTRNKAGSYTTEYAA |
| | | | | SVKGRFTISRDDSKNSLYLQMNSLKTEDTAVY |
| | | | | YCAREPKYWIDFDLWGRGTLVTVSSASTKGPS |
| | | | | VEPLAPSSKSTSGGTAALGCLVKDYFPEPVTV |
| | | | | SWNSGALTSGVHTCPAVLQSSGLYSLSSVVTV |
| | | | | PSSSLGTQTYICNVNHKPSNTKVDKKVEPKSS |
| | | | | DKTHTCGGHHHHHH |
| | | Light | 39 | DIQMTQSPSSLSASVGDRVTITCRASQSISSY |
| | | chain | | LNWYQQKPGKAPKLLIYAASSLQSGVPSRESG |
| | | | | SGSGTDFTLTISSLQPEDFATYYCQQSYIAPY |
| | | | | TEGGGTKVEIKRTVAAPSVEIFPPSDEQLKSG |
| | | | | TASVVCLLNNFYPREAKVQWKVDNALQSGNSQ |
| | | | | ESVTEQDSKDSTYSLCSTLTLSKADYEKHKVY |
| | | | | ACEVTHQGLSSPVTKSENRGES |
| Ab3 | CD105 | CDR-H1 | 19 | DAWMD |
| | | CDR-H2 | 20 | EIRSKASNHATYYAESVKG |
| | | CDR-H3 | 21 | WRRFFDS |
| | | CDR-L1 | 22 | RASSSVSYMH |
| | | CDR-L2 | 23 | ATSNLAS |
| | | CDR-L3 | 24 | QQWSSNPLT |
| | | VH | 25 | EVKLEESGGGLVQPGGSMKLSCAASGFTESDA |
| | | | | WMDWVRQSPEKGLEWVAEIRSKASNHATYYAE |
| | | | | SVKGRFTISRDDSKSSVYLQMNSLRAEDTGIY |
| | | | | YCTRWRRFFDSWGQGTTLTVSS |
| | | VL | 26 | QIVLSQSPAILSASPGEKVTMTCRASSSVSYM |
| | | | | HWYQQKPGSSPKPWIYATSNLASGVPVRESGS |
| | | | | GSGTSYSLTISRVEAEDAATYYCQQWSSNPLT |
| | | | | FGAGTKLELK |
| | | Heavy | 27 | EVKLEESGGGLVQPGGSMKLSCAASGFTESDA |
| | | chain | | WMDWVRQSPEKGLEWVAEIRSKASNHATYYAE |
| | | | | SVKGRFTISRDDSKSSVYLQMNSLRAEDTGIY |
| | | | | YCTRWRRFFDSWGQGTTLTVSSASTKGPSVFP |
| | | | | LAPSSKSTSGGTAALGCLVKDYFPEPVTVSWN |
| | | | | SGALTSGVHTCPAVLQSSGLYSLSSVVTVPSS |
| | | | | SIGTQTYICNVNHKPSNTKVDKKVEPKSSDKT |
| | | | | HTCGGHHHHHH |
| | | Light | 40 | QIVLSQSPAILSASPGEKVTMTCRASSSVSYM |
| | | chain | | HWYQQKPGSSPKPWIYATSNLASGVPVRESGS |
| | | | | GSGTSYSLTISRVEAEDAATYYCQQWSSNPLT |
| | | | | FGAGTKLELKRTVAAPSVEIFPPSDEQLKSGT |
| | | | | ASVVCLLNNFYPREAKVQWKVDNALQSGNSQE |
| | | | | SVTEQDSKDSTYSLCSTLTISKADYEKHKVYA |
| | | | | CEVTHQGLSSPVTKSENRGES |
| mutAb1 | N/A | CDR-H1 | 4 | FAAANYAMS |
| | (non- | | 28 | |
| | binding | CDR-H2 | 29 | AISGAAASTYYADSVKG |
| | neg | | | |
| | ctrl. for | CDR-H3 | 30 | AKGPPTYAAAYYYMDV |
| | Ab1) | | | |
| | | CDR-L1 | 31 | RASQAAASWIA |
| | | CDR-L2 | 32 | AAASLQS |
| | | CDR-L3 | 33 | QQTAAAPYT |
| | | VH | 34 | EVQLLESGGGLVQPGGSLRLSCAASGFAAANY |
| | | | | AMSWVRQAPGKGLEWVSAISGAAASTYYADSV |
| | | | | KGRFTISRDNSKNTLYLQMNSLRAEDTAVYYC |
| | | | | AKGPPTYAAAYYYMDVWGKGTTVTVSS |
| | | VL | 35 | DIQMTQSPSSVSASVGDRVTITCRASQAAASW |
| | | | | LAWYQQKPGKAPKLLIYAAASLQSGVPSRESG |
| | | | | SGSGTDFTLTISSLQPEDFATYYCQQTAAAPY |
| | | | | TEGGGTKVEIK |
| | | Heavy | 36 | EVQLLESGGGLVQPGGSLRLSCAASGFAAANY |
| | | chain | | AMSWVRQAPGKGLEWVSAISGAAASTYYADSV |
| | | | | KGRFTISRDNSKNTLYLQMNSLRAEDTAVYYC |
| | | | | AKGPPTYAAAYYYMDVWGKGTTVTVSSASTKG |
| | | | | PSVFPLAPSSKSTSGGTAALGCLVKDYFPEPV |
| | | | | TVSWNSGALTSGVHTCPAVLQSSGLYSLSSVV |
| | | | | TVPSSSLGTQTYICNVNHKPSNTKVDKKVEPK |
| | | | | SSDKTHTCGGHHHHHH |
| | | Light | 37 | DIQMTQSPSSVSASVGDRVTITCRASQAAASWLAWYQQ |
| | | chain | | KPGKAPKLLIYAAASLQSGVPSRFSGSGSGTDFTLTIS |
| | | | | SLQPEDFATYYCQQTAAAPYTFGGGTKVEIKRTVAAPS |
| | | | | VFIFPPSDEQLKSGTASVVCLLNNFYPREAKVQWKVDN |
| | | | | ALQSGNSQESVTEQDSKDSTYSLCSTLTLSKADYEKHK |
| | | | | VYACEVTHQGLSSPVTKSFNRGES |
| NA | NA | Constant | 41 | ASTKGPSVFPLAPSSKSTSGGTAALGCLVKDY |
| | | Domain | | FPEPVTVSWNSGALTSGVHTCPAVLQSSGLYS |
| | | Heavy | | LSSVVTVPSSSLGTQTYICNVNHKPSNTKVDK |
| | | Chain | | KV |
| | | (IgG1 | | |
| | | CH1) | | |
| NA | NA | C-terminal | 42 | EPKSSDKTHTCGGHHHHHH |
| | | sequence | | |
| NA | NA | Constant | 43 | RTVAAPSVFIFPPSDEQLKSGTASVVCLLNNE |
| | | Domain | | YPREAKVQWKVDNALQSGNSQESVTEQDSKDS |
| | | Light | | TYSLCSTLTISKADYEKHKVYACEVTHQGLSS |
| | | Chain | | PVTKSENRGES |
| | | (Kappa | | |
| | | CL) | | |

In some embodiments, the HSC targeting group (e.g., the antibody that binds to CD105 and/or CD117) comprises an amino acid spacer and/or linker. In some embodiments, the spacer is between two domains of the antibody or antigen-binding fragment thereof. In some embodiments, the spacer is between the V_{HH} domain and the C-terminal cysteine. In some embodiments, the spacer is between the antibody or antigen-binding fragment thereof and the lipid. In some embodiments, the spacer is between the antigen-binding single variable domain and the lipid. In some embodiments, the spacer is between the VHH and the lipid. In some embodiments, the HSC targeting group comprises an amino acid spacer and/or linker set forth in any one of the sequences in Table C. In some embodiments, the HSC targeting group (e.g., antibody) comprises an amino acid spacer and/or linker with the amino acid sequence of AAA, or an amino acid sequence set forth in any one of SEQ ID NOs: 45-60.

TABLE C
Spacer/Linker Sequences

| | SEQ | |
|---|---|---|
| | ID | |
| Name | NO | Amino acid sequence |
| 3A spacer | N/A | AAA |
| 5GS spacer | 45 | GGGGS |
| 7GS spacer | 46 | SGGSGGS |
| 8GS spacer | 47 | GGGGSGGS |
| 9GS spacer | 48 | GGGGSGGGS |
| 10GS spacer | 49 | GGGGSGGGGS |
| 15GS spacer | 50 | GGGGSGGGGSGGGGS |
| 18GS spacer | 51 | GGGGSGGGGSGGGGSGGS |
| 20GS spacer | 52 | GGGGSGGGGSGGGGSGGGGS |
| 25GS spacer | 53 | GGGGSGGGGSGGGGSGGGGSGGGGS |
| 30GS spacer | 54 | GGGGSGGGGSGGGGSGGGGSGGG |
| | | GSGGGGS |
| 35GS spacer | 55 | GGGGGGGGSGGGGSGGGGSGGGG |
| | | SGGGGSGGGGS |
| 40GS spacer | 56 | GGGGSGGGGSGGGGSGGGGSGGG |
| | | GSGGGGSGGGGSGGGGS |
| G1 hinge | 57 | EPKSCDKTHTCPPCP |
| 9GS-G1 hinge | 58 | GGGGSGGGSEPKSCDKTHTCPPCP |
| Llama upper long | 59 | EPKTPKPQPAAA |
| hinge region | | |
| G3 hinge | 60 | ELKTPLGDTTHTCPRCPEPKSCDT |
| | | PPPCPRCPEPKSCDTPPPCPRCPE |
| | | PKSCDTPPPCPRCP |

Examples of amino acid spacers include but are not limited to those set forth in SEQ ID NOs: 45-60 and the amino acid sequence AAA. Spacers of the present invention may comprise at least 3, 5, 10, 15, 20, 25 or 30 amino acids in length. Spacers of the present invention may comprise between 3 and 50, 5 and 45, 7 and 40, 10 and 35, 12 and 30, or 15 and 25 amino acids. In some embodiments, the spacer is 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids in length. The spacer of the fusion protein monomer described herein may be a flexible spacer or a rigid spacer. The spacer of the HSC targeting group (e.g., the antibody that binds to CD105 and/or CD117) described herein may be a short spacer or a long spacer. In some embodiments, the amino acid spacer comprises an amino acid sequence as set forth in Table C comprising 1, 2, 3, 4, or 5 amino acid substitutions, insertions, or deletions. In some embodiments, the amino acid spacer comprises an amino acid sequence as set forth in Table C. Spacers described herein can be used to link two or more amino acid domains together.

In some embodiments, the HSC targeting group (e.g., the antibody that binds to CD105 and/or CD117) comprises one or more complimentary-determining regions (CDR) sequences. CDR sequences of conventional antibodies are highly variable regions of the heavy and light chains in immunoglobulins antibodies that determine antigen specificity and represent the location where these molecules bind to their specific antigen. In some instances, antigen-binding single variable domains (i.e., a V_{HH}), only comprise one set of CDRs, located at the N-terminal portion of the structure. Described herein are HSC targeting groups comprising a polypeptide that comprises one or more CDR sequence that possess an amino acid length between 4 and 30, 6 and 28, 8 and 26, 10 and 24, 12 and 22, 14 and 20, or 16 and 18 residues. In some embodiments, the CDR sequences are 4, 5, 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the CDR sequences are between 4 and 20 amino acids in length. In some embodiments, the CDR sequences are between 5 and 15 amino acids in length. In some embodiments, the CDR sequences comprises an amino acid sequence set forth in Table B comprising 1, 2, 3, 4, or 5 amino acid substitutions, insertions, or deletions. In some embodiments, the CDR sequences comprises an amino acid sequence set forth in Table B.

In some embodiments, the HSC targeting group (e.g., the antibody that binds to CD105 and/or CD117) provided herein comprises a variable heavy domain comprising a CDR-H1, a CDR-H2, and a CDR-H3 sequence, and a variable light domain comprising a CDR-L1, a CDR-L2, and a CDR-L3 sequence. In some embodiments, the HSC targeting group that binds to a HSC surface antigen comprises a CDR-H1, a CDR-H2, a CDR-H3, a CDR-L1, a CDR-L2, and a CDR-L3 sequence, each having an amino acid sequence set forth in Table B, where one or more CDR sequence comprise 1, 2, 3, 4, or 5 amino acid substitutions, insertions, or deletions. In some embodiments, the HSC targeting group that binds to a HSC surface antigen comprises a CDR-H1 having the amino acid sequence set forth in SEQ ID NO:1, a CDR-H2 having the amino acid sequence set forth in SEQ ID NO:2, a CDR-H3 having the amino acid sequence set forth in SEQ ID NO:3, a CDR-L1 having the amino acid sequence set forth in SEQ ID NO:4, a CDR-L2 having the amino acid sequence set forth in SEQ ID NO:5, and a CDR-L3 having the amino acid sequence set forth in SEQ ID NO:6, where one or more CDR sequence comprise 1, 2, 3, 4, or 5 amino acid substitutions, insertions, or deletions. In some embodiments, the HSC targeting group that binds to a HSC surface antigen comprises a CDR-H1 having the amino acid sequence set forth in SEQ ID NO:1, a CDR-H2 having the amino acid sequence set forth in SEQ ID NO:2, a CDR-H3 having the amino acid sequence set forth in SEQ ID NO:3, a CDR-L1 having the amino acid sequence set forth in SEQ ID NO:4, a CDR-L2 having the amino acid sequence set forth in SEQ ID NO:5, and a CDR-L3 having the amino acid sequence set forth in SEQ ID NO:6.

In some embodiments, the HSC targeting group that binds to a HSC surface antigen comprises a CDR-H1 having the amino acid sequence set forth in SEQ ID NO:10, a CDR-H2 having the amino acid sequence set forth in SEQ ID NO:11, a CDR-H3 having the amino acid sequence set forth in SEQ ID NO:12, a CDR-L1 having the amino acid sequence set forth in SEQ ID NO:13, a CDR-L2 having the amino acid sequence set forth in SEQ ID NO:14, and a CDR-L3 having the amino acid sequence set forth in SEQ ID NO:15, where one or more CDR sequence comprise 1, 2, 3, 4, or 5 amino acid substitutions, insertions, or deletions. In some embodiments, the HSC targeting group that binds to a HSC surface antigen comprises a CDR-H1 having the amino acid sequence set forth in SEQ ID NO:10, a CDR-H2 having the amino acid sequence set forth in SEQ ID NO:11, a CDR-H3 having the amino acid sequence set forth in SEQ ID NO:12, a CDR-L1 having the amino acid sequence set forth in SEQ ID NO:13, a CDR-L2 having the amino acid sequence set forth in SEQ ID NO:14, and a CDR-L3 having the amino acid sequence set forth in SEQ ID NO:15.

In some embodiments, the HSC targeting group that binds to a HSC surface antigen comprises a CDR-H1 having the amino acid sequence set forth in SEQ ID NO:19, a CDR-H2 having the amino acid sequence set forth in SEQ ID NO:20, a CDR-H3 having the amino acid sequence set forth in SEQ ID NO:21, a CDR-L1 having the amino acid sequence set forth in SEQ ID NO:22, a CDR-L2 having the amino acid sequence set forth in SEQ ID NO:23, and a CDR-L3 having the amino acid sequence set forth in SEQ ID NO:24, where one or more CDR sequence comprise 1, 2, 3, 4, or 5 amino acid substitutions, insertions, or deletions. In some embodiments, the HSC targeting group that binds to a HSC surface antigen comprises a CDR-H1 having the amino acid sequence set forth in SEQ ID NO: 19, a CDR-H2 having the amino acid sequence set forth in SEQ ID NO:20, a CDR-H3 having the amino acid sequence set forth in SEQ ID NO:21, a CDR-L1 having the amino acid sequence set forth in SEQ ID NO:22, a CDR-L2 having the amino acid sequence set forth in SEQ ID NO:23, and a CDR-L3 having the amino acid sequence set forth in SEQ ID NO:24.

In some embodiments, the HSC targeting group (e.g., the antibody that binds to CD105 and/or CD117) provided herein comprises a variable heavy (VH) domain comprising CDR-H1, CDR-H2, and CDR-H3 sequences, and a variable light (VL) domain comprising CDR-L1, CDR-L2, and CDR-L3 sequences. In some embodiments, the HSC targeting group that binds to a HSC surface antigen comprises a VH domain comprising CDR-H1, CDR-H2, and CDR-H3 sequences, and a VL domain comprising CDR-L1, CDR-L2, and CDR-L3 sequences, where the VH and VL domains have at least 85, 90, 95, 96, 97, 98, 99, or 99.5% sequence identity with the amino acid sequences as described for Ab1 in Table B. In some embodiments, the HSC targeting group that binds to a HSC surface antigen comprises a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:7, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:8. In some embodiments, the HSC targeting group that binds to a HSC surface antigen comprises a VH domain comprising CDR-H1, CDR-H2, and CDR-H3 sequences, where the VH domain comprises the amino acid sequence set forth in any one of SEQ ID NO:7, and a VL domain comprising CDR-L1, CDR-L2, and CDR-L3 sequences, where the VL domain comprises the amino acid sequence set forth in any one of SEQ ID NO:8.

In some embodiments, the HSC targeting group that binds to a HSC surface antigen comprises a VH domain comprising CDR-H1, CDR-H2, and CDR-H3 sequences, and a VL domain comprising CDR-L1, CDR-L2, and CDR-L3 sequences, where the VH and VL domains have at least 85, 90, 95, 96, 97, 98, 99, or 99.5% sequence identity with the amino acid sequences as described for Ab2 in Table B. In some embodiments, the HSC targeting group that binds to a HSC surface antigen comprises a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:16, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:17. In some embodiments, the HSC targeting group that binds to a HSC surface antigen comprises a VH domain comprising CDR-H1, CDR-H2, and CDR-H3 sequences, where the VH domain comprises the amino acid sequence set forth in any one of SEQ ID NO:16, and a VL domain comprising CDR-L1, CDR-L2, and CDR-L3 sequences, where the VL domain comprises the amino acid sequence set forth in any one of SEQ ID NO:17.

In some embodiments, the HSC targeting group that binds to a HSC surface antigen comprises a VH domain comprising CDR-H1, CDR-H2, and CDR-H3 sequences, and a VL domain comprising CDR-L1, CDR-L2, and CDR-L3 sequences, where the VH and VL domains have at least 85, 90, 95, 96, 97, 98, 99, or 99.5% sequence identity with the amino acid sequences as described for Ab3 in Table B. In some embodiments, the HSC targeting group that binds to a HSC surface antigen comprises a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:25, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:26. In some embodiments, the HSC targeting group that binds to a HSC surface antigen comprises a VH domain comprising CDR-H1, CDR-H2, and CDR-H3 sequences, where the VH domain comprises the amino acid sequence set forth in any one of SEQ ID NO:25, and a VL domain comprising CDR-L1, CDR-L2, and CDR-L3 sequences, where the VL domain comprises the amino acid sequence set forth in any one of SEQ ID NO:26.

In some embodiments, the HSC targeting group (e.g., the antibody that binds to CD105 and/or CD117) provided herein comprises a Fab, wherein the Fab comprises a variable heavy (VH) domain comprising CDR-H1, CDR-H2, and CDR-H3 sequences, and a variable light (VL) domain comprising CDR-L1, CDR-L2, and CDR-L3 sequences. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences listed for Ab1 as set forth in Table B. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 7 and 8. In some embodiments, the HSC targeting group that binds to a HSC surface antigen comprises a Fab, where the VH and VL domains of the Fab comprise the amino acid sequences set forth in SEQ ID NOs: 7 and 8.

In some embodiments, the HSC targeting group that binds to a HSC surface antigen comprises a Fab, wherein the VH and VL domains of the Fab have at least 85, 90, 95, 96, 97, 98, 99, or 99.5% sequence identity with the amino acid sequences listed for Ab2 as set forth in Table B. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 16 and 17. In some embodiments, the HSC targeting group that binds to a HSC surface antigen comprises a Fab, where the VH and VL domains of the Fab comprise the amino acid sequences set forth in SEQ ID NOs: 16 and 17.

In some embodiments, the HSC targeting group that binds to a HSC surface antigen comprises a Fab, wherein the VH and VL domains of the Fab have at least 85, 90, 95, 96, 97, 98, 99, or 99.5% sequence identity with the amino acid sequences listed for Ab3 as set forth in Table B. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 25 and 26. In some embodiments, the HSC targeting group that binds to a HSC surface antigen comprises a Fab, where the VH and VL domains of the Fab comprise the amino acid sequences set forth in SEQ ID NOs: 25 and 26.

In some embodiments, the HSC targeting group (e.g., the antibody that binds to CD105 and/or CD117) provided herein comprises a Fab, wherein the Fab comprises a heavy chain domain and a light chain domain. In some embodiments, the HSC targeting group that binds to a HSC surface antigen comprises a Fab, where the heavy chain and light chain domains of the Fab have at least 85, 90, 95, 96, 97, 98, 99, or 99.5% sequence identity with the amino acid sequences listed for Ab1 as set forth in Table B. In some embodiments, the HSC targeting group that binds to a HSC surface antigen comprises a Fab, where the heavy chain and light chain domains of the Fab have at least 85, 90, 95, 96, 97, 98, 99, or 99.5% sequence identity with the amino acid sequences set forth in SEQ ID NOs: 9 and 38. In some embodiments, the HSC targeting group that binds to a HSC surface antigen comprises a Fab, where the heavy chain and light chain domains of the Fab comprise the amino acid sequences set forth in SEQ ID NOs: 9 and 38.

In some embodiments, the HSC targeting group that binds to a HSC surface antigen comprises a Fab, wherein the heavy chain and light chain domains of the Fab have at least 85, 90, 95, 96, 97, 98, 99, or 99.5% sequence identity with the amino acid sequences listed for Ab2 as set forth in Table B. In some embodiments, the HSC targeting group that binds to a HSC surface antigen comprises a Fab, where the heavy chain and light chain domains of the Fab have at least 85, 90, 95, 96, 97, 98, 99, or 99.5% sequence identity with the amino acid sequences set forth in SEQ ID NOs: 18 and 39. In some embodiments, the HSC targeting group that binds to a HSC surface antigen comprises a Fab, where the heavy chain and light chain domains of the Fab comprise the amino acid sequences set forth in SEQ ID NOs: 18 and 39.

In some embodiments, the HSC targeting group that binds to a HSC surface antigen comprises a Fab, where the heavy chain and light chain domains of the Fab have at least 85, 90, 95, 96, 97, 98, 99, or 99.5% sequence identity with the amino acid sequences listed for Ab3 as set forth in Table B. In some embodiments, the HSC targeting group that binds to a HSC surface antigen comprises a Fab, where the heavy chain and light chain domains of the Fab have at least 85, 90, 95, 96, 97, 98, 99, or 99.5% sequence identity with the amino acid sequences set forth in SEQ ID NOs: 27 and 40. In some embodiments, the HSC targeting group that binds to a HSC surface antigen comprises a Fab, where the heavy chain and light chain domains of the Fab comprise the amino acid sequences set forth in SEQ ID NOs: 27 and 40.

In some embodiments, the HSC targeting group (e.g., the antibody that binds to CD105 and/or CD117) comprises two or more antigen-binding domains (e.g., two V_{HH} domains). In some embodiments, the two or more antigen-binding domains are linked by an amino acid linker. In some embodiments, the two or more V_{HH} domains are linked by an amino acid linker. In some embodiments, the amino acid linker comprises one or more glycine and/or serine residues (e.g., one or more repeats of the sequence GGGGS). In some embodiments, the HSC targeting group comprises a first V_{HH} domain linked to an antibody CH1 domain and a second V_{HH} domain linked to an antibody light chain constant domain, and wherein the antibody CH1 domain and the antibody light chain constant domain are linked by one or more disulfide bonds (e.g., interchain disulfide bonds). In some embodiments, the HSC targeting group (e.g., the antibody that binds to CD105 and/or CD117) comprises a V_{HH} domain linked to an antibody CH1 domain, and wherein the antibody CH1 domain is linked to an antibody light chain constant domain by one or more disulfide bonds. In some embodiments, the CH1 domain comprises F174C and C233S substitutions, and the light chain constant domain comprises S176C and C214S substitutions, numbering according to Kabat. In some embodiments, the antibody is a ScFv, a V_{HH}, a 2×V_{HH}, a V_{HH}—CH1/empty Vk, or a V_{HH}1-CH₁/V_{HH}-2-Nb bDS, as demonstrated in FIG. 12.

In some embodiments, the HSC targeting group (e.g., the antibody that binds to CD105 and/or CD117) comprises a polypeptide that binds to a HSC surface antigen with a high binding affinity. In some embodiments, the HSC targeting group binding affinity for the HSC surface antigen is measured as an equilibrium dissociation constant (K_{D}). In some embodiments, the HSC targeting group binds to a HSC surface antigen with a binding affinity of less than 500, 400, 300, 200, 100, or 1 nM. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 7 and 8. In some embodiments, the HSC targeting group that binds to a HSC surface antigen comprises a Fab, where the VH and VL domains of the Fab comprise the amino acid sequences set forth in SEQ ID NOs: 7 and 8. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 16 and 17. In some embodiments, the HSC targeting group that binds to a HSC surface antigen comprises a Fab, where the VH and VL domains of the Fab comprise the amino acid sequences set forth in SEQ ID NOs: 16 and 17. In some embodiments, the HSC targeting group that binds to a HSC surface antigen comprises a Fab, where the VH and VL domains of the Fab have at least 85, 90, 95, 96, 97, 98, 99, or 99.5% sequence identity with the amino acid sequences set forth in SEQ ID NOs: 25 and 26. In some embodiments, the HSC targeting group that binds to a HSC surface antigen comprises a Fab, where the VH and VL domains of the Fab comprise the amino acid sequences set forth in SEQ ID NOs: 25 and 26.

In some embodiments, the HSC targeting group (e.g., the antibody that binds to CD105 and/or CD117) provided herein targets a human HSC surface antigen, including, for example, any of the HSC surface antigens described in this application. In some embodiments, the HSC targeting group targets human CD105 and/or human CD117. In some embodiments, the HSC targeting group targets more than one human HSC surface antigen.

In some embodiments, the conjugate (e.g., a lipid-antibody conjugate) comprising a HSC targeting group is capable of binding to a non-human HSC surface antigen. In some embodiments, the conjugate (e.g., a lipid-antibody conjugate) comprising a HSC targeting group is capable of binding to a human HSC surface antigen. In some embodiments, the conjugate (e.g., a lipid-antibody conjugate) comprising a HSC targeting group is capable of binding to a human HSC surface antigen described herein, for example, human CD105 and/or human CD117.

In some embodiments, the HSC targeting group (e.g., the antibody that binds to CD105 and/or CD117) comprises a polypeptide sequence as disclosed herein. In some embodiments, the targeting moiety comprises all six CDRs of a polypeptide sequence (e.g., an antibody polypeptide sequence, e.g., a Fab polypeptide sequence) as disclosed herein. In some embodiments, the HSC targeting group comprises CDR1, CDR2, and CDR3 of an immunoglobulin single variable domain (ISVD) as disclosed herein. In further embodiments, the HSC targeting group (e.g., the antibody that binds to CD105 and/or CD117) binds to the same epitope on the target molecule (e.g., CD105 and/or CD117) that a polypeptide sequence as disclosed herein binds to. In further embodiments, the HSC targeting group (e.g., the antibody that binds to CD105 and/or CD117) competes with a polypeptide sequence as disclosed herein to bind to the same epitope on the target molecule.

In certain embodiments, the HSC targeting group (e.g., the antibody that binds to CD105 and/or CD117) may be covalently coupled to a lipid via a polyethylene glycol (PEG) containing linker.

In other embodiments, the lipid used to create a conjugate (e.g., a lipid-antibody conjugate) may be selected from

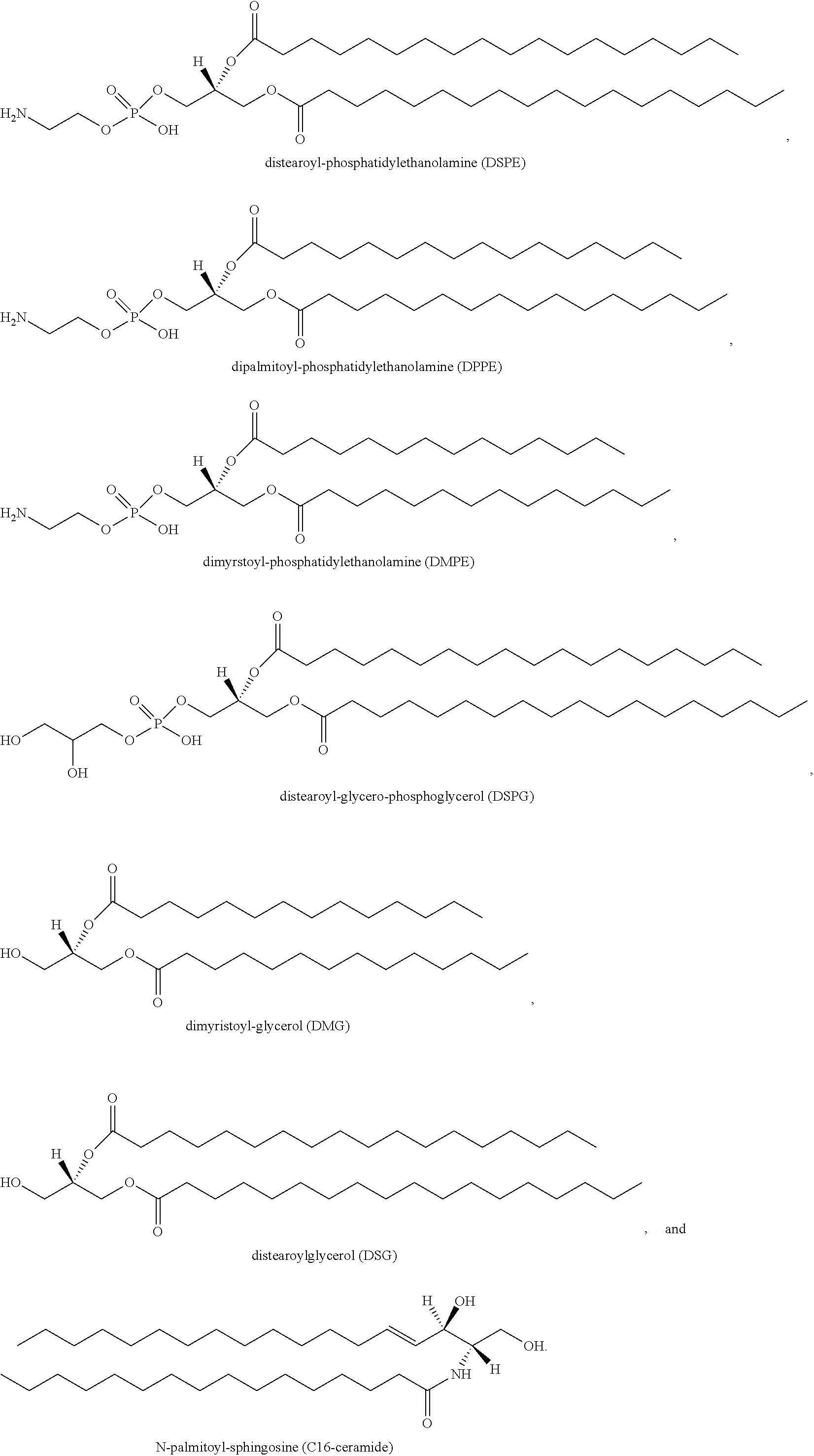

The HSC targeting group (e.g., the antibody that binds to CD105 and/or CD117) can be covalently coupled to a lipid either directly or via a linker, for example, a polyethylene glycol (PEG) containing linker. In certain embodiments, the PEG is PEG 1000, PEG 2000, PEG 3400, PEG 3000, PEG 3450, PEG 4000, or PEG 5000. In certain, embodiments, the PEG is PEG 2000.

In some embodiments, the lipid-HSC targeting group conjugate (e.g., the lipid-antibody conjugate) is present in the LNP in a range of 0.001-0.5 mole percent, 0.001-0.3 mole percent, 0.002-0.2 mole percent, 0.01-0.1 mole percent, 0.1-0.3 mole percent, or 0.1-0.2 mole percent.

In certain embodiments, the lipid-HSC targeting group conjugate (e.g., the lipid-antibody conjugate) comprises DSPE, a PEG component and a targeting antibody. In certain embodiments, HSC targeting group conjugate is an antibody that binds to an HSC surface antigen described herein, e.g., an antibody that binds to CD105 and/or CD117.

An exemplary lipid-HSC targeting group conjugate (e.g., the lipid-antibody conjugate) comprises DSPE and PEG 2000, for example, as described in Nellis et al. (2005) BIOTECHNOL. PROG. 21, 205-220. An exemplary conjugate comprises the structure of Formula (VII), where the scFv represents an engineered antibody binding site that binds to a target of interest. In certain embodiments, the engineered antibody binding site binds to any of the targets described herein. In certain embodiments, the engineered antibody binding site can be, for example, an engineered anti-CD105 antibody or an engineered anti-CD117 antibody.

An example of a compound of Formula (VII) is as shown below:

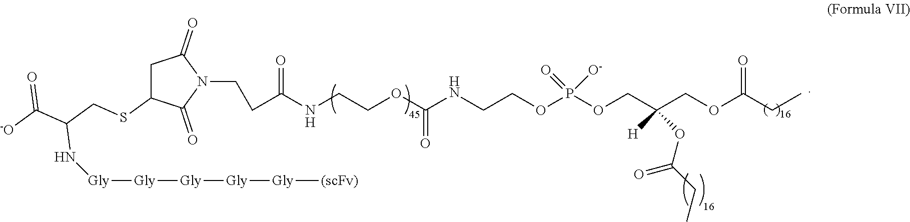

It is contemplated that the scFv in Formula (VII) may be replaced with an intact antibody or an antigen fragment thereof (e.g., a Fab).

Another example of a compound of Formula (VIII) is as shown below:

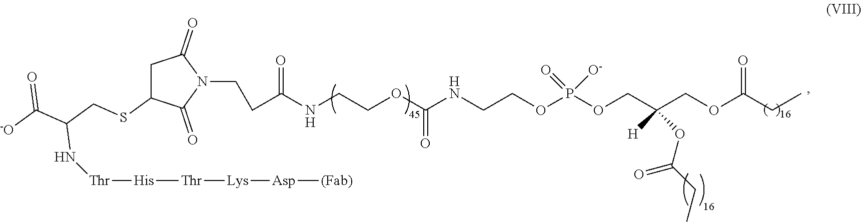

the production of which is described in Nellis et al. (2005) supra, or U.S. Pat. No. 7,022,336. It is contemplated that the Fab in Formula (VIII) may be replaced with an intact antibody or an antigen fragment thereof (e.g., an (Fab′)₂ fragment) or an engineering antibody binding site (e.g., an scFv).

Other lipid-antibody conjugates are described, for example, in U.S. Pat. No. 7,022,336, where the targeting group (e.g., antibody or antigen-binding fragment thereof) may be replaced with a targeting group of interest, for example, a targeting group that binds any HSC surface antigen described herein.

In certain embodiments, the lipid component of an exemplary conjugate of Formula (I) or Formula (VI) can be any of the lipids described herein. In some embodiments, the lipid component of a conjugate of Formula (I) or Formula (VI) is based on an ionizable, cationic lipid described herein, for example, an ionizable, cationic lipid of Formula (II′), Formula (II), Formula (IIa), Formula (Iib), Formula (IIIa), Formula (IIIb), Formula (IV), or Formula (V), or a salt thereof. For example, an exemplary ionizable, cationic lipid can be selected from Table A, or a salt thereof.

In certain embodiments, the conjugate (e.g., lipid-antibody conjugate) based on a lipid of the present disclosure may include:

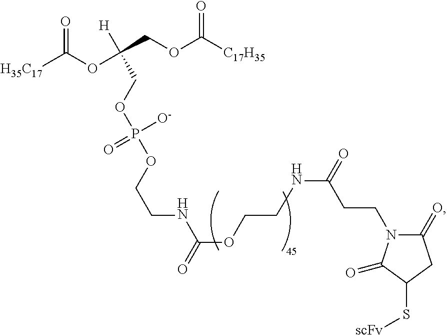

where scFv represents an engineered antibody binding site that binds a target (e.g., an HSC surface antigen) described herein, e.g., CD105 and/or CD117.

In certain embodiments, the LNP may further comprise free PEG-lipid so as to reduce the amount of non-specific binding via the HSC targeting group (e.g., antibody that binds to CD105 and/or CD117). The free PEG-lipid can be the same or different from the PEG-lipid included in the conjugate. In certain embodiments, the free PEG-lipid is selected from the group consisting of PEG-distearoyl-phosphatidylethanolamine (PEG-DSPE) or PEG-dimyrstoyl-phosphatidylethanolamine (PEG-DMPE), N-(Methylpolyoxyethylene oxycarbonyl)-1,2-dipalmitoyl-sn-glycero-3-phosphoethanolamine (DPPE-PEG) 1,2-Dimyristoyl-rac-glycero-3-methylpolyoxyethylene (PEG-DMG), 1,2-Dipalmitoyl-rac-glycero-3-methylpolyoxyethylene (PEG-DPG), 1,2-Dioleoyl-rac-glycerol, methoxypolyethylene Glycol (DOG-PEG) 1,2-Distearoyl-rac-glycero-3-methylpolyoxyethylene (PEG-DSG), N-palmitoyl-sphingosine-1-{succinyl[methoxy(polyethylene glycol)](PEG-ceramide), DSPE-PEG-cysteine, or a derivative thereof, all with average PEG lengths between 2000-5000, with 2000, 3400, or 5000. A final composition may contain a mixture of two or more of these pegylated lipids. In certain embodiments, the LNP composition comprises a mixture of PEG-lipids with myristoyl and stearic acyl chains. In certain embodiments, the LNP composition comprises a mixture of PEG-lipids with palmitoyl and stearoyl acyl chains.

In certain embodiments, the derivative of the PEG-lipid has a methyoxy, hydroxyl or a carboxylic acid end group at the PEG terminus.

The lipid-HSC targeting group conjugate (e.g., lipid-antibody conjugate) can be incorporated into LNPs as described below, for example, in LNPs containing, for example, an ionizable cationic lipid, a sterol, a neutral phospholipid and a PEG-lipid. It is contemplated that, in certain embodiments, the LNPs containing the lipid-HSC targeting group can contain an ionizable cationic lipid described herein or a cationic lipid described, for example, in U.S. Pat. Nos. 10,221,127, 10,653,780 or U.S. Published application No. US2018/0085474, US2016/0317676, International Publication No. WO2009/086558, or Miao et al. (2019) NATURE BIOTECH 37:1174-1185, or Jayaraman et al. (2012) ANGEW CHEM INT. 51: 8529-8533.

In some embodiments, the cationic lipid can be selected from an ionizable cationic lipid set forth in Table A, or a salt thereof. Any variation or embodiment of R¹, R², R³, R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{a1}, R^{a2}, R^{3B}, R^{3B1}, R^{3B2}, R^{3B3}, R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, or R^{s15} provided herein can be combined with every other variation or embodiment of R¹, R², R³, R^{1A}, R^{2A}, R^{3A}, R^{1A1}, R^{1A2}, R^{1A3}, R^{2A1}, R^{2A2}, R^{2A3}, R^{3A1}, R^{3A2}, R^{3A3}, R^{a1}, R^{a2}, R^{3B}, R^{3B1}, R^{3B2}, R^{3B3}, R^{s1}, R^{s2}, R^{s3}, R^{s4}, R^{s5}, R^{s6}, R^{s7}, R^{s8}, R^{s9}, R^{s10}, R^{s11}, R^{s12}, R^{s13}, R^{s14}, or R^{s15}, as if each combination had been individually and specifically described.

The LNPs can be formulated using the methods and other components described below in the following sections.

In certain embodiments, the LNP or lipid blend can also include a lipid-HSC targeting group conjugate (e.g., a lipid-antibody conjugate) as described herein.

The lipid-HSC targeting group conjugate (e.g., the lipid-antibody conjugate) may be present in the LNP or the lipid blend in a range of 0.001-0.5 mol percent, 0.001-0.1 mole percent, 0.01-0.5 mole percent, 0.05-0.5 mole percent, 0.1-0.5 mole percent, 0.1-0.3 mole percent, 0.1-0.2 mole percent, 0.2-0.3 mole percent, of about 0.01 mole percent, about 0.05 mole percent, about 0.1 mole percent, about 0.15 mole percent, about 0.2 mole percent, about 0.25 mole percent, about 0.3 mole percent, about 0.35 mole percent, about 0.4 mole percent, about 0.45 mole percent, or about 0.5 mole percent.

### (f) Payloads

The LNP compositions may comprise an agent, for example, a nucleic acid molecule for delivery to a cell (e.g., a hematopoietic stem cell (HSC)) or tissue, for example, a cell (e.g., an HSC) or tissue in a subject.

The LNP compositions of the present invention may include a nucleic acid, for example, a DNA or RNA, such as an mRNA, tRNA, microRNA, siRNA, guide RNA (gRNA), prime editing guide RNA (pegRNA), circRNA (circular RNA), ribozymes, decoy RNA, dicer substrate siRNA, or donor template DNA or RNA. The LNP compositions of the present invention may include single-stranded DNA (ssDNA), double-stranded DNA (dsDNA), single-stranded RNA (ssRNA), and/or double-stranded RNA (dsRNA). It is contemplated that nucleic acids can contain naturally occurring components, such as, naturally occurring bases, sugars or linkage groups (e.g., phosphodiester linkage groups) or may contain non-naturally occurring components or modifications, (e.g., thioester linkage groups). For example, the nucleic acid can be synthesized to contain base, sugar, linker modifications known to those skilled in the art. Furthermore, the nucleic acids can be linear or circular, or have any desired configuration. The LNP compositions can include multiple nucleic acid molecules, for example, multiple RNA molecules, which can be the same or different.

In certain embodiments, the payload is an mRNA. In certain embodiments, a particular LNP composition may contain a number of mRNA molecules that can be the same or different. In certain embodiments, one or more LNP compositions including one or more different mRNAs may be combined, and/or simultaneously contacted, with a cell. It is contemplated that an mRNA may include one or more of a stem loop, a chain terminating nucleoside, a polyA sequence, a polyadenylation signal, and/or a 5′ cap structure. The mRNA may encode a site-directed nuclease, a chemical base editor, a prime editor, or an epigenome editor as described herein.

In some embodiments, the one or more nucleic acids of the payload comprise an mRNA encoding a site-directed nuclease, a chemical base editor, a prime editor, or an epigenome editor. In some embodiments, the one or more nucleic acids comprise an mRNA encoding a site-directed nuclease. In some embodiments, the site-directed nuclease is a CRISPR-associated (Cas) nuclease, a zinc finger nuclease (ZFN), a transcription activator-like effector nuclease (TALEN), or a megaTAL. In some embodiments, the site-directed nuclease is a ZFN, TALEN, or megaTAL comprising an amino acid sequence that confers binding to a target nucleotide sequence.

In some embodiments, the one or more nucleic acids of the payload comprise an mRNA encoding a CRISPR-associated (Cas) nuclease or a chemical base editor; and a guide RNA (gRNA) comprising a nucleotide sequence that confers binding to a target nucleotide sequence.

In some embodiments, the one or more nucleic acids of the payload comprise an mRNA encoding a prime editor; and a prime editing guide RNA (pegRNA) comprising a nucleotide sequence that confers binding to a target nucleotide sequence.

In some embodiments, the payload comprises a gRNA or pegRNA. In some embodiments, the gRNA or pegRNA of the payload comprises a sequence having at least 80% identity to at least 15 consecutive nucleotides of the target nucleotide sequence. In some embodiments, the gRNA or pegRNA of the payload comprises a sequence having at least 80%, 85%, 90%, 91%, 92%, 93%, 94%, 95%, 96%, 97%, 98%, or 99% identity to at least 15 consecutive nucleotides of the target nucleotide sequence. In some embodiments, the gRNA or pegRNA of the payload comprises a sequence having at least 80% identity to at least 15, 16, 17, 18, 19, 20, 21, 22, 23, 24, 25, 26, 27, 28, 29, or 30 consecutive nucleotides of the target nucleotide sequence.

In some embodiments, the payload comprises a gRNA or pegRNA. In some embodiments, the gRNA or pegRNA of the payload comprises a sequence having at least 80% identity to at least 15 consecutive nucleotides of the target nucleotide sequence. In some embodiments, the one or more nucleic acids of the payload further comprise a donor template nucleic acid comprising a sequence having at least 80% identity to at least 15 consecutive nucleotides of the target nucleotide sequence. In some embodiments, the donor template nucleic acid of the payload comprises a sequence having at least 80%, 85%, 90%, 91%, 92%, 93%, 94%, 95%, 96%, 97%, 98%, or 99% identity to at least 15 consecutive nucleotides of the target nucleotide sequence. In some embodiments, the donor template nucleic acid of the payload comprises a sequence having at least 80% identity to at least 15, 16, 17, 18, 19, 20, 21, 22, 23, 24, 25, 26, 27, 28, 29, or 30 consecutive nucleotides of the target nucleotide sequence.

In some embodiments, the target nucleotide sequence comprises at least 15, 16, 17, 18, 19, 20, 21, 22, 23, 24, 25, or more consecutive nucleotides and is located within a coding region of a gene, an intronic region associated with a gene, an exon region associated with a gene, a 5′ untranslated region associated with a gene, or a 3′ untranslated region associated with a gene, wherein the gene is selected from the group consisting of gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, W4S, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence is within a regulatory region of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence is within an enhancer region or a repressor region of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F₈, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C.

In some embodiments, the target nucleotide sequence is within the BCL11A erythroid enhancer In some embodiments, the target nucleotide sequence comprises a polynucleotide sequence of the BCL11A erythroid enhancer. In some embodiments, the target nucleotide sequence comprises a polynucleotide sequence in intron-2 of the BCL11A gene. In some embodiments, the target nucleotide sequence comprises a polynucleotide sequence between about +54 kb and about +63 kb downstream (in the 3′ direction) of the BCL11A transcription start site (TSS). In certain embodiments, the target nucleotide sequence comprises a polynucleotide sequence between about +54 kb and about +56 kb, a polynucleotide between about +57 kb and about +59 kb, or a polynucleotide between about +62 kb and about +63 kb downstream of the BCL11A TSS, or any combination thereof. In certain embodiments, the target nucleotide sequence comprises a polynucleotide sequence between about +54 kb and about +56 kb downstream of the BCL11A TSS. In certain embodiments, the target nucleotide sequence comprises a polynucleotide sequence between about +57 kb and about +59 kb downstream of the BCL11A TSS. In certain embodiments, the target nucleotide sequence enhancer comprises a polynucleotide sequence between about +62 kb and about +63 kb downstream of the BCL11A TSS. In some embodiments, the target nucleotide sequence comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb downstream of the BCL11A TSS. In certain embodiments, the target nucleotide sequence comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +58 kb downstream of the BCL11A TSS. In certain embodiments, the target nucleotide sequence comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +62 kb downstream of the BCL11A TSS. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions.

In some embodiments, the target nucleotide sequence comprises at least 15 consecutive nucleotides of the coding region of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence comprises at least 15 consecutive nucleotides of the 5′ untranslated region or 3′ untranslated region surrounding a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence comprises at least 15 consecutive nucleotides of an intronic region or exon region associated with a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, 110, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence comprises at least 15 consecutive nucleotides of a regulatory region associated with a gene selected from the group consisting of HBB, HBG1, HBG2, HBA1, HBA2, HBD, BCL1 JA, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, 114, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence comprises at least 15 consecutive nucleotides of an enhancer region associated with a gene selected from the group consisting of HBB, HBG1, HBG2, HBA1, H1BA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C. In some embodiments, the target nucleotide sequence comprises at least 15 consecutive nucleotides of a repressor region associated with a gene selected from the group consisting of HBB, HBG1, HBG2, HBA1, HBA2, HBD, BCL11A, BACH2, KLF1, LRF, ADA, DCLREIC, IL2RG, RAG1, RAG2, J4K3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF, FANCA, FANCB, FANCC, FANCD1/BRACA2, FANCD2, MPL, CCR5, CXCR4, F5, F2, antithrombin III, and protein C. In some embodiments, the target nucleotide sequence comprises or is within the BCL11A erythroid enhancer. In certain embodiments, the target nucleotide sequence comprises or is within a polynucleotide sequence in intron-2 of the BCL11A gene. In certain embodiments, the target nucleotide sequence comprises or is within a polynucleotide sequence between about +54 kb and about +63 kb downstream of the BCL11A transcription start site (TSS). In certain embodiments, the target nucleotide sequence comprises or is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence comprises or is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions.

In some embodiments, the payload comprises a gRNA or pegRNA having at least 80% identity or complementarity to at least 15 consecutive nucleotides of a polynucleotide comprising the BCL11A erythroid enhancer. In some embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15 consecutive nucleotides of a polynucleotide comprising the BCL11A erythroid enhancer. In some embodiments, the gRNA or pegRNA of the payload comprises a sequence having at least 80% identity or complementarity to at least 15, 16, 17, 18, 19, 20, 21, 22, 23, 24, 25, 26, 27, 28, 29, or 30 consecutive nucleotides of a polynucleotide comprising the BCL11A erythroid enhancer. In some embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15, 16, 17, 18, 19, 20, 21, 22, 23, 24, 25, 26, 27, 28, 29, or 30 consecutive nucleotides of a polynucleotide comprising the BCL11A erythroid enhancer. In some embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15, 16, 17, 18, 19, 20, 21, 22, 23, 24, 25, 26, 27, 28, 29, or 30 consecutive nucleotides of a polynucleotide comprising the BCL11A erythroid enhancer. In certain embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15, 16, 17, 18, 19, 20, 21, 22, 23, 24, 25, 26, 27, 28, 29, or 30 consecutive nucleotides of a polynucleotide sequence in intron-2 of the BCL11A gene. In certain embodiments, the gRNA or pegRNA of the payload comprises a sequence having at least 80%, 85%, 90%, 91%, 92%, 93%, 94%, 95%, 96%, 97%, 98%, or 99/identity or complementarity to at least 15, 16, 17, 18, 19, 20, 21, 22, 23, 24, 25, 26, 27, 28, 29, or 30 consecutive nucleotides of a polynucleotide sequence between about +54 kb and about +63 kb downstream of the BCL11A transcription start site (TSS). In certain embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15, 16, 17, 18, 19, 20, 21, 22, 23, 24, 25, 26, 27, 28, 29, or 30 consecutive nucleotides of a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments, the gRNA or pegRNA of the payload comprises a sequence having at least 80%, 85%, 90%, 91%, 92%, 93%, 94%, 95%, 96%, 97%, 98%, or 99% identity or complementarity to at least 15, 16, 17, 18, 19, 20, 21, 22, 23, 24, 25, 26, 27, 28, 29, or 30 consecutive nucleotides of a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS. In certain embodiments, the gRNA comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions

In certain embodiments, the LNP composition may include one or more other components including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the payload (e.g., nucleic acid, e.g., mRNA) in the resulting LNP composition is from about 1:1 to about 50:1. In certain embodiments, the wt/wt ratio of the lipid component to the payload (e.g., nucleic acid, e.g., mRNA) in the resulting composition is from about 5:1 to about 50:1. In certain embodiments, the wt/wt ratio is from about 5:1 to about 40:1. In certain embodiments, the wt/wt ratio is from about 10:1 to about 40:1. In certain embodiments, the wt/wt ratio is from about 15:1 to about 25:1.

In certain embodiments, the encapsulation efficiency of the payload (e.g., nucleic acid, e.g., mRNA) in the lipid nanoparticles is at least 50%. In certain embodiments, the encapsulation efficiency is at least 80%, at least 90% or, or greater than 90%.

A lipid composition may be designed for one or more specific applications or targets. For example, an LNP composition may be designed to deliver nucleic acids (e.g., an mRNA, a gRNA, and/or a donor template nucleic acid) to a particular cell, tissue, organ, or system or group thereof in a mammal's body. Physiochemical properties of LNP compositions may be altered in order to increase selectivity for particular target site within a subject. For instance, particle sizes may be adjusted based on the fenestration sizes of different organs. The nucleic acids included in an LNP composition may also depend on the desired delivery target or targets. For example, an mRNA, a gRNA, and/or a donor template nucleic acid may be selected for a particular disease and/or for delivery to a particular cell, tissue, organ, or system or group thereof (e.g., localized or specific delivery).

The amount of nucleic acids (e.g., an mRNA, a gRNA, and/or a donor template nucleic acid) in an LNP composition may depend on the size, sequence, and other characteristics of the nucleic acids. The amount of nucleic acids in an LNP may also depend on the size, composition, desired target, and other characteristics of the LNP composition. The relative amounts of nucleic acids and other elements (e.g., lipids) may also vary. The amount nucleic acids in an LNP composition may, for example, be measured using absorption spectroscopy (e.g., ultraviolet-visible spectroscopy).

In some embodiments, the one or more nucleic acids (e.g., an mRNA, a gRNA, and/or a donor template nucleic acid), lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in nucleic acid. In general, a lower N:P ratio is preferred. A N:P ratio may be dependent on a specific lipid and its pKa. In certain embodiments, the nucleic acids (e.g., an mRNA, a gRNA, and/or a donor template nucleic acid) and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

The amount of nucleic acids (e.g., an mRNA, a gRNA, and/or a donor template nucleic acid) in a lipid nanoparticle composition may depend on the size, sequence, and other characteristics of the nucleic acids. The amount of nucleic acids in a lipid nanoparticle composition may also depend on the size, composition, desired target, and other characteristics of the nanoparticle composition. The relative amounts of nucleic acids and other elements (e.g., lipids) may also vary. In some embodiments, the wt/wt ratio of the lipid component to a nucleic acid (e.g., an mRNA, a gRNA, and/or a donor template nucleic acid) in a lipid nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of nucleic acid in a nanoparticle composition may, for example, be measured using absorption spectroscopy (e.g., ultraviolet-visible spectroscopy).

The efficiency of encapsulation of a nucleic acids (e.g., an mRNA, a gRNA, and/or a donor template nucleic acid) describes the amount of the nucleic acid that is encapsulated or otherwise associated with a lipid composition after preparation, relative to the initial amount provided. The encapsulation efficiency is desirably high (e.g., close to ¹⁰⁰%). The encapsulation efficiency may be measured, for example, by comparing the amount of nucleic acid in a solution containing the LNP composition before and after breaking up the LNP composition with one or more organic solvents or detergents. Fluorescence may be used to measure the amount of free nucleic acids in a solution. For the LNP compositions of the invention, the encapsulation efficiency of a nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In certain embodiments, the encapsulation efficiency may be at least 80%.

i. RNA Payload

In certain embodiments, the RNA payload comprises an mRNA, a guide RNA (gRNA), a prime editing guide RNA (pegRNA), a CRISPR-RNA (crRNA), a trans-activating CRISPR RNA (tracrRNA), a tRNA, a microRNA, and/or an siRNA.

In certain embodiments, the lipid nanoparticle compositions are optimized for the delivery of RNA, e.g., an mRNA for translation within the targeted cell (e.g., HSC) and/or a gRNA or pegRNA for complexing with a site-directed nuclease (e.g., CRISPR-associated (Cas) nuclease) within the targeted cell (e.g., HSC). An mRNA may be a naturally or non-naturally occurring mRNA. An mRNA, gRNA or pegRNA may include one or more modified nucleobases, nucleosides, or nucleotides.

The nucleobases may be selected from the non-limiting group consisting of adenine, guanine, uracil, cytosine, 7-methylguanine, 5-methylcytosine, 5-hydroxymethylcytosine, thymine, pseudouracil, dihydrouracil, N1-methylpseudouracil, hypoxanthine, and xanthine. In some embodiments, nucleobase is N1-methylpseudouracil.

A nucleoside of an RNA (e.g., an mRNA or gRNA) is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications.

A nucleotide of an RNA (e.g., an mRNA or gRNA) is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein.

An RNA (e.g., an mRNA or gRNA) may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an RNA (e.g., an mRNA or gRNA) may be 5-methylcytosine. In certain embodiments, one or more or all uridine bases may be N1-methylpseudouridines. An mRNA may include a 5′ untranslated region, a 3′ untranslated region, and/or a coding or translating sequence.

In certain embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or a cap analog. A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analog. A non-limiting list of possible cap species includes m7GpppG, m7Gpppm7G, m73′dGpppG, m7Gpppm7G, m73′dGpppG, and m27 02′GppppG.

Alternatively or in addition, an mRNA may include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

Alternatively or in addition, an mRNA may include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

Alternatively or in addition, an mRNA may include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell. In some embodiments, the mRNA may encode an antibody, enzyme, growth factor, hormone, cytokine, viral protein (e.g., a viral capsid protein), antigen, vaccine, or receptor. In some embodiments, the mRNA may encode one or more polypeptides capable of editing a genomic sequence within the target cells (e.g., within HSCs). Accordingly, in some embodiments, the mRNA encodes one or more polypeptides that function as part of a gene editing system. In certain embodiments, the mRNA encodes a site-directed nuclease, a chemical base editor, a prime editor, or an epigenome editor. In certain embodiments, the LNP comprises an mRNA encoding a CRISPR-associated (Cas) nuclease or a base editor and further comprises a gRNA. In other embodiments, the LNP comprises an mRNA encoding a prime editor and further comprises a prime editor and further comprises pegRNA.

In certain embodiments, a gRNA or pegRNA may comprise one or more chemically modified nucleotides or nucleosides, resulting in increased stability of the gRNA, and increased efficiency and decreased off-target editing of an RNA-guided gene editing system (e.g., a Cas nuclease/gRNA system, a base editor/gRNA system, or a prime editor/pegRNA system). For example, a gRNA may include one or more nucleotides with a 2′-ribose substitution, for example, a 2′-O-methyl substitution or a 2′-fluoro substitution. Additionally, a gRNA may include one or more linkage modifications, such as a phosphorothioate modification, a phosphonoacetate modification, or a thiophosphonoacetate modification. Typically, linkage modifications and 2′-ribose substitutions are combined, for example, 2′-O-methyl substitutions and phosphorothioate linkages, or 2′-O-methyl substitutions and thiophosphonoacetate linkages. In certain embodiments, the gRNA comprises both 2′-O-methyl substitutions and phosphorothioate linkages. In some instances, a gRNA may additionally or alternatively include modifications that create intramolecular linkages within the sugar moiety of the nucleotide, for example, locked nucleic acids (LNAs) and bridged nucleic acids (BNAs) with linkages between the 2′ oxygen and the 4′ carbon of ribose. LNAs and BNAs may be incorporated, for example, in the 20 nucleotide guide sequence of a gRNA. A gRNA may also include one or more DNA nucleotides. Such modifications can be incorporated at any nucleotide or linkage within the gRNA, for example, at the 5′ terminal residue(s) or the 3′ terminal residue(s) of the gRNA. A gRNA may comprise a modification described herein at one, two, three, four, five, ten or more 5′ terminal nucleotides and/or 3′ terminal nucleotides of a gRNA.

Additional modifications of RNAs, including mRNAs and gRNAs, are known in the art and are described, for example, in Chen et al. (“Recent advances in chemical modifications of guide RNA, mRNA and donor template for CRISPR-mediated genome editing.” Advanced Drug Delivery Reviews 168 (2021): 246-258) and in Qui et al. (“Lipid nanoparticle-mediated codelivery of Cas9 mRNA and single-guide RNA achieves liver-specific in vivo genome editing of Angptl3” Proc Natl Acad Sci, 2021; 118(10):e2020401118).

In addition to the lipids present in the LNP or in the lipid blend, the LNP compositions may further comprise a payload, for example, a payload described herein. In some embodiments, the payload is a nucleic acid, for example, DNA or RNA, for example, an mRNA, transfer RNA (tRNA), a microRNA, or small interfering RNA (siRNA). In certain embodiments, the payload is an mRNA, for example, an mRNA encoding a site-directed nuclease, a chemical base editor, a prime editor, or an epigenome editor as described herein.

In certain embodiments, the number of the nucleotides in the nucleic acid is from about 400 to about 6000.

### (g) Physical Properties of Lipid Nanoparticles

The characteristics of an LNP composition may depend on the components, their absolute or relative amounts, contained in a lipid nanoparticle (LNP) composition. Characteristics may also vary depending on the method and conditions of preparation of the LNP composition.

Physiochemical properties of LNP compositions may be altered in order to increase selectivity for particular target site within a subject. For instance, particle sizes may be adjusted based on the fenestration sizes of different organs. The mRNA RNA (e.g., mRNA and/or gRNA) included in an LNP composition may also depend on the desired delivery target or targets. For example, an mRNA and/or gRNA may be selected for a particular disease and/or for delivery to a particular cell, tissue, organ, or system or group thereof (e.g., localized or specific delivery).

The amount of RNA (e.g., mRNA and/or gRNA) mRNA in an LNP composition may depend on the size, sequence, and other characteristics of the mRNA. The amount of RNA (e.g., mRNA and/or gRNA) mRNA in an LNP may also depend on the size, composition, desired target, and other characteristics of the LNP composition. The relative amounts of RNA (e.g., mRNA and/or gRNA) mRNA and other elements (e.g., lipids) may also vary. The amount of RNA (e.g., mRNA and/or gRNA) mRNA in an LNP composition may, for example, be measured using absorption spectroscopy (e.g., ultraviolet-visible spectroscopy).

In some embodiments, the one or more mRNAs, lipids, and polymers and amounts thereof may be selected to provide a specific N:P ratio (the ratio of positively-chargeable lipid or polymer amine (N=nitrogen) groups to negatively-charged nucleic acid phosphate (P) groups). The N:P ratio of the composition refers to the molar ratio of nitrogen atoms in one or more lipids to the number of phosphate groups in an mRNA. In general, a lower N:P ratio is preferred. A N:P ratio may be dependent on a specific lipid and its pKa. In certain embodiments, the mRNA and LNP composition, and/or their relative amounts may be selected to provide an N:P ratio from about 1:1 to about 30:1, or from about 1:1 to about 20:1. In certain embodiments, the N:P ratio can be, for example, 1:1, 2:1, 3:1, 4:1, 5:1, 6:1, 7:1, or 8:1. In certain embodiments, the N:P ratio may be from about 2:1 to about 5:1. In certain embodiments, the N:P ratio may be about 4:1. In other embodiments, the N:P ratio is from about 4:1 to about 8:1. For example, the N:P ratio may be about 4:1, about 4.5:1, about 4.6:1, about 4.7:1, about 4.8:1, about 4.9:1, about 5.0:1, about 5.1:1, about 5.2:1, about 5.3:1, about 5.4:1, about 5.5:1, about 5.6:1, about 5.7:1, about 6.0:1, about 6.5:1, or about 7.0:1.

LNP compositions may be characterized by a variety of methods. For example, microscopy (e.g., transmission electron microscopy or scanning electron microscopy) may be used to examine the morphology and size distribution of an LNP composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of an LNP composition, such as particle size, polydispersity index, and zeta potential. RNA encapsulated efficiency is determined by a combination of methods relying on RNA binding dyes (ribogreen, cybergreen to determine dye accessible RNA fraction) and LNP de-formulation followed by HPLC analysis for total RNA content.

In some embodiments, the LNP may have a mean diameter in the range of 1-250 nm, 1-200 nm, 1-150 nm, 1-100 nm, 50-250 nm, 50-200 nm, 50-150 nm, 50-100 nm, 75-250 nm, 75-200 nm, 75-150 nm, 75-100 nm, 100-250 nm, 100-200 nm, 100-150 nm. In certain embodiments, the LNP compositions may have a mean diameter of about 1 nm, about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm. In some embodiments, the LNP has a mean diameter of about 100 nm.

Alternatively or in addition, the LNP compositions may have a polydispersity index in a range from 0.05-1, 0.05-0.75, 0.05-0.5, 0.05-0.4, 0.05-0.3, 0.05-0.2, 0.08-1, 0.08-0.75, 0.08-0.5, 0.08-0.4, 0.08-0.3, 0.08-0.2, 0.1-1, 0.1-0.75, 0.1-0.5, 0.1-0.4, 0.1-0.3, 0.1-0.2. In certain embodiments, the polydispersity index is in the range of 0.1-0.25, 0.1-0.2, 0.1-0.19, 0.1-0.18, 0.1-0.17, 0.1-0.16, or 0.1-0.15.

Alternatively or in addition, the LNP compositions may have a zeta potential of about −30 mV to about +30 mV. In certain embodiments, the LNP composition has a zeta potential of about −10 mV to about +20 mV. The zeta potential may vary as a function of pH. As a result, in certain embodiments, the LNP compositions may have a zeta potential of about 0 mV to about +30 mV or about +10 mV to +30 mV or about +20 mV to about +30 mV at pH 5.5 or pH 5, and/or a zeta potential of about −30 mV to about +5 mV or about −20 mV to about +15 mV at pH 7.4.

In some embodiments, the LNP provided herein comprises an ionizable cationic lipid and one or more of a sterol, a neutral phospholipid, a PEG-lipid, and a lipid-HSC targeting group conjugate (e.g., lipid-antibody conjugate). In some embodiments, the LNP comprises Lipid 15, a HSC targeting group that binds to a HSC surface antigen comprising a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:7, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the LNP comprises Lipid 15, a HSC targeting group that binds to a HSC surface antigen comprising a VH domain comprising CDR-H1, CDR-H2, and CDR-H3 sequences, where the VH domain comprises the amino acid sequence set forth in SEQ ID NO:7, and a VL domain comprising CDR-L1, CDR-L2, and CDR-L3 sequences, where the VL domain comprises the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the exemplary LNP provided herein is delivered to a subject with disease for in vivo gene editing and treatment of the disease. In some embodiments, the exemplary LNP provided herein is delivered to a subject with sickle cell disease or beta-thalessemia for in vivo gene editing and treatment of the subject. In some embodiments, use of the exemplary LNP provided herein for treatment of sickle cell disease in a subject is safe and effective. In some embodiments, use of the exemplary LNP provided herein for treatment of beta-thalessemia in a subject is safe and effective.

### III. Method of Producing Lipid Nanoparticles

In some embodiments, the LNPs are produced by using either rapid mixing by an orbital vortexer or by microfluidic mixing. Orbital vortexer mixing is accomplished by rapid addition of lipids solution in ethanol to the aqueous solution of a nucleic acid of interest followed immediately by vortexing at 2,500 rpm. In some embodiments, the LNPs are produced using a microfluidic mixing step. In some embodiments, microfluidic mixing is achieved mixing the aqueous and organic streams at a controlled flow rates in a microfluidic channel using, e.g., a NanoAssemblr device and microfluidic chips featuring optimized mixing chamber geometry (Precision Nanosystems, Vancouver, BC). In some embodiments, the LNPs are produced using a microfluidic mixing step to rapidly mix the ethanolic lipid solution and aqueous nucleic acid solution, resulting in encapsulation of the nucleic acid in the solid lipid nanoparticles. The nanoparticle suspension is then buffer exchanged into an all aqueous buffer using membrane filtration device of choice for ethanol removal and nanoparticle maturation.

In certain embodiments, the resulting LNP compositions comprise a lipid blend containing, for example, from about 40 mole percent to about 60 mole percent of one or more ionizable cationic lipids described herein, from about 35 mole percent to about 50 mole percent of one or more sterols, from about 5 mole percent to about 15 mole percent of one or more neutral lipids, and from about 0.5 mole percent to about 5 mole percent of one or more PEG-lipids.

### V. Formulation and Mode of Delivery

LNP compositions of the invention may be formulated in whole or in part as a pharmaceutical composition. The pharmaceutical compositions may further include one or more pharmaceutically acceptable excipients or accessory ingredients such as those described herein. General guidelines for the formulation and manufacture of pharmaceutical compositions and agents are available, for example, in Remington's (2006) supra. Conventional excipients and accessory ingredients may be used in any pharmaceutical composition of the invention, except insofar as any conventional excipient or accessory ingredient may be incompatible with one or more components of an LNP composition of the invention. An excipient or accessory ingredient may be incompatible with a component of an LNP composition if its combination with the component may result in any undesirable biological effect or otherwise deleterious effect.

In some embodiments, one or more excipients or accessory ingredients may make up greater than 50% of the total mass or volume of a pharmaceutical composition including an LNP composition of the invention. For example, the one or more excipients or accessory ingredients may make up 30%, 40%, 50%, 60%, 70%, 80%, 90%, or more of a pharmaceutical composition. In certain embodiments, the excipient is approved for use in humans and for veterinary use, for example, by United States Food and Drug Administration. In certain embodiments, the excipient is pharmaceutical grade. In certain embodiments, an excipient meets the standards of the United States Pharmacopoeia (USP), the European Pharmacopoeia (EP), the British Pharmacopoeia, and/or the International Pharmacopoeia.

Relative amounts of the one or more lipids or LNPs, one or more pharmaceutically acceptable excipients, and/or any additional ingredients in a pharmaceutical composition will vary, depending upon the identity, size, and/or condition of the subject treated and further depending upon the route by which the composition is to be administered.

Lipid compositions and/or pharmaceutical compositions including one or more LNP compositions may be administered to any subject, including a human patient that may benefit from a therapeutic effect provided by the delivery of a nucleic acid, e.g., an RNA (e.g., mRNA, gRNA, tRNA or siRNA) to one or more particular cells, tissues, organs, or systems or groups thereof, such as the renal system. Although the descriptions provided herein of LNP compositions and pharmaceutical compositions including LNP compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is understood.

A pharmaceutical composition in accordance with the present disclosure may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient (e.g., the payload).

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

### (a) Other Components

In addition, it is contemplated that the pharmaceutical compositions may include one or more components in addition to those described herein.

The pharmaceutical compositions may also include one or more permeability enhancer molecules, carbohydrates, polymers, therapeutic agents, surface altering agents, or other components. A permeability enhancer molecule may be a molecule described, for example, in U.S. patent application publication No. 2005/0222064. Carbohydrates may include simple sugars (e.g., glucose) and polysaccharides (e.g., glycogen and derivatives and analogs thereof).

The pharmaceutical compositions may also contain a surface altering agent, including for example, anionic proteins (e.g., bovine serum albumin), surfactants (e.g., cationic surfactants such as dimethyldioctadecyl-ammonium bromide), sugars or sugar derivatives (e.g., cyclodextrin), polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 04, dornase alfa, neltenexine, and erdosteine), and Dnases (e.g., rhDNase). A surface altering agent may be disposed within and/or upon the surface of a composition described herein.

In addition to these components, a pharmaceutical composition containing an LNP composition of the invention may include any substance useful in pharmaceutical compositions. For example, the pharmaceutical composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see, e.g., Remington's (2006) supra).

Dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and VEEGUM® [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN® 20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ 45], polyoxyethylene hydrogenated castor oil, polyethoxylated castor oil, polyoxymethylene stearate, and SOLUTOL®), sucrose fatty acid esters, polyethylene glycol fatty acid esters (e.g., CREMOPHOR®), polyoxyethylene ethers, (e.g., polyoxyethylene lauryl ether [BRU® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g., HEPES), magnesium hydroxide, aluminum hydroxide, alginic acid, pyrogen-free water, isotonic saline, Ringer's solution, ethyl alcohol, and/or combinations thereof.

In certain embodiments, the lipid nanoparticle compositions and formulations thereof are adapted for administration intravenously, intramuscularly, intradermally, subcutaneously, intraosseous infusion, intra-arterially, intra-tumor, or by inhalation. In certain embodiments, a dose of about 0.001 mg/kg to about 10 mg/kg is administered to a subject. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

The specific therapeutically effective, prophylactically effective, or otherwise appropriate dose level (e.g., for imaging) for any particular patient will depend upon a variety of factors including the severity and identify of a disease being treated, if any; the one or more nucleic acids (e.g., mRNAs, gRNAs, and/or donor template nucleic acids) employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

### VI. Methods of Delivering Nucleic Acids to Hematopoietic Stem Cells

The present disclosure provides methods of delivering a payload to a target cell or tissue, for example, a target cell or tissue in a subject, and LNPs or pharmaceutical compositions containing the LNPs for use in such methods. Any disclosure herein of a method of, e.g., delivering a nucleic acid to a cell or e.g., expressing a polypeptide of interest in a cell should be interpreted also as a disclosure of an LNP or pharmaceutical composition comprising said LNP for use in such methods.

In some aspects, provided herein is a method of delivering nucleic acids to hematopoietic stem cells (HSC). In certain embodiments, the of the method comprises producing a polypeptide of interest (e.g., a protein of interest, e.g., a site-directed nuclease, a chemical base editor, a prime editor, or an epigenome editor) in a mammalian HSC, and LNPs or pharmaceutical compositions containing the LNPs for use in such methods. Methods of producing polypeptides in HSCs involve contacting one or more HSCs with an LNP composition comprising an mRNA of interest (e.g., an mRNA encoding a site-directed nuclease, a chemical base editor, a prime editor, or an epigenome editor and, optionally, a gRNA or a pegRNA). Upon contacting the HSC with the LNP composition, the mRNA may be taken up and translated in the cell to produce the polypeptide of interest.

In general, the step of contacting a mammalian HSC with an LNP composition including an mRNA encoding a polypeptide of interest may be performed in vivo, ex vivo, or in vitro. The amount of an LNP composition contacted with a cell, and/or the amount of nucleic acid (e.g., mRNA) therein, may depend on the type of HSC or tissue being contacted, the means of administration, the physiochemical characteristics of the LNP composition and the mRNA (e.g., size, charge, and chemical composition) therein, and other factors. In general, an effective amount of the LNP composition will allow for efficient polypeptide production in the HSC. Metrics for efficiency may include polypeptide translation (indicated by polypeptide expression), level of mRNA degradation, and immune response indicators.

The step of contacting an LNP composition including an mRNA with a cell may involve or cause transfection where the LNP composition may fuse with the membrane of cell to permit the delivery of the mRNA into the cell. Upon introduction into the cytoplasm of the cell, the mRNA is then translated into a protein or peptide via the protein synthesis machinery within the cytoplasm of the cell.

The present disclosure provides methods of delivering a nucleic acid (e.g., an mRNA) to a mammalian HSC or tissue, for example, a mammalian HSC or tissue in a subject. Delivery of an nucleic acid (e.g., an mRNA) to such a cell or tissue involves administering an LNP composition including the nucleic acid (e.g., an mRNA) to a subject, for example, by injection, e.g., via intramuscular injection or intravascular delivery into the subject. After administration, the LNP can target and/or contact a HSC. Upon contacting the HSC with the LNP composition, a translatable mRNA may be translated in the cell to produce a polypeptide of interest (e.g., a polypeptide of a gene editing system).

In certain embodiments, an LNP composition of the invention may target a particular type or class of cells, e.g., HSCs. This targeting may be facilitated using the lipids described herein to form LNPs, which may also include a targeting group for targeting cells of interest. In certain, embodiments, specific delivery may result in a greater than 2 fold, 5 fold, 10 fold, 15 fold, or 20 fold increase in the amount of nucleic acid (e.g., mRNA) to the targeted destination (e.g., HSCs that express at high levels certain surface antigens (e.g., CD105 and/or CD117) which bind to the antibody-lipid conjugate of the LNPs) as compared to another destinations (e.g., cells that either do not express or only express at low levels said surface antigens).

In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of cells that are not meant to be the destination of the delivery are transfected by the LNP. In some embodiments, the cells that are not meant to be the destination of the delivery are any cells except hematopoietic stem cells. In some embodiments, no more than 1%, no more than 2%, no more than 3%, no more than 4%, no more than 5%, no more than 6%, no more than 7%, no more than 8%, no more than 9%, no more than 10%, no more than 15%, no more than 20%, no more than 25%, no more than 30%, no more than 35%, no more than 40%, no more than 45%, or no more than 50% of non-HSC cells that are not meant to be the destination of the delivery are transfected by the LNP. In some embodiments, the cells that are not meant to be the destination of the delivery are cells not targeted by the method. In some embodiments, the cells that are not meant to be the destination of the delivery are subject's cells not targeted by the method.

In some embodiments, the half-life of the nucleic acid delivered by the LNP described herein to the HSC or a polypeptide encoded by the nucleic acid delivered by the LNP and expressed in the HSC is at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, at least 1.5 times, at least 2 times, at least 3 times, at least 4 times, at least 5 times, or at least 10 times longer than the half-life of the nucleic acid delivered by a reference LNP to the HSCs or a polypeptide encoded by the nucleic acid delivered by the reference LNP and expressed in the HSC.

In some embodiments, the composition of the LNP differs from the composition of the reference LNP in the type of ionizable cationic lipid, relative amount of ionizable cationic lipid, length of the lipid anchor in PEG lipid, back bone or head group of the PEG lipid, relative amount of PEG lipid, or type of HSC targeting group (e.g., type of antibody that binds to CD105 and/or CD117), or any combination thereof. In some embodiments, the composition of the LNP differs from the composition of the reference LNP only in the type of ionizable cationic lipid. In some embodiments, the composition of the LNP differs from the composition of the reference LNP only in the amount of PEG lipid. In some embodiments, the reference LNP comprises cationic Lipid Dlin-KC3-DMA, but otherwise is the same as a tested LNP. In some embodiments, the reference LNP comprises cationic Lipid Dlin-KC2-DMA, but otherwise is the same as a tested LNP. In some embodiments, the reference LNP comprises cationic Lipid ALC-0315, but otherwise is the same as a tested LNP. In some embodiments, the reference LNP comprises cationic Lipid SM-102, but otherwise is the same as a tested LNP. In some embodiments, PEG lipid is a free PEG lipid.

In some embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more of the HSCs are transfected by the LNP. Embodiments, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% or more of the HSCs that are meant to be the destination of the delivery are transfected by the LNP. In some embodiments, the HSCs are a subject's HSCs. In some embodiments, the HSCs are HSCs targeted by the method (e.g., a subpopulation of HSCs targeted by the method). In some embodiments, the HSCs are a subject's HSCs targeted by the method (e.g., a subpopulation of the subject's HSCs targeted by the method).

In some embodiments, the expression level of the nucleic acid delivered by the LNP is at least at least 1%, at least 5%, at least 10%, at least 15%, at least 20%, at least 25%, at least 30%, at least 35%, at least 40%, at least 45%, at least 50%, at least 60%, at least 70%, at least 80%, at least 90%, at least 1.5 times, at least 2 times, at least 3 times, at least 4 times, at least 5 times, at least 6 times, at least 7 times, at least 8 times, at least 9 times, at least 10 times, at least 15 times, or at least 20 times higher than the expression level of the nucleic acid in the same HSCs delivered by a reference LNP. In some embodiments, the expression level is measured and compared with a method described herein. In some embodiments, the expression level is measured by the ratio of HSCs (e.g., transfected HSCs) expressing the encoded polypeptide. In some embodiments, the expression level is measured with FACS. In some embodiments, the expression level is measured by the average amount of the encoded polypeptide expressed in the HSCs. In some embodiments, the expression level is measured as mean fluorescence intensity. In some embodiments, the expression level is measured by the amount of the encoded polypeptide or other materials secreted by the HSCs.

In another aspect, provided herein are methods of targeting the delivery of a nucleic acid to a hematopoietic stem cell (HSC) of a subject. In some embodiments, the method comprises contacting the HSC with a lipid nanoparticle (LNP). In some embodiments, the LNP comprises an ionizable cationic lipid. In some embodiments, the LNP comprises a conjugate comprising the compound of the following formula: [Lipid]-[optional linker]-[HSC targeting group]. In certain embodiments, the LNP comprises a lipid-antibody conjugate comprising the compound of the following formula: [Lipid]-[optional linker]-[antibody] wherein the antibody binds to CD105 and/or CD117. In some embodiments, the antibody that binds to CD117 comprises the amino acid sequences of Ab1 as described in Table B. In some embodiments, the antibody that binds to CD117 comprises the amino acid sequences of Ab2 as described in Table B. In some embodiments, the antibody that binds to CD105 comprises the amino acid sequences of Ab3 as described in Table B. In some embodiments, the antibody that binds to CD117 is Ab1. In some embodiments, the antibody that binds to CD117 is Ab2. In some embodiments, the antibody that binds to CD105 is Ab3. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises one or more nucleic acid. In some embodiments, the LNP comprises one or more nucleic acid encoding a gene editing system targeting a locus (or loci) wherein gene editing results in increased HbF for the treatment of a disease (e.g., sickle cell disease and beta-thalessemia).

In some embodiments, an aspect of the disclosure relates to an LNP or a pharmaceutical composition containing thereof, as disclosed herein, for use in a method of targeting the delivery of a nucleic acid to a hematopoietic stem cell (HSC) of a subject. Such a method may be for the treatment of a disease as disclosed hereafter. In some embodiments, a method as disclosed herein can comprise contacting in vitro or ex vivo the HSC of a subject with a lipid nanoparticle (LNP). In a preferred embodiment, a method as disclosed herein can comprise contacting in vivo the HSC of a subject with a lipid nanoparticle (LNP). In some embodiments, the LNP is an LNP as described herein in the present disclosure.

In some embodiments, the LNP provides at least one of the following benefits:
(i) increased specificity of targeted delivery to HSCs compared to a reference LNP;
(ii) increased half-life of the nucleic acid or a polypeptide encoded by the nucleic acid in the HSC compared to a reference LNP;
(iii) increased transfection rate compared to a reference LNP; and
(iv) a low level of dye accessible nucleic acid (e.g., mRNA and/or gRNA; <15%) and high nucleic acid (e.g., mRNA and/or gRNA) encapsulation efficiencies, wherein at least 80% nucleic acid (e.g., mRNA and/or gRNA) was recovered in final formulation relative to the total nucleic acid (e.g., mRNA and/or gRNA) used in LNP batch preparation.

In some aspect, provided are methods of expressing a polypeptide of interest in a targeted HSC of a subject. In some embodiments, the method comprises contacting the HSC with a lipid nanoparticle (LNP). In some embodiments, the LNP comprises an ionizable cationic lipid. In some embodiments, the LNP comprises a conjugate comprising the following structure: [Lipid]-[optional linker]-[HSC targeting group]. In certain embodiments, the LNP comprises a lipid-antibody conjugate comprising the compound of the following formula: [Lipid]-[optional linker]-[antibody], wherein the antibody binds to CD105 and/or CD117. In some embodiments, the antibody that binds to CD117 comprises the amino acid sequences of Ab1 as described in Table B. In some embodiments, the antibody that binds to CD117 comprises the amino acid sequences of Ab2 as described in Table B. In some embodiments, the antibody that binds to CD105 comprises the amino acid sequences of Ab3 as described in Table B. In some embodiments, the antibody that binds to CD117 is Ab1. In some embodiments, the antibody that binds to CD117 is Ab2. In some embodiments, the antibody that binds to CD105 is Ab3. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding the polypeptide. In some embodiments, an aspect of the disclosure relates to an LNP or a pharmaceutical composition containing thereof, as disclosed herein, for use in a method of expressing a polypeptide of interest in a targeted HSC of a subject. Such a method may be for the treatment of a disease as disclosed hereafter. In some embodiments, a method as disclosed herein can comprise contacting in vitro or ex vivo the HSC of a subject with a lipid nanoparticle (LNP). In a preferred embodiment, a method as disclosed herein can comprise contacting in vivo the HSC of a subject with a lipid nanoparticle (LNP).

In some embodiments, the LNP provides at least one of the following benefits:
(i) increased expression level in the HSC compared to a reference LNP;
(ii) increased specificity of expression in the HSC compared to a reference LNP;
(iii) increased half-life of the nucleic acid or a polypeptide encoded by the nucleic acid in the HSC compared to a reference LNP;
(iv) increased transfection rate compared to a reference LNP; and
(v) a low level of dye accessible nucleic acid (e.g., mRNA and/or gRNA; <15%) and high nucleic acid (e.g., mRNA and/or gRNA) encapsulation efficiencies, wherein at least 80% nucleic acid (e.g., mRNA and/or gRNA) was recovered in final formulation relative to the total nucleic acid (e.g., mRNA and/or gRNA) used in LNP batch preparation. LNPs disclosed in the present disclosure and as claimed are suitable for the methods described above.

In some embodiments, the LNP delivered in the methods provided herein comprises Lipid 15, a HSC targeting group that binds to a HSC surface antigen comprising a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:7, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within The BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the LNP delivered in the methods provided herein comprises Lipid 15, a HSC targeting group that binds to a HSC surface antigen comprising a VH domain comprising CDR-H1, CDR-H2, and CDR-H3 sequences, where the VH domain comprises the amino acid sequence set forth in SEQ ID NO:7, and a VL domain comprising CDR-L1, CDR-L2, and CDR-L3 sequences, where the VL domain comprises the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, delivery of the exemplary LNP provided herein is used to edit HSC cells in vitro, ex vivo, and in vivo. In some embodiments, the exemplary LNP provided herein is used to edit HSC cells in vivo. In some embodiments, the exemplary LNP provided herein is delivered to a subject with disease for in vivo gene editing and treatment of the disease. In some embodiments, the exemplary LNP provided herein is delivered to a subject with sickle cell disease or beta-thalessemia for in vivo gene editing and treatment of the subject. In some embodiments, use of the exemplary LNP provided herein for treatment of sickle cell disease in a subject is safe and effective. In some embodiments, use of the exemplary LNP provided herein for treatment of beta-thalessemia in a subject is safe and effective.

### VII. Methods of Gene Editing in Hematopoietic Stem Cells

The present disclosure provides methods of delivering a payload encoding a gene editing system (e.g., a site-directed nuclease and, optionally, a guide RNA) to a target cell or tissue, for example, a target cell or tissue in a subject, and LNPs or pharmaceutical compositions containing the LNPs for use in such methods. The present disclosure further provides methods of genetically modifying hematopoietic stem cells (HSCs), both in vitro and in vivo in a subject. Any disclosure herein of a method of, e.g., treating a disease or, e.g., delivering a nucleic acid to a cell e.g., expressing a gene editing system in a cell or, e.g., genetically modifying a cell should be interpreted also as a disclosure of an LNP or pharmaceutical composition comprising said LNP for use in such methods.

### (a) Gene Editing Systems and Methods

In some embodiments, the LNPs disclosed herein may comprise one or more nucleic acids encoding components of gene editing systems. Gene editing systems are designed to specifically recognize a target nucleic acid sequence in a DNA molecule and thereby induce a modification in the DNA molecule. The modification may comprise a modification in the nucleotide sequence of the DNA molecule, or may comprise a chemical modification of one or more nucleotides in the DNA molecule (e.g., methylation). Gene editing systems useful in the methods disclosed herein include, for example, site-directed nuclease gene editing systems, chemical base editors, prime editors, and epigenome editors.

In particular embodiments, the methods disclosed herein utilize LNPs comprising one or more nucleic acids encoding components of site-directed nuclease gene editing systems, e.g., an mRNA encoding a site-directed nuclease. Site-directed nucleases may generate one or more single-stranded DNA nicks or double-stranded DNA breaks (DSB) in a target nucleotide sequence. In some instances, a DSB can be achieved in a DNA molecule comprising the target nucleotide sequence by the use of two nucleases generating single-stranded nicks (nickases). Each nickase can cleave one strand of the DNA, and the use of two or more nickases can create a DSB (e.g., a staggered DSB) in a target nucleotide sequence. In preferred embodiments, the site-directed nucleases are used in combination with a donor template nucleic acid, which is introduced into the target nucleotide sequence at the site of the DNA DSB via homologous recombination.

In some embodiments, the LNPs disclosed herein may comprise an mRNA encoding a site-directed nuclease. In the methods disclosed herein, site-directed nucleases may generate one or more single-stranded DNA nicks or double-stranded DNA breaks (DSB) in a target nucleotide sequence. In some instances, a DSB can be achieved in a DNA molecule comprising the target nucleotide sequence by the use of two nucleases generating single-stranded nicks (nickases). Each nickase can cleave one strand of the DNA, and the use of two or more nickases can create a DSB (e.g., a staggered DSB) in a target nucleotide sequence. In preferred embodiments, the site-directed nucleases are used in combination with a donor template nucleic acid, which is introduced into the target nucleotide sequence at the site of the DNA DSB via homologous recombination.

Site-directed nucleases may comprise one or more DNA binding domains and one or more DNA cleavage domains (e.g., one or more endonuclease and/or exonuclease domains), and optionally, one or more polypeptide linkers. The site-directed nuclease may be designed and/or modified from a naturally occurring site-directed nuclease or from a previously engineered site-directed nuclease. Engineered site-directed nucleases may further comprise one or more additional functional domains, e.g., an end-processing enzymatic domain of an end-processing enzyme that exhibits 3-5′ exonuclease (e.g., Trex2), 5-3′ alkaline exonuclease, 5-3′ exonuclease, 5′ flap endonuclease, helicase, or template-independent DNA polymerase activity.

The LNPs described herein may comprise an mRNA encoding any known site-directed nuclease including, for example, clustered regularly-interspaced short palindromic repeats (CRISPR)-associated (Cas) nucleases, zinc finger nucleases (ZFNs), transcription activator-like effector nucleases (TALENs), megaTALs, and homing endonucleases (meganucleases). In some instances, a site-directed nuclease is an RNA guided nuclease and requires an RNA sequence to target the nuclease to a target site (e.g., CRISPR/Cas). In other instances, site-directed nucleases comprise one or more heterologous DNA-binding and cleavage domains (e.g., ZFNs, TALENs, megaTALs). In yet other instances, the DNA-binding domain of a naturally-occurring nuclease may be altered to bind to a selected target site (e.g., a meganuclease that has been engineered to bind to site different than the cognate binding site).

i. CRISPR/Cas Gene Editing Systems

In some embodiments, the site-directed nuclease is a Cas nuclease. CRISPR (Clustered Regularly Interspaced Short Palindromic Repeats)/Cas (CRISPR Associated) nuclease systems can be introduced into a cell and engineered to bind to, and to introduce single-stranded nicks or double-strand breaks (DSBs), into a target nucleotide sequence. CRISPR/Cas gene editing systems are based on a natural bacterial system that has been utilized for mammalian genome engineering. CRISPR-Cas systems are known in the art and described in, for example, Jinek, (“A programmable dual-RNA-guided DNA endonuclease in adaptive bacterial immunity.” Science 337.6096 (2012): 816-821); Jinek (“RNA-programmed genome editing in human cells.” Elife 2 (2013): e00471); Mali (“RNA-guided human genome engineering via Cas9.” Science 339.6121 (2013): 823-826); Qi (“Repurposing CRISPR as an RNA-guided platform for sequence-specific control of gene expression.” Cell 152.5 (2013): 1173-1183); Ran (“Genome engineering using the CRISPR-Cas9 system.” Nature protocols 8.11 (2013): 2281-2308); Zetsche (“Cpf1 is a single RNA-guided endonuclease of a class 2 CRISPR-Cas system.” Cell 163.3 (2015): 759-771.).

In some embodiments, the LNP comprises an mRNA encoding a Cas nuclease and one or more RNAs that confer binding of the Cas nuclease to the target nucleotide sequence, for example, a transactivating cRNA (tracrRNA) and a CRISPR RNA (crRNA), or, more commonly, guide RNA (gRNA, also referred to as a single guide RNA (sgRNA)), in which crRNA and tracrRNA are engineered into one RNA molecule.

In some instances, the Cas nuclease is engineered as a double-stranded DNA endonuclease, a nickase, or a catalytically dead Cas (dCas), and forms a target complex with a gRNA or crRNA/tracrRNA for site specific DNA recognition at the target nucleotide sequence. gRNAs and cRNAs comprise a protospacer sequence that shares homology/complementarity with the protospacer target sequence of the target nucleotide sequence. The protospacer confers binding of the Cas/gRNA complex to the target nucleotide sequence. The protospacer target sequence abuts a short protospacer adjacent motif (PAM), which plays a role in recruiting a Cas/RNA complex to the target site. Different types of Cas nucleases recognize different specific PAM motifs. A CRISPR/Cas system can be used to target and cleave target nucleotide sequence flanked by particular 3′ PAM sequences specific to the particular Cas nuclease of the CRISPR/Cas system. PAMs for specific Cas nucleases are known in the art and may be also identified using bioinformatics or experimental methods described in the art, including, for example, in Esvelt (“Orthogonal Cas9 proteins for RNA-guided gene regulation and editing.” Nature methods 10.11 (2013): 1116-1121).

In certain embodiments, the Cas nuclease may comprise one or more heterologous DNA binding domains, which may increase the DNA cleavage efficiency and specificity at the target nucleotide sequence. A Cas nuclease may optionally comprise one or more linkers and/or additional functional domains, e.g., an end-processing enzymatic domain of an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. In some embodiments, a Cas nuclease can be introduced into a hematopoietic stem cell (HSC) with an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. The Cas nuclease and 3′ processing enzyme may be introduced separately, e.g., in different vectors or separate nucleic acids, or together, e.g., as a fusion protein, or in a polycistronic construct separated by a viral self-cleaving peptide or an IRES element.

In various embodiments, the Cas nuclease is Cas9 or Cpf1.

A Cas9 nuclease suitable for use in particular embodiments may be obtained, for example, from bacterial species including, but not limited to: Enterococcus faecium, Enterococcus italicus, Listeria innocua, Listeria monocytogenes, Listeria seeligeri, Listeria ivanovii, Streptococcus agalactiae, Streptococcus anginosus, Streptococcus bovis, Streptococcus dvsgalactiae, Streptococcus equinus, Streptococcus galolvticus, Streptococcus macacae, Streptococcus mutans, Streptococcus pseudoporcinus, Streptococcus pyogenes. Streptococcus thermophilus, Streptococcus gordonin, Streptococcus infantarius, Streptococcus macedonicus, Streptococcus mitis, Streptococcus pasteurianus, Streptococcus suis, Streptococcus vestibularis, Streptococcus sanguinis, Streptococcus downei, Neisseria bacilliformis. Neisseria cinerea, Neisseria flavescens, Neisseria lactamica, Neisseria meningitidis, Neisseria subflava, Lactobacillus brevis, Lactobacillus buchneri, Lactobacillus casei, Lactobacillus paracasei, Lactobacillus fermentum, Lactobacillus gasseri, Lactobacillus jensenii, Lactobacillus johnsonii, Lactobacillus rhamnosus, Lactobacillus ruminis, Lactobacillus salivarius, Lactobacillus sanfranciscensis, Corynebacterium accolens, Corynebacterium diphtheriae. Corynebacterium matruchotii, Campylobacter jejuni, Clostridium perfringens, Treponema vincentii, Treponema phagedenis, and Treponema denticola. In some instances, the nucleotide encoding the Cas9 nuclease comprises a portion of the Cas9 nuclease sequence from any one of the bacterial species described herein.

Likewise, a Cpf1 nuclease suitable for use in particular embodiments may be obtained from bacterial species including, but not limited to: Francisella spp., Acidaminococcus spp., Prevolella spp., Lachnospiraceae spp., among others. In some instances, the nucleotide encoding the Cpf1 nuclease comprises a portion of the Cas9 nuclease sequence from any one of the bacterial species described herein.

Conserved regions of Cas9 orthologs include a central HNH endonuclease domain and a split RuvC/Rnase H domain. Cpf1 orthologs possess a RuvC/Rnase H domain but no discernable HNH domain. The HNH and RuvC-like domains are each responsible for cleaving one strand of the double-stranded DNA target sequence. The HNH domain of the Cas9 nuclease cleaves the DNA strand complementary to the tracrRNA:crRNA or sgRNA. The RuvC-like domain of the Cas9 nuclease cleaves the DNA strand that is not-complementary to the tracrRNA:crRNA or sgRNA. Cpf1 is predicted to act as a dimer wherein each RuvC-like domain of Cpf1 cleaves either the complementary or non-complementary strand of the target site. In particular embodiments, a Cas9 nuclease variant (e.g., Cas9 nickase) is contemplated comprising one or more amino acids additions, deletions, mutations, or substitutions in the HNH or RuvC-like endonuclease domains that decreases or eliminates the nuclease activity of the variant domain.

In some embodiments, the methods described herein comprising modifying Cas9 nuclease activity. In some embodiments, Cas9 nuclease activity is decreased or eliminated. Illustrative examples of Cas9 HNH mutations that decrease or eliminate the nuclease activity in the domain include, but are not limited to: S. pyogenes (D10A); S. thermophilis (D9A); T. denticola (D13A); and N. meningitidis (D16A). Illustrative examples of Cas9 RuvC-like domain mutations that decrease or eliminate the nuclease activity in the domain include, but are not limited to: S. pyogenes (D839A, H840A, or N863A); S. thermophilis (D598A, H599A, or N622A); T: denticola (D878A, H879A, or N902A); and N. meningitidis (D587A, H588A, or N611A). In some instances, the methods described herein comprise decreasing the Cas9 nuclease activity and/or efficiency towards a biological target. In some instances, the methods described herein comprise decreasing the Cas9 nuclease activity and/or efficiency towards a disease target.

Similarly, Cas9 equivalents, variants, homologs, orthologs, or paralogs, whether naturally occurring or non-naturally occurring (e.g., engineered or recombinant), and a Cas9 equivalent from any Class 2 CRISPR system (e.g., Type II and V), including Cas12a (Cpf1), Cas12e (CasX), Cas12b1 (C2c1), Cas12b2, and Cas12c (C2c3), are suitable for use in particular embodiments of the current disclosure. Further Cas-equivalents are described in Makarova et al., “C2c2 is a single-component programmable RNA-guided RNA-targeting CRISPR effector,” Science 2016; 353(6299) and Makarova et al., “Classification and Nomenclature of CRISPR-Cas Systems: Where from Here?,” The CRISPR Journal, Vol. 1. No. 5, 2018

CasX (Cas12e) is a Cas9 equivalent that reportedly has the same function as Cas9 but which evolved through convergent evolution. Thus, the CasX (Cas12e) protein described in Liu et al., “CasX enzymes comprises a distinct family of RNA-guided genome editors,” Nature, 2019, Vol. 566: 218-223, is contemplated to be used with the gene editing system described herein. In addition, any variant or modification of CasX (Cas12e) is conceivable and within the scope of the present disclosure.

In various other embodiments, the Cas nuclease described herein (e.g., a Cas9, a Cas12a (Cpf1), a Cas12e (CasX), a Cas12d (CasY), a Cas12b1 (C2c1), a Cas12c (C2c3), a Cas12g, a Cas12h, a Cas14, or a variant thereof) are suitable for use in particular embodiments of the current disclosure.

ii. Homing Endonucleases/Meganucleases

In various embodiments, a plurality of homing endonucleases or meganucleases are introduced into a cell and engineered to bind to, and to introduce single-stranded nicks or double-strand breaks (DSBs) in a plurality of genome target sites including, but not limited to genes encoding proteins associated with specific disease (e.g., sickle cell disease). In some embodiments, homing endonucleases or meganucleases are suitable for use in particular embodiments of the current disclosure. In addition, any variant or modification of endonucleases or meganucleases are conceivable and within the scope of the present disclosure. “Homing endonuclease” and “meganuclease” are used interchangeably and refer to naturally-occurring nucleases or engineered meganucleases that recognize 12-45 base-pair cleavage sites and are commonly grouped into five families based on sequence and structure motifs: LAGLIDADG (SEQ ID NO: 61), GIY-YIG, HNH, His-Cys box, and PD-(D/E)XK.

Engineered Hes do not exist in nature and can be obtained by recombinant DNA technology or by random mutagenesis. Engineered Hes may be obtained by making one or more amino acid alterations, e.g., mutating, substituting, adding, or deleting one or more amino acids, in a naturally occurring HE or previously engineered HE. In particular embodiments, an engineered HE comprises one or more amino acid alterations to the DNA recognition interface.

Engineered Hes contemplated in particular embodiments may further comprise one or more linkers and/or additional functional domains, e.g., an end-processing enzymatic domain of an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. In particular embodiments, engineered Hes are introduced into a T cell with an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. The HE and 3′ processing enzyme may be introduced separately, e.g., in different vectors or separate mRNAs, or together, e.g., as a fusion protein, or in a polycistronic construct separated by a viral self-cleaving peptide or an IRES element.

A “DNA recognition interface” refers to the HE amino acid residues that interact with nucleic acid target bases as well as those residues that are adjacent. For each HE, the DNA recognition interface comprises an extensive network of side chain-to-side chain and side chain-to-DNA contacts, most of which is necessarily unique to recognize a particular nucleic acid target sequence. Thus, the amino acid sequence of the DNA recognition interface corresponding to a particular nucleic acid sequence varies significantly and is a feature of any natural or engineered HE. By way of non-limiting example, an engineered HE contemplated in particular embodiments may be derived by constructing libraries of HE variants in which one or more amino acid residues localized in the DNA recognition interface of the natural HE (or a previously engineered HE) are varied. The libraries may be screened for target cleavage activity against each predicted TCRa locus target sites using cleavage assays (see e.g., Jarjour et al., 2009. Nuc. Acids Res. 37(20): 6871-6880).

LAGLIDADG (SEQ ID NO: 61) homing endonucleases (LHEs) are the most well studied family of meganucleases, are primarily encoded in archaea and in organellar DNA in green algae and fungi, and display the highest overall DNA recognition specificity. LHEs comprise one or two LAGLIDADG (SEQ ID NO: 61) catalytic motifs per protein chain and function as homodimers or single chain monomers, respectively. Structural studies of LAGLIDADG (SEQ ID NO: 61) proteins identified a highly conserved core structure (Stoddard 2005), characterized by an αββαββα fold, with the LAGLIDADG (SEQ ID NO: 61) motif belonging to the first helix of this fold. The highly efficient and specific cleavage of LHE's represent a protein scaffold to derive novel, highly specific endonucleases. However, engineering LHEs to bind and cleave a non-natural or non-canonical target site requires selection of the appropriate LHE scaffold, examination of the target locus, selection of putative target sites, and extensive alteration of the LHE to alter its DNA contact points and cleavage specificity, at up to two-thirds of the base-pair positions in a target site.

Illustrative examples of LHEs from which engineered LHEs may be designed include, but are not limited to I-AabMI, I-AaeMI, I-AniI, I-ApaMI, I-CapIII, I-CapIV, I-CkaMI, I-CpaMI, I-CpaMII, I-CpaMIH, I-CpaMIV, I-CpaMV, I-CpaV, I-CraMI, I-EjeMI, I-GpeMI, I-GpiI, I-GzeMI, I-GzeMII, I-GzeMHI, I-HjeMI, I-LtrII, I-LtrI, I-LtrWI, I-MpeMI, I-MveMI, I-NcrII, I-NcrI, I-NcrMI, I-OheMI, I-OnuI, I-OsoMI, I-OsoMil, I-OsoMHI, I-OsoMIV, I-PanMI, I-PanMII, I-PanMIH, I-PnoMI, I-ScuMI, I-SmaMI, I-SscMI, and I-Vdi141I.

Other illustrative examples of LHEs from which engineered LHEs may be designed include, but are not limited to I-CreI and I-SceI.

In one embodiment, the engineered LHE is selected from the group consisting of: I-CpaMI, I-HjeMI, I-OnuI, I-PanMI, and SmaMI.

In one embodiment, the engineered LHE is I-OnuI.

In one embodiment, engineered I-OnuI LHEs targeting the human TCRa gene were generated from a natural I-OnuI. In a preferred embodiment, engineered I-OnuI LHEs targeting the human TCRa gene were generated from a previously engineered I-OnuI.

In a particular embodiment, the engineered I-OnuI LHE comprises one or more amino acid substitutions in the DNA recognition interface. In particular embodiments, the I-OnuI LHE comprises at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with the DNA recognition interface of I-OnuI (Taekuchi et al. 2011. Proc Natl Acad Sci U.S.A 2011 Aug. 9; 108(32): 13077-13082) or an engineered variant of I-OnuI.

In one embodiment, the I-OnuI LHE comprises at least 70%, more preferably at least 80%, more preferably at least 85%, more preferably at least 90%, more preferably at least 95%, more preferably at least 97%, more preferably at least 99% sequence identity with the DNA recognition interface of I-OnuI (Taekuchi et al. 2011. Proc Natl Acad Sci U.S.A 2011 Aug. 9; 108(32): 13077-13082) or an engineered variant of I-OnuI.

In a particular embodiment, an engineered I-OnuI LHE comprises one or more amino acid substitutions or modifications in the DNA recognition interface, particularly in the subdomains situated from positions 24-50, 68 to 82, 180 to 203 and 223 to 240 of I-OnuI.

In one embodiment, an engineered I-OnuI LHE comprises one or more amino acid substitutions or modifications at additional positions situated anywhere within the entire I-OnuI sequence. The residues which may be substituted and/or modified include but are not limited to amino acids that contact the nucleic acid target or that interact with the nucleic acid backbone or with the nucleotide bases, directly or via a water molecule. In one non-limiting example an engineered I-OnuI LHE contemplated herein comprises one or more substitutions and/or modifications, preferably at least 5, preferably at least 10, preferably at least 15, more preferably at least 20, even more preferably at least 25 in at least one position selected from the position group consisting of positions: 19, 24, 26, 28, 30, 32, 34, 35, 36, 37, 38, 40, 42, 44, 46, 48, 68, 70, 72, 75, 7677, 78, 80, 82, 168, 180, 182, 184, 186, 188, 189, 190, 191, 192, 193, 195, 197, 199, 201, 203, 223, 225, 227, 229, 231, 232, 234, 236, 238, 240 of I-OnuI.

iii. MegaTALs

In various embodiments, a plurality of megaTALs are introduced into a cell and engineered to bind and introduce DSBs in a plurality of genome target sites including. In some embodiments, megaTALs are suitable for use in particular embodiments of the current disclosure. In addition, any variant or modification of megaTALs are conceivable and within the scope of the present disclosure. A “megaTAL” refers to an engineered nuclease comprising an engineered TALE DNA binding domain and an engineered meganuclease, and optionally comprise one or more linkers and/or additional functional domains, e.g., an end-processing enzymatic domain of an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. In particular embodiments, a megaTAL can be introduced into a T cell with an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. The megaTAL and 3′ processing enzyme may be introduced separately, e.g., in different vectors or separate mRNAs, or together, e.g., as a fusion protein, or in a polycistronic construct separated by a viral self-cleaving peptide or an IRES element.

A “TALE DNA binding domain” is the DNA binding portion of transcription activator-like effectors (TALE or TAL-effectors), which mimics plant transcriptional activators to manipulate the plant transcriptome (see e.g., Kay et al., 2007. Science 318:648-651). TALE DNA binding domains contemplated in particular embodiments are engineered de novo or from naturally occurring TALEs, e.g., AvrBs3 from Xanthoronas campestris pv. vesicatoria, Xanthomonas gardneri, Xanihomonas lranslucens, Xanthomonas axonopodis, Xanthomonas perforans, Xanthomonas alfalfa, Xanthomonas citri, Xanthomonas euvesicaloria, and Xanihomonas oryzae and brg11 and hpx17 from Ralstonia solanacearum. Illustrative examples of TALE proteins for deriving and designing DNA binding domains are disclosed in U.S. Pat. No. 9,017,967, and references cited therein, all of which are incorporated herein by reference in their entireties.

In particular embodiments, a megaTAL comprises a TALE DNA binding domain comprising one or more repeat units that are involved in binding of the TALE DNA binding domain to its corresponding target DNA sequence. A single “repeat unit” (also referred to as a “repeat”) is typically 33-35 amino acids in length. Each TALE DNA binding domain repeat unit includes 1 or 2 DNA-binding residues making up the Repeat Variable Di-Residue (RVD), typically at positions 12 and/or 13 of the repeat. The natural (canonical) code for DNA recognition of these TALE DNA binding domains has been determined such that an HD sequence at positions 12 and 13 leads to a binding to cytosine I, NG binds to T, NI to A, NN binds to G or A, and NG binds to T. In certain embodiments, non-canonical (atypical) RVDs are contemplated.

Illustrative examples of non-canonical RVDs suitable for use in particular megaTALs contemplated in particular embodiments include, but are not limited to HH, KH, NH, NK, NQ, RH, RN, SS, NN, SN, KN for recognition of guanine (G); NI, KI, RI, HI, SI for recognition of adenine (A); NG, HG, KG, RG for recognition of thymine (T); RD, SD, HD, ND, KD, YG for recognition of cytosine (C); NV, HN for recognition of A or G; and H*, HA, KA, N*, NA, NC, NS, RA, S* for recognition of A or T or G or C, wherein (*) means that the amino acid at position 13 is absent. Additional illustrative examples of RVDs suitable for use in particular megaTALs contemplated in particular embodiments further include those disclosed in U.S. Pat. No. 8,614,092, which is incorporated herein by reference in its entirety.

In particular embodiments, a megaTAL contemplated herein comprises a TALE DNA binding domain comprising 3 to 30 repeat units. In certain embodiments, a megaTAL comprises 3, 4, 5, 6, 7, 8, 9, 10, 11, 12, 13, 14, 15, 16, 17, 18, 19, 20, 21, 22, 23, 24, 25, 26, 27, 28, 29, or 30 TALE DNA binding domain repeat units. In a preferred embodiment, a megaTAL contemplated herein comprises a TALE DNA binding domain comprising 5-16 repeat units, more preferably 7-15 repeat units, more preferably 9-12 patents are not obvious repeat units, and more preferably 9, 10, or 11 repeat units.

In particular embodiments, a megaTAL contemplated herein comprises a TALE DNA binding domain comprising 3 to 30 repeat units and an additional single truncated TALE repeat unit comprising 20 amino acids located at the C-terminus of a set of TALE repeat units, i.e., an additional C-terminal half-TALE DNA binding domain repeat unit (amino acids −20 to −1 of the C-cap disclosed elsewhere herein, infra). Thus, in particular embodiments, a megaTAL contemplated herein comprises a TALE DNA binding domain comprising 3.5 to 30.5 repeat units. In certain embodiments, a megaTAL comprises 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 TALE DNA binding domain repeat units. In a preferred embodiment, a megaTAL contemplated herein comprises a TALE DNA binding domain comprising 5.5-13.5 repeat units, more preferably 7.5-12.5 repeat units, more preferably 9.5-11.5 repeat units, and more preferably 9.5, 10.5, or 11.5 repeat units.

In particular embodiments, a megaTAL comprises an “N-terminal domain (NTD)” polypeptide, one or more TALE repeat domains/units, a “C-terminal domain (CTD)” polypeptide, and an engineered meganuclease.

As used herein, the term “N-terminal domain (NTD)” polypeptide refers to the sequence that flanks the N-terminal portion or fragment of a naturally occurring TALE DNA binding domain. The NTD sequence, if present, may be of any length as long as the TALE DNA binding domain repeat units retain the ability to bind DNA. In particular embodiments, the NTD polypeptide comprises at least 120 to at least 140 or more amino acids N-terminal to the TALE DNA binding domain (0 is amino acid 1 of the most N-terminal repeat unit). In particular embodiments, the NTD polypeptide comprises at least about 120, 121, 122, 123, 124, 125, 126, 127, 128, 129, 130, 131, 132, 133, 134, 135, 136, 137, 138, 139, or at least 140 amino acids N-terminal to the TALE DNA binding domain. In one embodiment, a megaTAL contemplated herein comprises an NTD polypeptide of at least about amino acids+1 to +122 to at least about +1 to +137 of a Xanthomonas TALE protein (0 is amino acid 1 of the most N-terminal repeat unit). In particular embodiments, the NTD polypeptide comprises at least about 122, 123, 124, 125, 126, 127, 128, 129, 130, 131, 132, 133, 134, 135, 136, or 137 amino acids N-terminal to the TALE DNA binding domain of a Xanthomonas TALE protein. In one embodiment, a megaTAL contemplated herein comprises an NTD polypeptide of at least amino acids+1 to +121 of a Ralstonia TALE protein (0 is amino acid 1 of the most N-terminal repeat unit). In particular embodiments, the NTD polypeptide comprises at least about 121, 122, 123, 124, 125, 126, 127, 128, 129, 130, 131, 132, 133, 134, 135, 136, or 137 amino acids N-terminal to the TALE DNA binding domain of a Ralstonia TALE protein.

As used herein, the term “C-terminal domain (CTD)” polypeptide refers to the sequence that flanks the C-terminal portion or fragment of a naturally occurring TALE DNA binding domain. The CTD sequence, if present, may be of any length as long as the TALE DNA binding domain repeat units retain the ability to bind DNA. In particular embodiments, the CTD polypeptide comprises at least 20 to at least 85 or more amino acids C-terminal to the last full repeat of the TALE DNA binding domain (the first 20 amino acids are the half-repeat unit C-terminal to the last C-terminal full repeat unit). In particular embodiments, the CTD polypeptide comprises at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, or at least 85 amino acids C-terminal to the last full repeat of the TALE DNA binding domain. In one embodiment, a megaTAL contemplated herein comprises a CTD polypeptide of at least about amino acids −20 to −1 of a Xanthomonas TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Xanthomonas TALE protein. In one embodiment, a megaTAL contemplated herein comprises a CTD polypeptide of at least about amino acids −20 to −1 of a Ralstonia TALE protein (−20 is amino acid 1 of a half-repeat unit C-terminal to the last C-terminal full repeat unit). In particular embodiments, the CTD polypeptide comprises at least about 20, 19, 18, 17, 16, 15, 14, 13, 12, 11, 10, 9, 8, 7, 6, 5, 4, 3, 2, or 1 amino acids C-terminal to the last full repeat of the TALE DNA binding domain of a Ralstonia TALE protein.

In particular embodiments, a megaTAL contemplated herein, comprises a fusion polypeptide comprising a TALE DNA binding domain engineered to bind a target sequence, a meganuclease engineered to bind and cleave a target sequence, and optionally an NTD and/or CTD polypeptide, optionally joined to each other with one or more linker polypeptides contemplated elsewhere herein. Without wishing to be bound by any particular theory, it is contemplated that a megaTAL comprising TALE DNA binding domain, and optionally an NTD and/or CTD polypeptide is fused to a linker polypeptide which is further fused to an engineered meganuclease. Thus, the TALE DNA binding domain binds a DNA target sequence that is within about 1, 2, 3, 4, 5, 6, 7, 8, 9, 10, 11, 12, 13, 14, or 15 nucleotides away from the target sequence bound by the DNA binding domain of the meganuclease. In this way, the megaTALs contemplated herein, increase the specificity and efficiency of genome editing.

In particular embodiments, a megaTAL contemplated herein, comprises one or more TALE DNA binding repeat units and an engineered LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-CreI, I-SceI, I-EjeMI, I-GpeMI, I-GpiI, I-GzeMI, I-GzeMII, I-GzeMII, I-HjeMI, I-LtrII, I-LtrI, I-LtrWI, I-MpeMI, I-MveMI, I-NcrII, I-NcrI, I-NcrMI, I-OheMI, I-OnuI, I-OsoMI, I-OsoMII, I-OsoMIII, I-OsoMIV, I-PanMII, I-PanMII, I-PanMIII, I-PnoMI, I-ScuMI, I-SmaMI, I-SscMI, and I-Vdi141I, or preferably I-CpaMI, I-HjeMI, I-OnuI, I-PanMI, and SmaMI, or more preferably I-OnuI.

In particular embodiments, a megaTAL contemplated herein, comprises an NTD, one or more TALE DNA binding repeat units, a CTD, and an engineered LHE selected from the group consisting of: I-AabMI, I-AaeMI, I-AniI, I-ApaMI, I-CapIII, l-CapIV, l-CkaMI, I-CpaMI, I-CpaMII, I-CpaMIII, I-CpaMIV, I-CpaMV, I-CpaV, I-CraMI, I-CreI, I-SceI, I-EjeMI, I-GpeMI, 1-GpiI, I-GzeMI, I-GzeMII, I-GzeMIII, I-HjeMI, I-LtrII, I-LtrI, I-LtrWI, I-MpeMI, I-MveMI, I-NcrII, I-NcrI, I-NcrMI, I-OheMI, I-OnuI, I-OsoMI, I-OsoMI, I-OsoMIII, I-OsoMIV, I-PanMI, I-PanMII, I-PanMIII, I-PnoMI, I-ScuMI, I-SmaMI, I-SscMI, and I-Vdi141I, or preferably I-CpaMI, I-HjeMI, I-OnuI, I-PanMI, and SmaMI, or more preferably I-OnuI.

In particular embodiments, a megaTAL contemplated herein, comprises an NTD, about 9.5 to about 11.5 TALE DNA binding repeat units, and an engineered I-OnuI LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-CreI, I-SceI, I-EjeMI, I-GpeMI, I-GpiI, I-GzeMI, I-GzeMII, I-GzeMII, I-HjeMI, I-LtrI, I-LtrI, I-LtrWI, I-MpeMI, I-MveMI, I-NcrII, I-NcrI, I-NcrMI, I-OheMI, I-OnuI, I-OsoMI, I-OsoMI, I-OsoMIII, I-OsoMIV, I-PanMI, I-PanMII, I-PanMIH, I-PnoMI, I-ScuMI, I-SmaMI, I-SscMI, and I-Vdi141I, or preferably I-CpaMI, I-HjeMI, I-OnuI, I-PanMI, and SmaMI, or more preferably I-OnuI.

In particular embodiments, a megaTAL contemplated herein, comprises an NTD of about 122 amino acids to 137 amino acids, about 9.5, about 10.5, or about 11.5 binding repeat units, a CTD of about 20 amino acids to about 85 amino acids, and an engineered I-OnuI LHE selected from the group consisting of: I-AabMI, I-AaeMI, I-AniI, I-ApaMI, I-CapIII, I-CapIV, I-CkaMI, I-CpaMI, I-CpaMII, I-CpaMIII, I-CpaMIV, I-CpaMV, I-CpaV, I-CraMI, I-CreI, I-SceI, I-EjeMI, I-GpeMI, I-GpiI, I-GzeMI, I-GzeMII, I-GzeMI, I-HjeMI, I-LtrII, I-LtrI, I-LtrWI, I-MpeMI, I-MveMI, I-NcrII, I-NcrI, I-NcrMI, I-OheMI, I-OnuI, I-OsoMI, I-OsoMI, I-OsoMII, I-OsoMIV, I-PanMI, I-PanMII, I-PanMIII, I-PnoMI, I-ScuMI, I-SmaMI, I-SscMI, and I-Vdi141I, or preferably I-CpaMI, I-HjeMI, I-OnuI, I-PanMI, and SmaMI, or more preferably I-OnuI.

MegaTals are further described in, e.g., Boisse “(“megaTALs: a rare-cleaving nuclease architecture for therapeutic genome engineering”,” Nucleic Acids Research, 2013, 42(4):2591-2601).

iv. Talens

In various embodiments, a plurality of transcription activator-like effector nucleases (TALENs) are introduced into a cell and engineered to bind to, and to introduce single-stranded nicks or double-strand breaks (DSBs) in a plurality of genome target sites. In some embodiments, TALENs are suitable for use in particular embodiments of the current disclosure. In addition, any variant or modification of TALENs are conceivable and within the scope of the present disclosure. A “TALEN” refers to an engineered nuclease comprising an engineered TALE DNA binding domain contemplated elsewhere herein and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. In particular embodiments, a TALEN can be introduced into a T cell with an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. The TALEN and 3′ processing enzyme may be introduced separately, e.g., in different vectors or separate mRNAs, or together, e.g., as a fusion protein, or in a polycistronic construct separated by a viral self-cleaving peptide or an IRES element.

In one embodiment, targeted double-stranded cleavage is achieved with two TALENs, each comprising am endonuclease half-domain can be used to reconstitute a catalytically active cleavage domain. In another embodiment, targeted double-stranded cleavage is achieved using a single polypeptide comprising a TALE DNA binding domain and two endonuclease half-domains.

TALENs contemplated in particular embodiments comprise an NTD, a TALE DNA binding domain comprising about 3 to 30 repeat units, e.g., about 3, 4, 5, 6, 7, 8, 9, 10, 11, 12, 13, 14, 15, 16, 17, 18, 19, 20, 21, 22, 23, 24, 25, 26, 27, 28, 29, or 30 repeat units, and an endonuclease domain or half-domain.

TALENs contemplated in particular embodiments comprise an NTD, a TALE DNA binding domain comprising about 3.5 to 30.5 repeat units, e.g., about 3.5, 4.5, 5.5, 6.5, 7.5, 8.5, 9.5, 10.5, 11.5, 12.5, 13.5, 14.5, 15.5, 16.5, 17.5, 18.5, 19.5, 20.5, 21.5, 22.5, 23.5, 24.5, 25.5, 26.5, 27.5, 28.5, 29.5, or 30.5 repeat units, a CTD, and an endonuclease domain or half-domain.

TALENs contemplated in particular embodiments comprise an NTD of about 121 amino acids to about 137 amino acids as disclosed elsewhere herein, a TALE DNA binding domain comprising about 9.5 to about 11.5 repeat units (i.e., about 9.5, about 10.5, or about 11.5 repeat units), a CTD of about 20 amino acids to about 85 amino acids, and an endonuclease domain or half domain.

In particular embodiments, a TALEN comprises an endonuclease domain of a type restriction endonuclease. Restriction endonucleases (restriction enzymes) are present in many species and are capable of sequence-specific binding to DNA (at a recognition site), and cleaving DNA at or near the site of binding. Certain restriction enzymes (e.g., Type-US) cleave DNA at sites removed from the recognition site and have separable binding and endonuclease domains. In one embodiment, TALENs comprise the endonuclease domain (or endonuclease half-domain) from at least one Type-IIS restriction enzyme and one or more TALE DNA-binding domains contemplated elsewhere herein.

Illustrative examples of Type-US restriction endonuclease domains suitable for use in TALENs contemplated in particular embodiments include endonuclease domains of the at least 1633 Type-US restriction endonucleases disclosed at “rebase.neb.com/cgi-bin/sublist?S.”

Additional illustrative examples of Type-IIS restriction endonuclease domains suitable for use in TALENs contemplated in particular embodiments include those of endonucleases selected from the group consisting of: Aar I, Ace III, Aci I, Alo I, A1w26 I, Bae I, Bbr7 I, Bbv I, Bbv II, BbvC I, Bcc I, Bce83 I, BceA I, Bcef I, Bcg I, BciV I, Bfi I, Bin I, Bmg I, Bpu10 I, BsaX I, Bsb I, BscA I, BscG I, BseRI, BseY I, Bsi I, Bsm I, BsmA I, BsmF I, Bsp24 I, BspG I, BspM I, BspNC I, Bsr I, BsrB I, BsrD I, BstF5 I, Btr I, Bts I, Cdi I, CjeP I, Drd II, Earl, Eci I, Eco31 I, Eco57 I, Eco57M I, Esp3 I, Fau I, Fin I, Fok I, Gdi II, Gsu I, Hga I, Hin4 II, Hph I, Ksp632 I, Mbo II, Mly I, Mme I, Mnl I, Pfl1108, I PIe I, Ppi I Psr I, RleA I, Sap I, SfaN I, Sim I, SspD5 I, Sth132 I, Sts I, TspDT I, TspGW I, Tth111 II, UbaP I, Bsa I, and BsmB I.

In one embodiment, a TALEN contemplated herein comprises an endonuclease domain of the Fok I Type-IIS restriction endonuclease.

In one embodiment, a TALEN contemplated herein comprises a TALE DNA binding domain and an endonuclease half-domain from at least one Type-US restriction endonuclease to enhance cleavage specificity, optionally wherein the endonuclease half-domain comprises one or more amino acid substitutions or modifications that minimize or prevent homodimerization.

Illustrative examples of cleavage half-domains suitable for use in particular embodiments contemplated in particular embodiments include those disclosed in U.S. Patent Publication Nos. 20050064474; 20060188987, 20080131962, 20090311787; 20090305346; 20110014616, and 20110201055, each of which are incorporated by reference herein in its entirety.

TALENs are further described in e.g., Christia (“Targeting DNA Double-Strand Breaks with TAL Effector Nuclease,” Genetics. October 2010; 186(2):757-61).

v. Zinc Finger Nucleases

In various embodiments, a plurality of zinc finger nucleases (ZFNs) are introduced into a cell and engineered to bind to, and to introduce single-stranded nicks or double-strand breaks (DSBs) in a plurality of genome target sites. In some embodiments, ZFNs are suitable for use in particular embodiments of the current disclosure. In addition, any variant or modification of ZFNs are conceivable and within the scope of the present disclosure. A “ZFN” refers to an engineered nuclease comprising one or more zinc finger DNA binding domains and an endonuclease domain (or endonuclease half-domain thereof), and optionally comprise one or more linkers and/or additional functional domains, e.g., an end-processing enzymatic domain of an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. In particular embodiments, a ZFN can be introduced into a T cell with an end-processing enzyme that exhibits 5-3′ exonuclease, 5-3′ alkaline exonuclease, 3-5′ exonuclease (e.g., Trex2), 5′ flap endonuclease, helicase or template-independent DNA polymerases activity. The ZFN and 3′ processing enzyme may be introduced separately, e.g., in different vectors or separate mRNAs, or together, e.g., as a fusion protein, or in a polycistronic construct separated by a viral self-cleaving peptide or an IRES element.

In one embodiment, targeted double-stranded cleavage is achieved using two ZFNs, each comprising an endonuclease half-domain can be used to reconstitute a catalytically active cleavage domain. In another embodiment, targeted double-stranded cleavage is achieved with a single polypeptide comprising one or more zinc finger DNA binding domains and two endonuclease half-domains.

In one embodiment, a ZNF comprises a TALE DNA binding domain contemplated elsewhere herein, a zinc finger DNA binding domain, and an endonuclease domain (or endonuclease half-domain) contemplated elsewhere herein.

In one embodiment, a ZNF comprises a zinc finger DNA binding domain, and a meganuclease contemplated elsewhere herein.

In particular embodiments, the ZFN comprises a zinger finger DNA binding domain that has one, two, three, four, five, six, seven, or eight or more zinger finger motifs and an endonuclease domain (or endonuclease half-domain). Typically, a single zinc finger motif is about 30 amino acids in length. Zinc fingers motifs include both canonical C₂H₂ zinc fingers, and non-canonical zinc fingers such as, for example, C₃H zinc fingers and C₄ zinc fingers.

Zinc finger binding domains can be engineered to bind any DNA sequence. Candidate zinc finger DNA binding domains for a given 3 bp DNA target sequence have been identified and modular assembly strategies have been devised for linking a plurality of the domains into a multi-finger peptide targeted to the corresponding composite DNA target sequence. Other suitable methods known in the art can also be used to design and construct nucleic acids encoding zinc finger DNA binding domains, e.g., phage display, random mutagenesis, combinatorial libraries, computer/rational design, affinity selection, PCR, cloning from cDNA or genomic libraries, synthetic construction and the like. (See, e.g., U.S. Pat. No. 5,786,538; Wu et al., PNAS 92:344-348 (1995); Jamieson et al., Biochemistry 33:5689-5695 (1994); Rebar & Pabo, Science 263:671-673 (1994); Choo & Klug, PNAS 91:11163-11167 (1994); Choo & Klug, PNAS 91: 11168-11172 (1994); Desjarlais & Berg, PNAS 90:2256-2260 (1993); Desjarlais & Berg, PNAS 89:7345-7349 (1992); Pomerantz et al., Science 267:93-96 (1995); Pomerantz et al., PNAS 92:9752-9756 (1995); Liu et al., PNAS 94:5525-5530 (1997); Griesman & Pabo, Science 275:657-661 (1997); Desjarlais & Berg, PNAS 91:11-99-11103 (1994)).

Individual zinc finger motifs bind to a three or four nucleotide sequence. The length of a sequence to which a zinc finger binding domain is engineered to bind (e.g., a target sequence) will determine the number of zinc finger motifs in an engineered zinc finger binding domain. For example, for ZFNs in which the zinc finger motifs do not bind to overlapping subsites, a six-nucleotide target sequence is bound by a two-finger binding domain; a nine-nucleotide target sequence is bound by a three-finger binding domain, etc. In particular embodiments, DNA binding sites for individual zinc fingers motifs in a target site need not be contiguous, but can be separated by one or several nucleotides, depending on the length and nature of the linker sequences between the zinc finger motifs in a multi-finger binding domain.

In particular embodiments, ZNFs contemplated herein comprise, a zinc finger DNA binding domain comprising two, three, four, five, six, seven or eight or more zinc finger motifs, and an endonuclease domain or half-domain from at least one Type-IIS restriction enzyme and one or more TALE DNA-binding domains contemplated elsewhere herein.

In particular embodiments, ZNFs contemplated herein comprise, a zinc finger DNA binding domain comprising three, four, five, six, seven or eight or more zinc finger motifs, and an endonuclease domain or half-domain from at least one Type-IIS restriction enzyme selected from the group consisting of: Aar I, Ace III, Aci I, Alo 1, A1w26 I, Bae I, Bbr7 I, Bbv I, Bbv II, BbvC I, Bcc I, Bce83 I, BceA L Bcef I, Bcg I, BciV I, Bfi I, Bin 1, Bmg I, Bpul0 I, BsaX I, Bsb I, BscA 1, BscG I, BseR 1, BseY I, Bsi I, Bsm I, BsmA I, BsmF I, Bsp24 I, BspG I, BspM I, BspNC I, Bsr L BsrB I, BsrD I, BstF5 I, Btr L Bts I, Cdi i, CjeP I, Drd II, Earl, Eci I, Eco31 I, Eco57 I, Eco57M I, Esp3 I, Fau I, Fin I, Fok I, Gdi II, Gsu I, Hga I, Hin4 II, Hph I, Ksp632 I, Mbo II, Mly I, Mme I, Mnl I, Pfll108, I Ple I, Ppi I Psr I, RIeA I, Sap I, SfaN I, Sim I, SspD5 I, Sth132 I, Sts I, TspDT I, TspGW I, Tth111 II, UbaP I, Bsa I, and BsmB I.

In particular embodiments, ZNFs contemplated herein comprise, a zinc finger DNA binding domain comprising three, four, five, six, seven or eight or more zinc finger motifs, and an endonuclease domain or half-domain from the Fok I Type-IIS restriction endonuclease.

In one embodiment, a ZFN contemplated herein comprises a zinc finger DNA binding domain and an endonuclease half-domain from at least one Type-US restriction endonuclease to enhance cleavage specificity, optionally wherein the endonuclease half-domain comprises one or more amino acid substitutions or modifications that minimize or prevent homodimerization.

### (b) In Vitro Gene Editing in Hematopoietic Stem Cells

In certain embodiments, the LNP compositions described herein may be used to deliver one or more nucleic acids encoding a gene editing system targeting a locus (or loci) within a cell. For example, an mRNA included in an LNP composition may encode a polypeptide and produce the gene editing upon contacting and/or entry (e.g., transfection) into a cell. In certain embodiments, an mRNA included in an LNP composition of the invention may encode a polypeptide that may improve the function or health of a cell by targeting the nucleotide sequence of one or more targets of a dysfunctional protein or desired target described herein.

Provided herein is a method of genetically modifying a hematopoietic stem cell (HSC) in vitro in a cell, the method comprising administering to the cell the LNP of any one of the preceding embodiments. In some embodiment, the method comprises contact the cell with an LNP comprising a lipid-antibody conjugate, an ionizable cationic lipid, and one or more nucleic acids disposed therein. In some embodiments, the one or more nucleic acids disposed therein comprise an mRNA encoding a site-directed nuclease, a chemical base editor, a prime editor, or an epigenome editor.

In some embodiments, the LNP compositions described herein target specific cell-surface markers of hematopoietic stem cells (HSCs). In some embodiment, the LNP comprises a HSC targeting group (e.g., an antibody or lipid-antibody conjugate) that specifically targets a HSC surface antigen. In some embodiments, the LNP comprises an antibody or antigen-binding fragment thereof that targets CD105 and/or CD117. In some embodiments, the LNP comprises an antibody or antigen-binding fragment thereof that targets CD117. In some embodiments, the LNP comprises an antibody or antigen-binding fragment thereof that targets CD105.

In some embodiments, the one or more nucleic acids disposed therein encode a gene editing system targeting the nucleotide sequence of one or more targets described herein. In some embodiments, the one or more nucleic acids disposed therein comprise an mRNA encoding a gene editing system targeting the nucleotide sequence of one or more targets described herein. In some embodiments, the target is a locus (or loci) within the cell associated with protein dysfunction in the cell and/or disease in a subject. In some embodiments, the LNP of any one of the preceding embodiments targeting a locus (or loci) within the cell results in increased HbF. In some embodiments, use of the LNP of any one of the preceding embodiments to increase HbF in a cell may be to the treat sickle cell disease or beta-thalessemia in a subject.

In some embodiments, the method comprises treating HSCs with the LNPs described herein, wherein the RNA concentration remains constant. In some embodiments, the RNA concentration delivered by the LNPs is between about 0.1 and 10 μg/mL. In some embodiments, the RNA concentration delivered by the LNPs is between about 0.5 and 8 μg/mL, 0.6 and 7 μg/mL, 0.7 and 6 μg/mL, 0.8 and 5 μg/mL, 0.9 and 4 μg/mL or 1 and 3 μg/mL. In some embodiments, the RNA concentration delivered by the LNPs is 0.1, 0.2, 0.3, 0.4, 0.5, 0.6, 0.7, 0.8, 0.9. 1, 2, 3, 4, 5, 6, 7, 8, 9, 10, or more μg/mL. In some embodiments, the RNA concentration delivered by the LNPs is 1 μg/mL.

In some embodiments, the method comprises incubating the HSCs with the LNPs described herein, wherein the HSCs are incubated with the LNPs for at least 4 hours. In some embodiments, the HSCs are incubated with the LNPs for between about 4 to 96 hours. In some embodiments, the HSCs are incubated with the LNPs for between about 6 to 90 hours, 8 to 80 hours, 10 to 70 hours, 12 to 60 hours, 18 to 50 hours, 24 to 48 hours, or 30 to 36 hours.

### (c) In Vivo Gene Editing in Hematopoietic Stem Cells

In certain embodiments, the LNP compositions described herein may be used to deliver therapeutic or prophylactic agents to a subject. For example, an mRNA included in an LNP composition may encode a polypeptide and produce the therapeutic or prophylactic polypeptide upon contacting and/or entry (e.g., transfection) into a cell. In certain embodiments, an mRNA included in an LNP composition of the invention may encode a polypeptide that may improve the health of a subject by targeting the nucleotide sequence of one or more targets of the diseases described herein.

Provided herein is a method of genetically modifying a hematopoietic stem cell (HSC) in vivo in a subject, the method comprising administering to the subject the LNP of any one of the preceding embodiments. In some embodiment, the method comprises administering to the subject an LNP comprising a lipid-antibody conjugate, an ionizable cationic lipid, and one or more nucleic acids disposed therein. In some embodiments, the one or more nucleic acids disposed therein comprise an mRNA encoding a site-directed nuclease, a chemical base editor, a prime editor, or an epigenome editor.

In some aspects, the method of genetically modifying a hematopoietic stem cell (HSC) in vivo in a subject further comprises administering to the subject an HSC mobilization agent. In some embodiments, the method comprises administering the LNP is to the subject intravenously. In some embodiments, the HSC mobilization agent is administered to the subject before, during, or before and during administration of the LNP. In some embodiments, the HSC mobilization agent is administered to the subject before administration of the LNP. In some embodiments, the HSC mobilization agent is administered to the subject during administration of the LNP. In some embodiments, the HSC mobilization agent is administered to the subject before and during administration of the LNP. In some embodiments, the HSC mobilization agent comprises plerixafor, granulocyte colony stimulating factor (G-CSF), granulocyte-macrophage colony stimulating factor (GM-CSF), or any combination thereof. In some embodiments, the HSC mobilization agent comprises plerixafor and G-CSF.

Also provided herein is a method of treating a disease in a subject in need thereof. In some embodiments, the method comprises administering to the subject the LNP of any one of the preceding embodiments. In some embodiment, the method comprises administering to the subject an LNP comprising a lipid-antibody conjugate, an ionizable cationic lipid, and one or more nucleic acids disposed therein. In some embodiments, the one or more nucleic acids disposed therein comprise an mRNA encoding a site-directed nuclease, a chemical base editor, a prime editor, or an epigenome editor.

In some aspects, the method of treating a disease further comprises administering to the subject an HSC mobilization agent. In some embodiments, the method comprises administering the LNP is to the subject intravenously. In some embodiments, the HSC mobilization agent is administered to the subject before, during, or before and during administration of the LNP. In some embodiments, the HSC mobilization agent is administered to the subject before administration of the LNP. In some embodiments, the HSC mobilization agent is administered to the subject during administration of the LNP. In some embodiments, the HSC mobilization agent is administered to the subject before and during administration of the LNP. In some embodiments, the HSC mobilization agent comprises plerixafor, granulocyte colony stimulating factor (G-CSF), granulocyte-macrophage colony stimulating factor (GM-CSF), or any combination thereof. In some embodiments, the HSC mobilization agent comprises plerixafor and G-CSF.

In some aspects, provided are methods of modulating cellular function of a target hematopoietic stem cell (HSC) of a subject. In some embodiments, the method comprises administering to the subject a lipid nanoparticle (LNP). In some embodiments, the LNP comprises an ionizable cationic lipid. In some embodiments, the LNP comprises a conjugate comprising the following structure: [Lipid]-[optional linker]-[antibody]. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises a nucleic acid encoding a polypeptide for modulating the cellular function of the HSC. In some embodiments, an aspect of the disclosure relates to an LNP or a pharmaceutical composition containing thereof, as disclosed herein, for use in a method of modulating cellular function of a targeted HSC cell of a subject. Such a method may be for the treatment of a disease as disclosed hereafter. In some embodiments, a method as disclosed herein can comprise contacting in vitro or ex vivo the HSC cell of a subject with a lipid nanoparticle (LNP).

In some embodiments, the LNP provided herein is used in method of editing a HSC in vivo, wherein the LNP comprises Lipid 15, a HSC targeting group that binds to a HSC surface antigen comprising a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:7, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the LNP provided herein is used in method of editing a HSC in vivo, wherein the LNP comprise Lipid 15, a HSC targeting group that binds to a HSC surface antigen comprising a VH domain comprising CDR-H1, CDR-H2, and CDR-H3 sequences, where the VH domain comprises the amino acid sequence set forth in SEQ ID NO:7, and a VL domain comprising CDR-L1, CDR-L2, and CDR-L3 sequences, where the VL domain comprises the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the exemplary LNP provided herein is used to edit HSC cells in vivo. In some embodiments, the exemplary LNP provided herein is delivered to a subject with disease for in vivo gene editing and treatment of the disease. In some embodiments, the exemplary LNP provided herein is delivered to a subject with sickle cell disease or beta-thalessemia for in vivo gene editing and treatment of the subject. In some embodiments, use of the exemplary LNP provided herein for treatment of sickle cell disease in a subject is safe and effective. In some embodiments, use of the exemplary LNP provided herein for treatment of beta-thalessemia in a subject is safe and effective.

The therapeutic and/or prophylactic compositions described herein may be administered to a subject using any reasonable amount and any route of administration effective for preventing, treating, diagnosing, or imaging a disease and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more mRNAs may be administered by a variety of routes, for example, orally, intravenously, intramuscularly, intra-arterially, intramedullary, intrathecally, subcutaneously, intraventricularly, trans- or intra-dermally, intradermally, rectally, intravaginally, intraperitoneally, topically, mucosally, nasally, intratumorally. In certain embodiments, an LNP composition may be administered intravenously, intramuscularly, intradermally, intra-arterially, intratumorally, or subcutaneously. However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition including one or more mRNAs (e.g., its stability in various bodily environments such as the bloodstream and gastrointestinal tract), the condition of the patient (e.g., whether the patient is able to tolerate particular routes of administration), etc.

LNP compositions including one or more mRNAs may be used in combination with one or more other therapeutic, prophylactic, diagnostic, or imaging agents. “y “in combination wi”h,” it is not intended to imply that the agents must be administered at the same time and/or formulated for delivery together, although these methods of delivery are within the scope of the present disclosure. For example, one or more LNP compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disease (for example, a composition useful for treating cancer may be administered concurrently with a chemotherapeutic agent), or they may achieve different effects (e.g., control of any adverse effects).

### VIII. Methods of Treating Diseases Associated with Hematopoietic Stem Cells

The present disclosure provides methods of treating a disease in a subject by delivering a payload encoding one or more nucleic acids (e.g., a gene editing system, e.g., a site-directed nuclease and, optionally, a guide RNA) to HSCs in vivo in a subject, thereby treating the disease. In some embodiments, delivery of a payload comprising one or more nucleic acids encoding a site-directed nuclease to HSCs in vivo in a subject may result in the modification of a biological target. In some embodiments, the methods provided herein include the delivery of a payload further comprising a guide RNA. In some embodiments, the delivery of a payload may result in the silencing of a biological target. The biological target may be associated with a disease to be treated by the methods described herein. Any disclosure herein of a method of treating a disease should be interpreted also as a disclosure of an LNP or pharmaceutical composition comprising said LNP for use in such methods.

In some aspects, provided are method of treating, ameliorating, or preventing a symptom of a disease in a subject in need thereof. In some embodiments, the method comprises administering to the subject a lipid nanoparticle (LNP) provided herein. LNP compositions of the invention may be useful for treating a disease characterized by missing or aberrant protein or polypeptide activity in HSCs or cells differentiated from HSCs (e.g., monocytes, neutrophils, platelets, red blood cells, and immune cells such as natural killer (NK) cells, B-cells, T-cells, and the like). Upon delivery of one or more nucleic acids encoding a gene editing system to HSCs of the subject, expression of the gene editing system may induce a genetic modification in the HSCs, thereby reducing or eliminating an issue caused by the absence of or aberrant activity caused by the polypeptide. The genetic modification may modify the gene encoding the missing or aberrant protein, for example, to correct a mutation in a protein-coding sequence of the gene, or to modify a regulatory sequence associated with the gene to increase expression of the native functional protein. In some instances, the genetic modification may replace a gene encoding the missing or aberrant protein, for example, by inserting a transgene encoding a gene encoding the native protein. Any gene editing system known in the art or described herein may be used in the methods of treatment described herein.

Diseases characterized by dysfunctional or aberrant protein or polypeptide activity for which a composition of the invention may be administered include, but are not limited to, a blood disease, hemoglobinopathy, a primary immune deficiency (PID), a congenital cytopenia, a hemophilia, a thrombophilia, an inborn error of metabolism, or a neuropathy. In some embodiments, the blood disease is a α-hemoglobinopathy, a β-hemoglobinopathy (e.g., β-thalassemia), or sickle cell disease. In some embodiments, the PID may comprise, for example, a severe combined immunodeficiency (SCID), Wiskott-Aldrich syndrome, chronic granulomatous disease, immunodysregulation polyendocrinopathy enteropathay X-linked (IPEX), a hyper IgM syndrome, or X-linked agammaglobulinemia. In some embodiments, the SCID is Artemis-SCID (ART-SCID), recombination activating gene SCID (RAG-SCID), X-linked SCID (X-SCID), adenosine deaminase-deficient SCID, interleukin 7 receptor deficiency SCID, or JAK3 SCID. In some embodiments, the congenital cytopenia is Fanconia anemia, Shwachman-Diamond syndrome, Blackfan-Diamond anemia, dyskeratosis congenita, congenital amegakaryocytic thrombocytopenia, or reticular dysgenesis. In some embodiments, the hemophilia is hemophilia A, hemophilia B, or hemophilia C. In some embodiments, the thrombophilia is amegakaryocytic thrombocytopenia or factor X deficiency. In some embodiments, the inborn error of metabolism is phenylketoneuria, medium-chain acyl-CoA dehydrogenase (MCAD) deficiency, a lysosomal storage disease, a glycogen storage disorder, a peroxisomal disorder, Fabry disease, Gaucher disease, Hurler syndrome, Hunter syndrome, Wolman disease, or pyruvate kinase deficiency. In some embodiments, the peroxisomal disorder is X-linked adrenoleukodystrophy. In some embodiments, the lysosomal storage disease is metachromatic leukodystrophy, mucopolysaccharidosis I, or mucopolysaccharidosis II. In some embodiments, the neuropathy is Friedrich's ataxia. In some embodiments, the viral disease is HIV/AIDS.

Multiple diseases may be characterized by missing (or substantially diminished such that proper protein function does not occur) protein activity. Such proteins may not be present, or they may be essentially non-functional. In some embodiments, the payload delivered by the targeted lipid nanoparticle to HSCs comprises a site-directed nuclease resulting in the treatment of human disease. For example, β-hemoglobinopathies such as Sickle cell disease and β-thalassemia are caused by mutations in the β-globin (HBB) gene resulting in lower than normal adult HbA hemoglobin (a heterotetramer made up of two α-globin and two β-globin subunits), and/or the production of abnormal hemoglobin (e.g., HbS, a heterotetramer of two α-globin and two aberrant β-globin subunits). An alternative form of hemoglobin is fetal hemoglobin (HbF), a heterotetramer made up of two α-globin and two γ-globin subunits. Throughout post-natal life, the expression of the HBG (HBG1 and HBG2) genes, which encodes γ-globin, is suppressed by the silencing factors B-cell lymphoma 11A (BCL11A), Kruppel-like factor 1 (KLF1) and ZBTB7A. Without wishing to be bound by theory, genetic modifications that disrupt or silence the BCL11A gene in HSCs may result in the development of erythrocytes which express the HBG1 and/or HBG2 genes encoding γ-globin and produce HbF, thereby restoring hemoglobin function in cells which may otherwise have expressed aberrant β-globin and/or insufficient amounts of normal β-globin. For example, disruption of one or more of the BCL11A intronic erythroid-specific enhancer sequences present in intron-2 of the BCL11A gene (referred to herein to as “the BCL11A erythroid enhancer”) may result in decreased expression and activity of BLC11A protein and thus increased expression of HbF in red blood cells. The term “BCL11A erythroid enhancer” refers to a polynucleotide comprising one or more of the BCL11A erythroid enhancer sequences in intron-2, the intronic region between exon 2 and exon 3 of the BCL11A gene. BCL11A erythroid enhancer sequences include, for example, the nucleotide sequences at distances of about +55 kilobases (kb) through about +62 kb (e.g., at about +55 kb, about +58 kb, and/or about +62 kb) nucleotides downstream (in the 3′ direction) of the BCL11A transcription start site. BCL1A erythroid enhancer sequences are further described, for example, in Bauer et al. (201“. “An erythroid enhancer of BCL1I A subject to genetic variation determines fetal hemoglobin lev”.” Science 342.6155: 253-257), Lettre and Bauer (201”. “Fetal haemoglobin in sickle-cell disease: from genetic epidemiology to new therapeutic strategies.” The Lancet 387.10037: 2554-2564), and Antoniani et al. (201”. “Concise review: epigenetic regulation of hematopoiesis: biological insights and therapeutic applications.” Stem cells translational medicine 6.12; 2106-2114).

The BCL11A erythroid enhancer includes polynucleotide sequences in intron-2 of the BCL11A gene. For example, in some embodiments, the BCL11A erythroid enhancer comprises a polynucleotide sequence between about +54 kb and about +63 kb downstream (in the 3′ direction) of the BCL1A transcription start site (TSS). In certain embodiments, the BCL11A erythroid enhancer comprises a polynucleotide sequence between about +54 kb and about +56 kb, a polynucleotide between about +57 kb and about +59 kb, or a polynucleotide between about +62 kb and about +63 kb downstream of the BCL11A TSS, or any combination thereof. In certain embodiments, the BCL1 I A erythroid enhancer comprises a polynucleotide sequence between about +54 kb and about +56 kb downstream of the BCL11A TSS. In certain embodiments, the BCL11A erythroid enhancer comprises a polynucleotide sequence between about +57 kb and about +59 kb downstream of the BCL11A TSS. In certain embodiments, the BCL11A erythroid enhancer comprises a polynucleotide sequence between about +62 kb and about +63 kb downstream of the BCL11A TSS. In some embodiments, the BCL11A erythroid enhancer comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the BCL11A erythroid enhancer comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb downstream of the BCL11A TSS. In certain embodiments, the BCL11A erythroid enhancer comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +58 kb downstream of the BCL11A TSS. In certain embodiments, the BCL11A erythroid enhancer comprises a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +62 kb downstream of the BCL11A TSS. In certain embodiments, the BCL11A erythroid enhancer comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions.

In some embodiments, delivery of targeted lipid nanoparticles to HSCs thereby results in targeted editing of the BCL11A erythroid enhancer (e.g., editing of a polynucleotide sequence in intron-2 of the BCL11A gene, e.g., deletion, insertion, or substitution of one or more polynucleotides between about +54 kb and about +63 kb downstream of the BCL11A transcription start site (TSS), e.g., deletion, insertion, or substitution of one or more polynucleotides between about +54 kb and about +56 kb, a polynucleotide between about +57 kb and about +59 kb, or a polynucleotide between about +62 kb and about +63 kb downstream of the BCL11A TSS, or a combination thereof) for the treatment of β-hemoglobinopathies. In certain embodiments, delivery of targeted lipid nanoparticles to HSCs thereby results in targeted editing of one or more BCL11A erythroid enhancer nucleotide sequences in intron-2 of the BCL11A gene (e.g., deletion, insertion, or substitution of one or more polynucleotides between about +54 kb and about +63 kb downstream of the BCL11A transcription start site (TSS), e.g., deletion, insertion, or substitution of one or more polynucleotides between about +54 kb and about +56 kb, a polynucleotide between about +57 kb and about +59 kb, or a polynucleotide between about +62 kb and about +63 kb downstream of the BCL11A TSS, or a combination thereof), resulting in decreased expression of the BCL11A gene (e.g., a reduction in BCL11A mRNA and/or protein) and/or a reduction in fetal hemoglobin.

The present disclosure provides a method for treating such diseases in a subject by administering an LNP composition comprising an ionizable cationic lipid, a conjugate comprising the following structure: [Lipid]-[optional linker]-[HSC targeting group], and one or more nucleic acids encoding a gene editing system (e.g., an mRNA encoding a site-directed nuclease, a chemical base editor, a prime editor, or an epigenome editor and, optionally, a gRNA or pegRNA), wherein the gene editing system is configured to target a target modify a target nucleotide sequence associated with the particular disease to be treated.

The therapeutic and/or prophylactic compositions described herein may be administered to a subject using any reasonable amount and any route of administration effective for preventing, treating, diagnosing, or imaging a disease and/or any other purpose. The specific amount administered to a given subject may vary depending on the species, age, and general condition of the subject, the purpose of the administration, the particular composition, the mode of administration, and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment.

A LNP composition including one or more nucleic acids may be administered by a variety of routes, for example, intravenously, intraosseously (into bone marrow), orally, intramuscularly, intra-arterially, trans- or intra-dermally, intradermally, rectally, intraperitoneally, or mucosally. In some embodiments, an LNP composition may be administered intravenously, intraosseously, or intra-arterially. In certain embodiments, an LNP composition may be administered intravenously or intra-arterially during or after the administration of an HSC mobilization agent (e.g., plerixafor and/or G-CSF). However, the present disclosure encompasses the delivery of LNP compositions of the invention by any appropriate route taking into consideration likely advances in the sciences of drug delivery. In general, the most appropriate route of administration will depend upon a variety of factors including the nature of the LNP composition, the disease to be treated, the condition of the patient (e.g., whether the patient is able to tolerate particular routes of administration), etc.

LNP compositions including one or more mRNAs may be used in combination with one or more other therapeutic, prophylactic, diagnostic, or imaging agents. “y “in combination wi”h,” it is not intended to imply that the agents must be administered at the same time and/or formulated for delivery together, although these methods of delivery are within the scope of the present disclosure. For example, one or more LNP compositions including one or more different m RNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disease or they may achieve different effects (e.g., control of any adverse effects).

In some aspect, provided are methods of treating, ameliorating, or preventing a symptom of a disease in a subject in need thereof. In some embodiments, the method comprises administering to the subject a lipid nanoparticle (LNP) for delivering a nucleic acid into an hematopoietic stem cell (HSC) in vivo in the subject. In some embodiments, the LNP comprises an ionizable cationic lipid. In some embodiments, the LNP comprises a conjugate comprising the following structure: [Lipid]-[optional linker]-[HSC targeting group]. In certain embodiments, the LNP comprises a lipid-antibody conjugate comprising the following structure: [Lipid]-[optional linker]-[antibody], wherein the antibody binds to CD105 and/or CD117. In some embodiments, the antibody that binds to CD117 is Ab1. In some embodiments, the antibody that binds to CD117 is Ab2. In some embodiments, the antibody that binds to CD105 is Ab3. In some embodiments, the LNP comprises a sterol or other structural lipid. In some embodiments, the LNP comprises a neutral phospholipid. In some embodiments, the LNP comprises a free Polyethylene glycol (PEG) lipid. In some embodiments, the LNP comprises one or more nucleic acids encoding a gene editing system. In certain embodiments, the one or more nucleic acids comprise an mRNA encoding a site-directed nuclease, a chemical base editor, a prime editor, or an epigenome editor and, optionally, a gRNA or pegRNA. In one embodiment, the one or more nucleic acids comprise an mRNA encoding a Cas nuclease and a guide RNA.

In some embodiments, the gene editing system induces a genetic modification in one or more genes within the HSC, thereby treating the disease. In some embodiments, an aspect of the disclosure relates to an LNP or a pharmaceutical composition containing thereof, as disclosed herein, for use in a method of treating, ameliorating, or preventing a symptom of a disease in a subject in need thereof. A disease may be as disclosed herein. In some embodiments, a method as disclosed herein can comprise contacting an HSC in vivo in a subject with an LNP described herein.

In some embodiments, the LNP provides at least one of the following benefits:
(i) increased specificity of delivery of the nucleic acid into the HSC compared to a reference LNP;
(ii) increased transfection rate compared to a reference LNP;
(iii) the LNP can be administered at a lower dose compared to a reference LNP to reach the same treatment efficacy;
(iv) a low level of dye accessible mRNA (<15%) and high RNA encapsulation efficiencies, wherein at least 80% mRNA was recovered in final formulation relative to the total RNA used in LNP batch preparation; and
(v) reduction of the occurrence and/or the severity of a symptom of the disease in the subject.

The LNPs provided herein are useful for the treatment of any disease associated with hematopoietic stem cells (HSCs), or for which HSC replacement therapy may serve as a viable treatment method. In some embodiments, the disease is a blood disease. In certain embodiments, the disease is a hemoglobinopathy, a primary immune deficiency (PID), a congenital cytopenia, a hemophilia, a thrombophilia, an inborn error of metabolism, or a neuropathy.

In some embodiments, provided herein is a method of treating an α-hemoglobinopathy or a β-hemoglobinopathy. In some embodiments, provided herein is a method of treating a α-hemoglobinopathy. In some embodiments, provided herein is a method of treating a β-hemoglobinopathy. In certain embodiments, the β-hemoglobinopathy is a β-thalassemia. In certain embodiments, the β-hemoglobinopathy is sickle cell disease. In some embodiments, administration of the LNPs results in one or more of: a) insertion of an HBB transgene, or a fragment thereof, into at least one HSC of the subject; b) increased expression of β-globin in the subject; b) an increased amount of α2β2 adult hemoglobin (HbA) in the subject; c) insertion of an HBG1 transgene, or a fragment thereof, into at least one HSC of the subject; d) insertion of an HBG2 transgene, or a fragment thereof, into at least one HSC of the subject; e) increased expression of γ-globin in the subject: f) an increased amount of α272 fetal hemoglobin (HbF) in the subject; g) disruption of the HBA1 gene, the HBA2 gene, or a combination thereof in at least one HSC of the subject; h) decreased expression of α-globin in the subject; and i) a decreased amount of α4 α-globin heterotetramers the subject. In some embodiments, the method comprises administration of an LNP described herein to the subject, wherein the LNP comprises one or more nucleic acids encoding a gene editing system configured to induce a genetic modification in a target nucleotide sequence in the HSC. In some embodiments, the LNP comprises an mRNA encoding a Cas nuclease and a gRNA comprising a nucleotide sequence that confers binding to a target nucleotide sequence (e.g., a gRNA comprising a nucleotide sequence having at least 80%, at least 90%, at least 95%, or 100% identity to at least 15, at least 16, at least 17, at least 18, at least 19, or at least 20 consecutive nucleotides of the target nucleotide sequence). In certain embodiments wherein the disease is β-thalassemia or sickle cell disease, the target nucleotide sequence comprises at least 15, at least 16, at least 17, at least 18, at least 19, or at least 20 consecutive nucleotides and is located within the coding region of the gene, the intronic region associated with the gene, the exon region associated with the gene, the 5′ untranslated region associated with the gene, or the 3′ untranslated region associated with the gene, wherein the gene is the HBB gene, the HBG1 gene, the HBG2 gene, the HBA1 gene, the HBA2 gene, the HBD gene, the BCL11A gene, the BACH2 gene, the KLF1 gene, or the LRF gene. In certain embodiments wherein the disease is β-thalassemia or sickle cell disease, the target nucleotide sequence comprises at least 15, at least 16, at least 17, at least 18, at least 19, or at least 20 consecutive nucleotides and is located within the regulatory region of the gene, wherein the gene is the HBB gene, the HBG1 gene, the HBG2 gene, the HBA1 gene, the HBA2 gene, the HBD gene, the BCL11A gene, the BACH2 gene, the KLF1 gene, or the LRF gene. In certain embodiments wherein the disease is β-thalassemia or sickle cell disease, the target nucleotide sequence comprises at least 15, at least 16, at least 17, at least 18, at least 19, or at least 20 consecutive nucleotides and is located within the enhancer region of the gene or within the repressor region of the gene, wherein the gene is the HBB gene, the HBG1 gene, the HBG2 gene, the HBA1 gene, the HBA2 gene, the HBD gene, the BCL11A gene, the BACH2 gene, the KLF1 gene, or the LRF gene. In certain embodiments wherein the disease is β-thalassemia or sickle cell disease, the target nucleotide sequence comprises at least 15, at least 16, at least 17, at least 18, at least 19, or at least 20 consecutive nucleotides within the BCL11A gene. In certain embodiments wherein the disease is β-thalassemia or sickle cell disease, the target nucleotide sequence comprises at least 15, at least 16, at least 17, at least 18, at least 19, or at least 20 consecutive nucleotides and is located within a polynucleotide sequence in intron-2 of the BCL11A gene. In certain embodiments, the target nucleotide sequence comprises at least 15, at least 16, at least 17, at least 18, at least 19, or at least 20 consecutive nucleotides and is located within a polynucleotide sequence between about +54 kb and about +63 kb downstream of the BCL11A transcription start site (TSS). In certain embodiments, the target nucleotide sequence comprises at least 15, at least 16, at least 17, at least 18, at least 19, or at least 20 consecutive nucleotides and is located within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence comprises at least 15, at least 16, at least 17, at least 18, at least 19, or at least 20 consecutive nucleotides and is located within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments wherein the disease is β-thalassemia or sickle cell disease, the target nucleotide sequence comprises at least 15, at least 16, at least 17, at least 18, at least 19, or all 20 consecutive nucleotides located within the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, provided herein is a method of treating sickle cell disease.

In some embodiments, provided herein is a method of treating a disease, where the disease is a PID. In some embodiments, the PID is a severe combined immunodeficiency (SCID), Wiskott-Aldrich syndrome, chronic granulomatous disease, immunodysregulation polyendocrinopathy enteropathay X-linked (IPEX), a hyper IgM syndrome, or X-linked agammaglobulinemia. In some embodiments, the PID is a SCID. In some embodiments, the SCID is Artemis-SCID (ART-SCID), recombination activating gene SCID (RAG-SCID), X-linked SCID (X-SCID), adenosine deaminase-deficient SCID, interleukin 7 receptor deficiency SCID, or JAK3 SCID. In some embodiments, the SCID is ART-SCID, and wherein administration of the LNP results in insertion of a DCLREIC transgene, or a fragment thereof, into at least one HSC of the subject; increased expression of functional Artemis protein in the subject; or a combination thereof. In some embodiments, the SCID is RAG-SCID, and wherein administration of the LNP results in insertion of a RAG1 transgene or a RAG2 trangene, or a fragment thereof, into at least one HSC of the subject; increased expression of functional RAG1 protein or RAG2 protein in the subject; or a combination thereof. In some embodiments, the SCID is X-SCID, and wherein administration of the LNP results in insertion of an IL2RG transgene, or a fragment thereof, in at least one HSC of the subject; increased expression of functional IL2RG protein in the subject; or a combination thereof. In some embodiments, the PID is Wiskott-Aldrich syndrome. In some embodiments, the PID is Wiskott-Aldrich syndrome, and wherein administration of the LNP results in insertion of a WAS transgene, or a fragment thereof, into at least one HSC of the subject; increased expression of functional WASP protein expression in the subject; or a combination thereof. In some embodiments, the PID is chronic granulomatous disease. In some embodiments, the PID is X-linked chronic granulomatous disease. In some embodiments, the PID is chronic granulomatous disease, and wherein administration of the LNP results in one or more of (i) insertion of a CYBA transgene, a CYBB transgene, an NCF1 transgene, NCF2 transgene, or an NCF4 transgene, or a fragment thereof, into at least one HSC of the subject; (ii) introduction of a point 676C>T pointe mutation in the CYBB gene of at least one HSC in the subject; (iii) increased expression of functional CYBA protein, CYBB protein, NCF1 protein, NCF2 protein, or NCF4 protein in the subject; and (v) an increased amount of functional NADPH oxidase enzyme complex in the subject. In some embodiments, the PID is IPEX. In some embodiments, the PID is IPEX, and wherein administration of the LNP results in insertion of an FOXP3 transgene, or a fragment thereof, into at least one HSC of the subject; increased expression of functional FOXP3 protein in the subject; or a combination thereof. In some embodiments, the PID is hyper IgM syndrome. In some embodiments, the PID is hyper IgM syndrome, and wherein administration of the LNP results in one or more of (i) insertion of a AICDA transgene, a UNG transgene, an CD40 transgene, or a CD40LG transgene, or a fragment thereof, into at least one HSC of the subject; (ii) increased expression of functional AICDA protein, UNG protein, CD40 protein, or CD40LG protein in the subject; (iii) a decreased amount of IgM antibodies in the subject; and (iv) an increased amount of IgG, IgA, or IgE antibodies in the subject.

In some embodiments, provided herein is a method of treating a disease, where the disease is a congenital cytopenia. In some embodiments, the congenital cytopenia is Fanconia anemia, Shwachman-Diamond syndrome, Blackfan-Diamond anemia, dyskeratosis congenita, congenital amegakaryocytic thrombocytopenia, or reticular dysgenesis. In some embodiments, the congenital cytopenia is Fanconia anemia, and wherein administration of the LNP results in insertion of one or more FANC genes, or a fragment thereof, into at least one HSC in the subject; increased expression of one or more functional FANC proteins in the subject; or a combination thereof. In some embodiments, the congenital cytopenia is Fanconia anemia, and wherein administration of the LNP insertion of a FANCA transgene, or a fragment thereof, into at least one HSC of the subject; increased expression of functional FANCA in the subject; or a combination thereof.

In some embodiments, provided herein is a method of treating a disease, where the disease is a hemophilia. In some embodiments, the hemophilia is hemophilia A, hemophilia B, or hemophilia C. In some embodiments, the disease is a hemophilia, and wherein administration of the LNP results in (i) insertion of a F8 transgene, a F9 transgene, or an F11, or a fragment thereof, into at least one HSC of the subject; (ii) increased expression of functional factor VIII protein, factor IX protein, or factor XI protein in the subject; and (iii) increased blood clotting in the subject.

In some embodiments, provided herein is a method of treating a disease, where the disease is a thrombophilia. In some embodiments, the thrombophilia is amegakaryocytic thrombocytopenia or factor X deficiency. In some embodiments, the disease is a thrombophilia, and wherein administration of the LNP results in one or more of (i) insertion of a F5 transgene, a F2 transgene, a transgene encoding antithrombin III, a transgene encoding protein C, or a transgene encoding protein S, or a fragment thereof, into at least one HSC of the subject; (ii) increased expression of functional factor V protein, factor II protein, antithrombin III protein, protein C, or protein S in the subject; and (iii) reduced blood clotting in the subject.

In some embodiments, provided herein is a method of treating a disease, where the disease is an inborn error of metabolism. In some embodiments, the inborn error of metabolism is phenylketoneuria, medium-chain acyl-CoA dehydrogenase (MCAD) deficiency, a lysosomal storage disease, a glycogen storage disorder, a peroxisomal disorder, Fabry disease, Gaucher disease, Hurler syndrome, Hunter syndrome, Wolman disease, or pyruvate kinase deficiency. In some embodiments, the peroxisomal disorder is X-linked adrenoleukodystrophy. In some embodiments, the lysosomal storage disease is metachromatic leukodystrophy, mucopolysaccharidosis I, or mucopolysaccharidosis II.

In some embodiments, provided herein is a method of treating a disease, where the disease is a neuropathy. In some embodiments, the neuropathy is Friedrich's ataxia.

In some embodiments, provided herein is a method of treating a disease, where the disease is a viral disease. In some embodiments, the viral disease is HIV/AIDS. In some embodiments, the viral disease is HIV/AIDS, and wherein administration of the LNP prevents infection by HIV, progression of HIV/AIDS, or a combination thereof.

LNPs described in the present disclosure are suitable for the methods described.

In some embodiments, the methods of treatment provided herein comprises delivering an LNP comprising Lipid 15, a HSC targeting group that binds to a HSC surface antigen comprising a VH domain comprising CDR-H1, CDR-H2, and CDR-H3 sequences, where the VH domain has at least 85, 90, 95, 96, 97, 98, 99, or 99.5% sequence identity with the amino acid sequence set forth in SEQ ID NO:7, and a VL domain comprising CDR-L1, CDR-L2, and CDR-L3 sequences, where the VL domain has at least 85, 90, 95, 96, 97, 98, 99, or 99.5% sequence identity with the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL11A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, the methods of treatment provided herein comprises delivering an LNP comprising Lipid 15, a HSC targeting group that binds to a HSC surface antigen comprising a VH domain comprising CDR-H1, CDR-H2, and CDR-H3 sequences, where the VH domain comprises the amino acid sequence set forth in SEQ ID NO:7, and a VL domain comprising CDR-L1, CDR-L2, and CDR-L3 sequences, where the VL domain comprises the amino acid sequence set forth in SEQ ID NO:8, and a payload comprising one or more nucleic acids encoding components of a gene editing system targeting a locus (or loci) wherein gene editing results in increased HbF for the treatment of sickle cell disease or beta-thalessemia. In some embodiments, the target nucleotide sequence is within the BCL11A erythroid enhancer. In certain embodiments, the target nucleotide sequence is within a polynucleotide sequence in intron-2 of the BCL11A gene. In certain embodiments, the target nucleotide sequence is within a polynucleotide sequence between about +54 kb and about +63 kb downstream of the BCL11A transcription start site (TSS). In certain embodiments, the target nucleotide sequence is within a polynucleotide sequence between about +54 kb and about +56 kb, a polynucleotide sequence between about +57 kb and about +59 kb, or a polynucleotide sequence between about +62 kb and about +63 kb downstream of the BCL1/A TSS, or a combination thereof. In certain embodiments the target nucleotide sequence is within a polynucleotide sequence within a distance of about 100 bp, 200 bp, 300 bp, 400 bp, 500 bp, 600 bp, 700 bp, 800 bp, 900 bp, 1.0 kb, 1.1 kb, 1.2 kb, 1.3 kb, 1.4 kb, or 1.5 kb of the nucleotide position at +55 kb, +58 kb, or +62 kb downstream of the BCL11A TSS, or a combination thereof. In certain embodiments, the target nucleotide sequence comprises the polynucleotide sequence of GTGATAAAAGCAACTGTTAG (SEQ ID NO: 62), or a variant thereof comprising up to 10 (e.g., 1, 2, 3, 4, 5, 6, 7, 8, 9, or 10) nucleotide substitutions. In some embodiments, delivery of the exemplary LNP provided herein is used to edit HSC cells in vivo and treat disease in a subject, wherein the disease is sickle cell disease or beta-thalessemia. In some embodiments, the gene editing of the BCL11A erythroid enhancer by delivery of the exemplary LNP provided herein results in the treatment of sickle cell disease in a subject. In some aspects, the method of treating a disease further comprises administering to the subject an HSC mobilization agent, wherein the HSC mobilization agent comprises plerixafor and G-CSF. In some embodiments, use of the exemplary LNP provided herein for treatment of sickle cell disease in a subject is safe and effective. In some embodiments, use of the exemplary LNP provided herein for treatment of beta-thalessemia in a subject is safe and effective.

### IX. Kits for Use in Medical Applications

Another aspect of the invention provides a kit for treating a disease. The kit may comprise one or more of: an ionizable cationic lipid, a lipid-HSC targeting group or conjugate thereof (e.g., a lipid-antibody conjugate, e.g., wherein the antibody binds to CD105 and/or CD117), a lipid nanoparticle composition comprising an ionizable cationic lipid and/or a lipid-HSC targeting group or conjugate thereof (e.g., a lipid-antibody conjugate, e.g., wherein the antibody binds to CD105 and/or CD117) with or without an encapsulated payload (e.g., a nucleic acid, e.g., an mRNA encoding a site-directed nuclease, a chemical base editor, a prime editor, or an epigenome editor and, optionally, a gRNA or pegRNA), and instructions for treating a medical disease described herein (e.g., sickle cell disease).

### EXAMPLES

The presently disclosed subject matter will be better understood by reference to the following Examples, which are provided as exemplary of the invention, and not by way of limitation.

### Example 1: Preparation of Ionizable Cationic Lipids

This Example describes the synthesis of various cationic lipids

### General Scheme for the Synthesis of Lipids 1 Through Lipid 25

General scheme for the synthesis of Lipids 1 through Lipid 25 is provided in Scheme 1 below. Corresponding R and R′ for each lipid is provided in Tables 1 to 3 below.

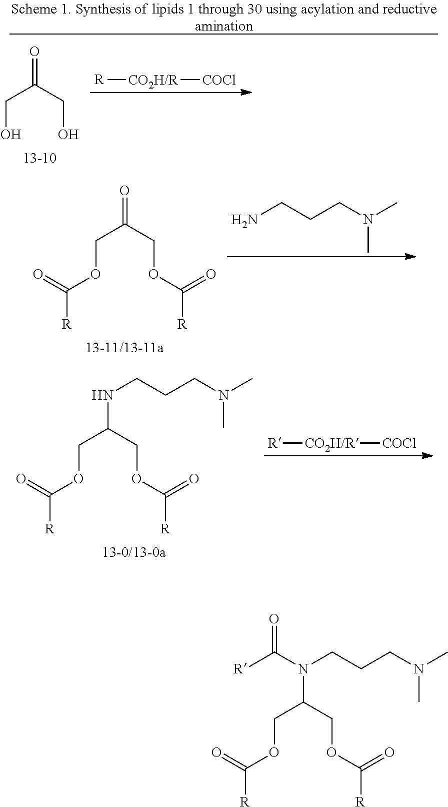

### Synthesis of Intermediates 13-11 and 13-11a

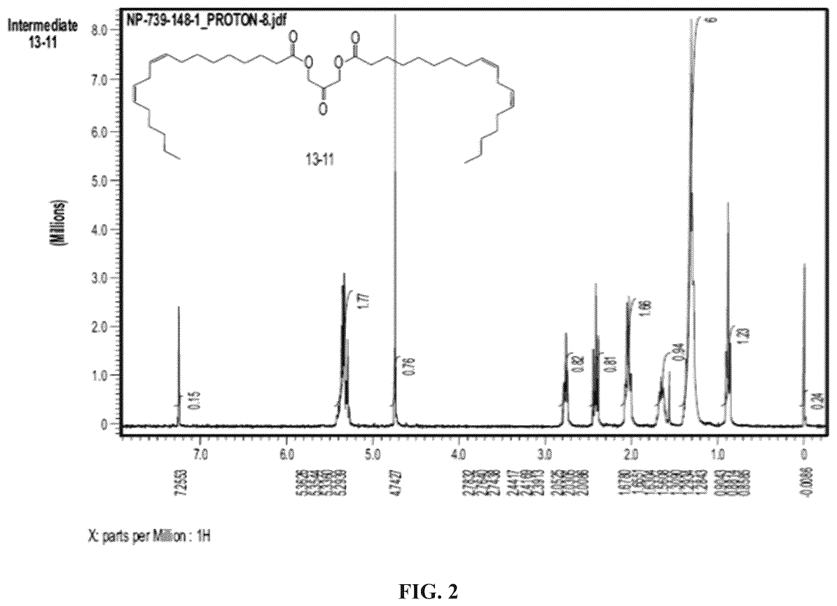

Intermediate 13-11 (Scheme 2) was synthesized by acylation of dihydroxyacetone (13-10) with linoleic acid. Dihydroxyacetone (22 mmol, 2 g, 1 eq.) was reacted with linoleic acid 1-5 (55 mmol, 15.4 g, 2.5 eq.) using EDCI (55 mmol, 10.5 g, 2.5 eq.) activation in 50 mL DCM, in the presence of DIPEA (55 mmol, 9.6 mL, 2.5 eq.), DMAP (4.4 mmol, 540 mg, 0.2 eq.) at room temperature yielding 11.1 g (79%) crude product. Purified product was obtained by column chromatography and characterized by proton NMR spectroscopy (FIG. 2).

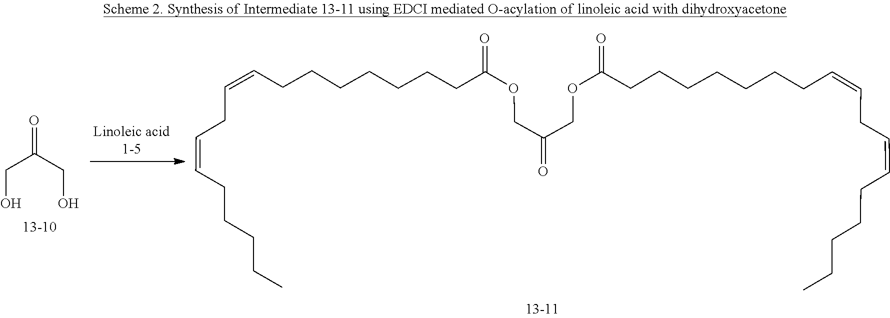 [SMILES: O=C(CO)CO ; CCCCC/C=C\C/C=C\CCCCCCCC(=O)OCC(=O)COC(=O)CCCCCCC/C=C\C/C=C\CCCCC]

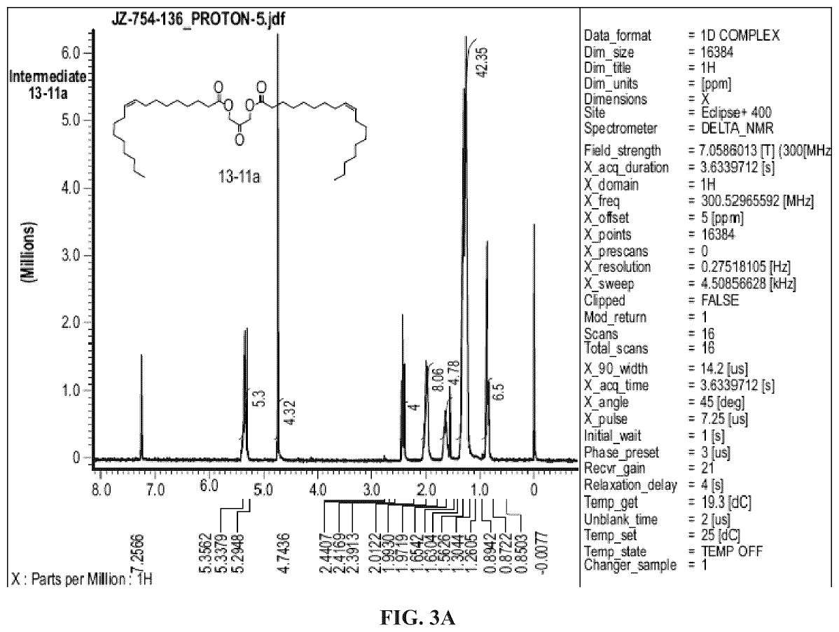

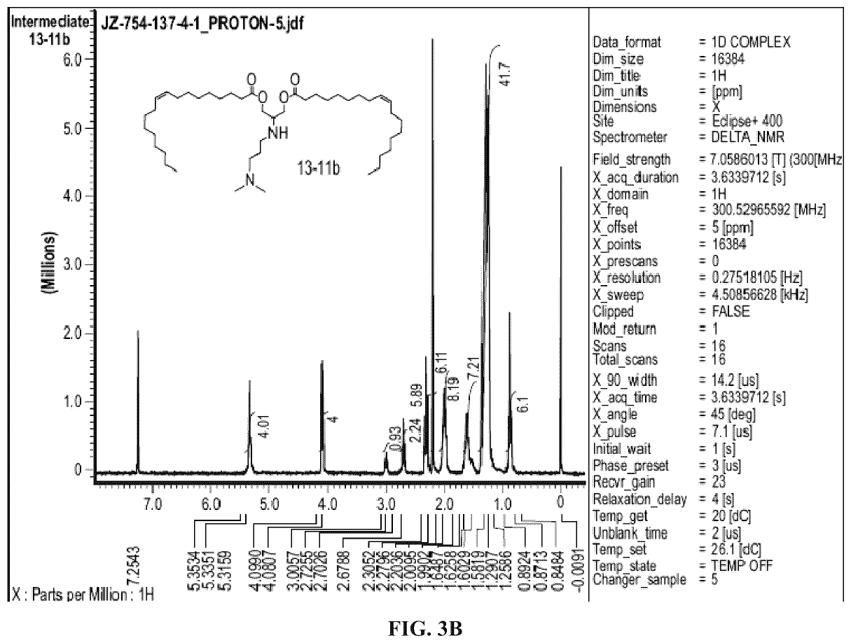

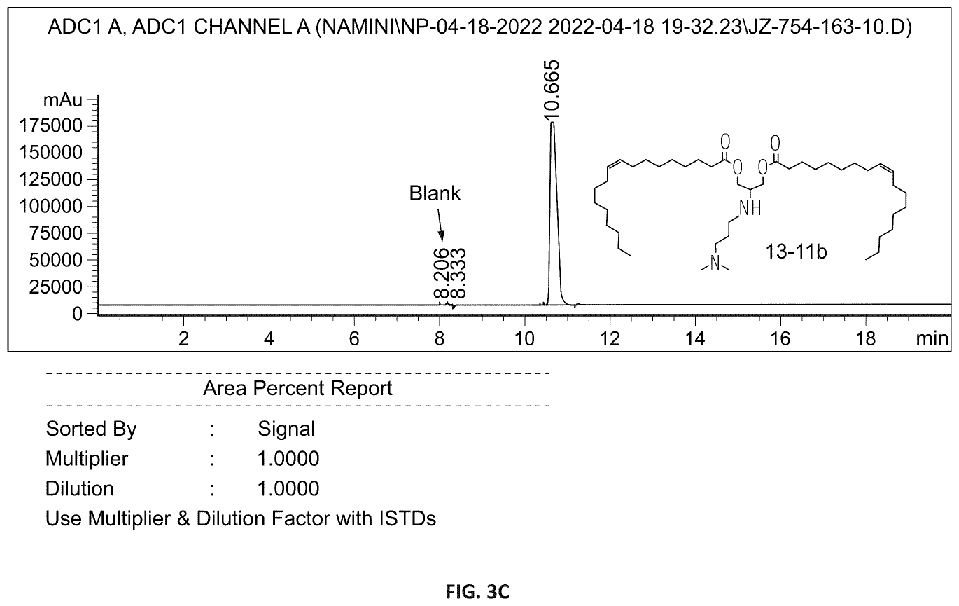

Intermediates 13-11a (Scheme 3) was synthesized by acylation of dihydroxyacetone (13-10) with oleoyl chloride. Dihydroxyacetone (44.4 mmol, 4 g, 1 eq.) was reacted with oleoyl chloride 1-6a (111 mmol, 36.7 mL, 2.5 eq.) in the presence of Pyridine (133.3 mmol, 11 mL, 3 eq.), DMAP (13.3 mmol, 1.63 g, 0.3 eq.) in 80 mL DCM, at room temperature yielding 14.9 g (54%) crude product. Crude product was purified by column chromatography and characterized by proton NMR spectroscopy (FIG. 3A).

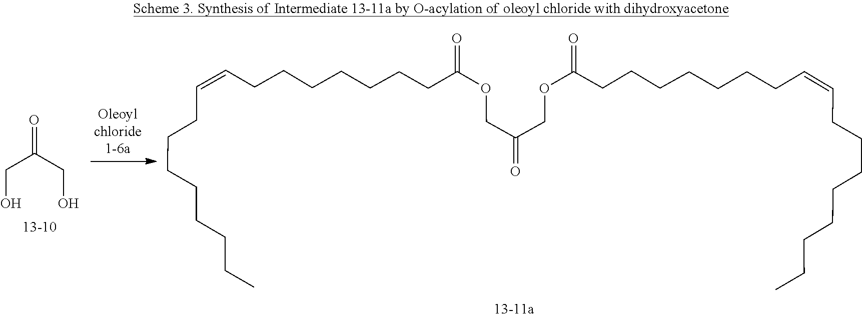 [SMILES: O=C(CO)CO ; CCCCCCCC/C=C\CCCCCCCC(=O)OCC(=O)COC(=O)CCCCCCC/C=C\CCCCCCCC]

### Synthesis of Intermediates 13-0a and 13-11b

Intermediates 13-0a and 13-11b were synthesized by reductive amination of intermediates 13-11 and 13-11a respectively

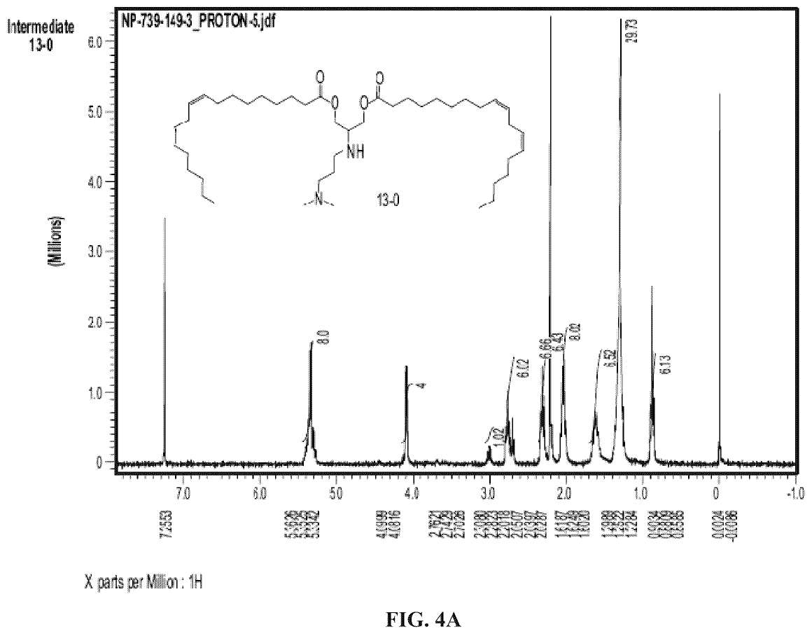

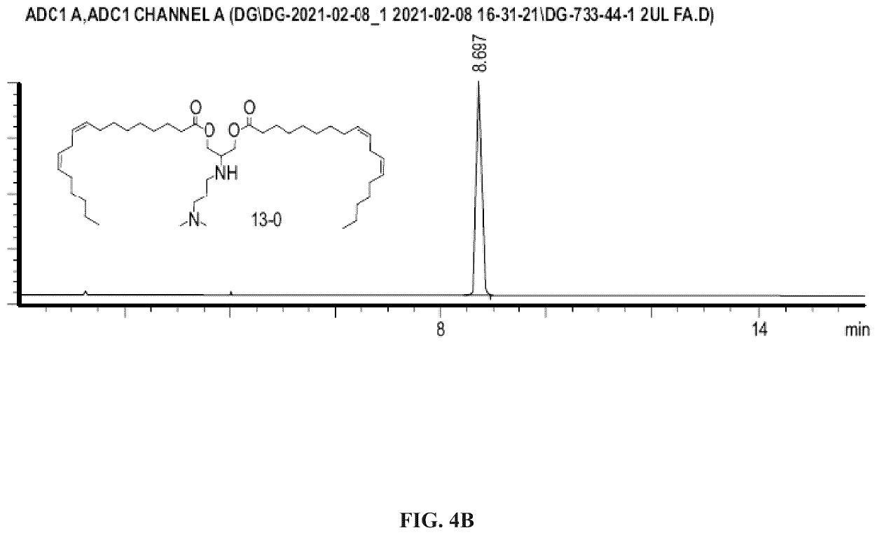

Intermediate 13-0 was produced by reductive amination (scheme 4) of intermediate 13-11 (13.1 mmol, 8.1 g, 1.0 eq) using N1,N1-dimethylpropane-1,3-diamine 15-3 (26 mmol, 3.2 mL, 2.0 eq.) in DCM (10 mL) using acetic acid (26.0 mmol, 1.50 mL, 2 eq.) and sodium borohydride triacetate (4.32 mmol, 3.3 g, 1.2 eq.) yielding 3.1 g (32%) crude product. Column purification resulted in pure product (Proton NMR Spectrum and LC-CAD chromatogram shown in FIG. 4A and FIG. 4B, respectively).

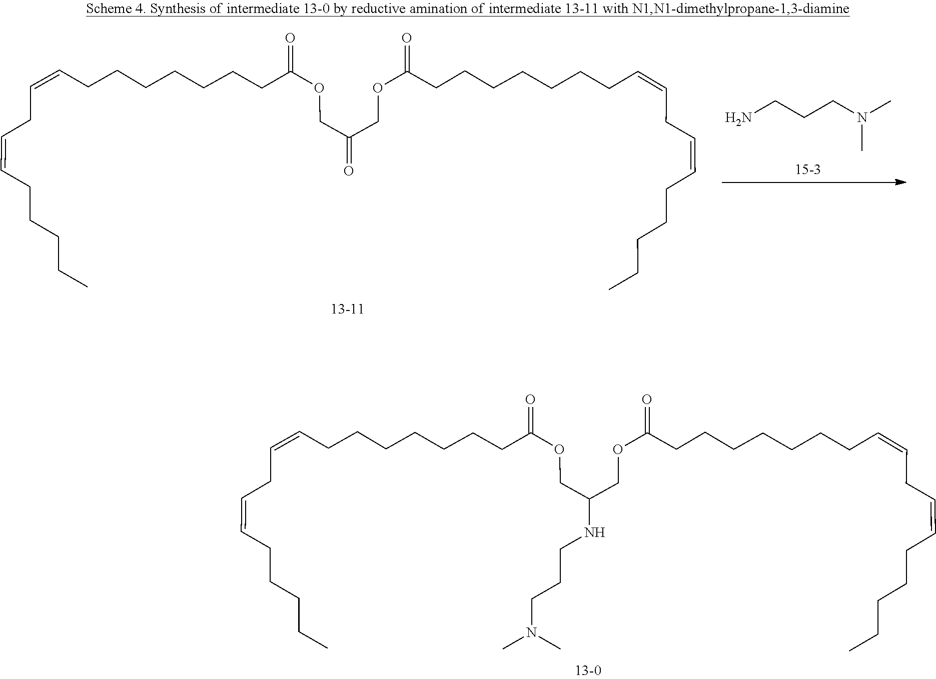

Intermediate 13-11b was produced by reductive amination (scheme 5) of intermediate 13-1 Ia (24.2 mmol, 14.9 g, 1.0 eq) using N1,N1-dimethylpropane-1,3-diamine 15-3 (48.4 mmol, 6.05 mL, 2.0 eq.) in DCM (60 mL) using acetic acid (48.4 mmol, 2.8 mL, 2 eq.) and sodium borohydride triacetate (29.1 mmol, 6.05 g, 1.2 eq.) yielding 6 g (35%) crude product. Column purification resulted in purified product (Proton NMR Spectrum and LC-ELSD chromatogram shown in FIG. 3B and FIG. 3C, respectively).

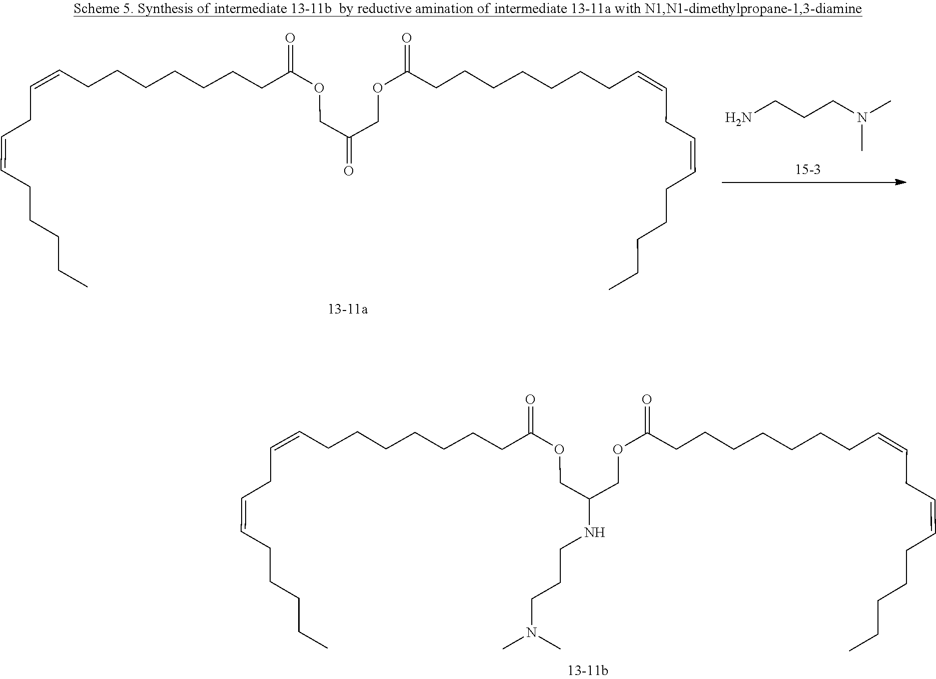

TABLE 1
R (O-acyl) an′ R′ (N-acyl) groups of lipids 1 through 8

| Lipid # | R (O-acyl groups) | R′ (N-acyl group) |
|---|---|---|
| Lipid 1 | 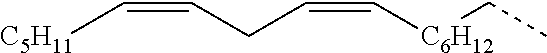 | 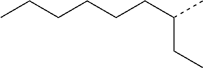 |
| Lipid 2 | 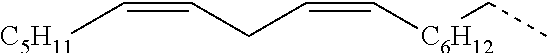 | 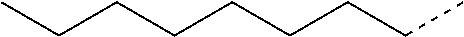 |
| Lipid 3 | 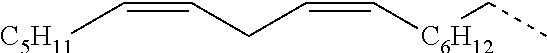 | 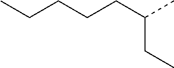 |
| Lipid 4 | 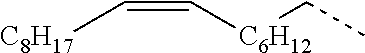 | 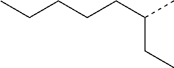 |
| Lipid 5 | 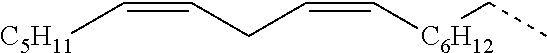 | 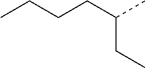 |
| Lipid 6 | 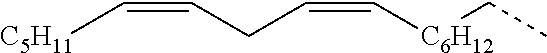 | 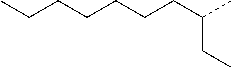 |
| Lipid 7 | 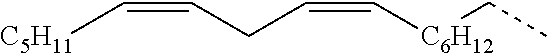 | 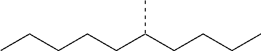 |
| Lipid 8 | 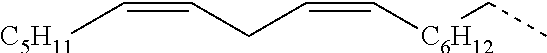 | 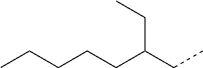 |

TABLE 2
R (O-acyl) an′ R′ (N-acyl) groups of Lipids 9 through 16

| Lipid | | |
|---|---|---|
| # | R (O-acyl groups) | R′ (N-acyl group) |
| Lipid 9 | 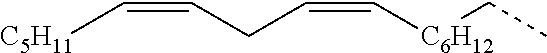 | 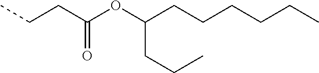 |
| Lipid 10 | 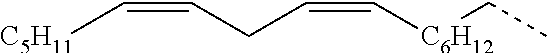 | 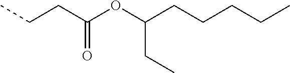 |
| Lipid 11 | 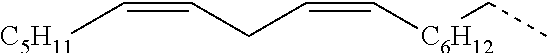 | 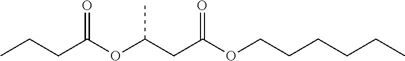 [SMILES: CCCCCCOC(=O)C[C@@H](C)OC(=O)CCC] |
| Lipid 12 | 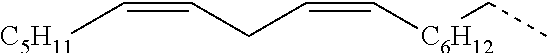 | 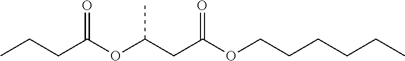 [SMILES: CCCCCCOC(=O)C[C@@H](C)OC(=O)CCC] |
| Lipid 13 | 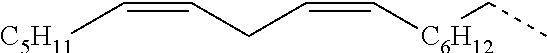 | 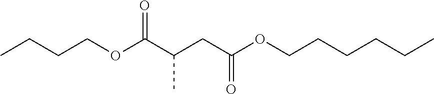 [SMILES: CCCCCCOC(=O)C[C@H](C)C(=O)OCCCC] |
| Lipid 14 | 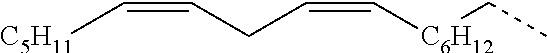 | 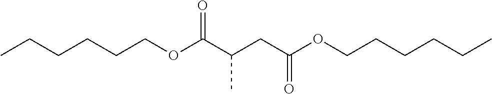 [SMILES: CCCCCCOC(=O)C[C@H](C)C(=O)OCCCCCC] |
| Lipid 15 | 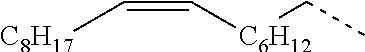 | 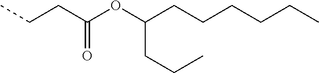 |
| Lipid 16 | 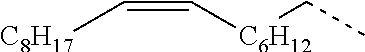 | 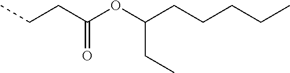 |

TABLE 3
R (O-acyl) and R′ (N-acyl) groups of Lipids 17 through 25

| Lipid # | R (O-acyl groups) | R′ (N-acyl group) |
|---|---|---|
| Lipid 17 | 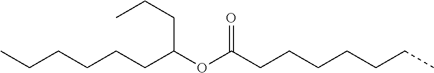 | 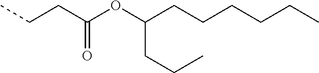 |
| Lipid 18 | 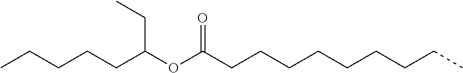 | 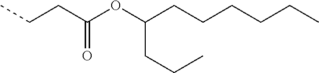 |
| Lipid 19 | 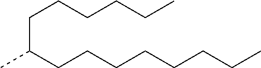 | 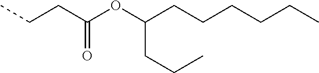 |
| Lipid 20 | 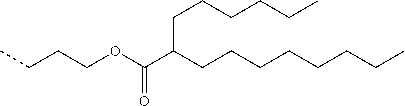 | 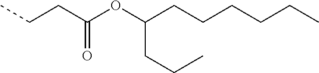 |
| Lipid 21 | 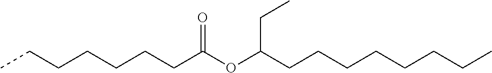 | 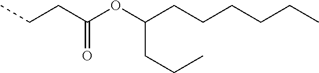 |
| Lipid 22 | 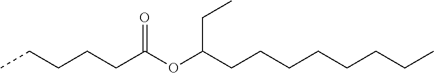 | 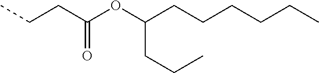 |
| Lipid 23 | 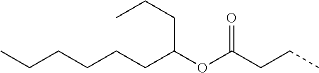 | 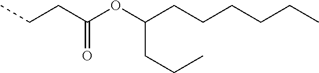 |
| Lipid 24 | 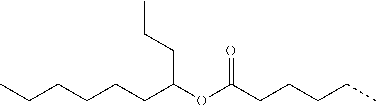 | 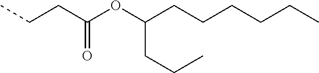 |
| Lipid 25 | 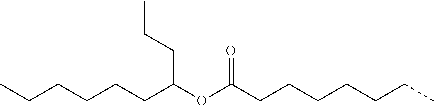 | 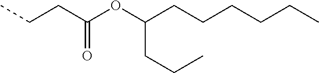 |

TABLE 4
Expected and observed mass (m/z) of named Ionizable lipids

| | | Expected mass | |
|---|---|---|---|
| Entry | Compound code | (g/mol) | Observed mass (m/z) |
| 1 | Lipid 1 | 854.75 | 855.7, 856.7, 857.7 |
| | | | (M + 1, M + 2, M + 3) |
| 2 | Lipid 2 | 840.73 | 841.7, 842.7, 843.7 |
| | | | (M + 1, M + 2, M + 3) |
| 3 | Lipid 3 | 840.73 | 841.7. 842.7, 843.7 |
| | | | (M + 1, M + 2, M + 3) |
| 4 | Lipid 4 | 845.39 | 845.7, 846.7, 847.7 |
| | | | (M, M + 1, M + 2) |
| 5 | Lipid 5 (S) isomer | 827.33 | 827.7, 828.7, 829.7 |
| | | | (M, M + 1, M + 2) |
| 6 | Lipid 6 | 868.76 | 869.7, 870.7, 871.7 |
| | | | (M + 1, M + 2, M + 3) |
| 7 | Lipid 7 | 868.76 | 869.7, 870.7, 871.7 |
| | | | (M + 1, M + 2, M + 3) |
| 8 | Lipid 8 | 854.75 | 855.7, 856.7, 857.7 |
| | | | (M + 1, M + 2, M + 3) |
| 9 | Lipid 9 | 940.78 | 941.7, 942.7, 943.7 |
| | | | (M + 1, M + 2, M + 3) |
| 10 | Lipid 10 (S) isomer | 912.75 | 913.7, 914.7, 915.7 |
| | | | (M + 1, M + 2, M + 3) |
| 11 | Lipid 11 (S) isomer | 970.76 | 971.7, 972.7, 973.7 |
| | | | (M + 1, M + 2, M + 3) |
| 12 | Lipid 12 | 999.51 | 999.0, 1001, 1002 |
| | | | (M + 1, M + 2, M + 3) |
| 13 | Lipid 13 | 984.77 | 985.7, 986.7, 987.6 |
| | | | (M + 1, M + 2, M + 3) |
| 15 | Lipid 15 | 944.82 | 945.1, 946.1, 947.1 |
| | | | (M + 1, M + 2, M + 3) |
| 16 | Lipid 16 | 916.78 | 917.2, 918.2, 919.2 |
| | | | (M + 1, M + 2, M + 3) |
| 17 | Lipid 19 | 892.78 | 893.7, 894.7, 895.7 |
| | | | (M + 1, M + 2, M + 3) |
| 18 | Lipid 20 | 1064.86 | 1065.1, 1066.1, |
| | | | 1067.1, 1068.1 (M + 1, |
| | | | M + 2, M + 3, M + 4) |
| 19 | Lipid 31 | 854.75 | 855.1, 856.1, 857.1 |
| | | | (M + 1, M + 2, M + 3) |
| 20 | Lipid 32 | 854.75 | 855.7, 856.7, 857.7 |
| | | | (M + 1, M + 2, M + 3) |
| 21 | Lipid 33 | 840.73 | 841.7, 842.7, 843.7 |
| | | | (M + 1, M + 2, M + 3) |
| 22 | Lipid 34 | 868.76 | 869.7. 870.7, 871.7 |
| | | | (M + 1, M + 2, M + 3) |
| 23 | Lipid 35 | 884.76 | |

### Synthesis of Lipids 1 Through 24 by N-Acylation of Intermediates 13-0 or 13-11b

N-acylation of intermediates 13-0 and 13-11b with compounds R′CO₂H or R′COCl (R′ structures shown in Table 1 to Table 3 yielded lipids 1 through 24 as described in examples below.

### Synthesis of Lipids 1, 3, 4, 5, 6 and 7 by N-Acylation of Intermediate 13-0 Using the Corresponding Acid Chlorides

### Synthesis of Lipid 1

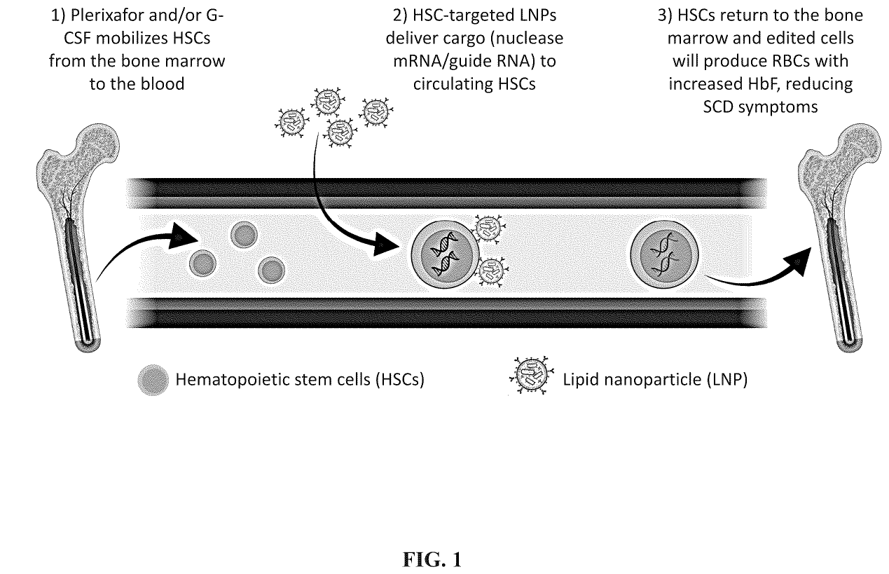

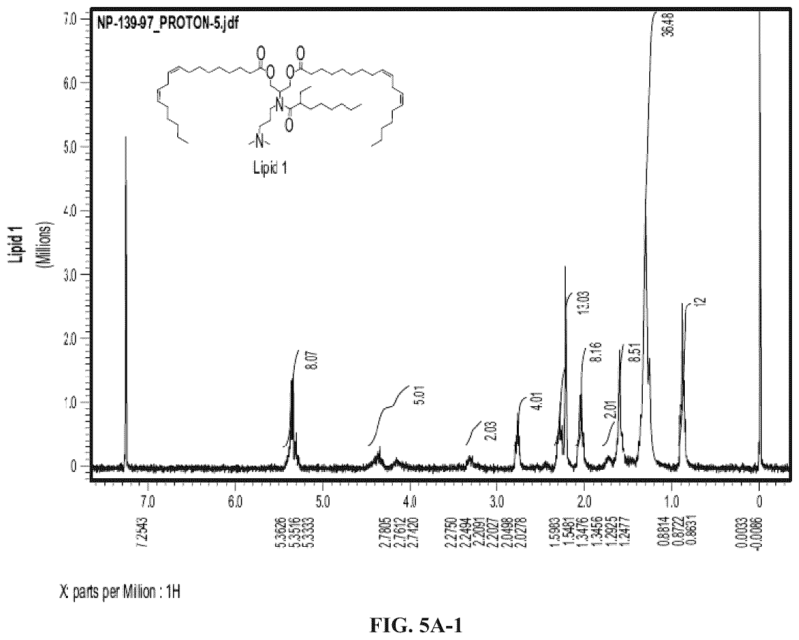

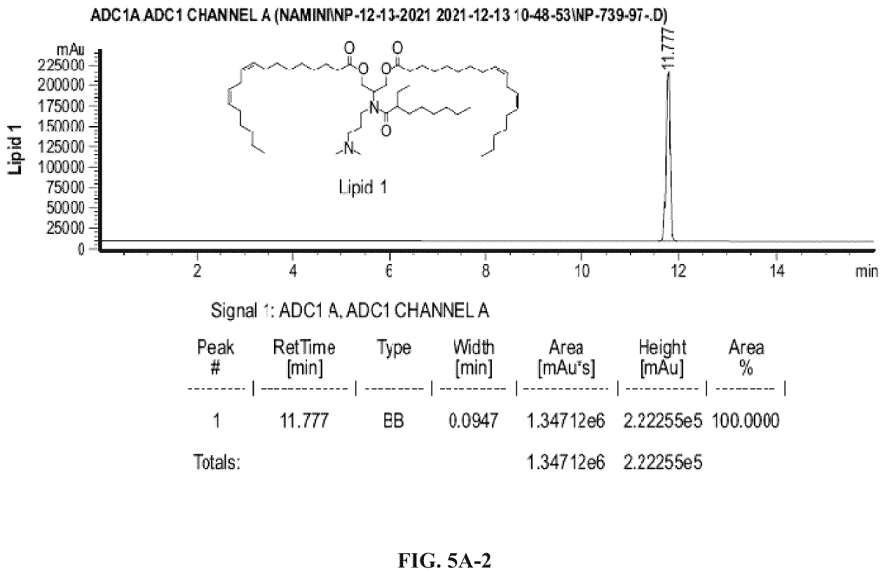

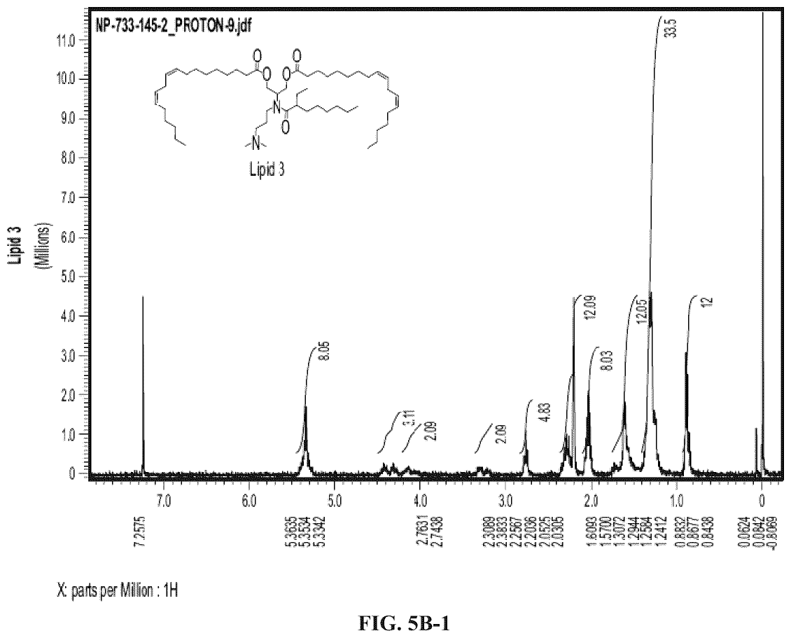

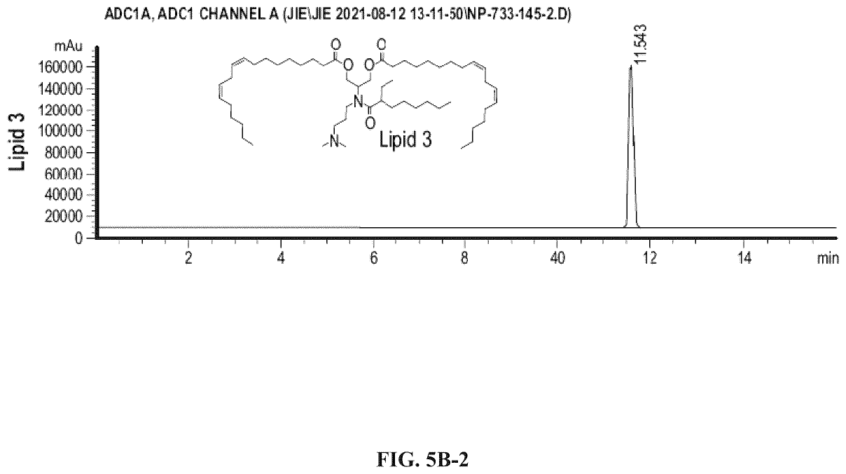

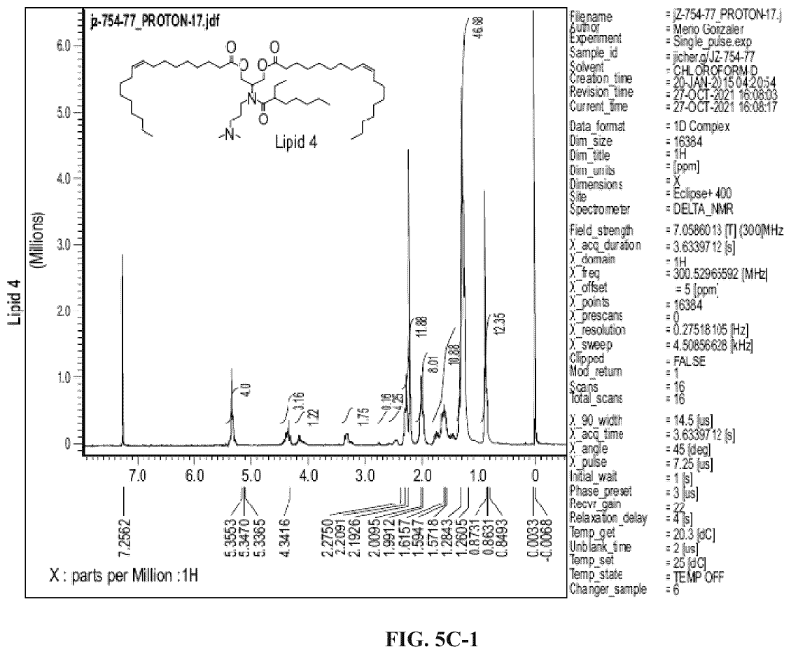

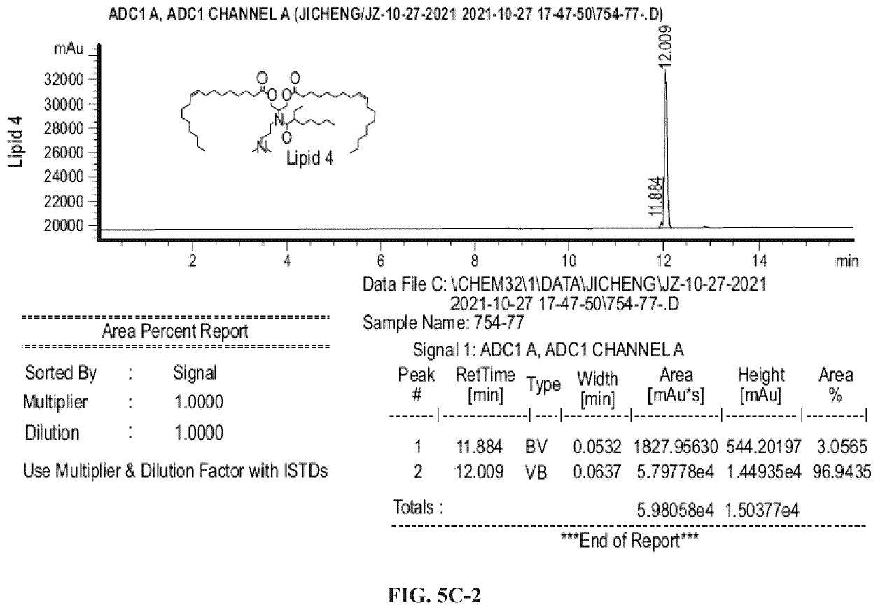

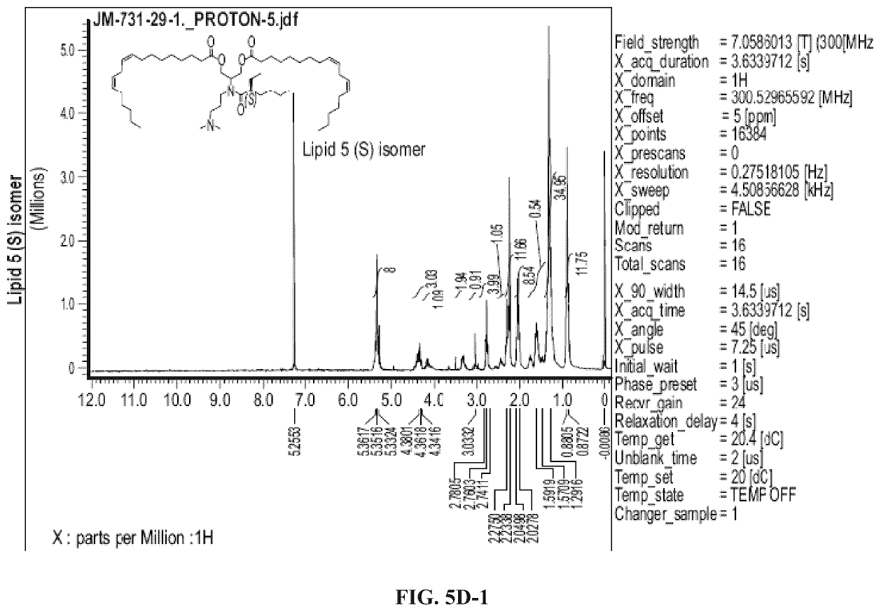

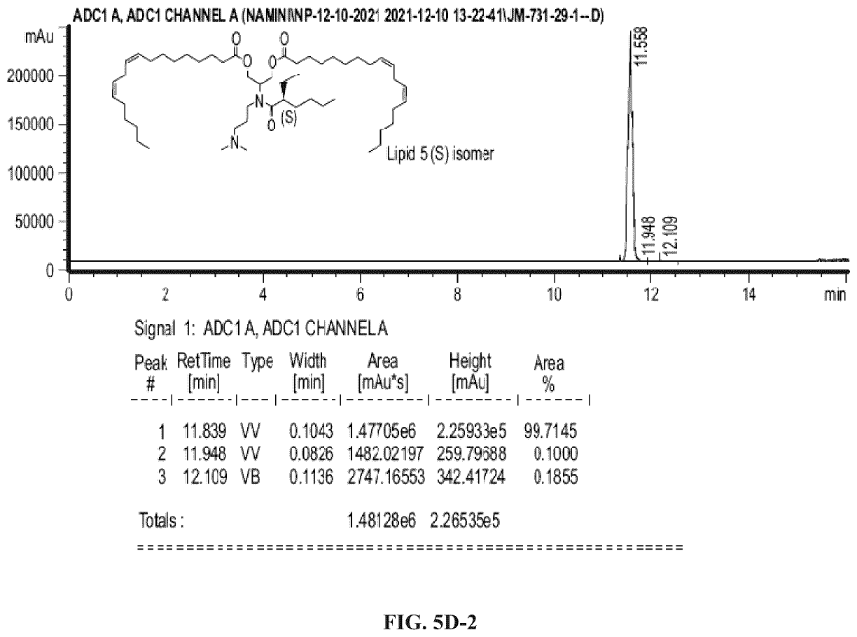

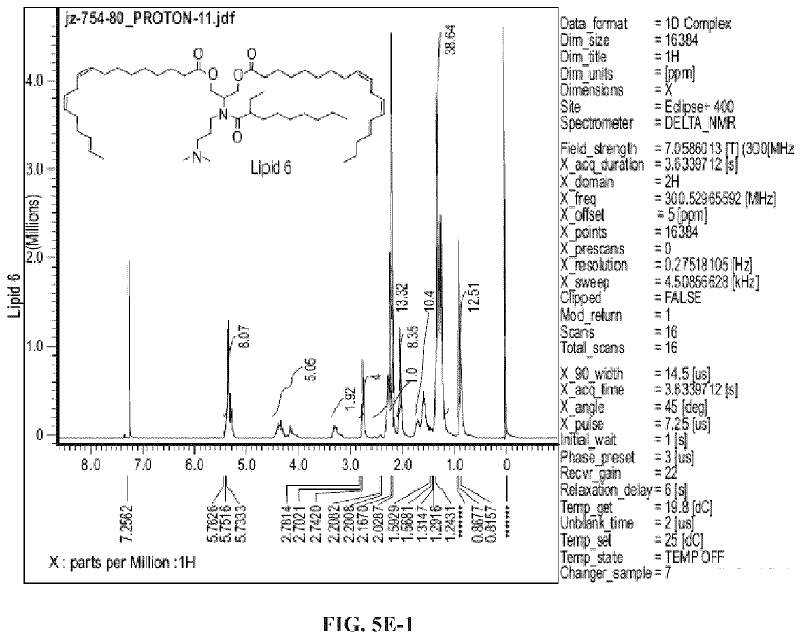

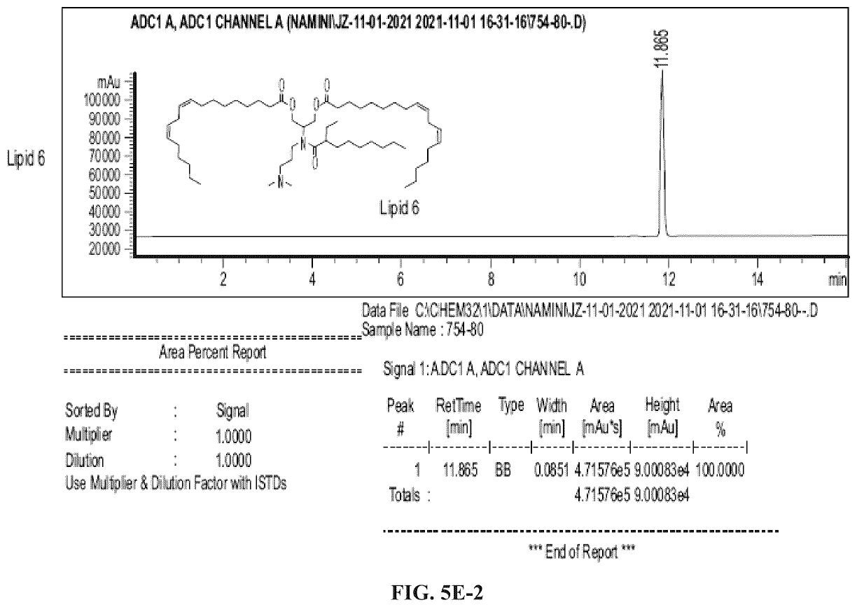

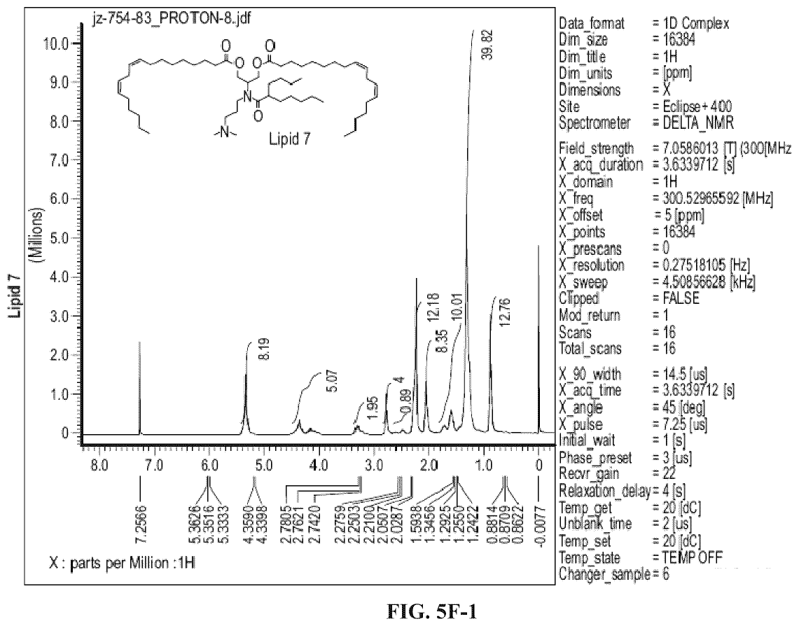

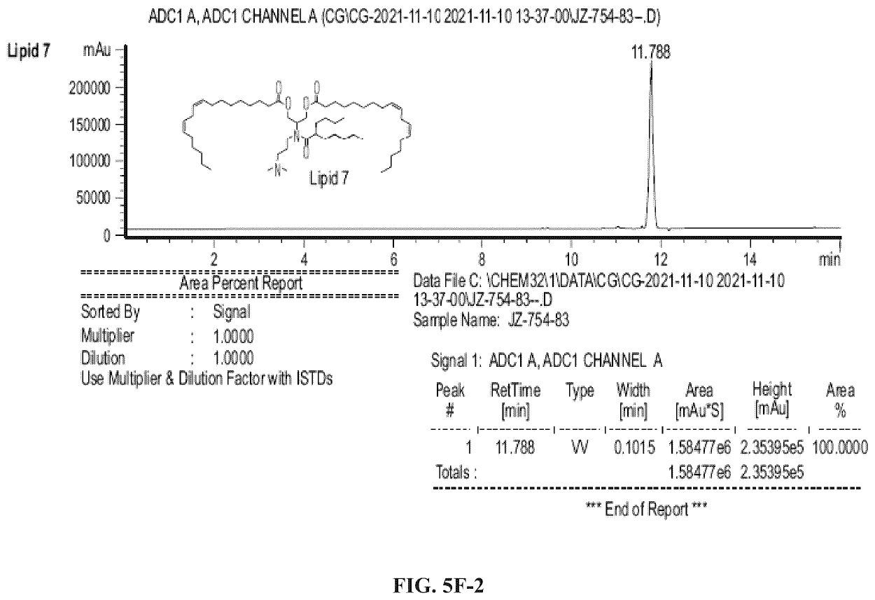

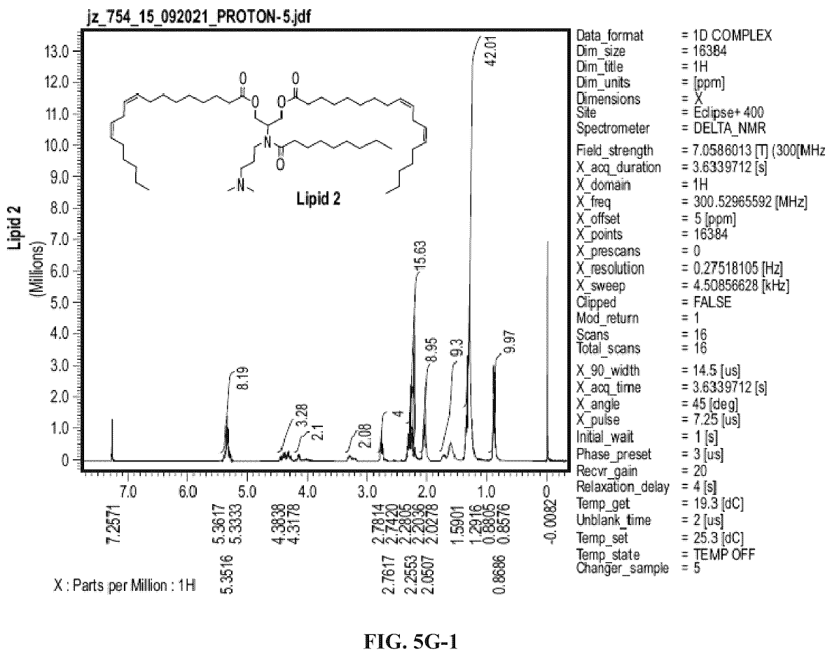

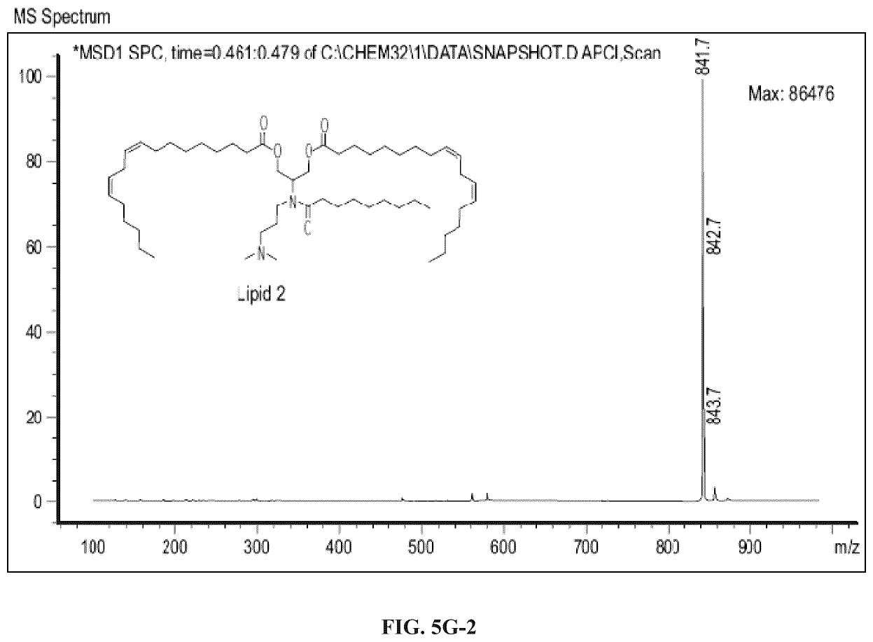

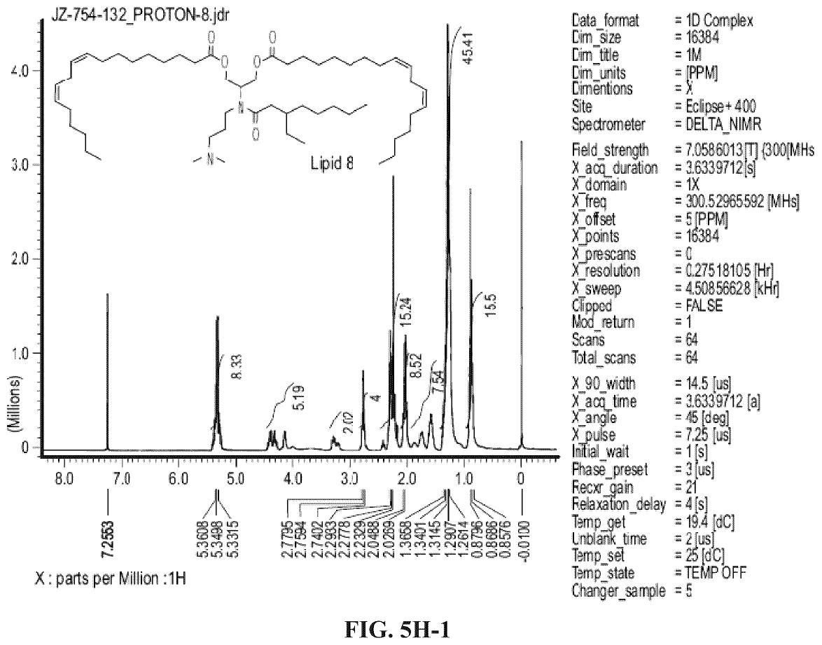

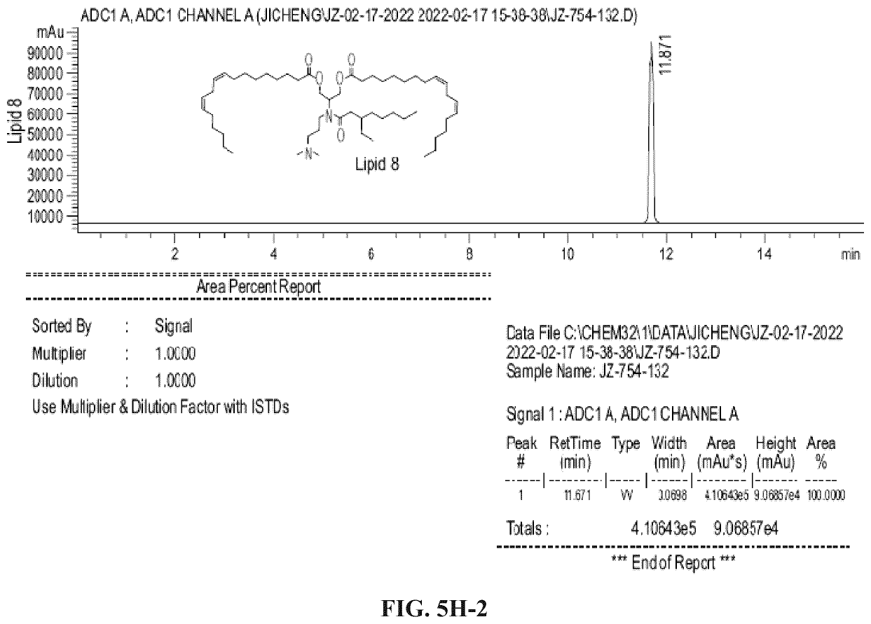

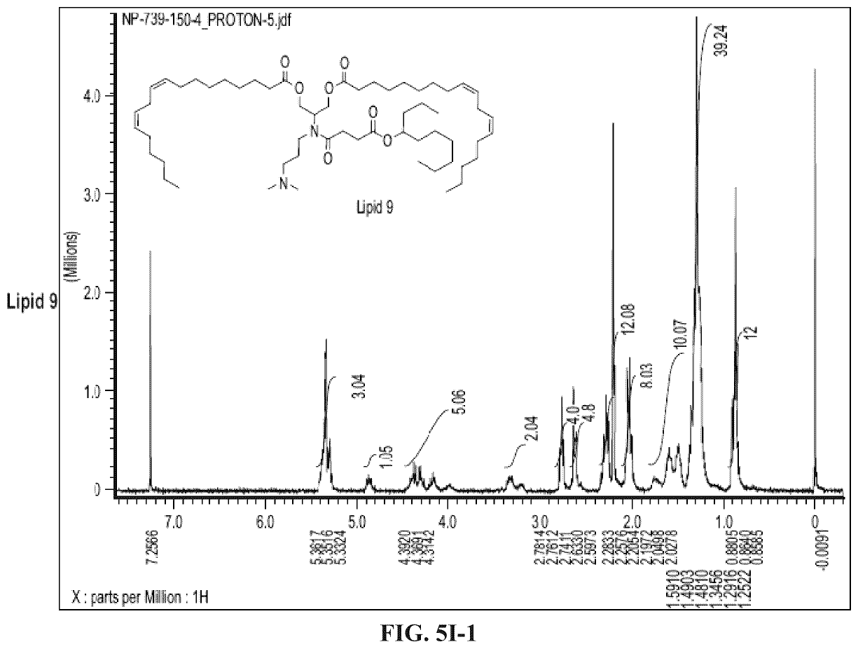

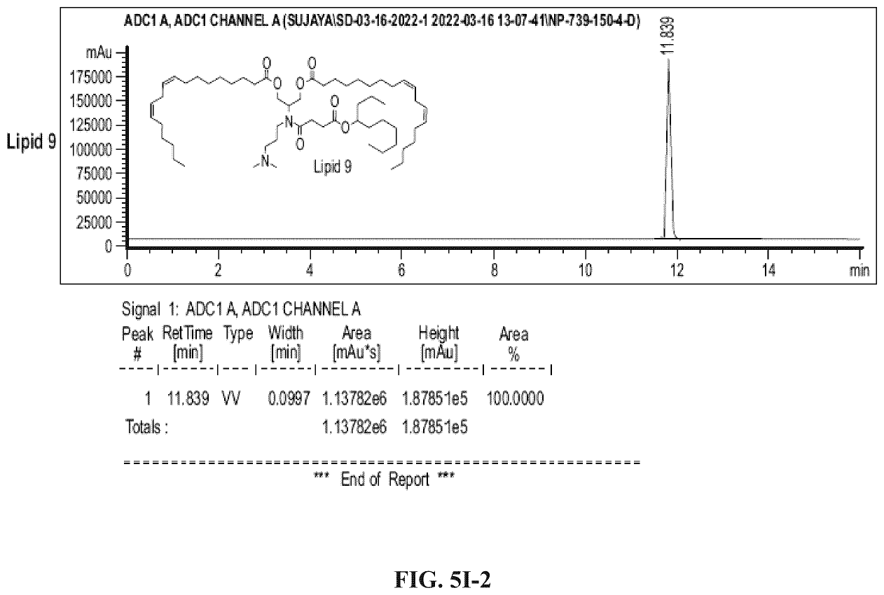

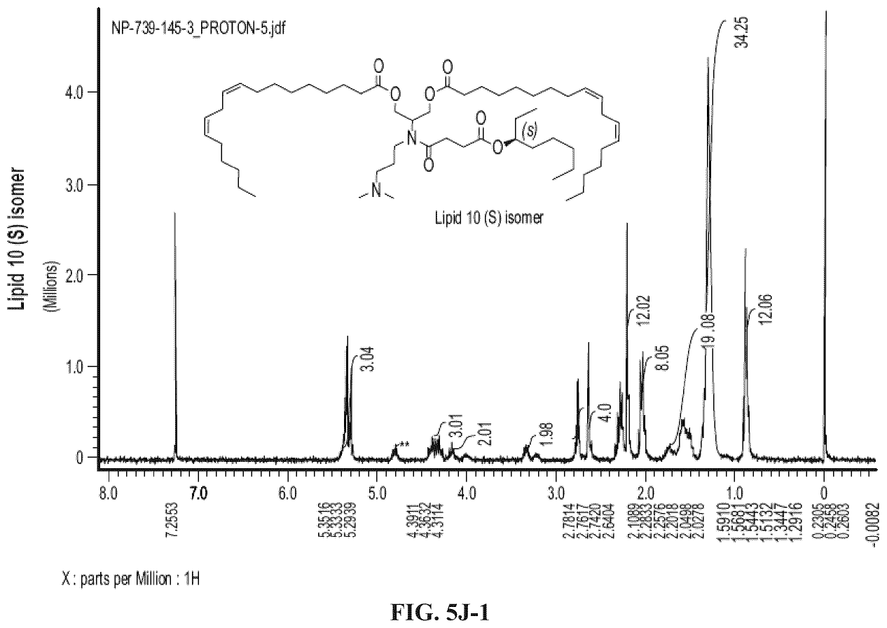

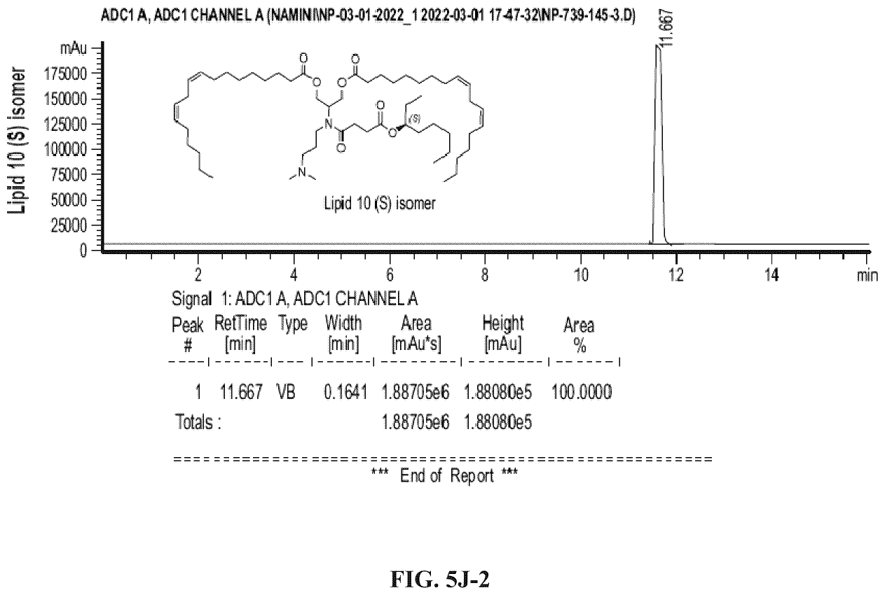

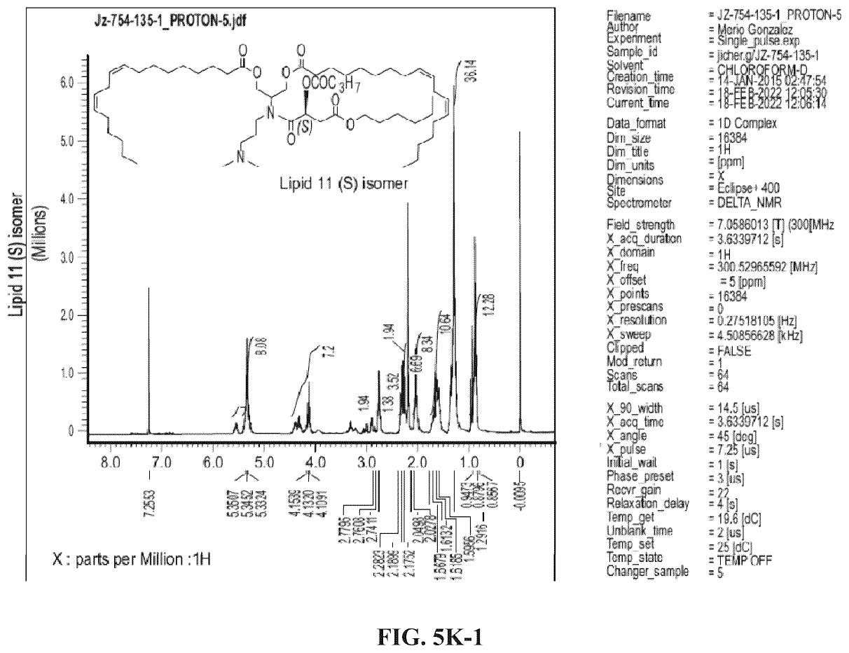

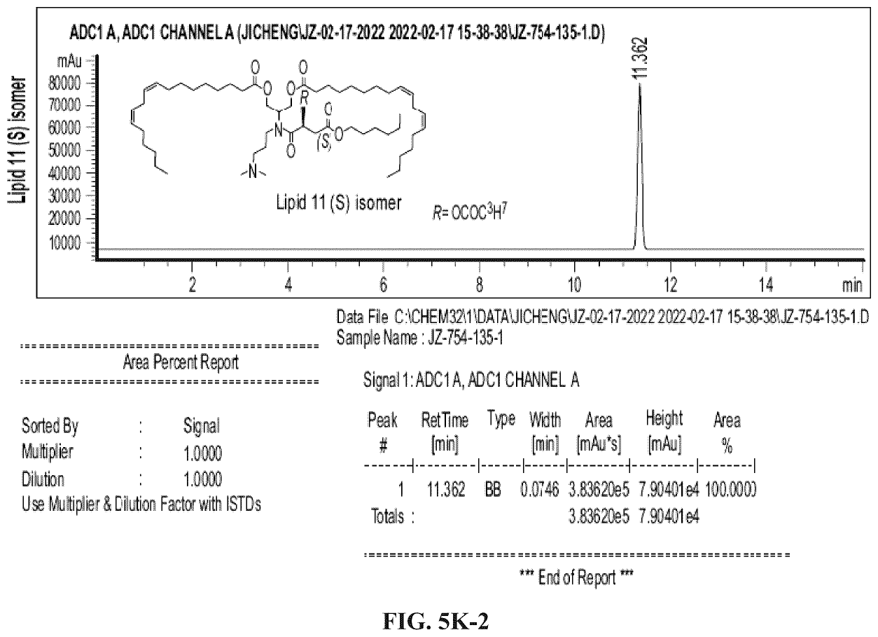

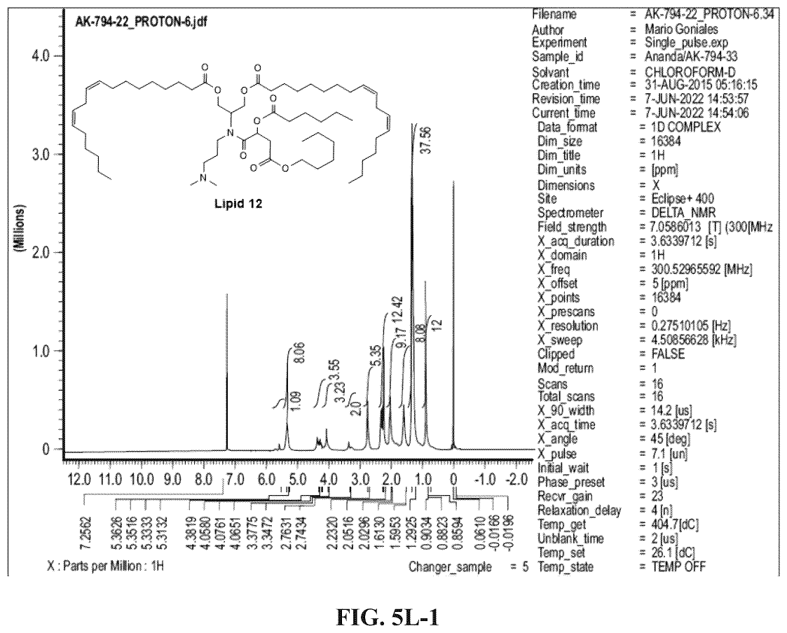

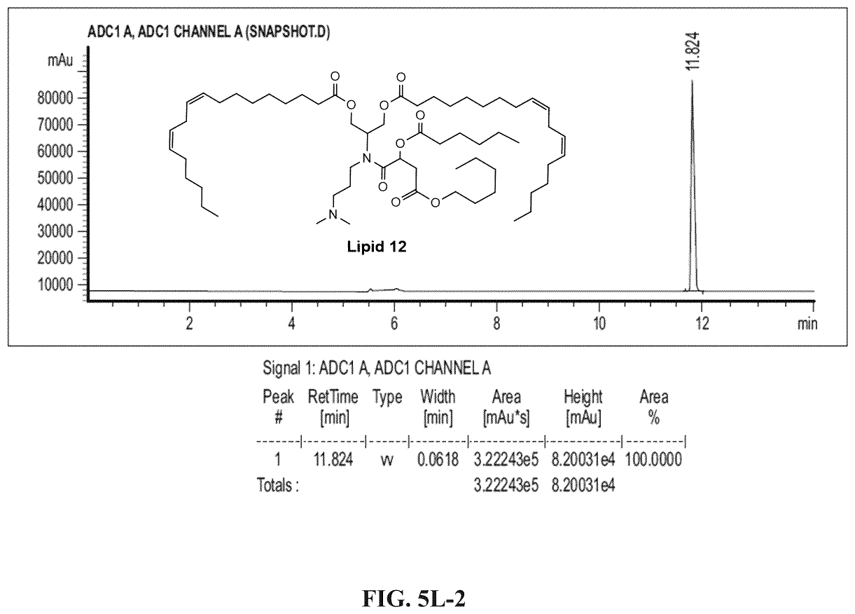

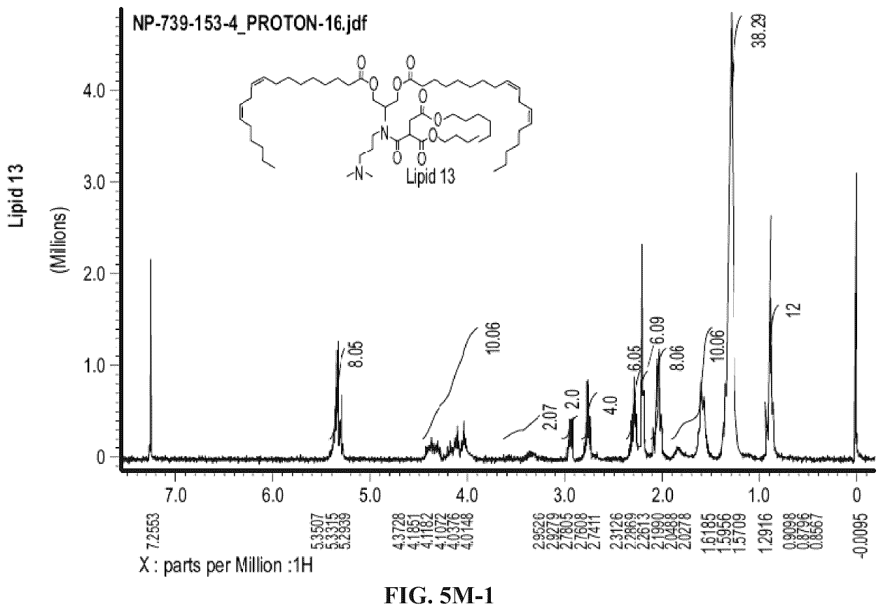

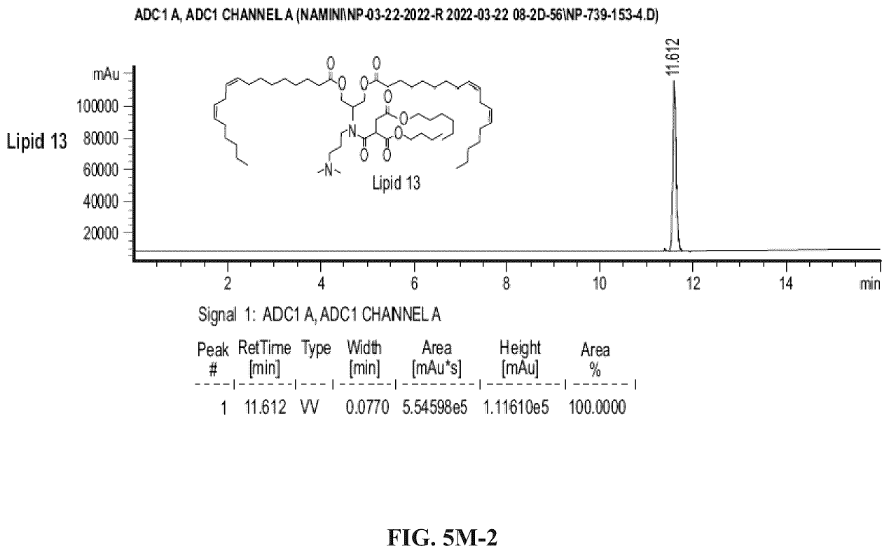

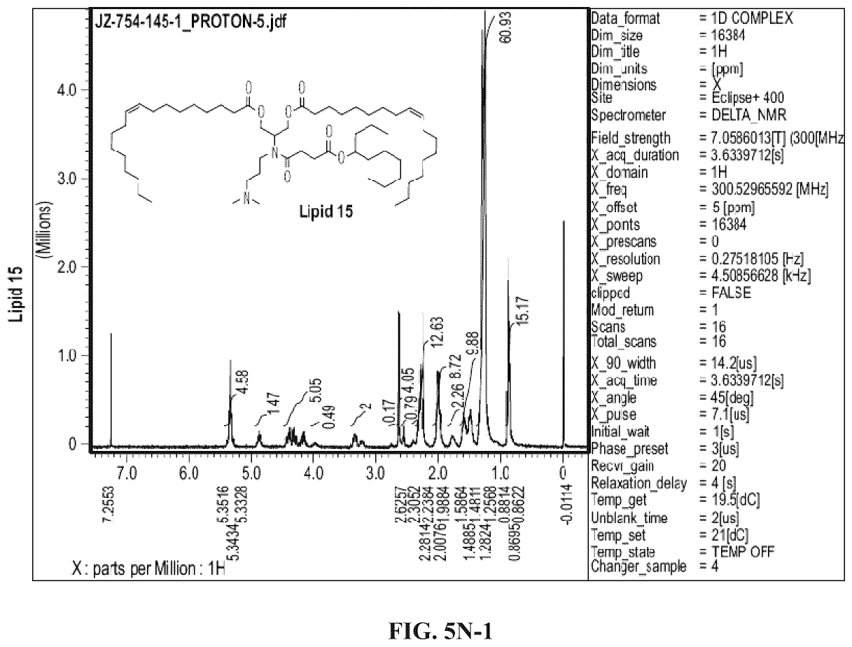

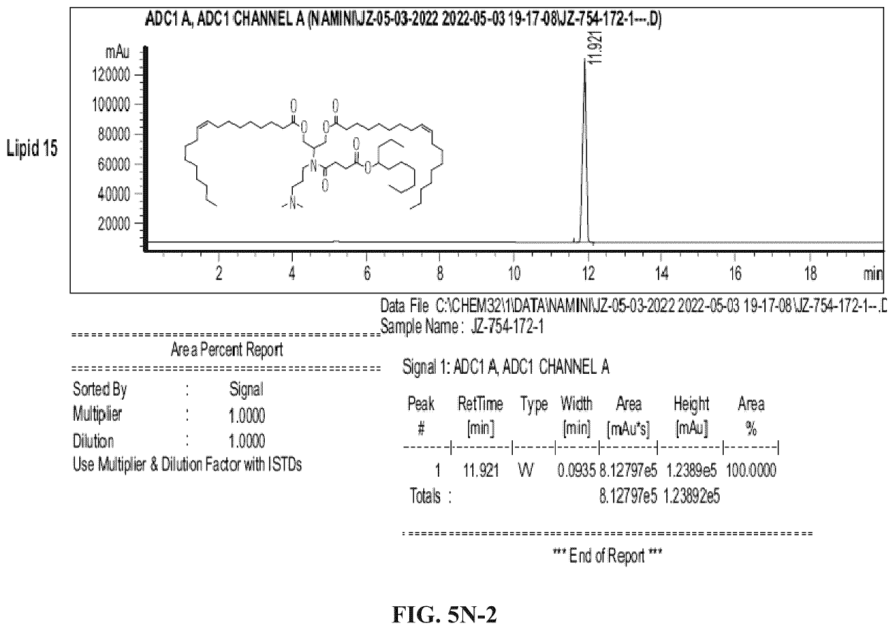

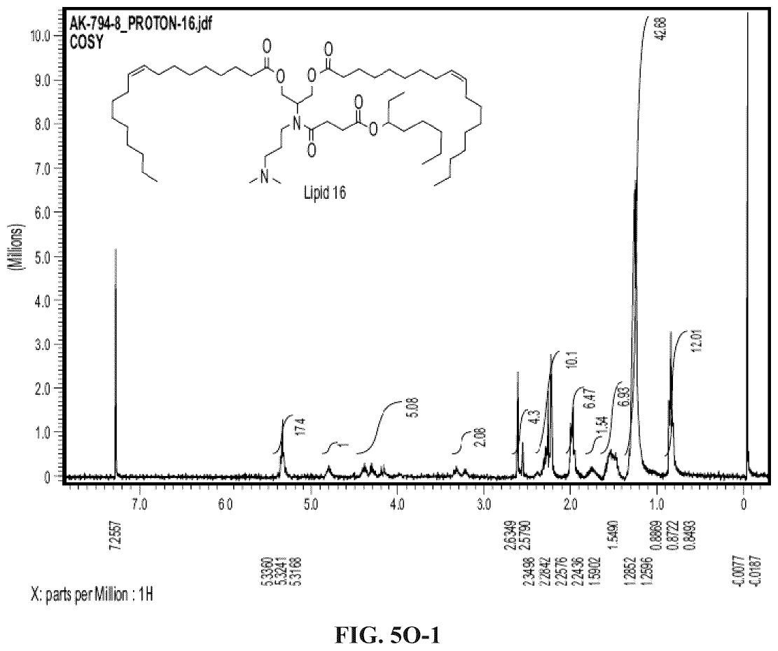

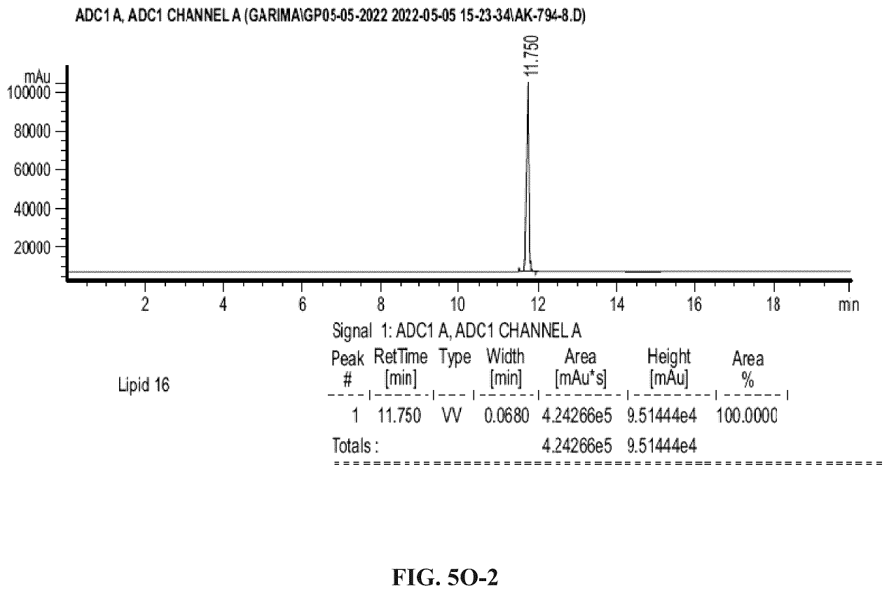

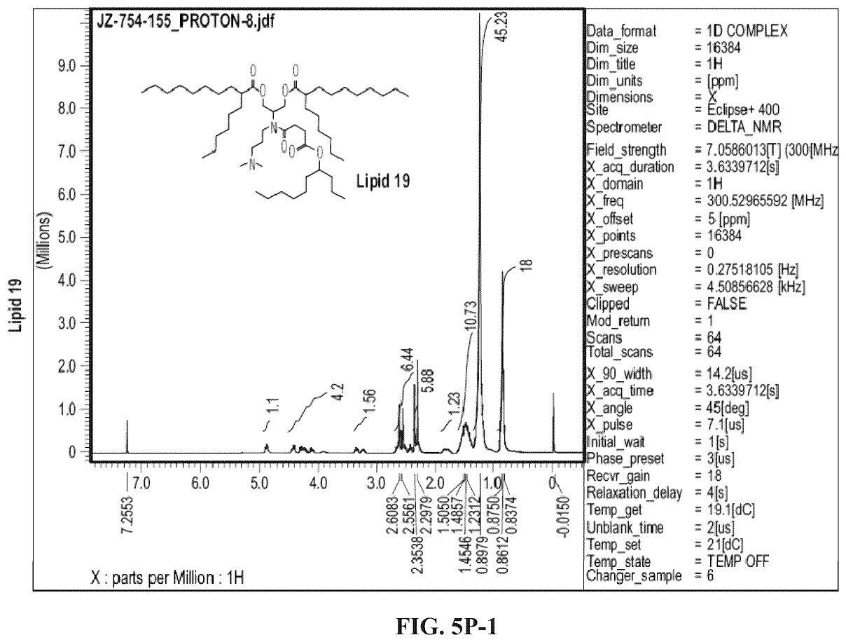

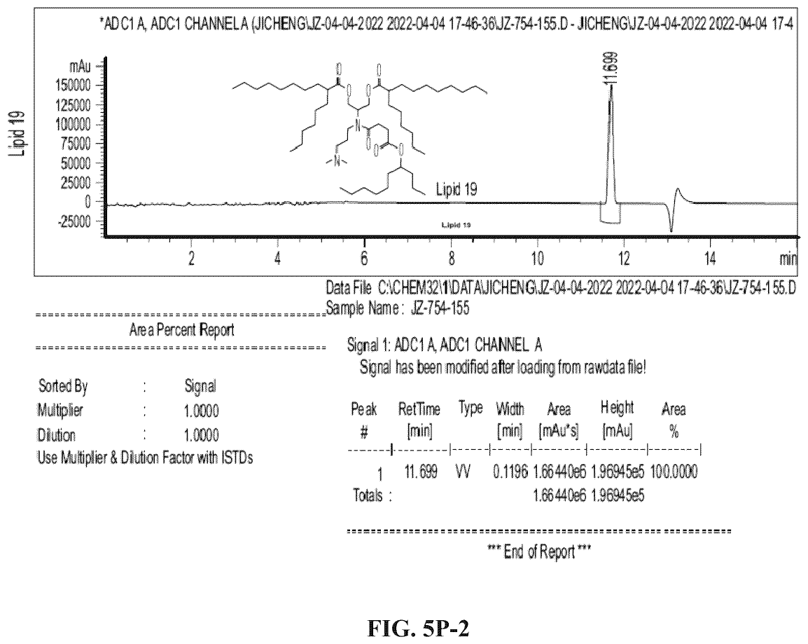

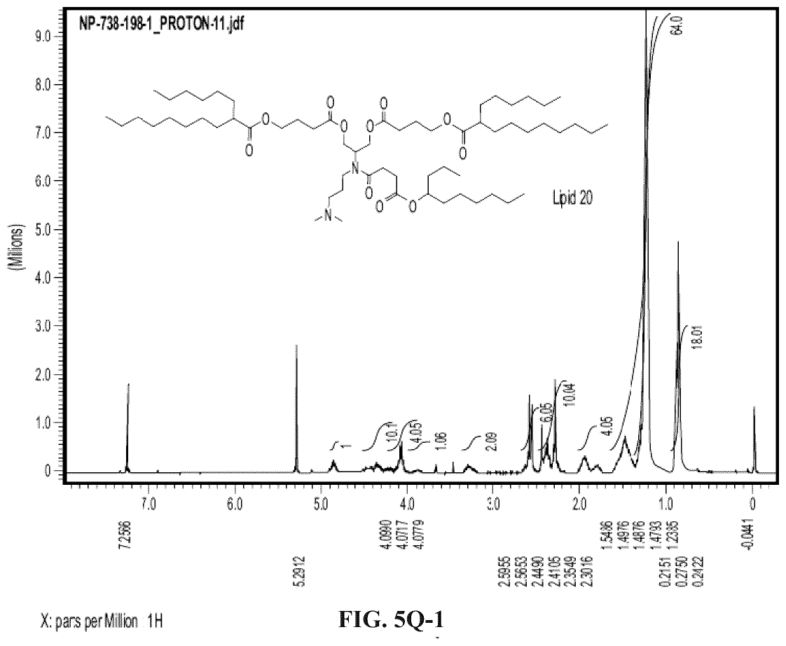

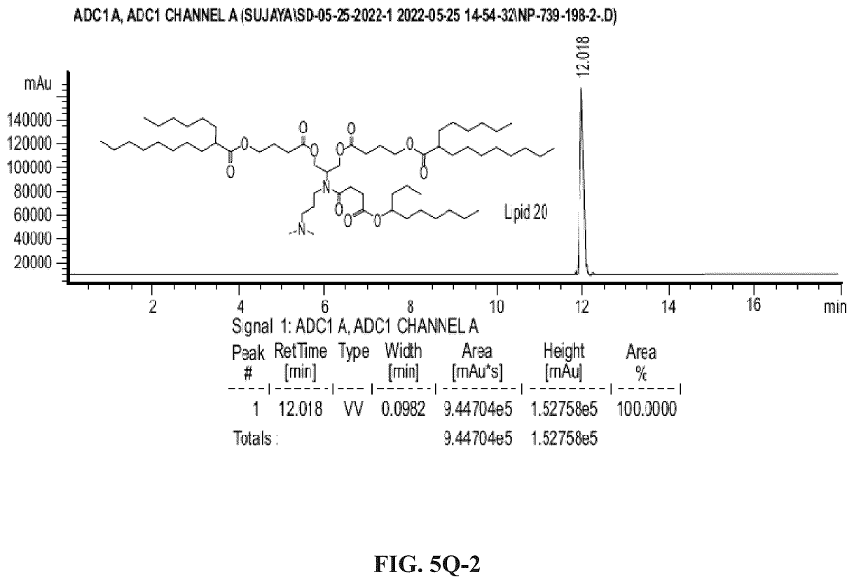

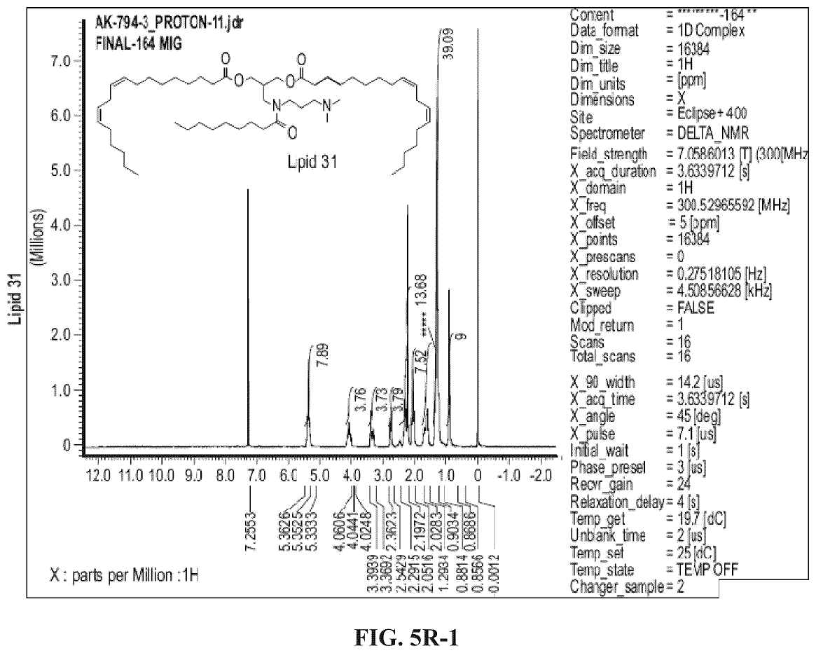

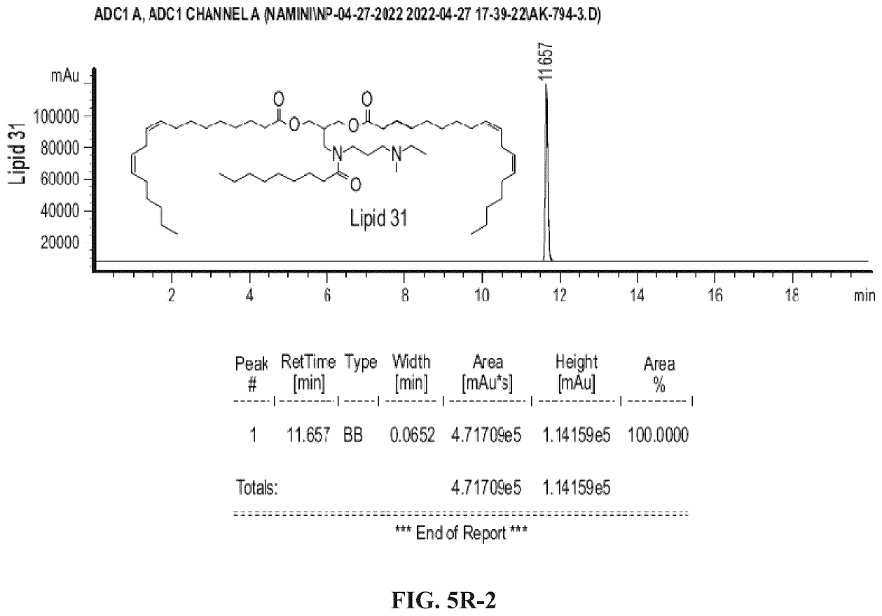

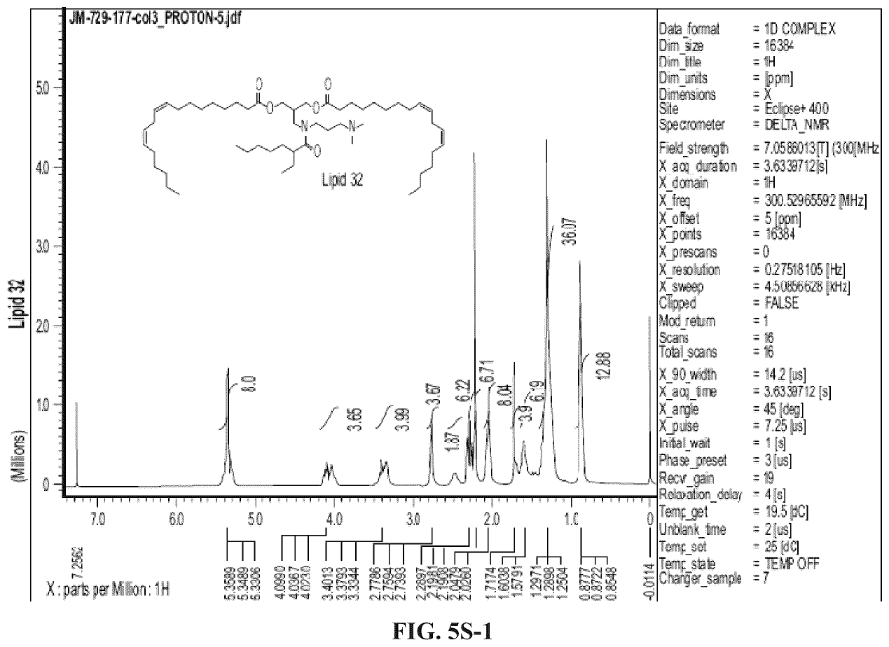

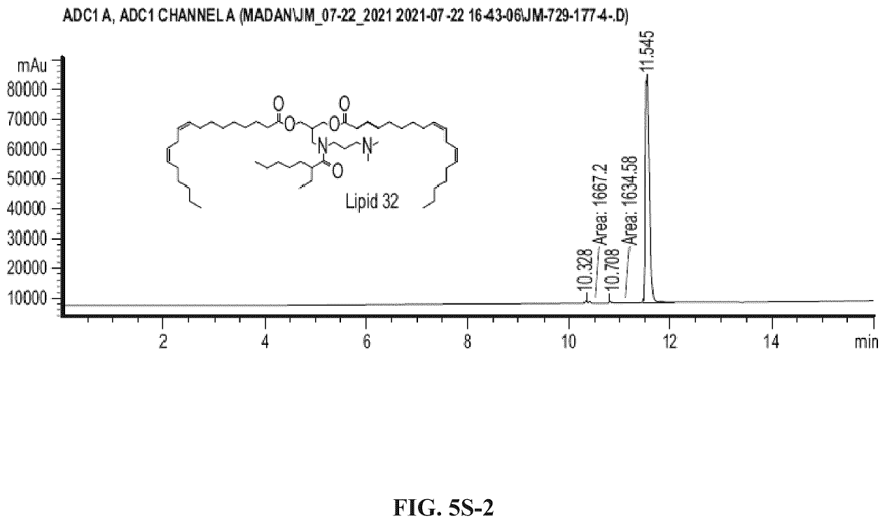

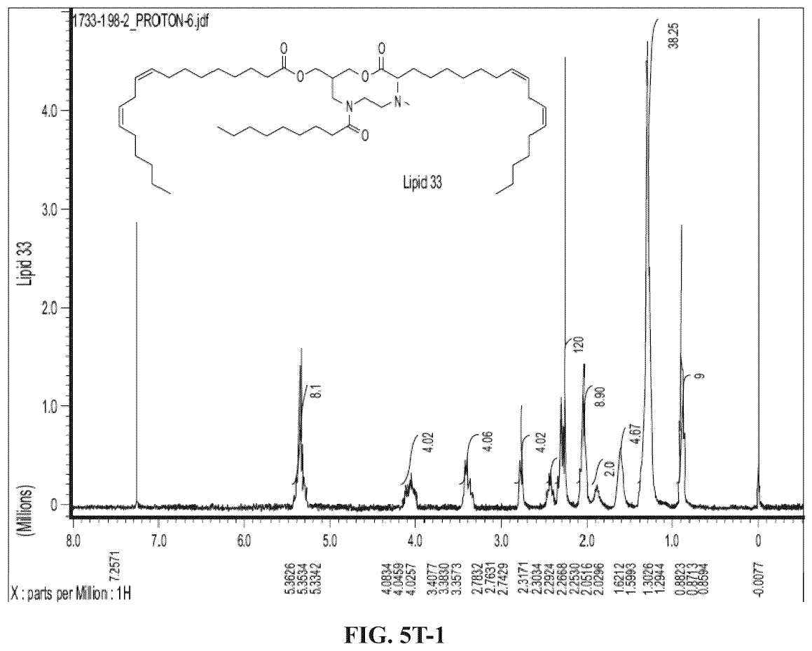

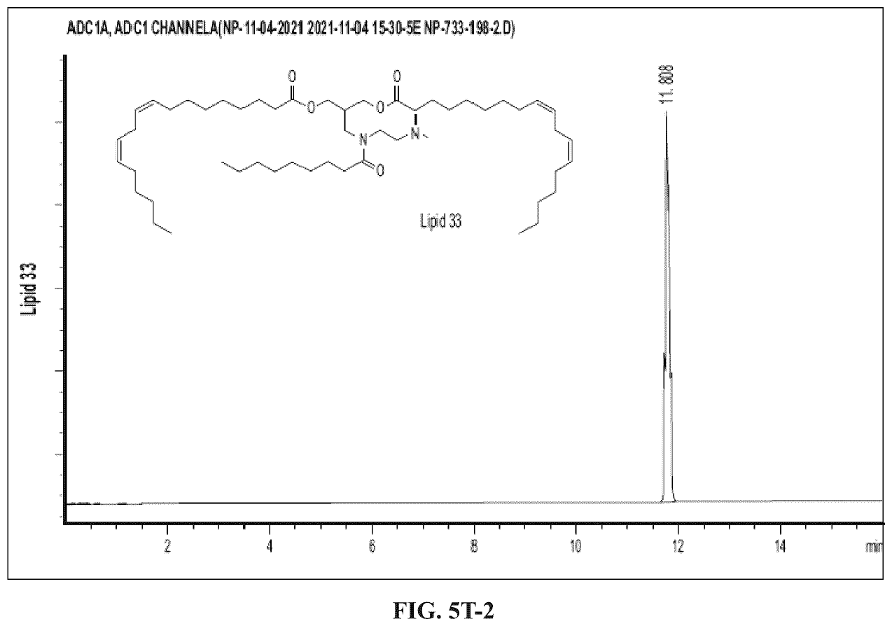

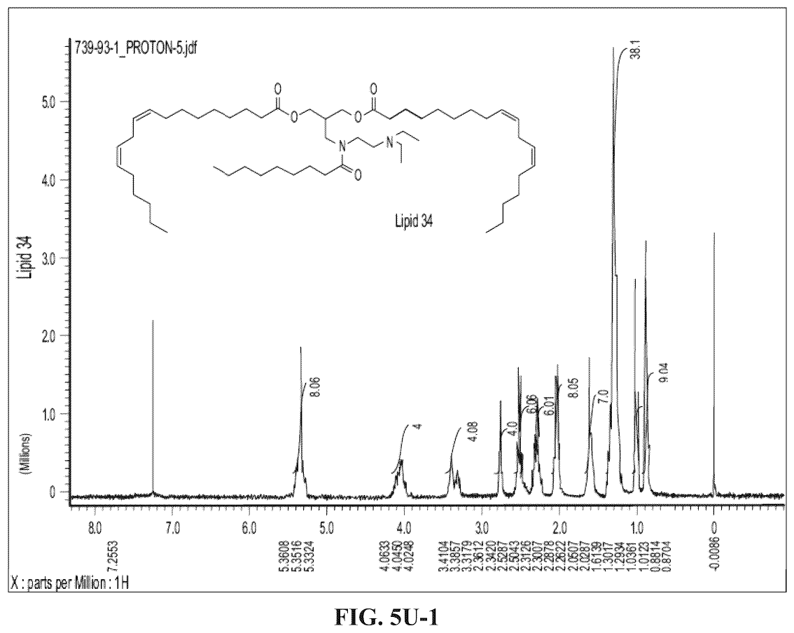

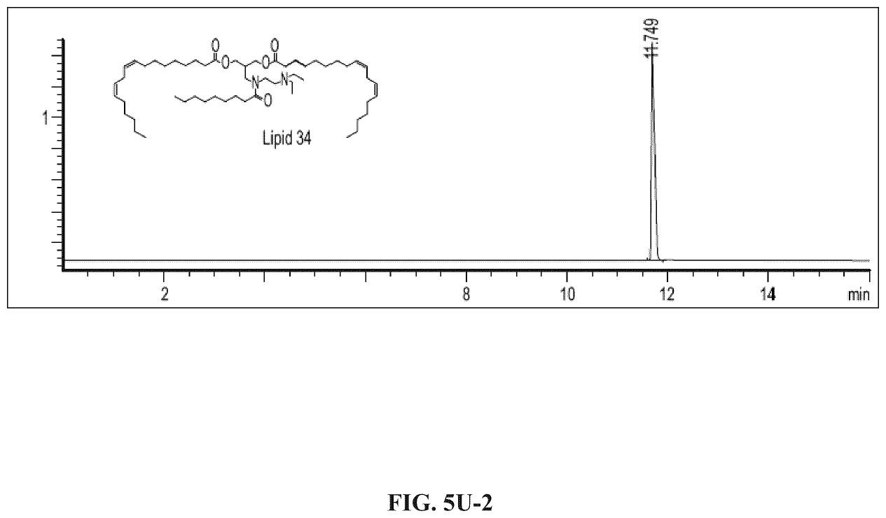

Lipid 1 was synthesized as provided in scheme 6 below and as follows. Starting material, 131-1 (0.75 mmol, 130 mg, 1.0 eq) was converted to the acid chloride (Step 1) using oxalyl chloride (3.7 mmol, 320 μl, 5 eq,) and DMF (10 μl, catalytic) in 6 mL of benzene. Product (143 mg, 98%) showed only one spot on TLC (as methyl ester) and was used without further purification for acylation (step 2) of intermediate 13-0. Intermediate 13-0 (0.35 mmol, 250 mg, 1.0 eq.) was acylated with crude acid chloride, 131-1 (0.75 mmol, 143 mg, 1.7 eq.) using TEA (240 μL, 5 eq, 1.8 mmol) and DMAP (10 mg, catalytic amount). Crude product was purified by column chromatography (2×) yielding 124 mg (76%) of pure Lipid 1 (≥99% purity by LC-ELSD) and characterized by proton NMR and Mass Spectrometry (see FIG. 5A-1 for Lipid 1 NMR spectrum, and Table 4 for product mass).

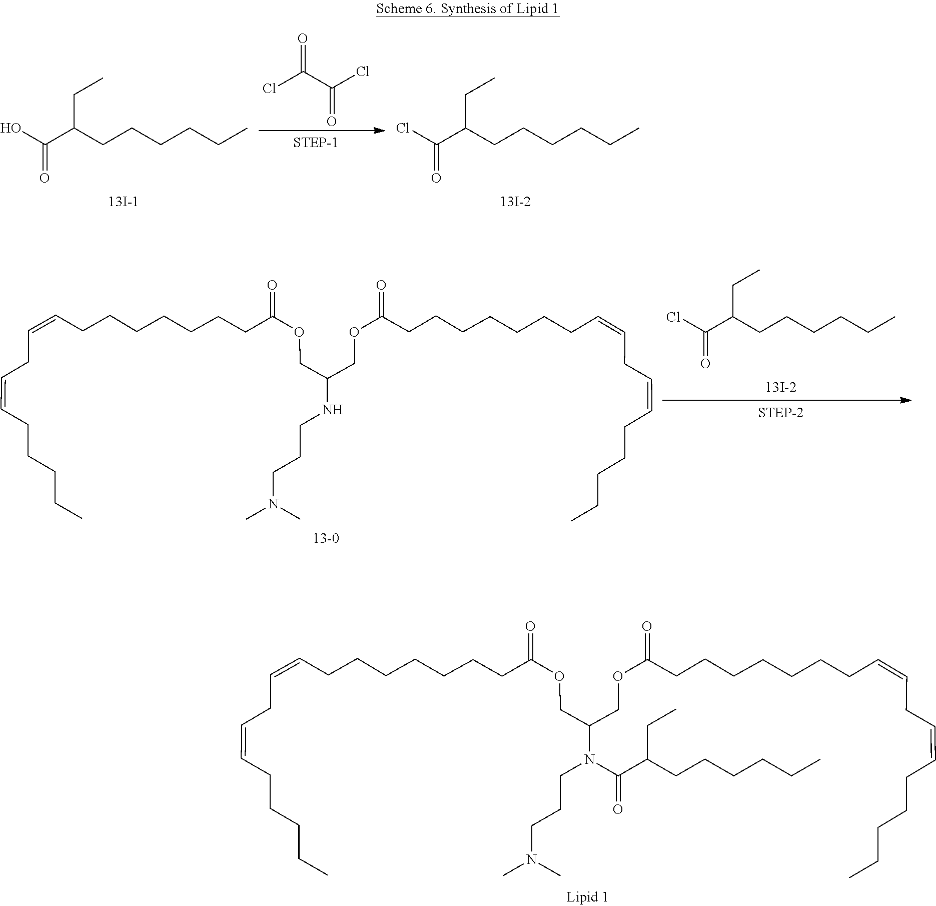 [SMILES: CCCCCCC(CC)C(=O)O ; O=C(Cl)C(=O)Cl ; CCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCC]

### Synthesis of Lipid 3

Lipid 3 was synthesized as provided in scheme 7 below and as follows. Starting material, 13-13 (8.3 mmol, 1.30 g, 1.0 eq) was converted to the acid chloride, 13-13a (Step 1) using oxalyl chloride (2.8 mmol, 2.4 ml, 5 eq.) and DMF (100 μl, catalytic) in 60 mL of benzene. Product (1.44 g, 98%) showed only one spot on TLC (as methyl ester) and was used without further purification for acylation (step 2) of intermediate 13-0. Intermediate 13-0 (5.4 mmol, 3.78 g, 1.0 eq.) was acylated with crude acid chloride, 13-13a (1.44 g, 1.5 eq, 8.1 mmol) using TEA (3.76 mL, 5 eq, 27 mmol) and DMAP (50 mg, cat, catalytic amount) in benzene (100 mL). Crude product was purified by column chromatography (2×) yielding 2.1 g (46.3%) of pure Lipid 3 (≥99% purity by LC-ELSD) and characterized by proton NMR and Mass Spectrometry (see FIG. 5B-1 for Lipid 3 NMR spectrum, FIG. 5B-2 for Lipid 3 LC-MS, and Table 4 for product mass).

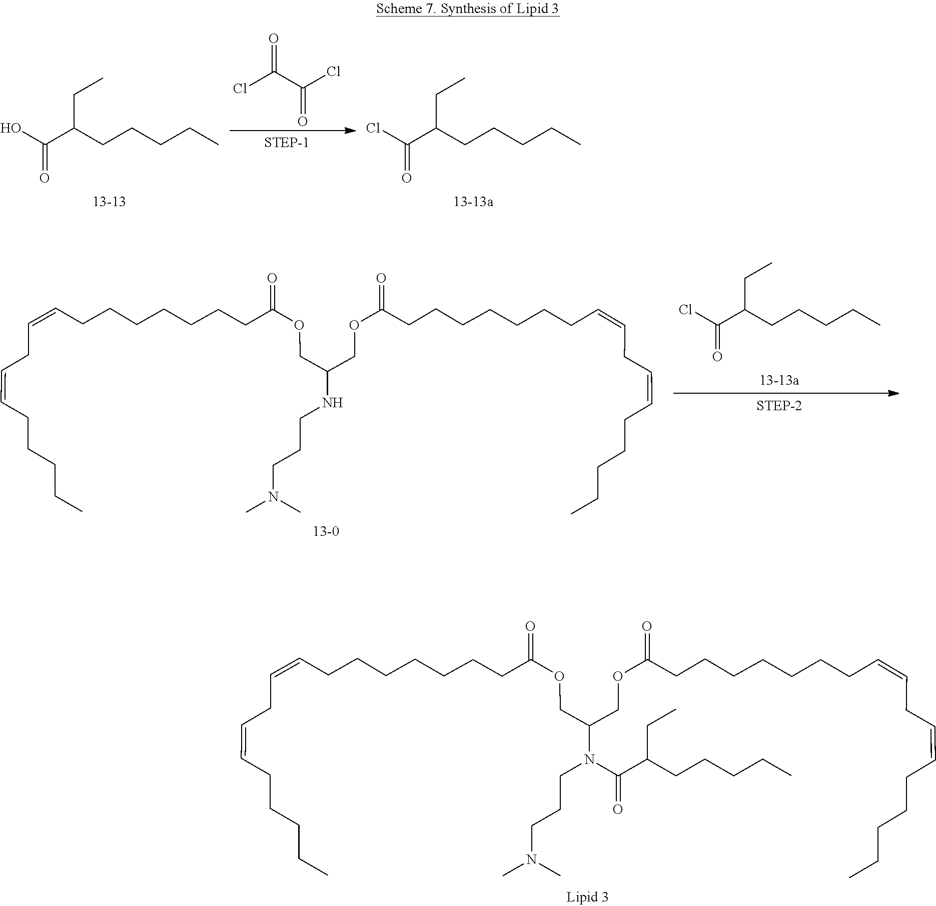 [SMILES: CCCCCC(CC)C(=O)O ; O=C(Cl)C(=O)Cl ; CCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCC(CC)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCC]

### Synthesis of Lipid 4

Lipid 4 was synthesized as provided in scheme 7 below and as follows. Starting material, 13-18 (0.95 mmol, 150 mg, 1 eq.) was converted to the acid chloride, 13-18′ (Step 1) using oxalyl chloride (3.23 mmol, 227 μl, 3.4 eq.) and DMF (10 μl, catalytic) in 6 mL of benzene. Product showed only one spot on TLC (as methyl ester) and was used without further purification for acylation (step 2) of intermediate 13-11b. Intermediate 13-11b (0.63 mmol, 444 mg, 1.0 eq.) was acylated with crude acid chloride, 13-18′ (167 mg, 1.5 eq, 0.95 mmol) using TEA (445 μL, 5.0 eq, 3.2 mmol) and DMAP (10 mg, catalytic amount) in benzene (10 mL). Crude product was purified by column chromatography (5×) yielding 140 mg (26%) of pure Lipid 4 (97% purity by LC-ELSD) and characterized by proton NMR and Mass Spectrometry (see FIG. 5C-I for Lipid 4 NMR spectrum, FIG. 5C-2 for Lipid 4 LC-MS, and Table 4 for product mass).

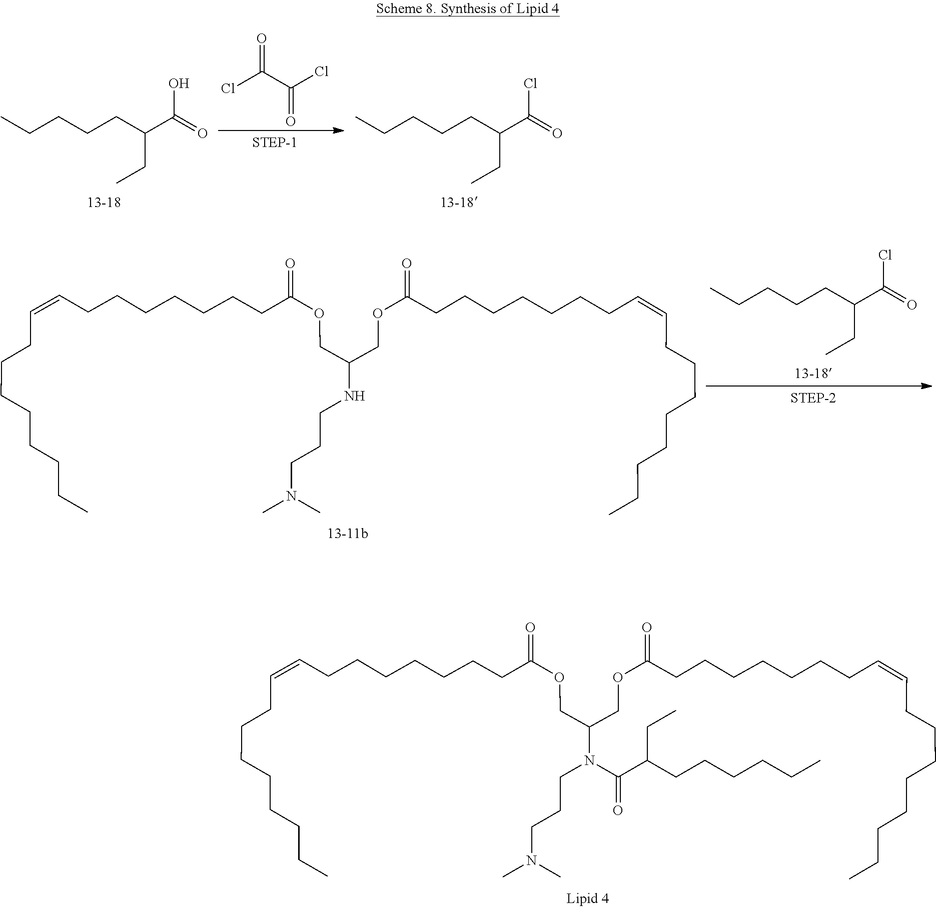 [SMILES: CCCCCC(CC)C(=O)O ; O=C(Cl)C(=O)Cl ; CCCCCC(CC)C(=O)Cl ; CCCCCCCC/C=C\CCCCCCCC(=O)OCC(COC(=O)CCCCCCC/C=C\CCCCCCCC)NCCCN(C)C ; CCCCCC(CC)C(=O)Cl ; CCCCCCCC/C=C\CCCCCCCC(=O)OCC(COC(=O)CCCCCCC/C=C\CCCCCCCC)N(CCCN(C)C)C(=O)C(CC)CCCCCC]

### Synthesis of Lipid 5 and its (S) Isomer

The (S) isomer of lipid 5 was synthesized as provided in scheme 9-1 below and as follows. Starting material, Ethyl hexenoic acid 13m-1 (110 mg, 1.0 eq, 0.75 mmol) was converted to the acid chloride, 13m-2 (Step 1) using oxalyl chloride (320 μL, 1.0 eq, 3.7 mmol) and DMF (20 μl, catalytic) under reflux for 2 hours in 3 mL of benzene. Product showed only one spot on TLC (as methyl ester) and was used without further purification for acylation (step 2) of intermediate 13-0. Intermediate 13-0 (250 mg, 1.0 eq, 0.35 mmol) was acylated with crude acid chloride, 13m-2 (120 mg, 1.8 eq, 0.75 mmol) using TEA (240 μL, 5.0 eq, 1.8 mmol) and DMAP (10 mg, catalytic amount) in 10 mL benzene, overnight at room temperature. Crude product was purified by column chromatography (2×) yielding 95 mg (32%) of pure Lipid 5 (≥99% purity by LC-ELSD) and characterized by proton NMR and Mass Spectrometry (see FIG. 5D-1 for Lipid 5 NMR spectrum, FIG. 5D-2 for Lipid 5 LC-MS, and Table 4 for product mass).

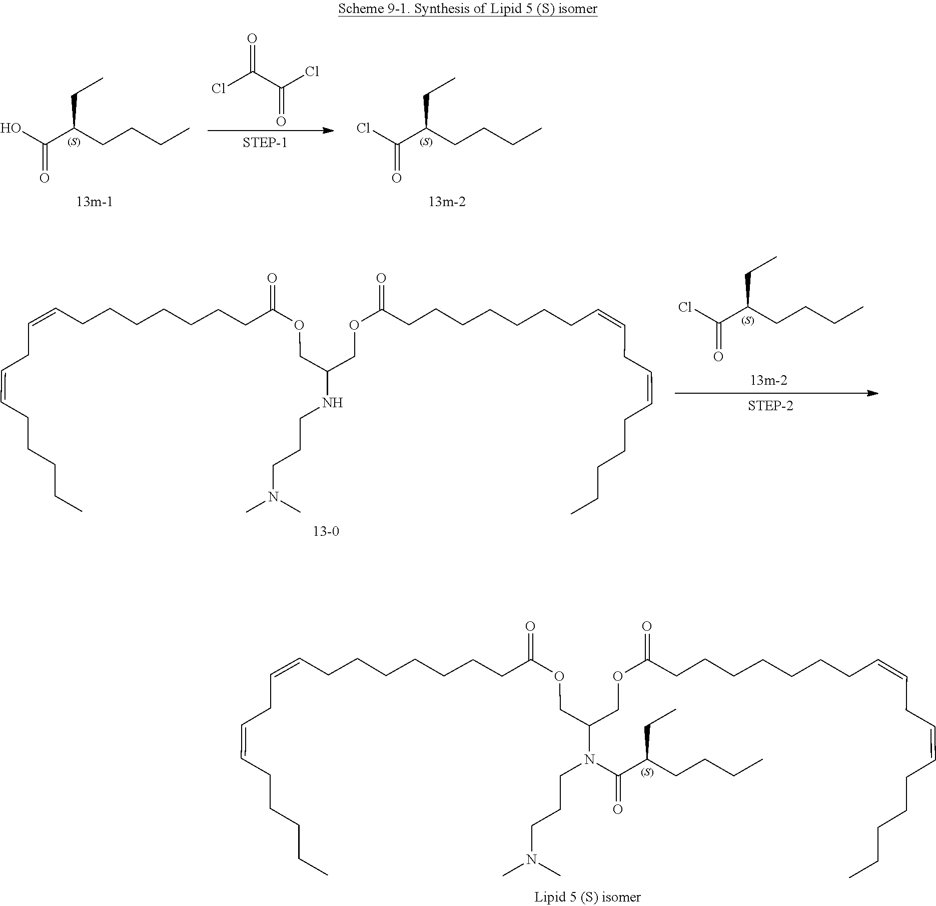

Lipid 5 as a racemic mixture was synthesized similarly as provided in scheme 9-2 below.

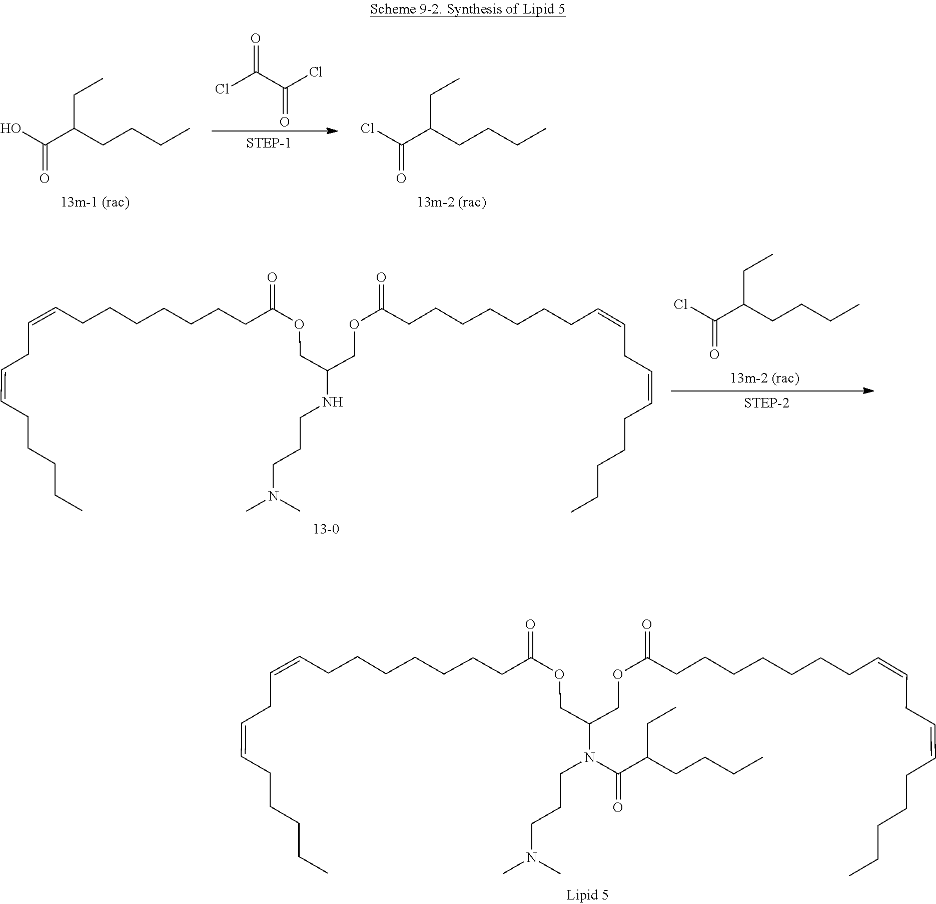

### Synthesis of Lipid 6

Lipid 6 was synthesized as provided in scheme 10 below and as follows. Starting material, 2-ethylnonanoic acid 13-14 (132 mg, 0.17 mmol, 1 eq.) was converted to the acid chloride, 13-14′ (Step 1) using oxalyl chloride (207 μl, 3.4 eq, 2.4 mmol) and DMF (10 μl, catalytic quantity) in 6 mL of benzene. Product showed only one spot on TLC (as methyl ester) and was used without further purification for acylation (step 2) of intermediate 13-0. Intermediate 13-0 (0.47 mmol, 330 mg, 1 eq.) was acylated with crude acid chloride, 13-14′ (145 mg, 1.5 eq, 0.7 mmol) using TEA (327 μL, 5.0 eq, 2.4 mmol) and DMAP (10 mg, catalytic amount) in 10 mL benzene. Crude product was purified by column chromatography (2×) yielding 75 mg (18%) of pure Lipid 6 (≥99% purity by LC-ELSD) and characterized by proton NMR and Mass Spectrometry (see FIG. 5E-1 for Lipid 6 NMR spectrum, FIG. 5E-2 for Lipid 6 LC-MS, and Table 4 for product mass).

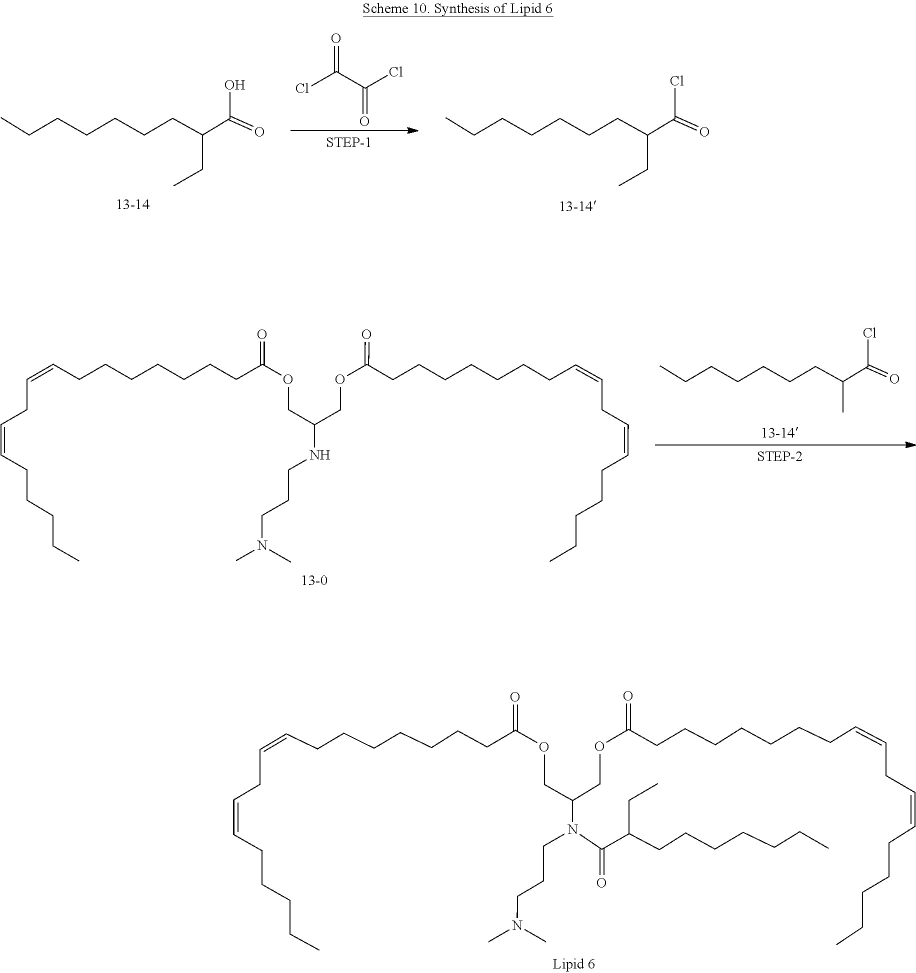 [SMILES: CCCCCCCC(CC)C(=O)O ; O=C(Cl)C(=O)Cl ; CCCCCCCC(CC)C(=O)Cl ; CCCCC/C=C\C/C=C\CCCCCCCC(=O)OCC(COC(=O)CCCCCCC/C=C\C/C=C\CCCCC)NCCCN(C)C ; CCCCCCCC(C)C(=O)Cl ; CCCCC/C=C\C/C=C\CCCCCCCC(=O)OCC(COC(=O)CCCCCCC/C=C\C/C=C\CCCCC)N(CCCN(C)C)C(=O)C(CC)CCCCCCC]

### Synthesis of Lipid 7

Lipid 7 was synthesized as provided in scheme 11 below and as follows. Starting material, heptanoic acid, 13-15 (23.1 mmol, 3.0 g, 1 eq.) was alkylated (step 1) with n-butyl bromide, 13-16 ((2.5 mL, 1.0 eq, 23.1 mmol) and 2.5 M n-butyl lithium in hexane (20.0 mL, 2.2 eq, 51 mmol) using diisopropylamine (7.2 mL, 2.2 eq, 51 mmol) in HMPA (4.4 mL) and 30 mL THF. 1.5 g (35%) of 2-butyl heptanoic acid, 13-17, was isolated from reaction mixture by flash chromatography. Intermediate 13-17 (360 mg, 0.94 mmol, 1 eq.) was converted to the acid chloride, 13-17′ (Step 2) using oxalyl chloride (6.6 mmol, 568 μl, 3.4 eq.) and DMF (5 μl, catalytic) in 3 mL of benzene. Product showed only one spot on TLC (as methyl ester) and was used without further purification for acylation (step 3) of intermediate 13-0. Intermediate 13-0 (0.64 mmol, 450 mg, 1 eq.) was acylated with crude acid chloride, 13-17′ (395 mg, 3.0 eq, 1.94 mmol), TEA (446 μL, 5.0 eq, 3.2 mmol), DMAP (10 mg) in 10 mL of benzene. Crude product was purified by column chromatography (2×) yielding 228 mg (41%) of pure Lipid 7 (≥99% purity by LC-ELSD) and characterized by proton NMR and Mass Spectrometry (see FIG. 5F-1 for Lipid 7 NMR spectrum, FIG. 5F-2 for Lipid 7 LC-MS, and Table 4 for product mass).

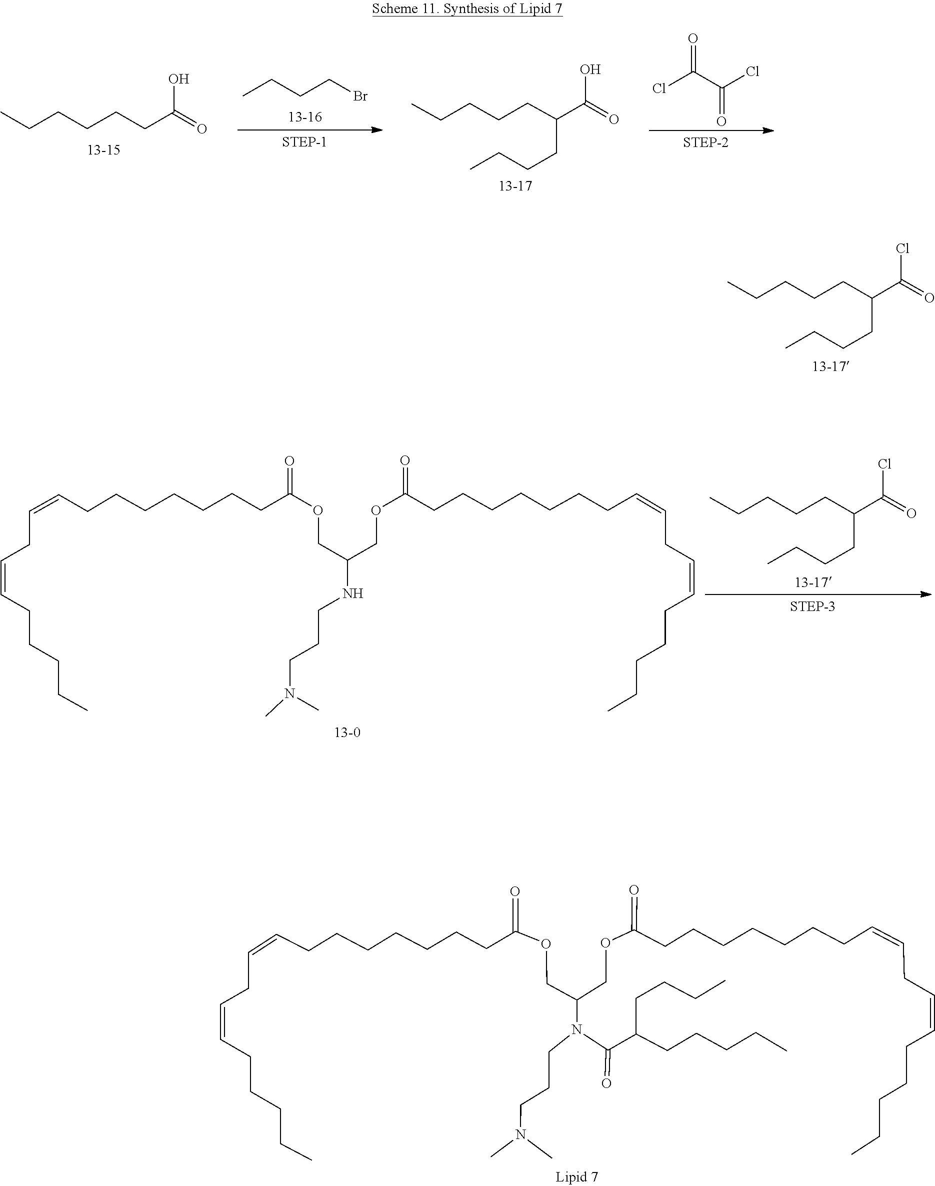

### Synthesis of Lipids 2, 8, 9 and 10 by N-Acylation of Intermediate 13-0 Using Carbodiimide Activation of the Corresponding Carboxylic Acids

Lipid 2 was synthesized as provided in scheme 12 below and as follows. Intermediate 13-0 (0.14 mmol, 320 mg, 1.0 eq.) was acylated with nonanoic acid 13-12 (1.15 mmol, 198 μL, 2.5 eq.), EDCI (1.15 mmol, 221 mg, 2.5 eq.), DIPEA (1.15 mmol, 198 μL, 2.5 eq.), and DMAP (0.05 mmol, 6.4 mg, 0.1 eq.) in 5 mL DCM. Crude product was purified by column chromatography (3×) yielding 107 mg (%) of pure Lipid 2 (≥99% purity by LC-ELSD) and characterized by proton NMR and Mass Spectrometry (see FIG. 5G-1 for Lipid 2 NMR spectrum, FIG. 5G-2 for Lipid 2 LC-MS, and Table 4 for product mass).

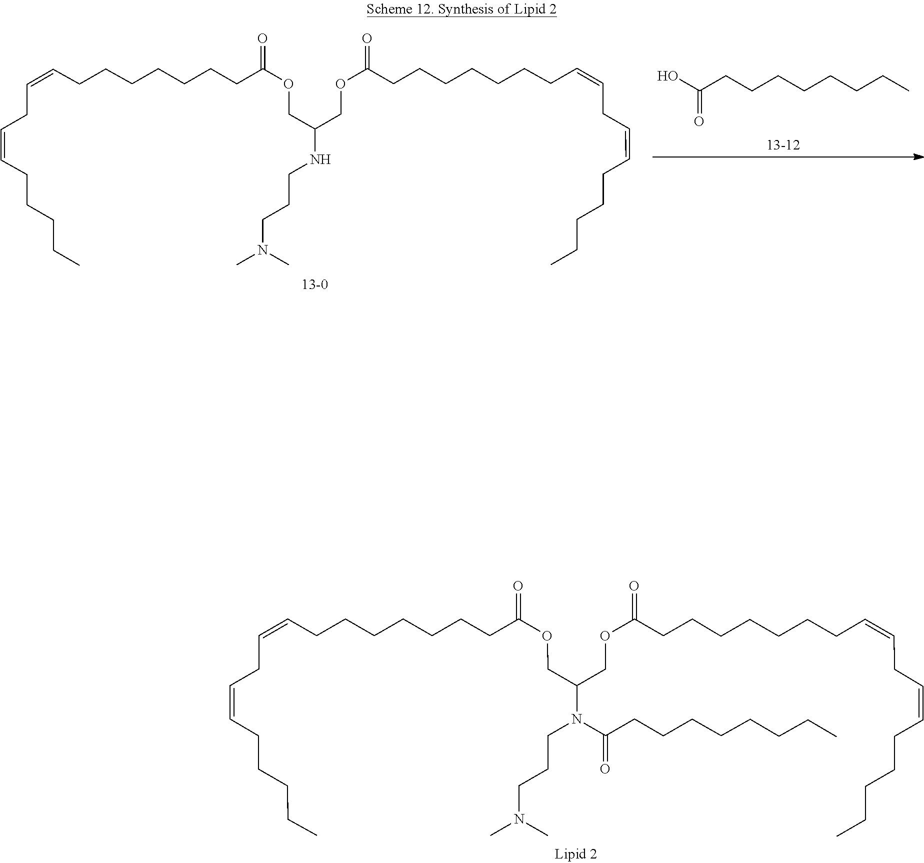 [SMILES: CCCCC/C=C\C/C=C\CCCCCCCC(=O)OCC(COC(=O)CCCCCCC/C=C\C/C=C\CCCCC)NCCCN(C)C ; CCCCCCCCC(=O)O ; CCCCC/C=C\C/C=C\CCCCCCCC(=O)OCC(COC(=O)CCCCCCC/C=C\C/C=C\CCCCC)N(CCCN(C)C)C(=O)CCCCCCCC]

### Synthesis of Lipid 8

Lipid 8 was synthesized as provided in scheme 13 below and as follows. Alkene, 13-48 was accessed via the HWE reaction (step 1) of octan-3-one, 13-46 (2 g, 15.6 mmol) with ethyl 2-(diethoxyphosphoryl)acetate, 13-47 (7.0 g, 2.0 eq, 31.2 mmol), 2M NaHMDS in THF (15.6 mL, 2.0 eq, 31.2 mmol), and 9 ml THF solvent. Reaction workup yielded 2.38 g (77%) of 13-48 confirmed by NMR, product mass and single TLC spot. Alkene, 13-48 (5.1 mmol, 1 g, 1 eq.) was hydrogenated (step 2) using Pd/C (50 mg) in 8 mL ethyl acetate yielding intermediate 13-48 (958 mg, 77%). Ester hydrolysis (step 3) of 13-49 (5.1 mmol, 412 mg) using THF/MeOH/1M LiOH (3.0/2.0/3.0 mL) yielded carboxylic acid intermediate 13-50 (336 mg, 95%). Intermediate 13-0 (0.33 mmol, 234 mg) was acylated with 13-50 (0.66 mmol, 115 mg, 2.0 eq.) using EDCI (0.66 mmol, 102 mg, 2.0 eq.), DIPEA (0.66 mmol, 114 μL, 2.0 eq.), DMAP (0.33 mmol, 41 mg, 1.0 eq.), in 2 mL DCM yielding 77 mg (27%) of pure Lipid 8 (299% purity by LC-ELSD) and characterized by proton NMR and Mass Spectrometry (see FIG. 5H-1 for Lipid 8 NMR spectrum, FIG. 5H-2 for Lipid 8 LC-MS, and Table 4 for product mass).

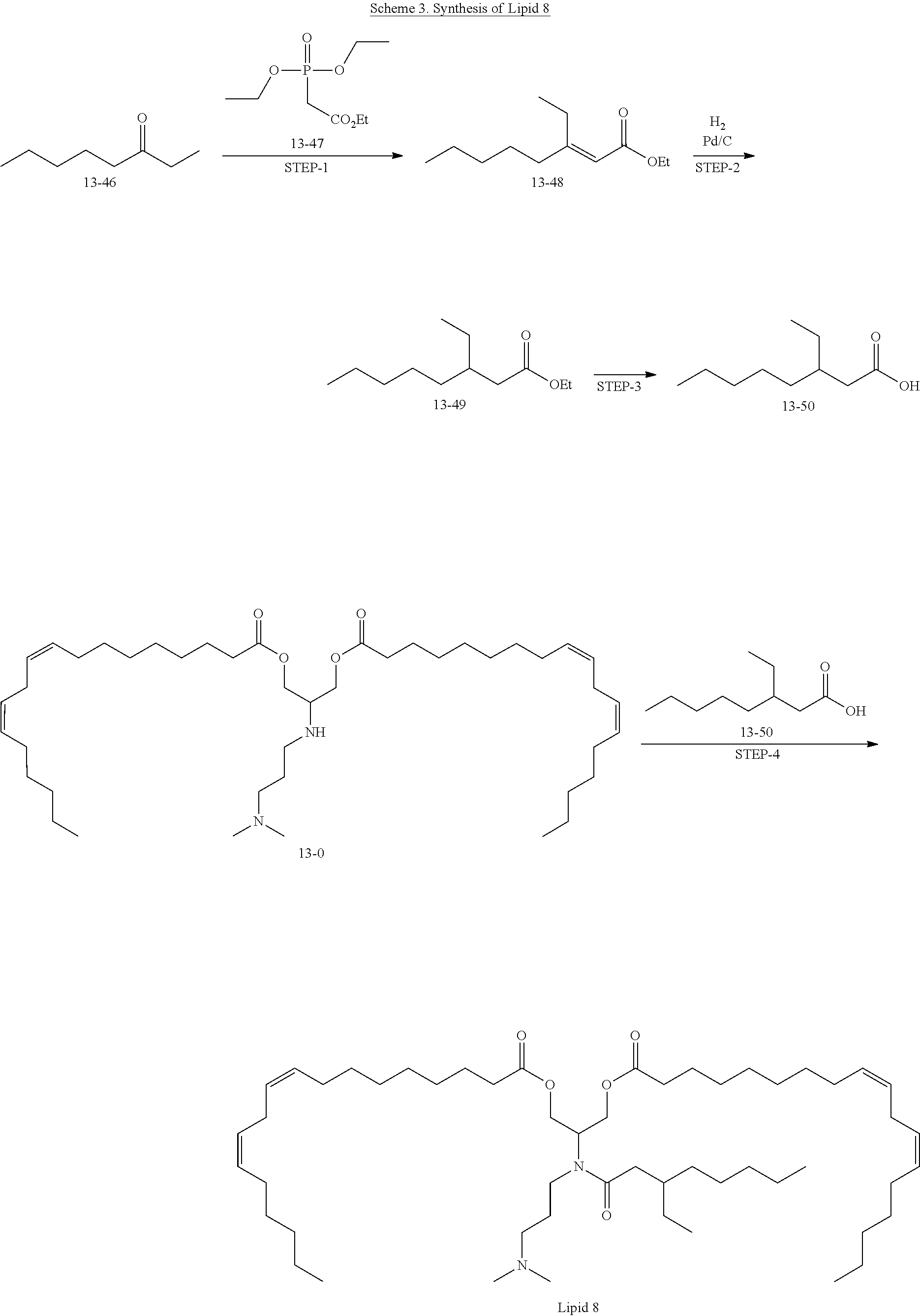

### Synthesis of Lipid 9

Lipid 9 was synthesized as provided in scheme 14 below and as follows. Starting material, decan-4-ol, 13-29 (32.0 mmol, 5.0 g, 1.0 eq.) was acylated with succinic acid, 13-30 (6.3 g, 2.0 eq, 63.0) using DMAP (3.55 g, 1.0 eq, 32.0 mmol) and pyridine (5.0 ml) in 5 mL THF. Crude product was purified by column chromatography (1×) to obtain 4.26 g (81%) of pure acid intermediate 13-31. Intermediate 13-0 (2.1 mmol, 1.5 g, 1 eq.) was acylated with 13-31 (2.13 mmol, 0.554 g, 1.1 eq), using DIPEA (745 μL, 4.26 mmol, 2.5 eq), EDCI (820 mg, 4.26 mmol, 2.5 eq), and DMAP (480 mg, 0.43 mmol, 0.25 eq), in 50 mL DCM. Crude product was purified by column chromatography (3×) yielding 1.4 g (73%) of pure Lipid 9 (≥99% purity by LC-ELSD) and characterized by proton NMR and Mass Spectrometry (see FIG. 5I-1 for Lipid 9 NMR spectrum, FIG. 51-2 for Lipid 9 LC-MS, and Table 4 for product mass).

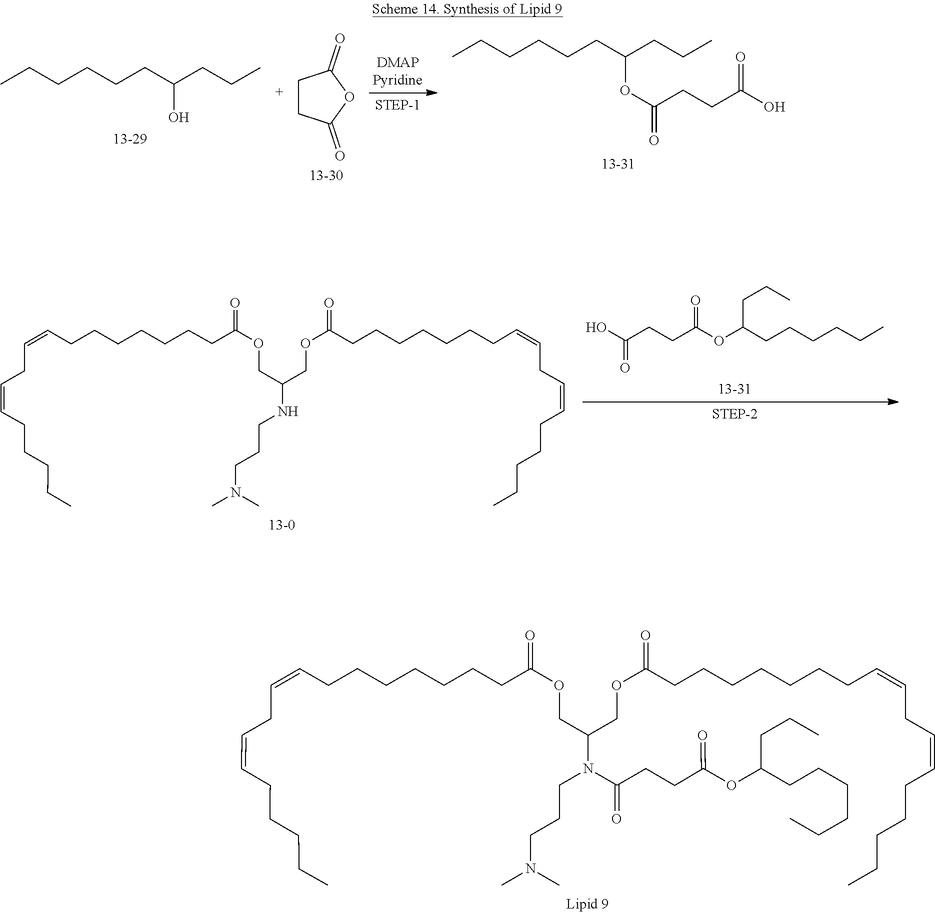

### Synthesis of Lipid 10 and its (S) Isomer

The (S) isomer of lipid 10 was synthesized as provided in scheme 15-1 below and as follows. Starting material, Octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM. Crude product was purified by column chromatography (1×) to obtain 1.1 g (31%) of pure acid intermediate 13-47. Intermediate 13-0 (250 mg, 1.0 eq, 0.36 mmol) was acylated with 13-47 (123 mg, 1.5 eq, 0.53 mmol), using EDCI (207 mg, 3.0 eq, 1.80 mmol), DIPEA (188 μL, 3.0 eq, 1.8 mmol) and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 5J-1 for Lipid 10 NMR spectrum, FIG. 5J-2 for Lipid 10 LC-MS, and Table 4 for product mass).

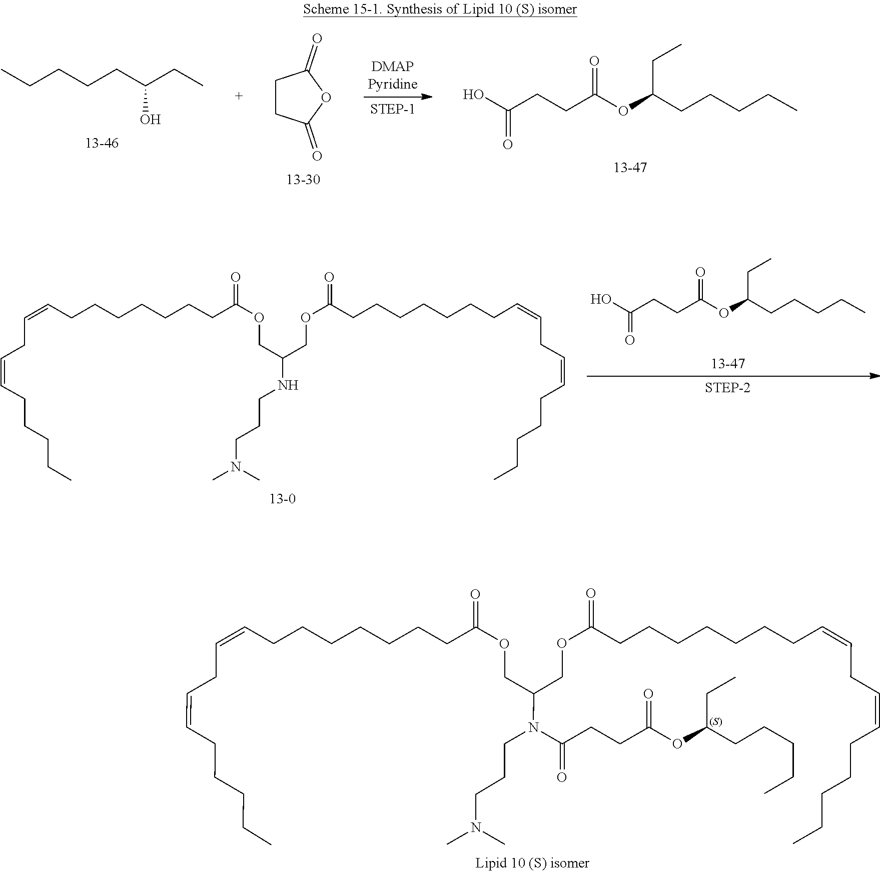

Lipid 10 as a racemic mixture was synthesized similarly as provided in scheme 15-2 below. Starting material, octan-3-ol, 13-46 (2.0 g, 1.0 eq, 15.3 mmol) was acylated with succinic acid, 13-30 (3.1 g, 2.0 eq, 30.6 mmol) using DMAP (1.72 g, 1.0 eq, 15.3 mmol) and pyridine (2.0 ml) in 2 mL THF and 6 mL DCM to obtain intermediate 13-47. Crude product was purified by column chromatography (1×) to obtain 1.1 g (31%) of pure acid intermediate 13-47. 13-0 (250 mg, 1.0 eq, 0.36 mmol) was acylated with 13-38 (123 mg, 1.5 eq, 0.53 mmol) using DIPEA (188 μL, 3.0 eq, 1.8 mmol), EDCI (207 mg, 3.0 eq, 1.80 mmol), and DMAP (15.0 mg, 3.0 eq, 0.018 mmol), in 5 mL DCM. Crude product was purified by column chromatography (2×) yielding 261 mg (54%) of pure Lipid 10 (≥99% purity by LC-ELSD) and characterized by proton NMR and Mass Spectrometry (see FIG. 5J-1 for Lipid 10 NMR spectrum, FIG. 5J-2 for Lipid 10 LC-MS, and Table 4 for product mass).

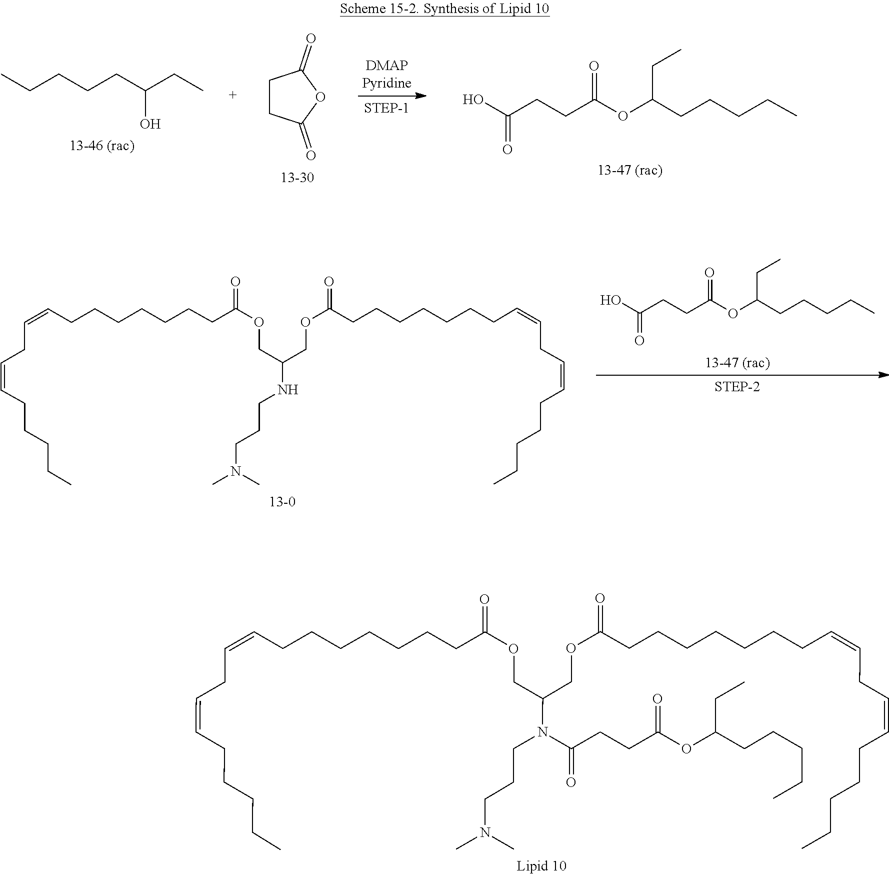

### Synthesis of Lipid 11 by N-Acylation of Intermediate 13-0 Using the Corresponding Acid Chloride

### Synthesis of Lipid 11 and its (S) Isomer

The (S) isomer of lipid 5 was synthesized as provided in scheme 16-1 below and as follows. Starting material, benzyl alcohol, 13-39′ (18.5 mmol, 2 g) was used to acylate compound 13-39 (4.8 g, 1.5 eq, 27.8 mmol) using EDCI (5.4 g, 1.5 eq, 27.8 mmol), DIPEA (4.6 mL, 1.5 eq, 27.8 mmol), and DMAP (463 mg, 0.2 eq, 3.7 mmol) yielding 3.6 g (74%) of column purified intermediate 13-40 (product confirmed by mass spectrometry and proton NMR). Intermediate, 13-40 (684 mg, 2.6 mmol, 1 eq.) was deprotected in acetic acid to obtain intermediate, 13-41 (~600 mg, quantitative and product structure was confirmed by mass spectrometry and proton NMR). Additional quantity of intermediate 13-41 was generated and 1.68 g, 7.5 mmol of 13-41 was selectively protected at the hydroxyl group using TBSCl (1.7 g, 11.25 mmol, 1.5 eq), TEA (5.3 mL, 5.0 eq, 37.5 mmol), and DMAP (92 mg, 0.75 mmol, 0.1 eq), in 20 mL DCM yielding protected intermediate 13-41a (~2.5 g, quantitative) (product mass was confirmed by mass spectrometry and proton NMR). Intermediate 13-41a (1.61 g, 4.76 mmol) was esterified with n-hexyl alcohol 13-34 (2.94 mL, 23.8 mmol, 5.0 eq) using EDCI (2.76 g, 14.2 mmol, 3.0 eq), DIPEA (1.6 mL, 2.0 eq, 9.52 mmol), and DMAP (580 mg, 4.76 mmol, 1.0 eq) in 11.0 mL of DCM to obtain 13-41b (0.95 g, 48%). Additional quantity of 13-41b was generated and total of 1.36 g (3.2 mmol) was deprotected using HF-pyridine (5.8 mL, 80.6 mmol, 25 eq.) in 30 mL THE to obtain intermediate 13-41c (837 mg, 84%). Intermediate 13-41c (456 mg, 1.48 mmol) was acylated with n-butanoyl chloride, 13-42 (760 μL, 7.4 mmol, 5.0 eq) in 4.0 mL pyridine (4.0 mL) yielding compound 13-44 (505 mg, 90%). Intermediate 13-44 (505 mg, 1.34 mmol) was deprotected using Pd/C (30 mg) in 3.0 mL ethyl acetate yielding compound 13-45 (370 mg, 96%). Compound 13-45 (188 mg, 0.65 mmol) was converted to the acid chloride intermediate using oxalyl chloride (190 μg, 3.4 eq, 2.2 mmol) and DMF (10 μL, catalytic quantity), in 3 mL Benzene. Product showed only one spot on TLC (as methyl ester) and was used without further purification for acylation (step 9) of intermediate 13-0. Intermediate 13-0 (152 mg, 0.22 mmol, 1 eq.) was acylated with crude acid chloride, 13-45′ (200 mg, 3.0 eq, 0.65 mmol), TEA (152 μL, 5.0 eq, 1.1 mmol), DMAP (10 mg) in 5 mL of benzene to obtain Lipid 11. Crude product was purified by column chromatography to yield 77 mg (37%) of pure Lipid 11 (≥99% purity by LC-ELSD) and characterized by proton NMR and Mass Spectrometry (see FIG. 5K-1 for Lipid 11 NMR spectrum, FIG. 5K-2 for Lipid 11 LC-MS, and Table 4 for product mass).

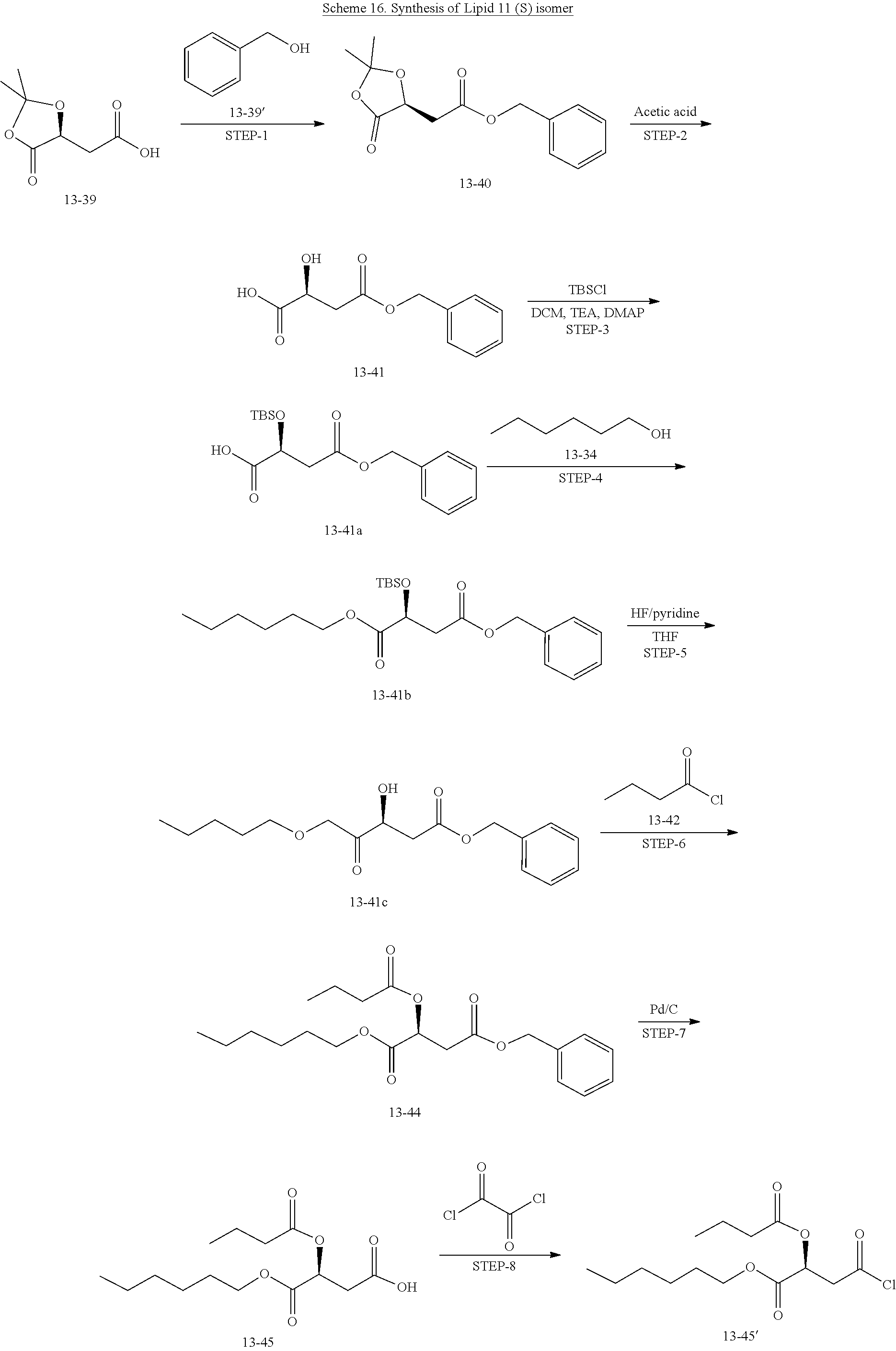

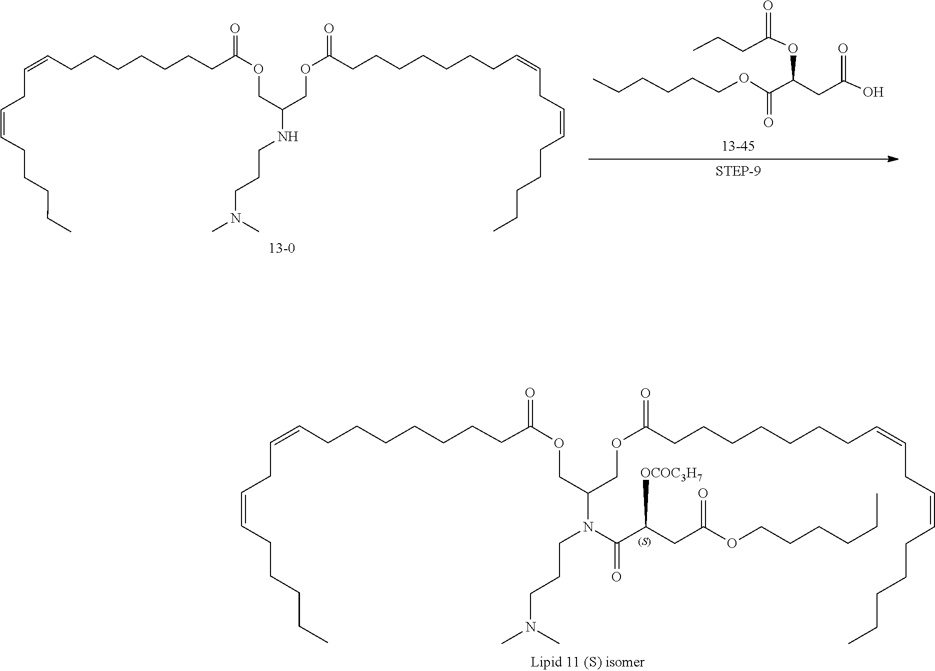

Lipid 11 as a racemic mixture was synthesized similarly as provided in scheme 16-2 below.

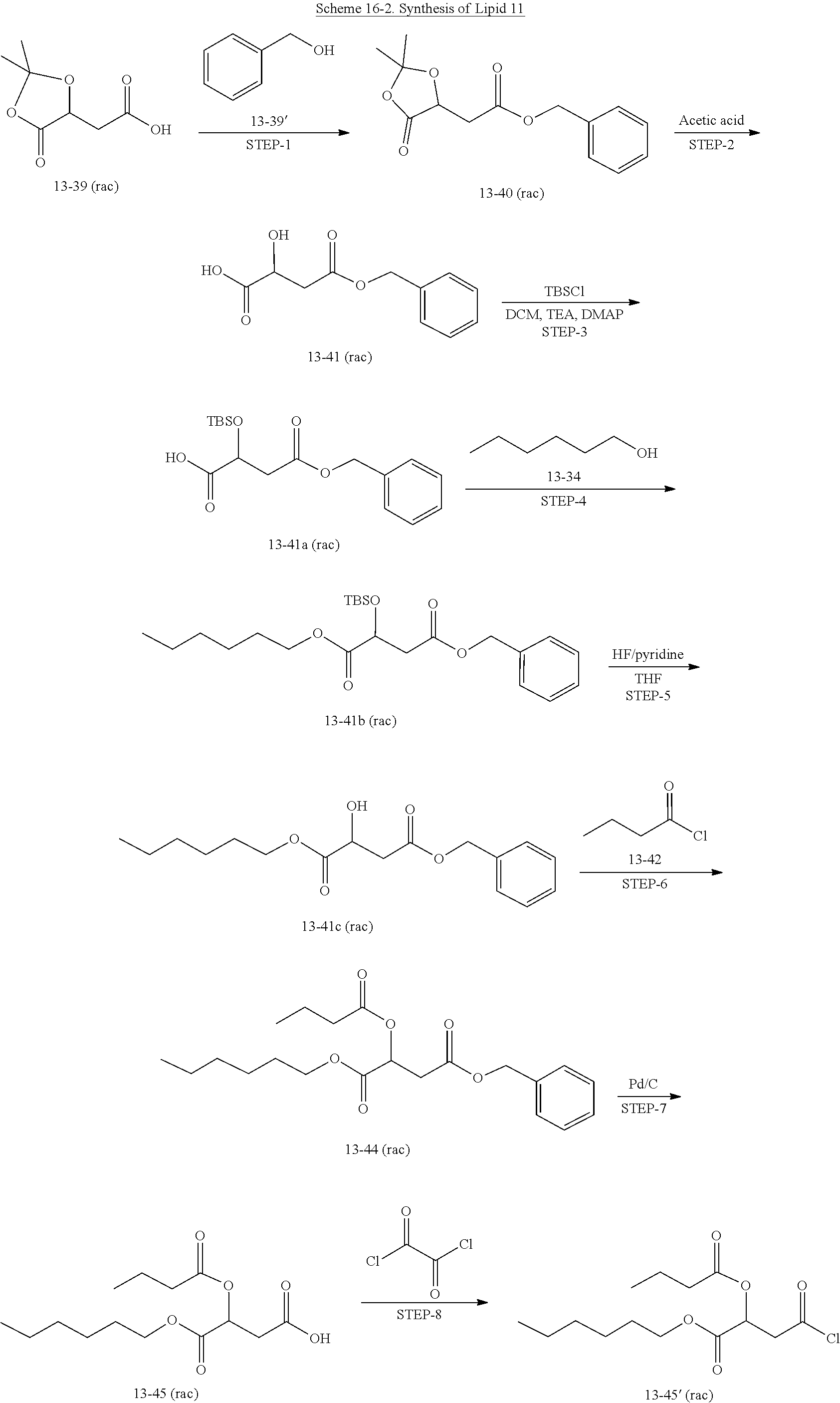

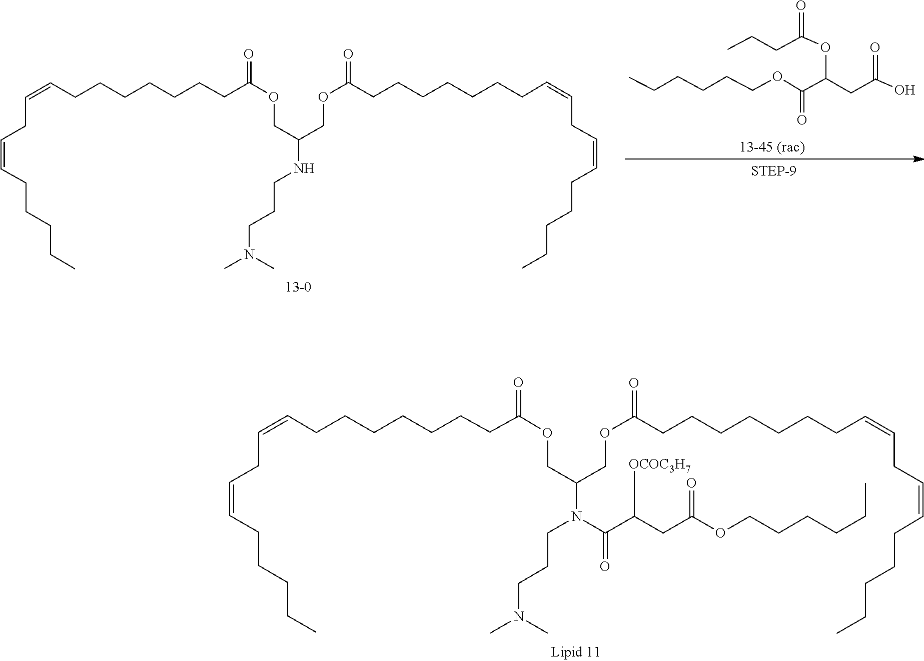

### Synthesis of Lipid 12

Lipid 12 was synthesized as provided in scheme 34 below and as follows. Starting material, 14-3 (3 g, 1.0 eq, 22.37 mmol) was selectively protected in trifluoroacetic anhydride (11.27 g, 2.4 eq, 53.69 mmol) and Benzyl alcohol (15 mL) at room temperature, overnight yielding intermediate 14-4. Crude product was purified by column chromatography (1×) to obtain 4.7 g (96%) purified 14-4. Subsequent acylation of 14-4 (1.0 eq, 4.44 mmol) with n-butanol, 13-34 (4.55 g, 10.0 eq, 44.60 mmol) using EDCI (1.71 g, 2 eq, 8.92 mmol) and DMAP (1.089 g, 2 eq, 8.92 mmol) in 10 mL DCM at RT, overnight yielded 14-5. Crude product was purified by column chromatography (1×) to obtain 800 mg (58%) purified 14-5. Acylation of the free hydroxyl of 14-5 (800 mg, 1.0 eq, 2.59 mmol) with hexanoyl chloride (1.39 g, 4.0 eq, 10.37 mmol) using TEA (1.31 g, 5 eq, 12.97 mmol) and DMAP (10 mg, catalytic amount) in 10 mL toluene at room temperature, overnight yielded intermediate 14-7. Purification of crude product by column chromatography (1×) yielded 470 mg (46%) purified 14-7. Intermediate 14-7 (470 mg, 1 eq., 3.4 mmol) yielded 340 mg (93%) of free acid 14-8. Crude 14-8 (56 mg. 1 eq., 0.18 mmol) was converted to the corresponding chloride, 1′-8′, using Oxalyl Chloride (50 μL, 3.4 eq, 0.60 mmol) and DMF (0.2 μL, Catalytic amount) in 1 mL Toluene at room temperature, overnight to afford 56 mg of crude chloride 1′-8′. N-acylation of 13-0 (42 mg, 1 eq., 0.059 mmol) with 1′-8′ (56.0 mg, 3.0 eq, 0.17 mmol) using TEA (39.0 μL, 5.0 eq, 0.29 mmol) and DMAP (10 mg, Catalytic amount) in 3 mL Toluene yielded Lipid 12. Crude product was purified by column chromatography (1×) to obtain pure Lipid 12 (23 mg, 39%) (≥99% purity by LC-ELSD) and characterized by proton NMR and Mass Spectrometry (see FIG. 5L-1 for Lipid 12 NMR spectrum, FIG. 51-2 for Lipid 12 LC-ELSD chromatogram, and Table 4 for product mass).

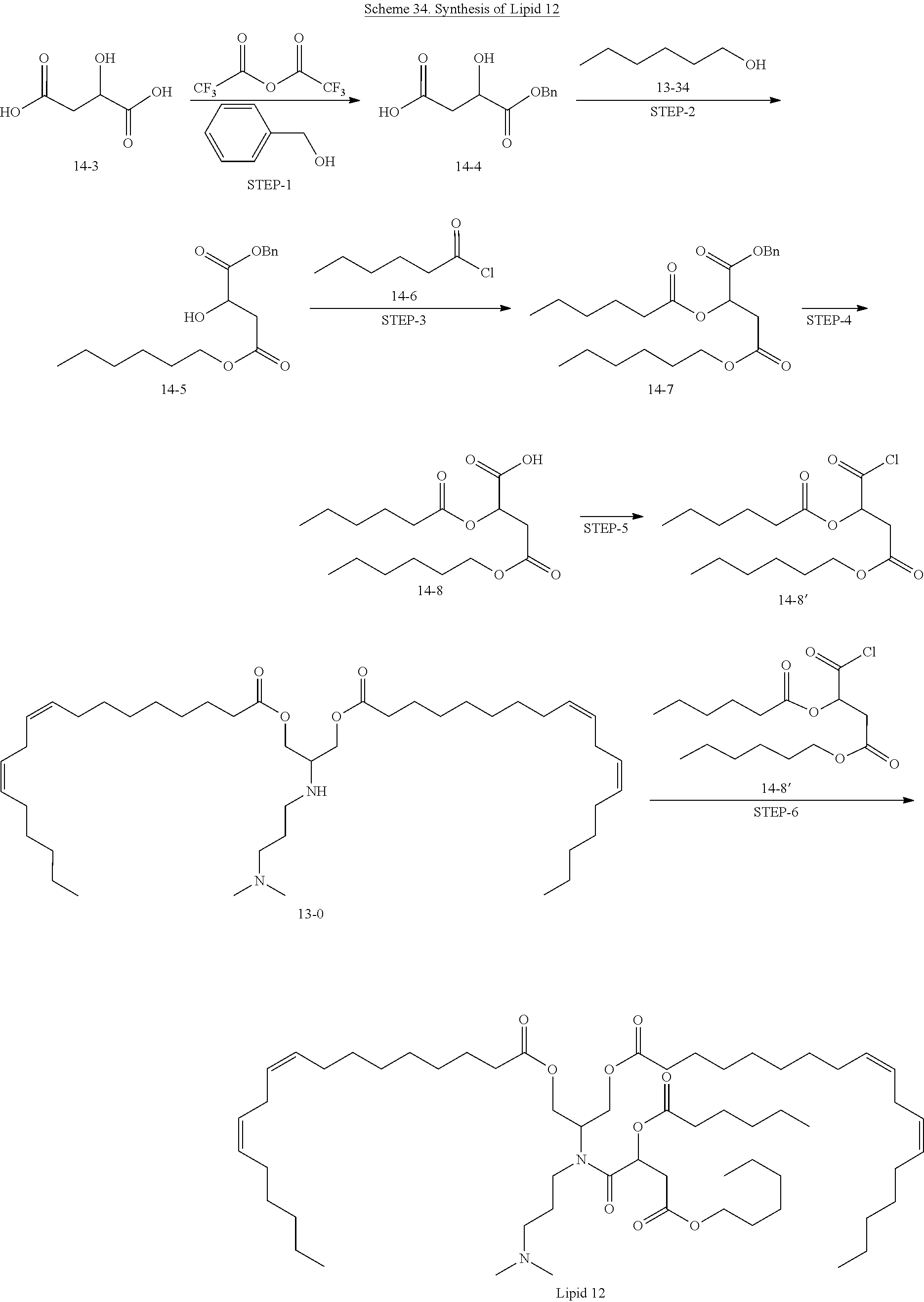

### Synthesis of Lipids 13 by N-Acylation of Intermediate 13-0 Carbodiimide Activation of the Corresponding Carboxylic Acid

### Synthesis of Lipid 13

Lipid 13 was synthesized as provided in scheme 17 below and as follows. Starting material 13-32 (4.8 g, 2.0 eq, 25.0 mmol) was esterified with 1-Butanol (1.13 mL, 1 eq, 12.4 mmol) using EDCI (4.8 g, 2 eq, 25.0 mmol), DIPEA (4.35 mL, 2 eq, 25.0 mmol), and DMAP (280 mg, 0.2 eq, 2.5 mmol) in 20 mL DCM to obtain intermediate 13-33. Crude product was purified by column chromatography to obtain 2.78 g (44%) of pure intermediate 13-33. Intermediate 13-36 was accessed by acylation of n-hexanol (2 g, 1.0 eq, 19.6 mmol) with 2-bromoacetyl bromide, 13-35 (5.05 g, 1.3 eq, 25.0 mmol) using NaHCO₃ (3.95 g, 2.4 eq, 47.0 mmol) in 50 mL acetonitrile. Crude product was purified by column chromatography (1×) to obtain 4.32 g (97%) of pure intermediate 13-36.

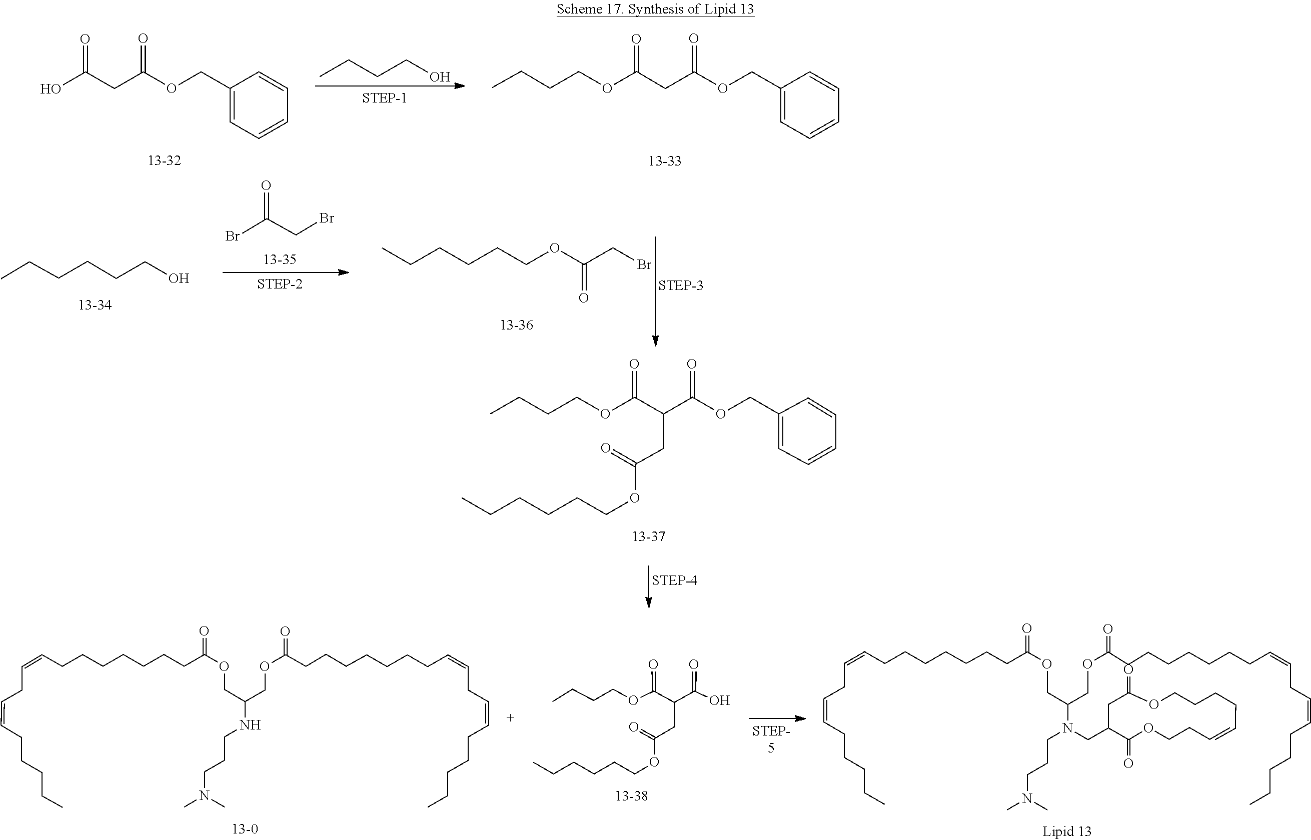

Intermediate 13-37 was accessed by in situ generation of the nucleophilic carbanion of 13-33 (1.25 g, 1.0 eq., 5.0 mmol) using NaH (200 mg, 1.0 eq, 5.0 mmol) in 8 mL DMF and displacement reaction with intermediate 13-36 (1.1 g, 1.0 eq, 5.0 mmol). Crude product was purified by column chromatography (2×) to 1.15 g (58%) of pure intermediate 13-37. Free acid intermediate 13-38 was obtained by deprotection (Pd/C, 230 mg catalyst and hydrogen gas in methanol) of intermediate 13-37 (1.15 g, 1.0 eq, 2.9 mmol). Crude product was purified by column chromatography (4×) to obtain 88 mg (9% 0/6) of pure intermediate 13-38. Intermediate 13-0 (105 mg, 1.0 eq, 0.04 mmol) was acylated with 13-38 (2.13 mmol, 0.554 g, 1.1 eq), using DIPEA (78 μL, 3.0 eq, 0.45 mmol), EDCI (87 mg, 3.0 eq, 0.45 mmol), and DMAP (5 mg, 0.3 eq, 0.04 mmol), in 2 mL DCM. Crude product was purified by column chromatography (3×) yielding 41 mg (27%) of pure Lipid 13 (≥99% purity by LC-ELSD) and characterized by proton NMR and Mass Spectrometry (see FIG. SM-1 for Lipid 13 NMR spectrum, FIG. 5M-2 for Lipid 13 LC-MS, and Table 4 for product mass).

### Synthesis of Lipid 15 by N-Acylation of Intermediate 13-11a Using the Corresponding Acid Chloride

### Synthesis of Lipid 15

Lipid 15 was synthesized as provided in scheme 18 below and as follows. Starting material, decan-4-ol, 13-29 (10.0 g, 63.0 mmol) was acylated with succinic acid, 13-30 (12.6 g, 126 mmol, 2.0 eq) using DMAP (7.7 g, 63 mmol, 1 eq) and pyridine (5.0 ml) in 5 mL THF and 15 mL DCM to obtain intermediate 13-31. Crude product was purified by column chromatography (3×) to obtain 8.9 g (55%) of pure acid intermediate 13-31. Intermediate 13-31 (1.26 g, 4.9 mmol) was converted to the acid chloride intermediate 13-3′ using oxalyl chloride (1.43 mL, 3.4 eq, 16.66 mmol) and DMF (50 μL, catalytic quantity), in 5 mL Benzene. Product showed only one spot on TLC (as methyl ester) and was used without further purification for acylation (step 3) of intermediate 13-11b. Intermediate 13-11b (275 mg, 0.39 mmol) was acylated with crude acid chloride, 13-31′ (324 mg, 3.0 eq, 1.17 mmol), TEA (270 μL, 5.0 eq, 1.95 mmol), DMAP (20 mg, catalytic quantity) in 10 mL of benzene to obtain Lipid 15. Crude product was purified by column chromatography (2×) to yield 230 mg g (64%) of pure Lipid 15 (99% purity by LC-ELSD) and characterized by proton NMR and Mass Spectrometry (see FIG. 5N-1 for Lipid 15 NMR spectrum, FIG. 5N-2 for Lipid 15 LC-MS, and Table 4 for product mass).

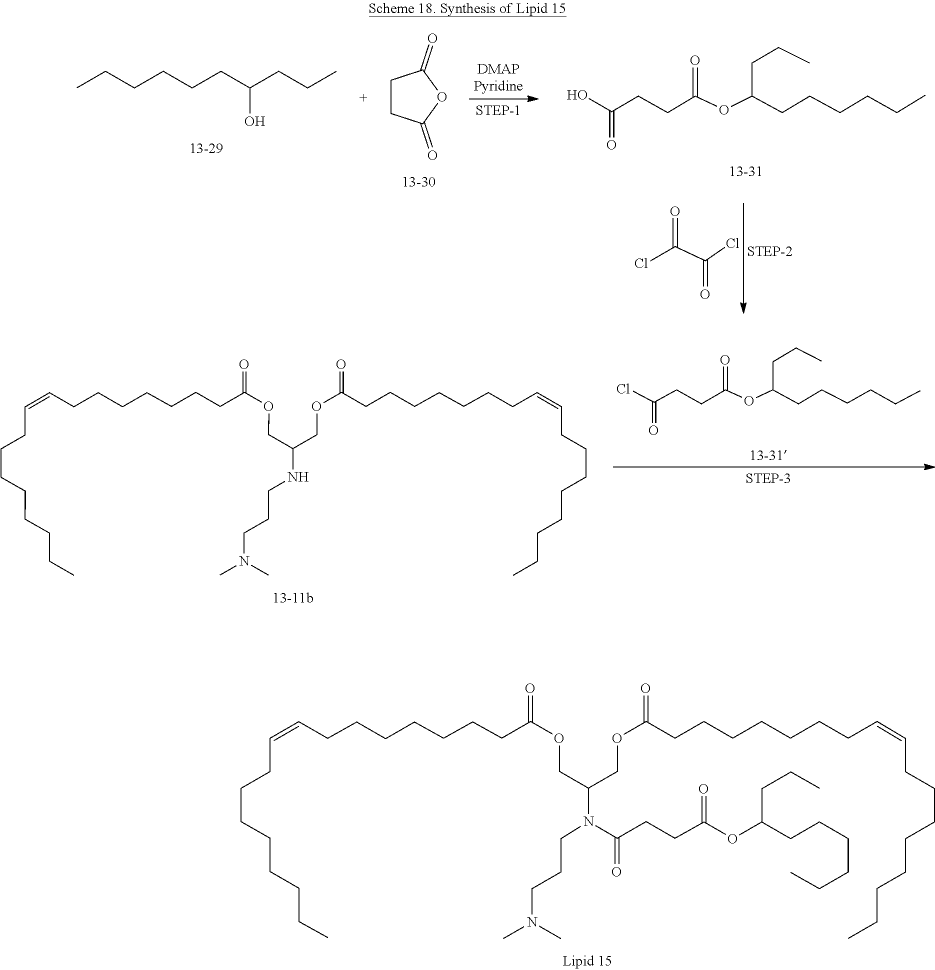

### Synthesis of Lipid 16

Lipid 16 was synthesized as provided in scheme 19 below and as follows. Starting material, octan-3-ol, 13-48 rac (3 g, 23 mmol) was acylated with succinic acid, 13-30 (46.08 mmol, 4.61 g, 2.0 eq) using DMAP (23.04 mmol, 2.8 g, 1.0 eq,) and pyridine (5.0 ml) in 5 mL THF and 15 mL DCM to obtain intermediate 13-31. Crude product was purified by column chromatography (1×) to obtain 3.4 g (64%) of pure acid intermediate 13-47 rac. Intermediate 13-47 rac (300 mg, 0.42 mmol) was converted to the acid chloride intermediate 13-47′ rac using oxalyl chloride (0.38 mL, 4.4 mmol, 3.4 eq.) and DMF (2 μL, catalytic quantity). Product showed only one spot on TLC (as methyl ester) and was used without further purification for acylation (step 3) of intermediate 13-11b. Intermediate 13-11b (270 mg, 0.38 mmol) was acylated with crude acid chloride, 13-47′ rac (0.42 mmol, 300 mg, 3.0 eq.), TEA (260 μL, 5.0 eq, 1.9 mmol), DMAP (20 mg, catalytic quantity) in 5 mL of toluene to obtain Lipid 16. Crude product was purified by column chromatography (1×) to yield 165 mg (47%) of pure Lipid 16 (99% purity by LC-ELSD) and characterized by proton NMR and Mass Spectrometry (see FIG. 5O-1 for Lipid 16 NMR spectrum, FIG. 5O-2 for Lipid 16 LC-MS, and Table 4 for product mass).

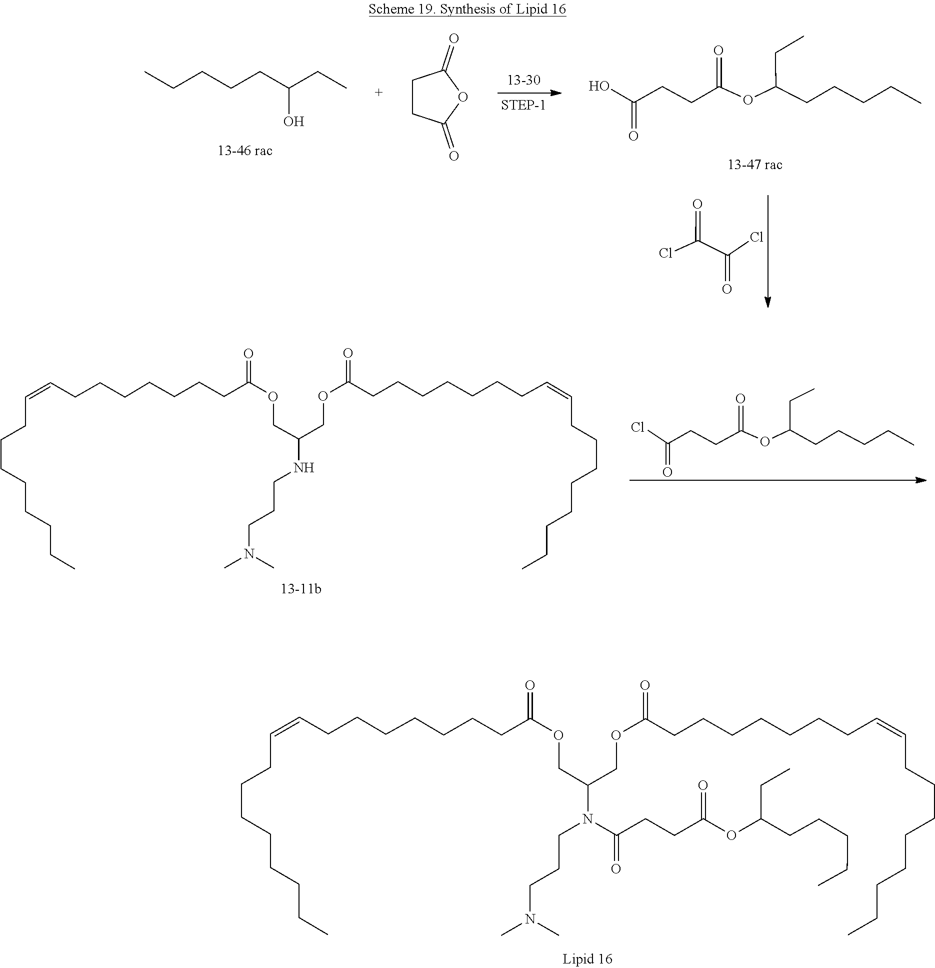

### Synthesis of Lipid 17

Lipid 17 was synthesized as provided in scheme 20 below. Octanedioic acid, 13-51 (5.0 g, 2.0 eq, 28.5 mmol) was mono-acylated with decane-3-ol, 13-29 (2.75 mL, 1.0 eq, 14.3 mmol) using EDCI (3.29 g, 1.2 eq, 17.2 mmol), DMAP (160 mg, 0.12 eq, 1.72 mmol) and TEA (9.96 mL, 5.0 eq, 71.5 mmol) in 50 mL of DCM/DMF (1:1 v/v) (50 mL) at room temperature overnight to obtain free acid 13-53. Crude product was purified by column chromatography (1×) to obtain 1.06 g (28%) of purified 13-53. Acid 13-53 (1.06 g, 2 eq., 3.7 mmol) was reacted with dihydroxyacetone (152 mg, 1.0 eq, 1.7 mmol) using EDCI (816 mg, 2.5 eq, 4.25 mmol), DMAP (50 mg, 0.25 eq, 0.43 mmol) and DIPEA (740 μL, 2.5 eq, 4.3 mmol) in 15 mL DCM at room temperature overnight to obtain ketone 13-54. Crude product was purified by column chromatography (I X) to obtain 890 mg (69%) of purified 13-54. Reductive amination of 13-54 (890 mg, 1.0 eq, 1.3 mmol) with amine, 15-3 (327 μl, 2.0 eq, 2.6 mmol) using acetic acid (150 μL, 2.0 eq, 2.6 mmol) and sodium borohydride triacetate, Na(OAc)₃BH (331 mg, 1.2 eq, 1.5 mmol) in 20 mL DCM (20 ml) at room temperature for 3 hours yielded intermediate 13-55. Crude product was purified by column chromatography (1×) to obtain purified 13-55 (470 mg, 47%). N-acylation of intermediate 13-55 using acid 13-31 and reaction conditions reported for N-acylation of Lipid 15 synthesis resulted in Lipid 17.

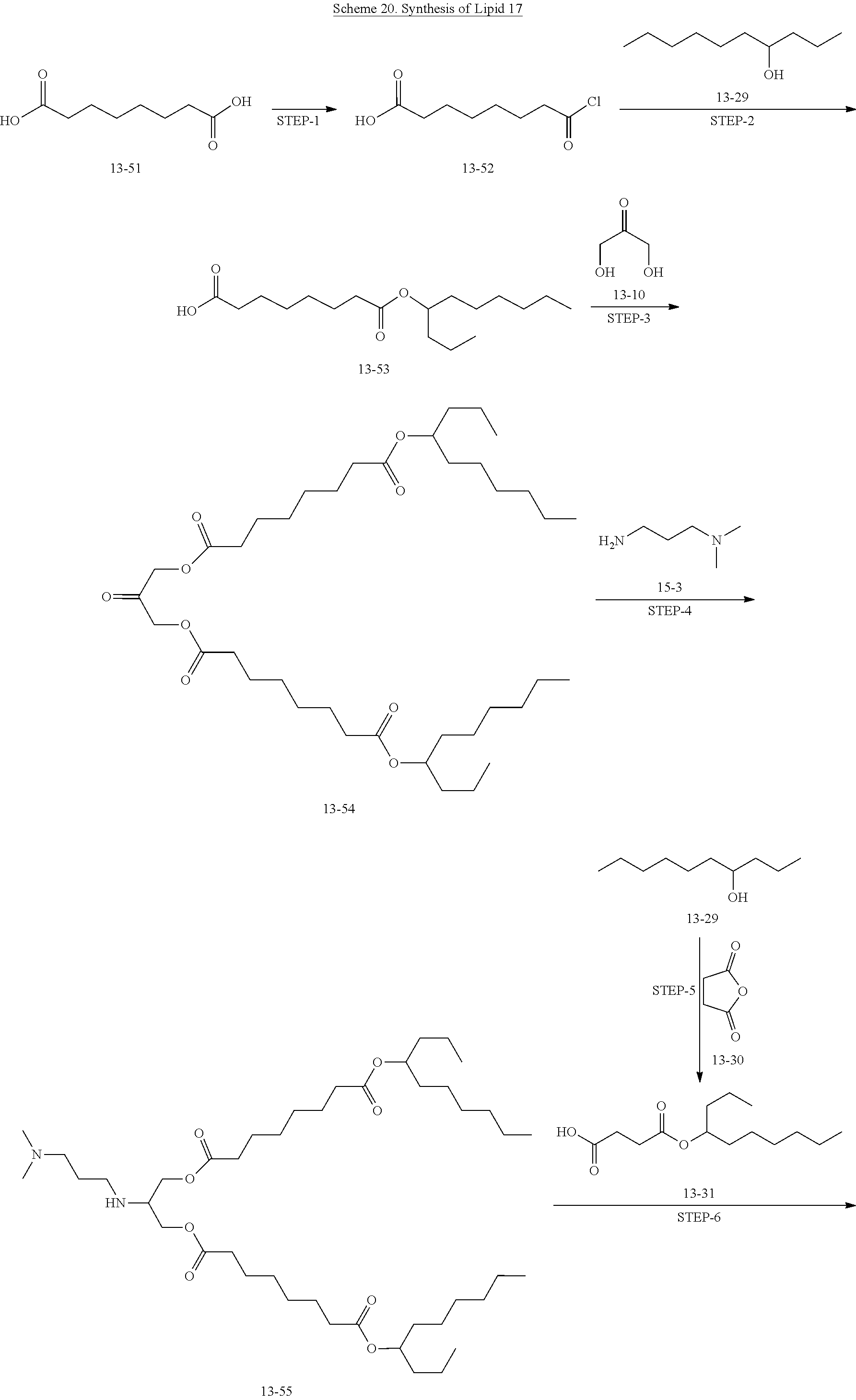

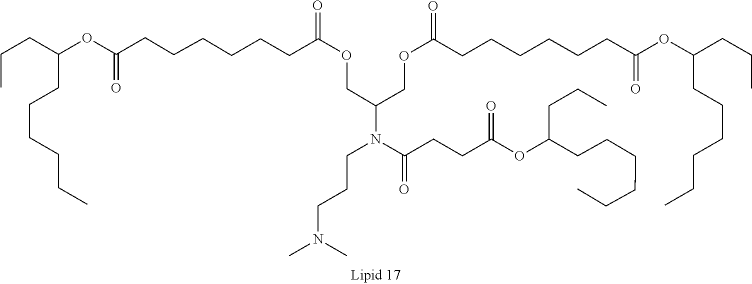 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

### Synthesis of Lipid 18 and its Isomer

An isomer of lipid 18 was synthesized as provided in scheme 21-1 below. Lipid 18 was synthesis using methods analogous to those reported for Lipid 17 by replacing decan-3-ol with octane-2-ol in the Step 1.

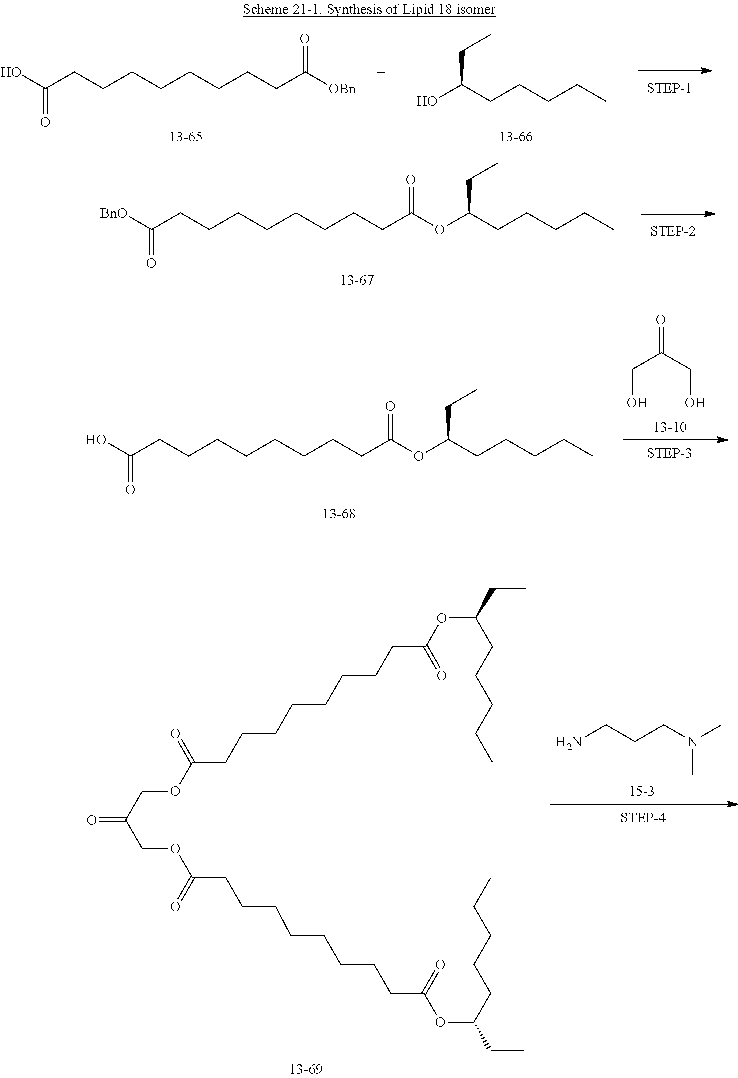

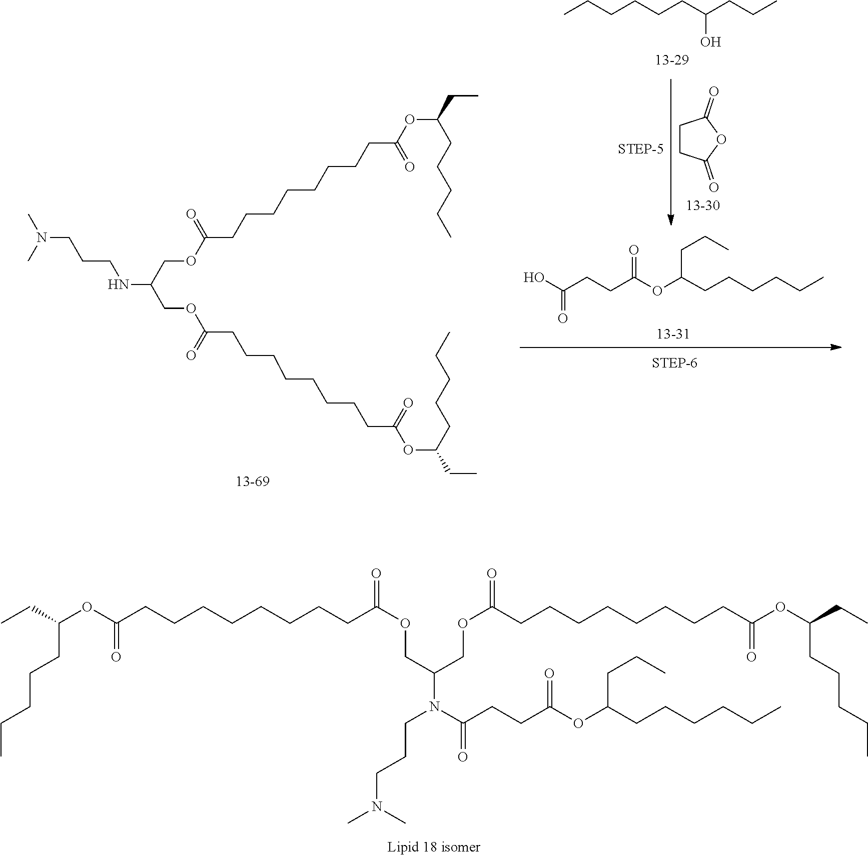 [SMILES: CCCCCCC(O)CCC ; CCCCC[C@H](CC)OC(=O)CCCCCCCCC(=O)OCC(COC(=O)CCCCCCCCC(=O)O[C@@H](CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)O[C@@H](CC)CCCCC)COC(=O)CCCCCCCCC(=O)O[C@@H](CC)CCCCC]

Lipid 18 as a racemic mixture was synthesized as provided in scheme 21-2 below.

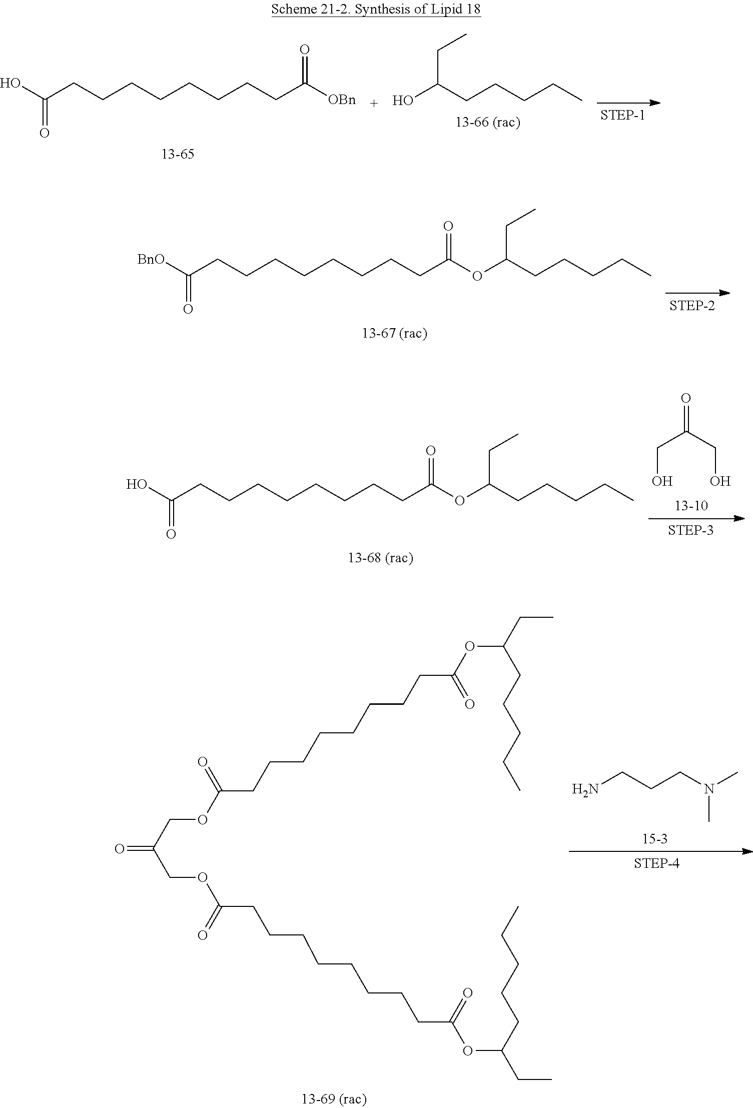

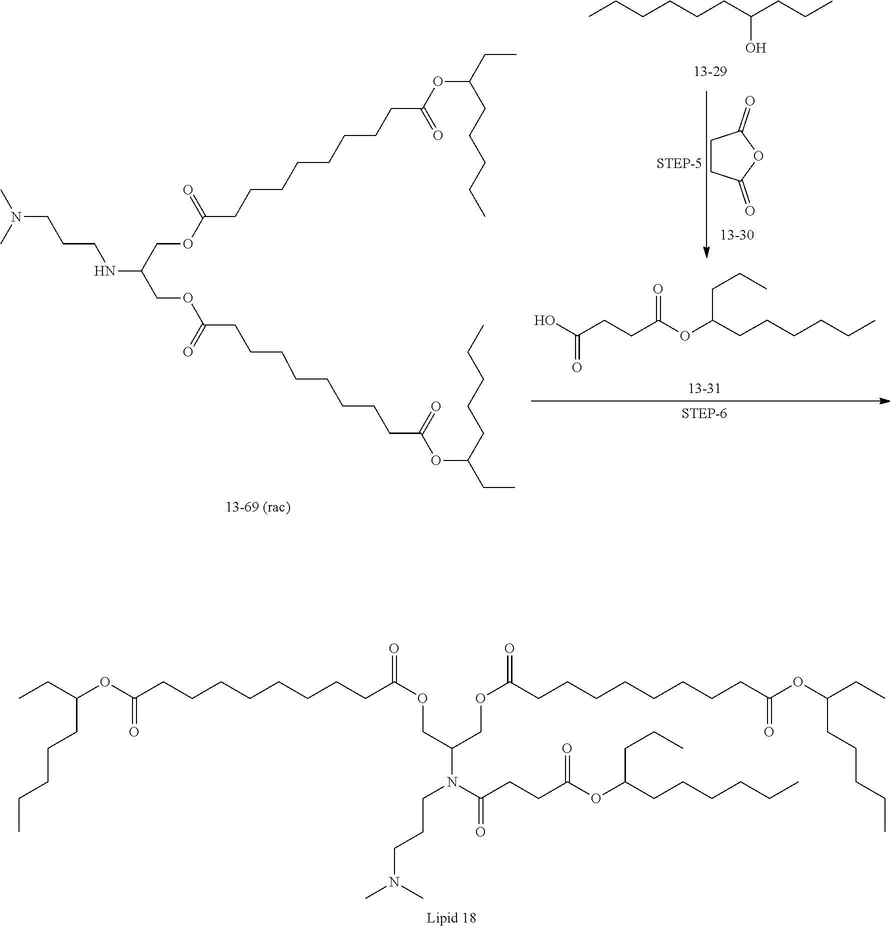 [SMILES: CCCCCCC(O)CCC ; CCCCCC(CC)OC(=O)CCCCCCCCC(=O)OCC(COC(=O)CCCCCCCCC(=O)OC(CC)CCCCC)NCCCN(C)C ; CCCCCCC(CCC)OC(=O)CCC(=O)O ; O=C1CCC(=O)O1 ; CCCCCCC(CCC)OC(=O)CCC(=O)N(CCCN(C)C)C(COC(=O)CCCCCCCCC(=O)OC(CC)CCCCC)COC(=O)CCCCCCCCC(=O)OC(CC)CCCCC]

### Synthesis of Lipid 19

Lipid 19 was synthesized as provided in scheme 22 below and as follows. Starting material dihydroxyacetone (422 mg, 4.7 mmol) was acylated with compound 13-56 (3.0 g, 2.5 eq, 11.71 mmol) using EDCI (2.24 g, 2.5 eq, 11.71 mmol), DIPEA (2.0 mL, 2.5 eq, 11.71 mmol), and DMAP (115 mg, 0.2 eq, 0.94 mmol) in 10 mL DCM yielding 2.1 g (79%) of intermediate 13-57. Reductive amination of 13-57 (2.1 g, 1.0 eq, 3.7 mmol) with amine 15-3 (925 μL, 2.0 eq, 7.4 mmol) using acetic acid (430 μL, 2.0 eq, 7.4 mmol), Na(OAc)₃BH (923 mg, 1.2 eq, 4.44 mmol) in 10.0 mL DCM yielding 1.55 g (65%) of intermediate 13-58. Intermediate 13-31 was produced as described in the synthesis of Lipid 9 and Lipid 15 earlier. N-acylation of intermediate 13-58 (484 mg, 1.0 eq, 0.74 mmol) with 13-31 (380 mg, 2.0 eq, 1.48 mmol) using EDCI (291 mg, 2.0 eq, 1.48 mmol), DIPEA (247 μL, 2.0 eq, 1.48 mmol), and DMAP (45 mg, 0.5 eq, 0.37 mmol) in 4.0 mL DCM at room temperature, overnight yielded 423 mg (63%) of pure lipid 19 (≥99% purity).

See FIG. 5P-1 for Lipid 19 NMR spectrum, FIG. SP-2 for Lipid 19 reverse phase LC-ELSD chromatogram, and Table 4 for product mass.

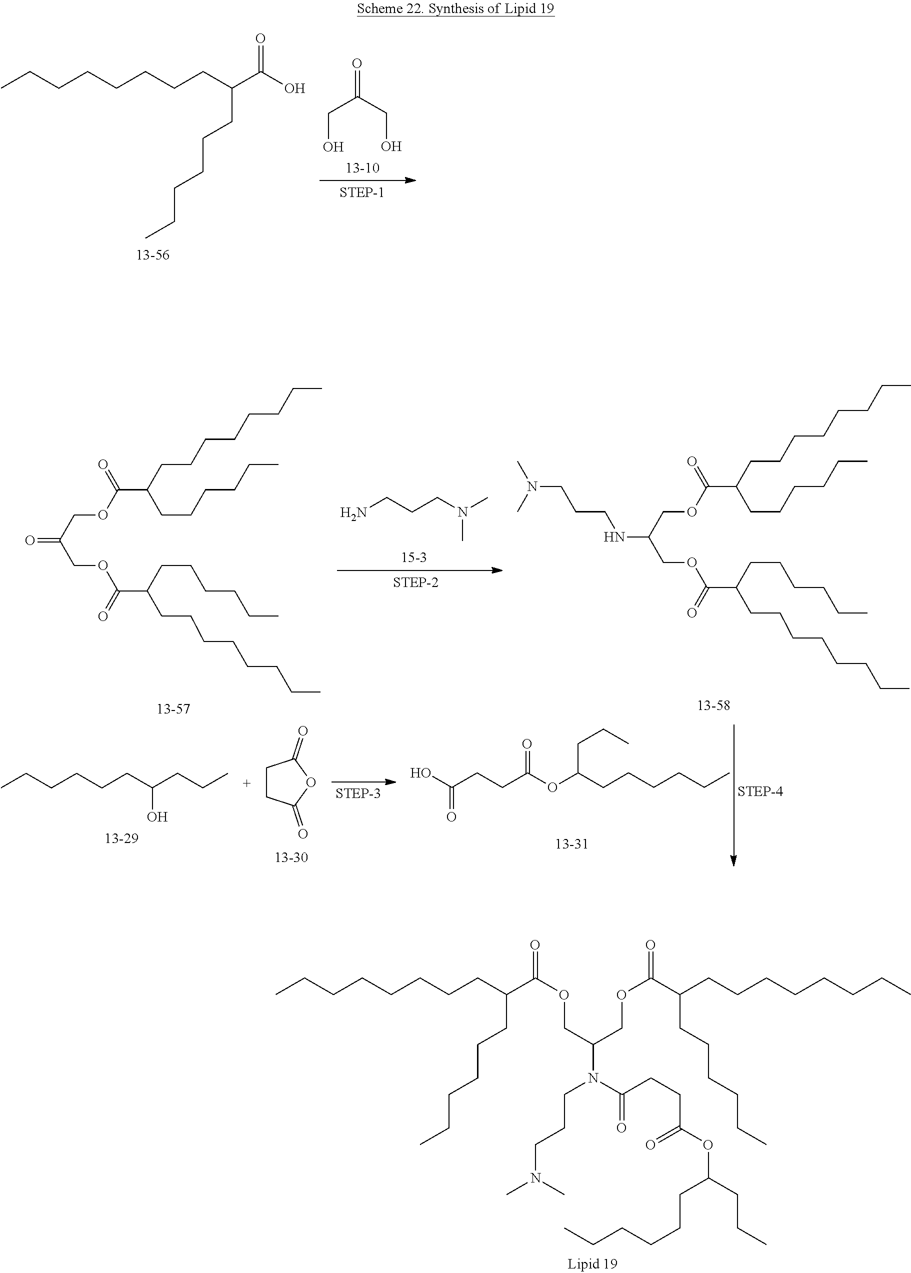

### Synthesis of Lipid 20

Lipid 20 was synthesized as provided in scheme 23 below and as follows. Monoprotected succinic acid, 13-59 (2.0 g, 1.0 eq, 9.65 mmol) was reduced to the corresponding alcohol using Borane-dimethyl sulfide (6.2 mL, 7.0 eq, 67.0 mmol) at 0-5° C., 1 hr followed by room temperature reaction overnight. Crude product was purified by column chromatography (2×) yielding 1.3 g (71%) of pure compound 13-60. Intermediate 13-60 (1.3 g, 1.3 eq, 6.7 mmol) was used to acylate acid 13-56 (1.51 mL, 1.0 eq, 5.0 mmol) using EDCI (1.63 g, 1.7 eq, 8.5 mmol), DIPEA (1.48 mL, 1.7 eq, 8.5 mmol) and DMAP (98 mg, 0.17 eq, 0.85 mmol) in 10.0 mL DCM at room temperature overnight. Crude product was purified by column chromatography (1×) yielding 1.88 g (65%) of pure intermediate 13-61. Subsequent deprotection by hydrogenation on Pd/C/Hydrogen gas (400 mg) in methanol yielded 1.42 g of free acid 13-62 (99%) of crude product. Crude 13-62 (1.32 g, 2.2 eq, 4.2 mmol) was used to acylate dihydroxyacetone, 13-10 (172 mg, 1.0 eq, 1.9 mmol), EDCI (958 mg, 2.6 eq, 5.0 mmol), DIPEA (870 μL, 2.6 eq, 5.0 mmol), and DMAP (56 mg, 0.26 eq, 0.5 mmol) in 10.0 mL DCM at room temperature, overnight to obtain ketone 13-63. Crude product was purified by column chromatography to obtain 120 mg (3.8%) of pure 13-63. Reductive amination of 13-63 (120 mg, 1.0 eq, 0.16 mmol) with amine 15-3 (42 μl, 2.0 eq, 0.32 mmol) using acetic acid (18 μL, 2.0 eq, 7.8 mmol) and Na(OAc)₃BH (41 mg, 1.2 eq, 0.19 mmol) in 3 mL DCM at room temperature of 3 hours yielded intermediate 13-64. Crude product was purified by column chromatography (1×) to obtain 23 mg (17%) of purified intermediate 13-64. N-acylation of 13-64 (23 mg, 1.0 eq, 0.028 mmol) with acid 13-31 (8.7 mg, 1.2 eq, 0.034 mmol) using EDCI (6.4 mg, 1.2 eq, 0.034 mmol), DIPEA (5.8 μL, 1.2 eq, 0.034 mmol) and DMAP (I mg, cat) in 1.5 mL DCM at room temperature overnight yielded Lipid 20. Crude product was purified by column chromatography (1×) to obtain 21 mg (70%) of pure lipid 20 (99%).

See FIG. 5Q-1 for Lipid 20 NMR spectrum, FIG. 5Q-2 for Lipid 20 reverse phase LC-ELSD chromatogram, and Table 4 for product mass.

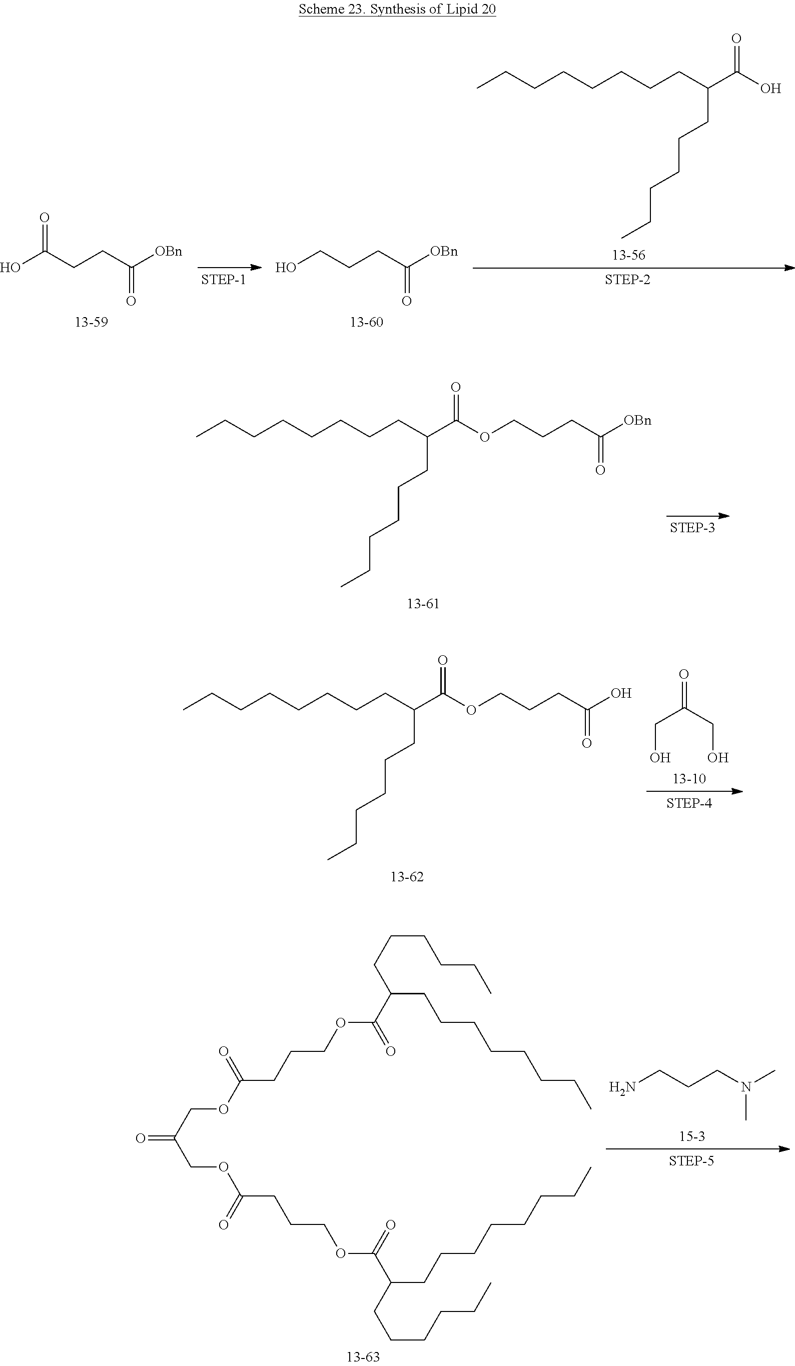

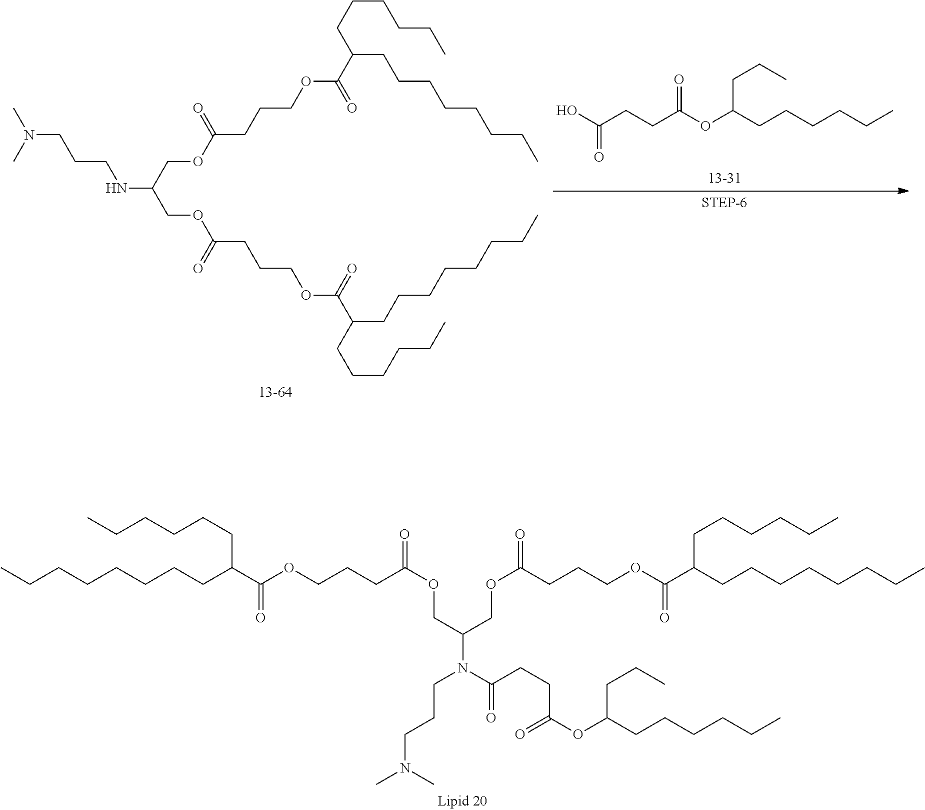 [SMILES: CCCCCCCCC(CCCCCC)C(=O)OCCCC(=O)OCC(COC(=O)CCCOC(=O)C(CCCCCC)CCCCCCCC)NCCCN(C)C ; CCCCCCC(CCC)OC(=O)CCC(=O)O ; CCCCCCCCC(CCCCCC)C(=O)OCCCC(=O)OCC(COC(=O)CCCOC(=O)C(CCCCCC)CCCCCCCC)N(CCCN(C)C)C(=O)CCC(=O)OC(CCC)CCCCCC]

### Synthesis of Lipid 21 and its Isomer

An isomer of lipid 21 was synthesized as provided in scheme 24-1 below. Briefly, alcohol 13-78 was accessed by nucleophilic addition to aldehyde 13-77 using diethyl zinc (Step 1) and subsequently used in the ring opening addition to cyclic anhydride 13-52 to access intermediate 13-79. O-acylation of dihydroxyacetone with intermediate 13-79 using conditions described in Lipid 17 synthesis yielded ketone 13-80. Reductive amination of 13-80 with amine 15-3 using conditions described in the Lipid 17 synthesis yielded intermediate 13-81. Subsequent N-acylation of intermediate 13-81 with acid 13-31 using conditions analogous to those used for Lipid 9 synthesis afforded Lipid 21.

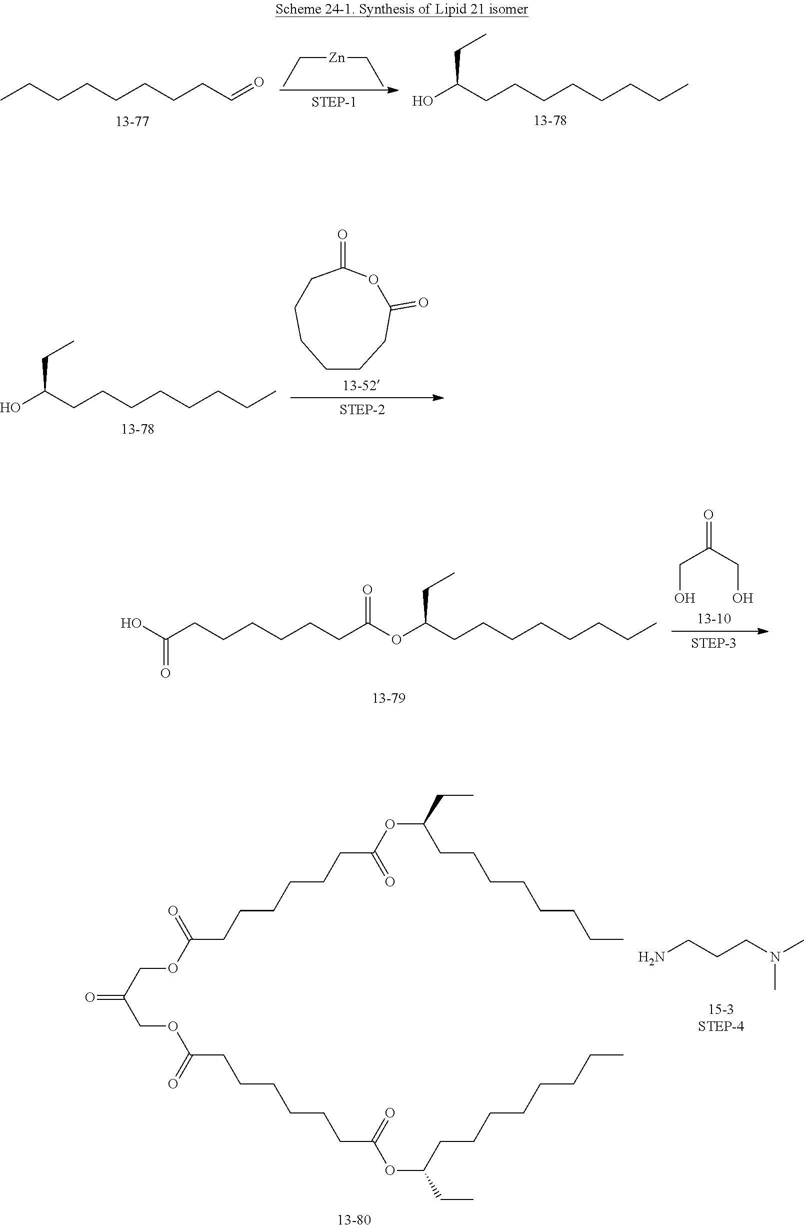

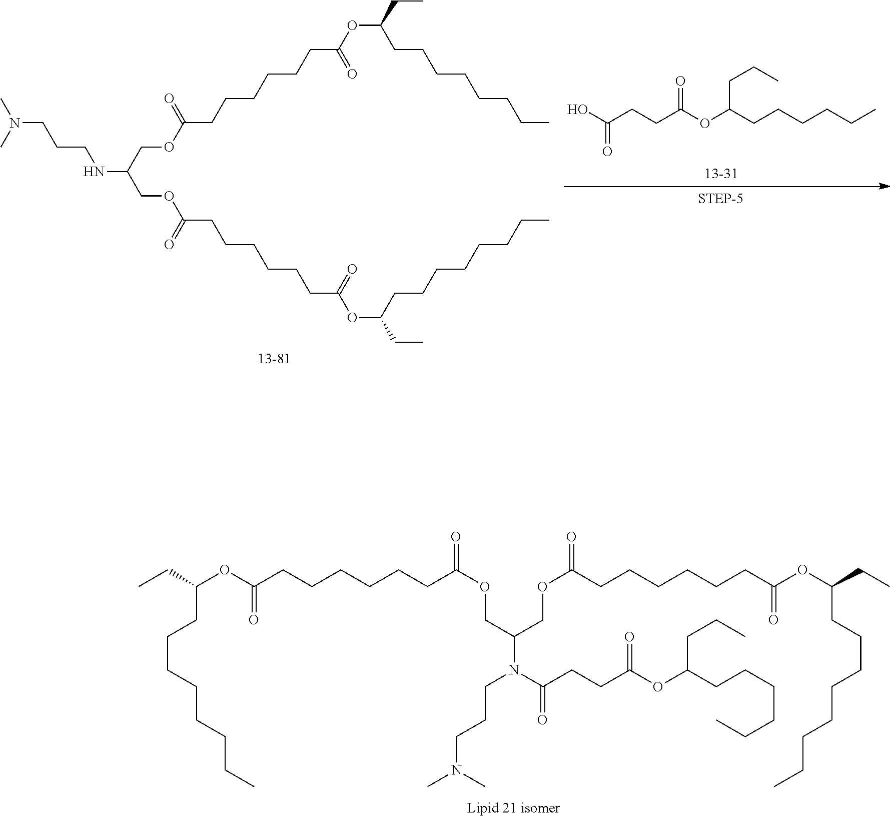

Lipid 21 as a racemic mixture was synthesized as provided in scheme 24-2 below. Briefly, Lipid 21 (racemate) was accessed using methods analogous to those described for Lipid 21 isomer except using ethyl lithium for accessing the racemic alcohol in Step 1.

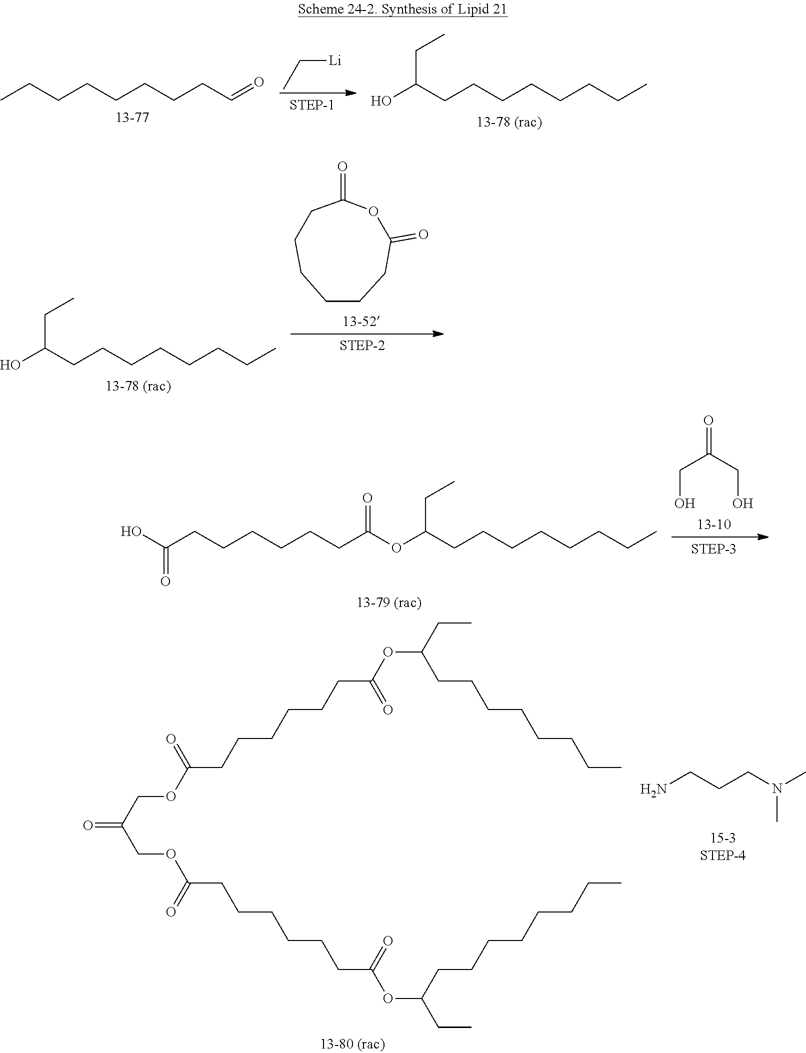

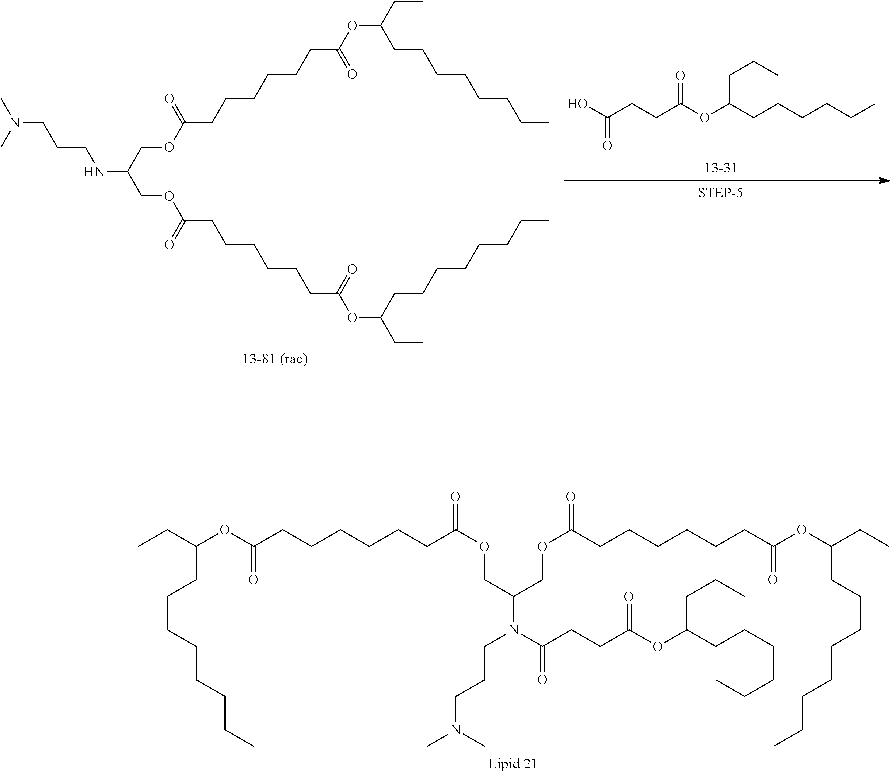

### Synthesis of Lipid 22 and its Isomer

An isomer of lipid 22 was synthesized as provided in scheme 25-1 below. Briefly, alcohol 13-78 (accessed as described for Lipid 21 synthesis above) was used in the ring opening addition to cyclic anhydride 13-73′ to access intermediate 13-82. O-acylation of dihydroxyacetone with intermediate 13-82 using conditions described in Lipid 17 synthesis yielded ketone 13-83. Reductive amination of 13-83 with amine 15-3 using conditions described in the Lipid 17 synthesis yielded intermediate 13-84. Subsequent N-acylation of intermediate 13-84 with acid 13-31 using conditions analogous to those used for Lipid 9 synthesis afforded Lipid 22 isomer.

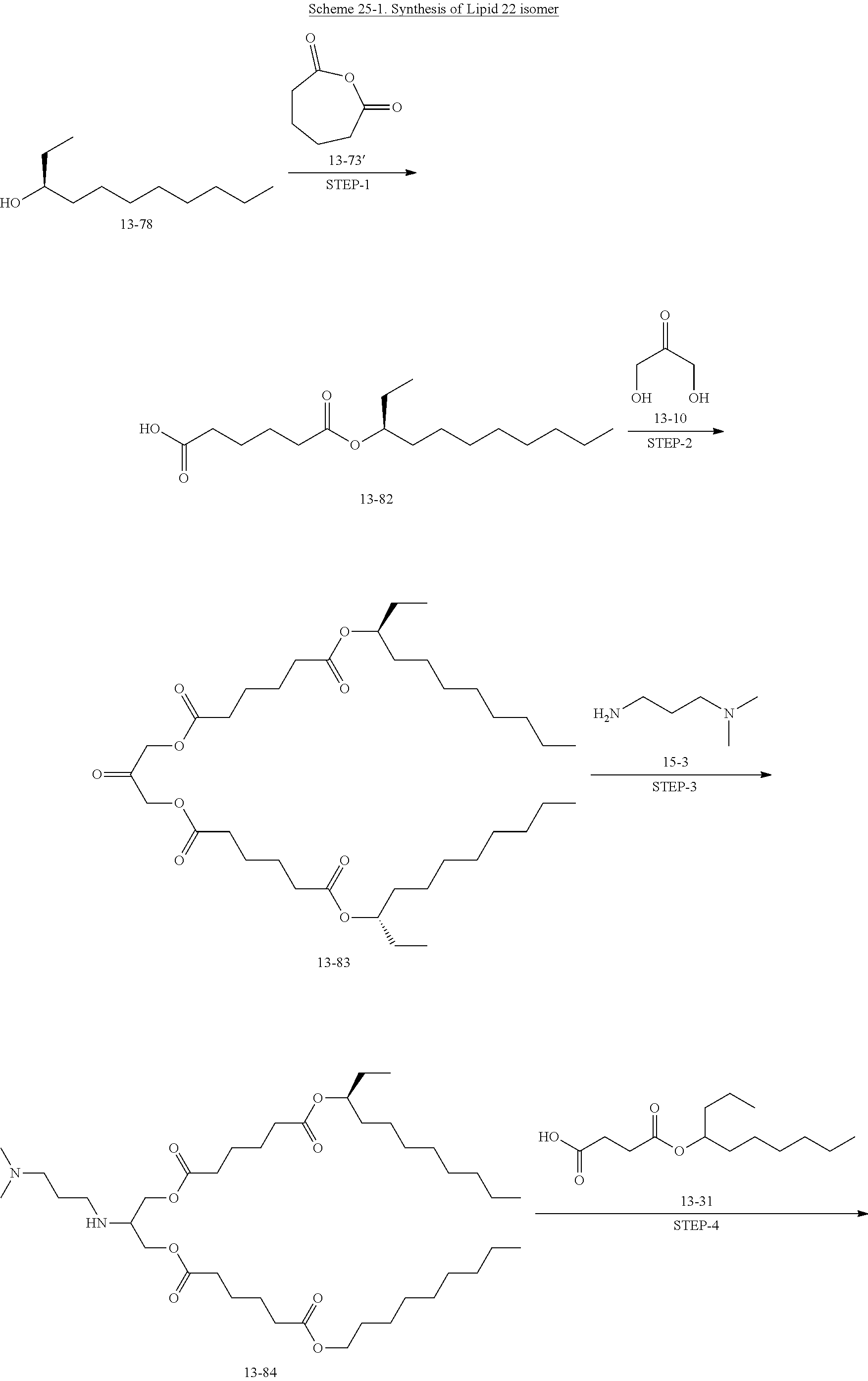

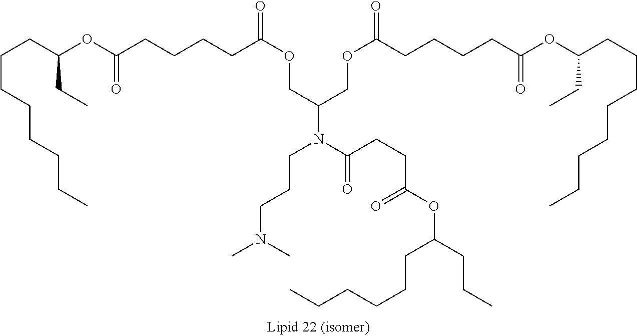 [SMILES: CCCCCCCC[C@H](CC)OC(=O)CCCCC(=O)OCC(COC(=O)CCCCC(=O)O[C@@H](CC)CCCCCCCC)N(CCCN(C)C)C(=O)CCC(=O)OC(CCC)CCCCCC]

Lipid 22 as a racemic mixture was synthesized as provided in scheme 25-2 below. Lipid 22 was accessed using methods described for Lipid 22 isomer above by replacing alcohol isomer 13-78 with racemic alcohol 13-78 rac.

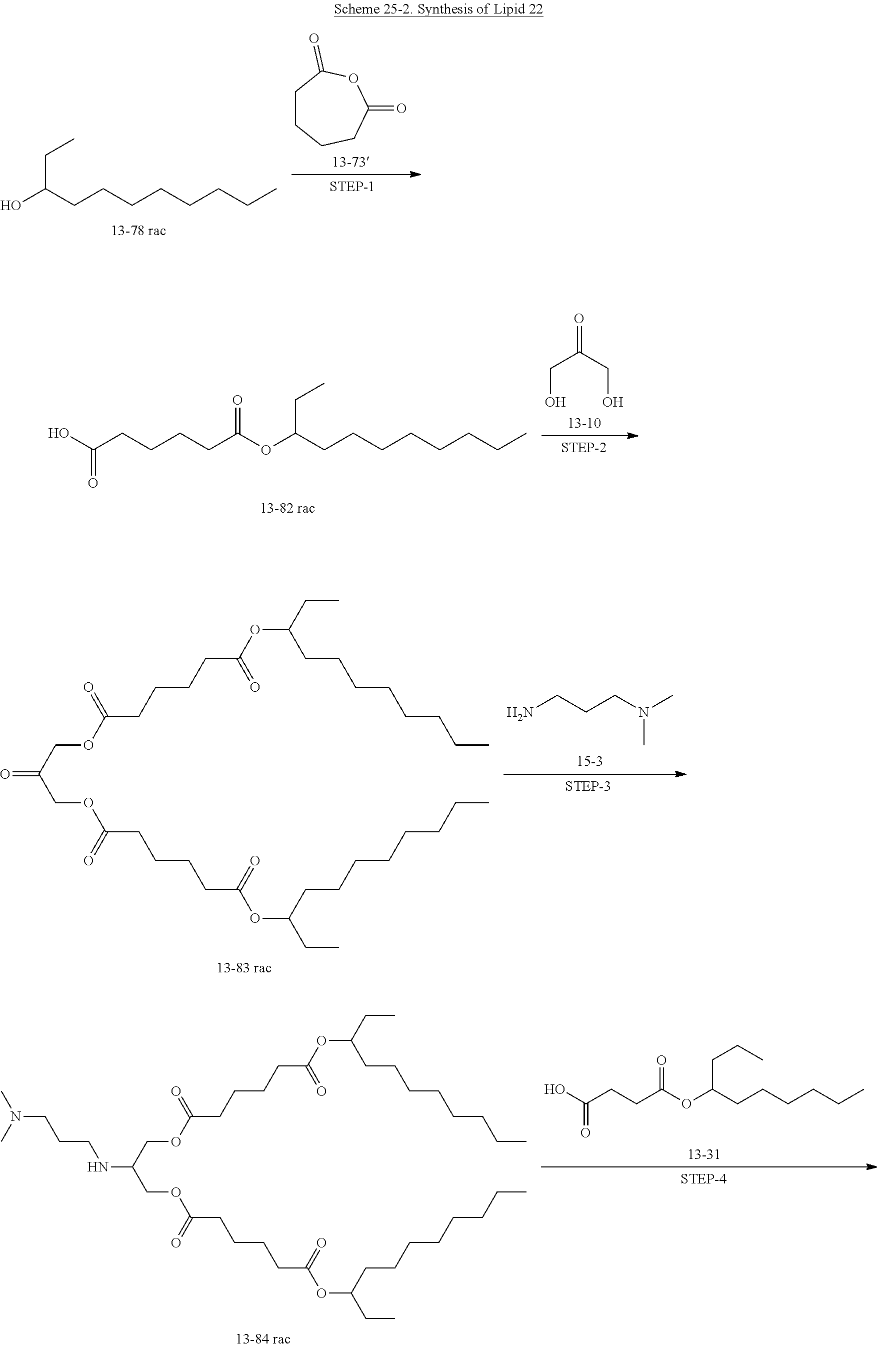

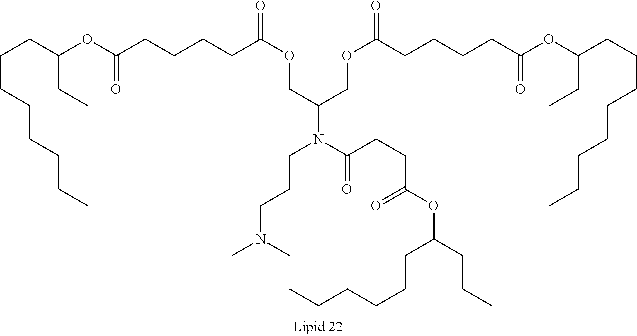 [SMILES: CCCCCCCCC(CC)OC(=O)CCCCC(=O)OCC(COC(=O)CCCCC(=O)OC(CC)CCCCCCCC)N(CCCN(C)C)C(=O)CCC(=O)OC(CCC)CCCCCC]

### Synthesis of Lipid 23

Lipid 23 was synthesized as provided in scheme 26 below. Briefly, O-acylation of dihydroxyacetone with acid 13-31 using conditions described in Lipid 9 synthesis yielded ketone 13-70. Reductive amination of 13-70 with amine 15-3 using conditions described in the Lipid 9 synthesis yielded intermediate 13-71. Subsequent N-acylation of intermediate 13-71 with acid 13-31 using conditions analogous to those used for Lipid 9 synthesis afforded Lipid 23.

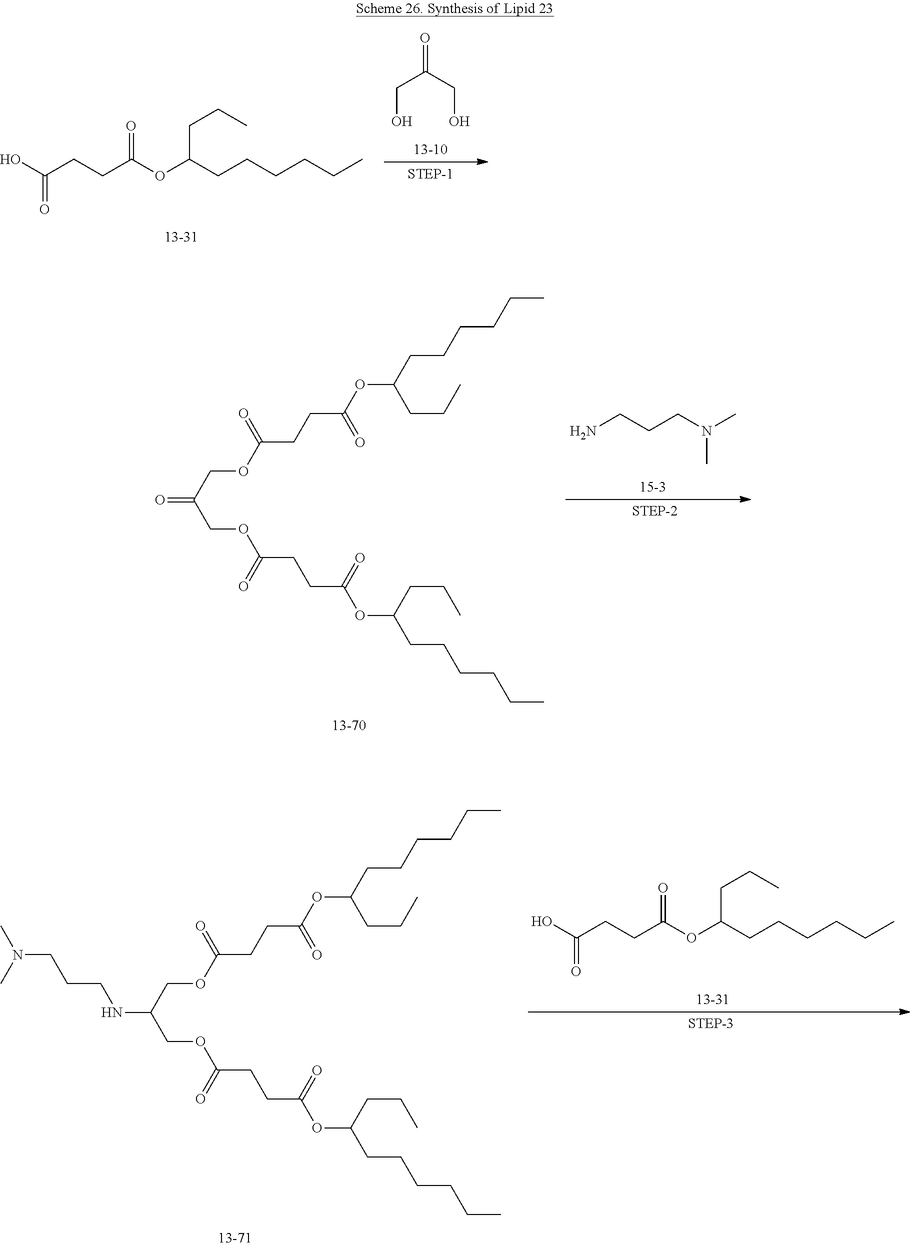

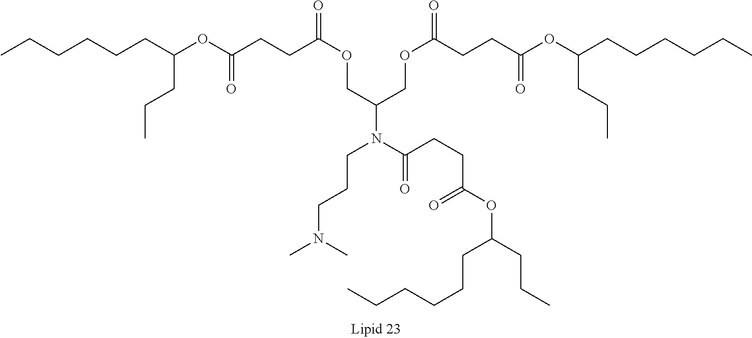 [SMILES: CCCCCCC(CCC)OC(=O)CCC(=O)OCC(COC(=O)CCC(=O)OC(CCC)CCCCCC)N(CCCN(C)C)C(=O)CCC(=O)OC(CCC)CCCCCC]

### Synthesis of Lipid 24

Lipid 24 was synthesized as provided in scheme 27 below. Briefly, acid 13-34 was accessed by O-acylation of mono-protected di-acid 13-72 with alcohol 13-29 and subsequent deprotection of intermediate 13-73 to yield acid, 13-74. O-acylation of dihydroxyacetone with intermediate 13-74 using conditions described in Lipid 17 synthesis yielded ketone 13-75. Reductive amination of 13-75 with amine 15-3 using conditions described in the Lipid 17 synthesis yielded intermediate 13-76. Subsequent N-acylation of intermediate 13-76 with acid 13-31 using conditions analogous to those used for Lipid 9 synthesis afforded Lipid 24.

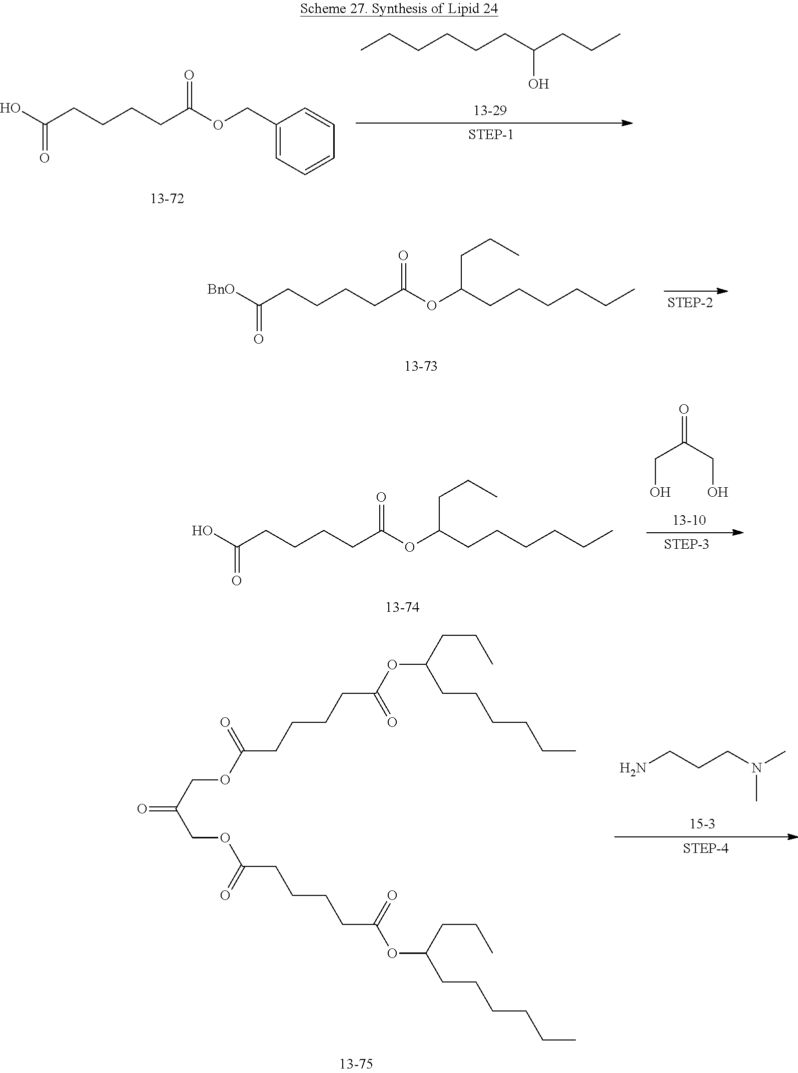

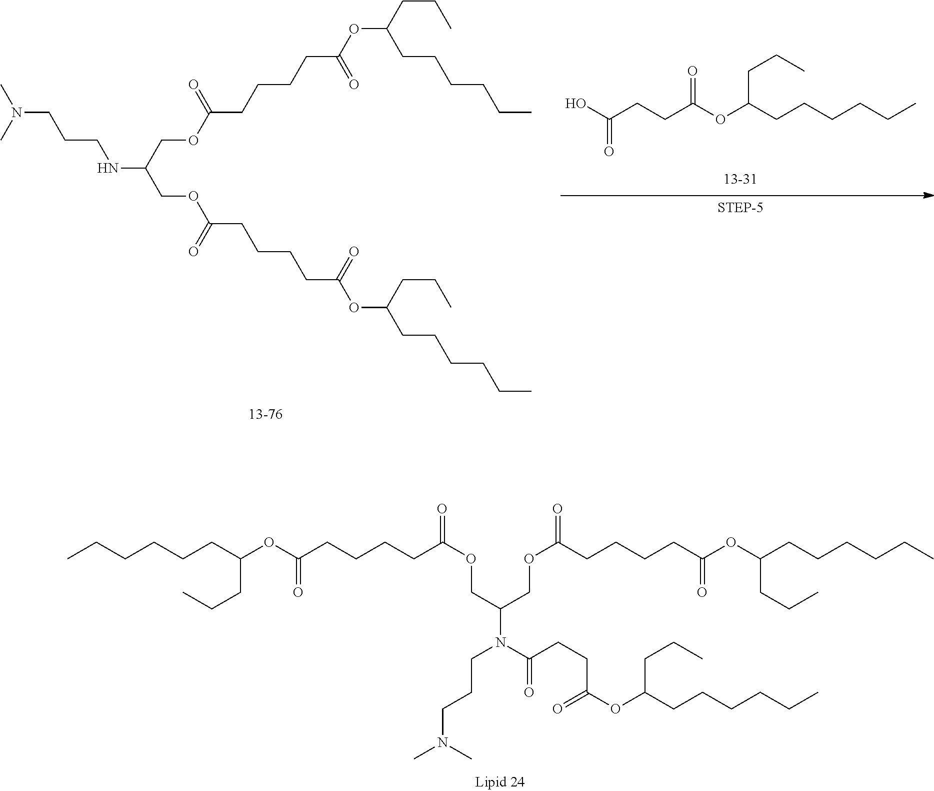 [SMILES: CCCCCCC(CCC)OC(=O)CCCCC(=O)OCC(COC(=O)CCCCC(=O)OC(CCC)CCCCCC)NCCCN(C)C ; CCCCCCC(CCC)OC(=O)CCC(=O)O ; CCCCCCC(CCC)OC(=O)CCCCC(=O)OCC(COC(=O)CCCCC(=O)OC(CCC)CCCCCC)N(CCCN(C)C)C(=O)CCC(=O)OC(CCC)CCCCCC]

### Synthesis of Lipid 25

Lipid 25 was synthesized as provided in scheme 28 below. Briefly, ring opening addition of alcohol 13-29 to cyclic anhydride 13-52′ yielded acid intermediate 13-85. O-acylation of dihydroxyacetone with intermediate 13-85 using conditions described in Lipid 17 synthesis yielded ketone 13-86. Reductive amination of 13-86 with amine 15-3 using conditions described in the Lipid 17 synthesis yielded intermediate 13-87. Subsequent N-acylation of intermediate 13-87 with acid 13-31 using conditions analogous to those used for Lipid 9 synthesis afforded Lipid 25.

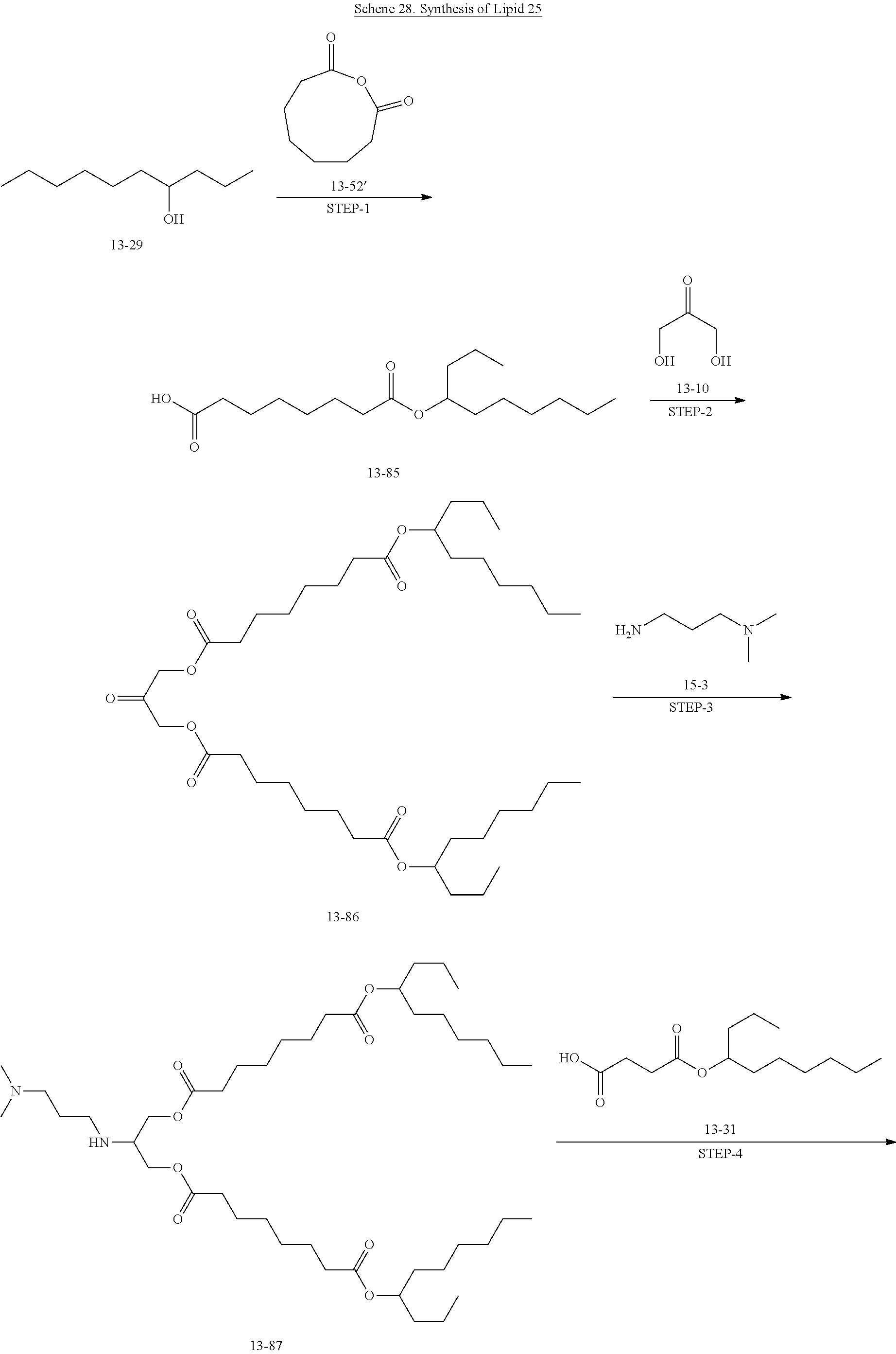

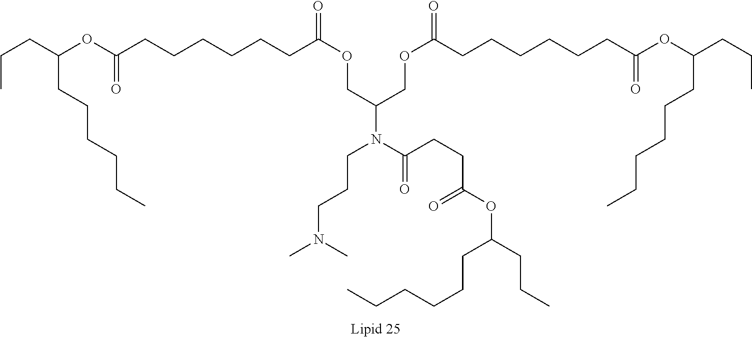 [SMILES: CCCCCCC(CCC)OC(=O)CCCCCCC(=O)OCC(COC(=O)CCCCCCC(=O)OC(CCC)CCCCCC)N(CCCN(C)C)C(=O)CCC(=O)OC(CCC)CCCCCC]

### Synthesis of Lipid 31

D Lipid 31 was synthesized as provided in scheme 29 below and as follows. Starting material 15-1 (68 mmol, 10 g, 1 eq.) was treated with p-toluene sulfonyl chloride (70 mmol, 13.3 g, 1.03 eq.) using Pyridine (80 mmol, 10.1 ml, 1.2 eq.) in 150 mL DCM to obtain protected intermediate 15-2. Crude product was recrystallized in ethyl acetate and hexane yielding 20.4 g (99%) of pure intermediate 15.2. Intermediate 15-4 was accessed by reaction of 15-2 (16.5 mmol, 5 g, 1.2 eq.) and diamine 15-3 (33 mmol, 3.35 g, 2 eq.) in 40 mL dioxane under reflux conditions. Crude product was purified by column chromatography to obtain 3.5 g (91%) of pure intermediate 15-4. N-acylation of 15-4 (108 mg, 0.268 mmol) using nonanoic acid 13-12 (0.67 mmol, 106 mg, 2.5 eq) using EDCI (0.67 mmol, 128 mg, 2.5 eq.) and DIEA (0.67 mmol, 86 mg, 2.5 eq) and DMAP (3 mg) in 10 mL DCM yielded amine 15-5. Crude product was purified by column chromatography to obtain 113 mg (65%) of purified diamine 15-5. Diol intermediate 15-6 was accessed by deprotection of 15-5 (113 mg) in 4 mL of 1M HCl and THF (1:3 v/v) at room temperature for 8 hours in quantitative yield (102 mg). Intermediate 15-6 (0.3 mmol, 100 mg, 1 eq) was acylated with linoleic acid 1-5 (0.9 mmol, 250 mg, 3 eq) using EDCI (0.9 mmol, 172 mg, 3 eq), DIPEA (0.9 mmol, 116 mg) and DMAP (10 mg, catalytic quantity) to obtain Lipid 31. Crude product was purified by column chromatography yielding 120 mg (46%) of pure Lipid 31 (≥99% purity by LC-ELSD) and characterized by proton NMR and Mass Spectrometry (see FIG. 5R-1 for Lipid 31 NMR spectrum, FIG. 5R-2 for Lipid 31 LC-MS, and Table 4 for product mass).

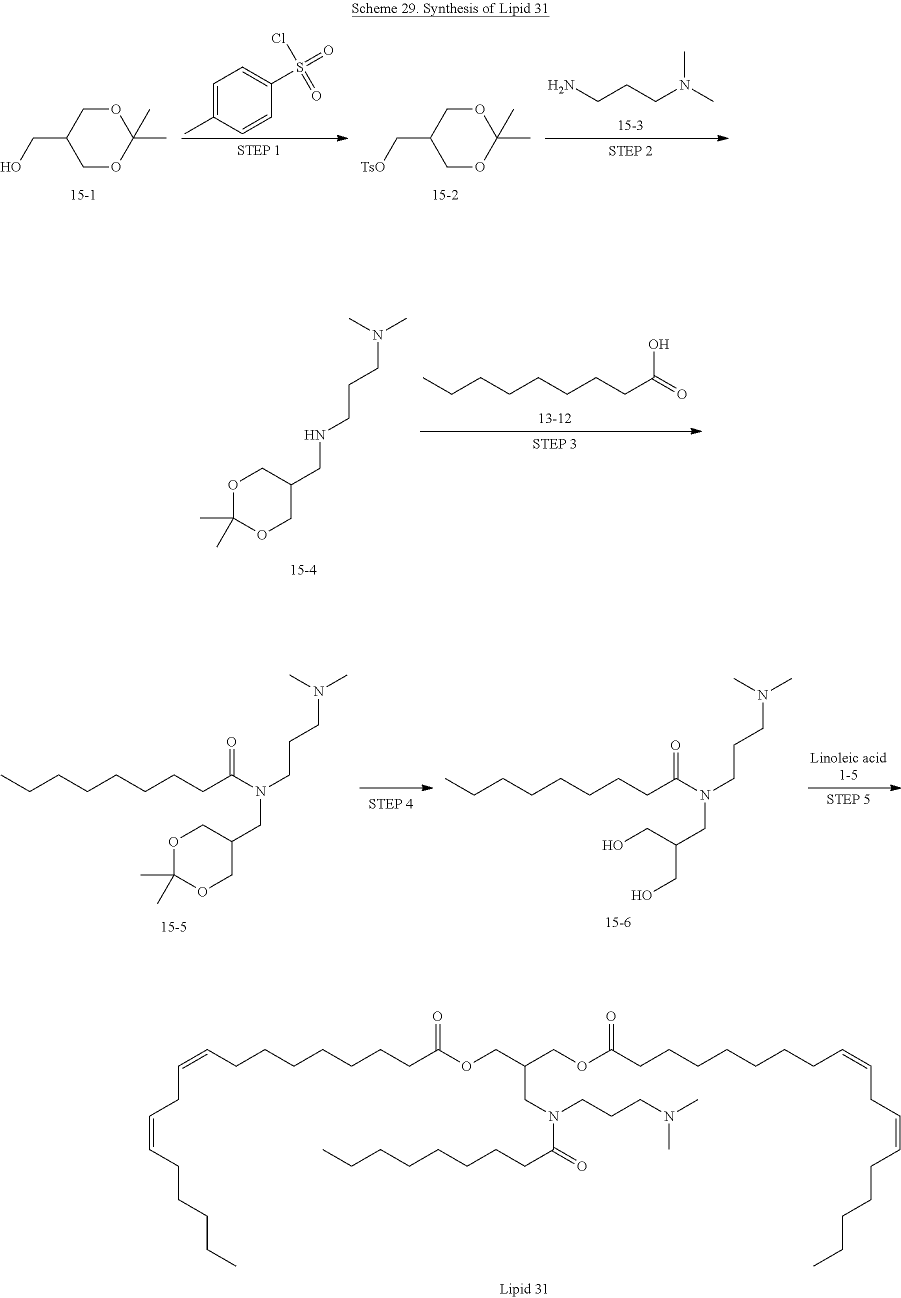

### Synthesis of Lipid 32

Lipid 32 was synthesized as provided in scheme 30 below and as follows. Intermediate 15-4 was produced as described for Lipid 31 above (steps 1 and 2, Scheme 30). N-acylation of 15-4 (4.34 mmol, 1 g, 1.0 eq) using 2-ethyl heptanoic acid 13-13 (10.85 mmol, 1.71 g, 2.5 eq) using EDCI (10.85 mmol, 2.07 g, 2.5 eq), DIEA (10.85 mmol, 1.40 g, 2.5 eq) and DMAP (10 mg) in 100 mL DCM yielded amine 15-7. Crude product was purified by column chromatography to obtain 724 mg (52%) of purified diamine 15-7. Diol intermediate 15-8 was accessed by deprotection of 15-7 (714 mg) in 3 mL of 1M HCl and 7 mL THF at room temperature for 1 hour in quantitative yield. Intermediate 15-8 (1.9 mmol, 630 mg, 1 eq) was acylated with linoleic acid 1-5 (6.49 mmol, 1.82 g, 3.4 eq) using EDCI (6.49 mmol, 1.23 g, 3.4 eq), DIPEA (6.49 mmol, 830 mg, 3.4 eq) and DMAP (20 mg, catalytic quantity) to obtain Lipid 32. Crude product was purified by column chromatography (4×) yielding 27 mg of pure fraction (≥98% purity by LC-ELSD) of Lipid 32 and characterized by proton NMR and Mass Spectrometry (see FIG. 5S-1 for Lipid 32 NMR spectrum, FIG. 5S-2 for Lipid 32 LC-MS, and Table 4 for product mass).

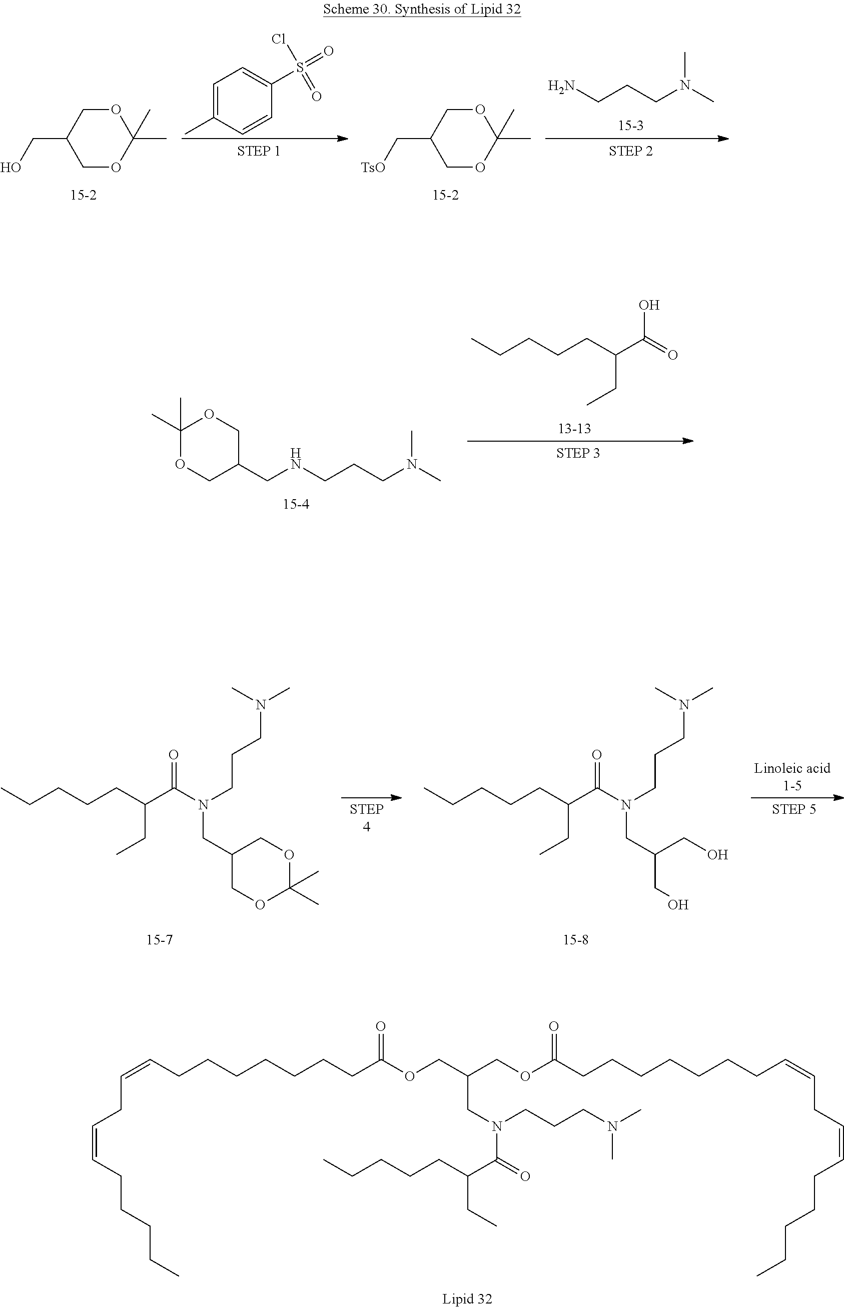

Lipid 33 was synthesized as provided in scheme 31 below and as follows. Starting material 15-1 (34.3 mmol, 5 g, 1 eq.) was tosylated using p-toluene sulfonyl chloride, TsCl (6.52 g, 1 eq, 34.3 mmol) using TEA (19.01 mL, 4 eq, 137 mmol) and DMAP (30 mg) in 200 mL DCM. Crude product was purified by column chromatography (1×) to obtain 10.2 g (98%) of reactive intermediate 15-2. Nucleophilic displacement of 15-2 (10.0 mmol, 2.3 g, 1 eq.) with diamine 15-9 (9.24 mmol, 1.0 mL, 1.2 eq.) in 10 mL dioxane (10 mL) yielded 1.6 g (97%) of compound 15-10. Nucleophilic displacement reaction was repeated using additional 15-2 (8.3 mmol, 2.5 g, 1 eq.) and diamine 15-9 (9.9 mmol, 1.1 mL, 1.2 eq.) in 50 mL dioxane for an additional quantity of compound 15-10. Crude products from the two reactions were purified by column chromatography to obtain a total of 1.7 g (~50%) pure 15-10. N-acylation of 15-10 (4.05 mmol, 875 mg, 1 eq.) with nonanoic acid 13-12 (7.1 mmol, 1.24 mL, 1.8 eq.) using EDCI (1.4 g, 1.8 eq, 7.1 mmol), DIPEA (1.3 mL, 1.8 eq, 7.1 mmol) and DMAP (90 mg, 0.2 eq, 0.81 mmol) in 8 mL DCM yielded intermediate 15-11. Crude product was purified by column chromatography (2×) to obtain 230 mg (16%) of pure intermediate 15-11. Deprotection of 15-11 (0.64 mmol, 230 mg, 1 eq.) in 5 mL of 4M HCl in dioxane yielded intermediate 15-12. Crude product was purified by column chromatography (IX) to obtain 74 mg (37%) of pure intermediate 15-12. Intermediate 15-12 (0.24 mmol, 74 mg, 1 eq.) was acylated with linoleic acid 1-5 (169 mg, 2.5 eq, 0.58 mmol) using EDCI (120 mg, 2.5 eq, 0.58 mmol), DIPEA (102 μL, 2.5 eq, 0.58 mmol) and DMAP (6 mg, 0.2 eq, 0.048 mmol) in 5 mL DCM to obtain Lipid 33. Crude product was purified by column chromatography (2×) to obtain 64 mg (32%) of pure Lipid 33 (≥99% purity by LC-ELSD) and characterized by proton NMR and Mass Spectrometry (see FIG. 5T-1 for Lipid 33 NMR spectrum, FIG. 5T-2 for Lipid 33 LC-MS, and Table 4 for product mass).

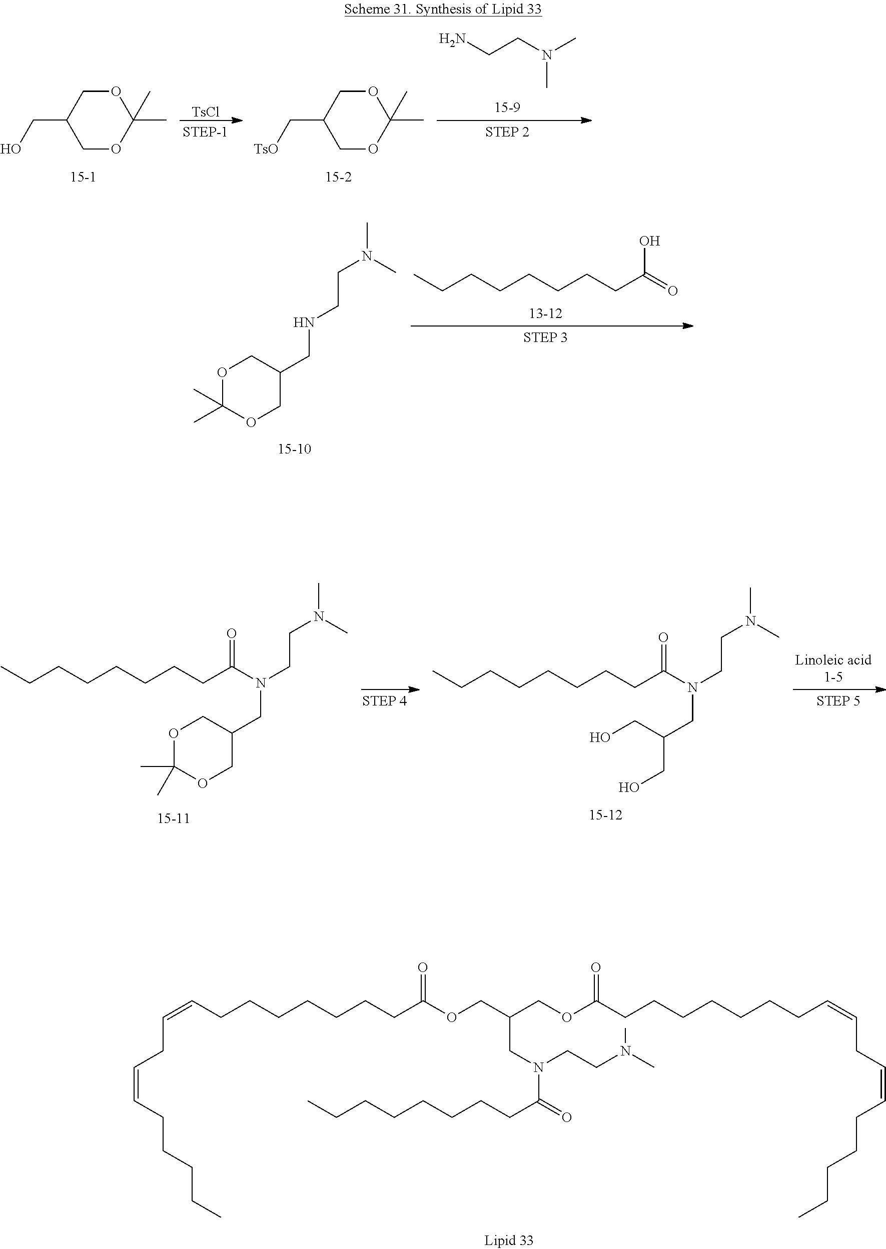

### Synthesis of Lipid 34

Lipid 34 was synthesized as provided in scheme 32 below and as follows. Intermediate 15-2 was accessed as described for Lipid 33. Nucleophilic displacement of 15-2 (3.3 mmol, 1 g, 1 eq.) with diamine 15-13 (3.9 mmol, 0.46 mL, 1.2 eq.) in 6 mL dioxane (10 mL) yielded 520 mg (64%) of compound 15-14. Reaction was repeated to access an additional 400 mg of pure compound 15-14. N-acylation of 15-14 (2.6 mmol, 620 mg, 1 eq.) with nonanoic acid 13-12 (5.3 mmol, 915 μL, 2.0 eq.) using EDCI (5.3 mmol, 1.06 g, 2.0 eq.), DIPEA (923 μL, 2.0 eq, 5.3 mmol) and DMAP (58 mg, 0.2 eq, 0.05 mmol) in 10 mL DCM yielded intermediate 15-15. Crude product was purified by column chromatography (2×) to obtain 355 mg (35%) of pure intermediate 15-15. Deprotection of 15-15 (1.03 mmol, 355 mg, 1 eq.) in 7 mL of 4M HCl in dioxane yielded intermediate 15-16. Crude product was purified by column chromatography (2×) to obtain 40 mg (13%) of pure intermediate 15-16. Intermediate 15-16 (0.116 mmol, 40 mg, 1 eq.) was acylated with linoleic acid 1-5 (81 mg, 2.5 eq, 0.29 mmol) using EDCI (55 mg, 2.5 eq, 0.29 mmol), DIPEA (3.2 μL, 2.5 eq, 0.29 mmol) and DMAP (2 mg, 0.2 eq, 0.05 mmol) in 10 mL DCM to obtain Lipid 34. Crude product was purified by column chromatography (2×) to obtain 73 mg (73%) of pure Lipid 34 (≥99% purity by LC-ELSD) and characterized by proton NMR and Mass Spectrometry (see FIG. 5U-1 for Lipid 34 NMR spectrum, FIG. 5U-2 for Lipid 34 LC-MS, and Table 4 for product mass).

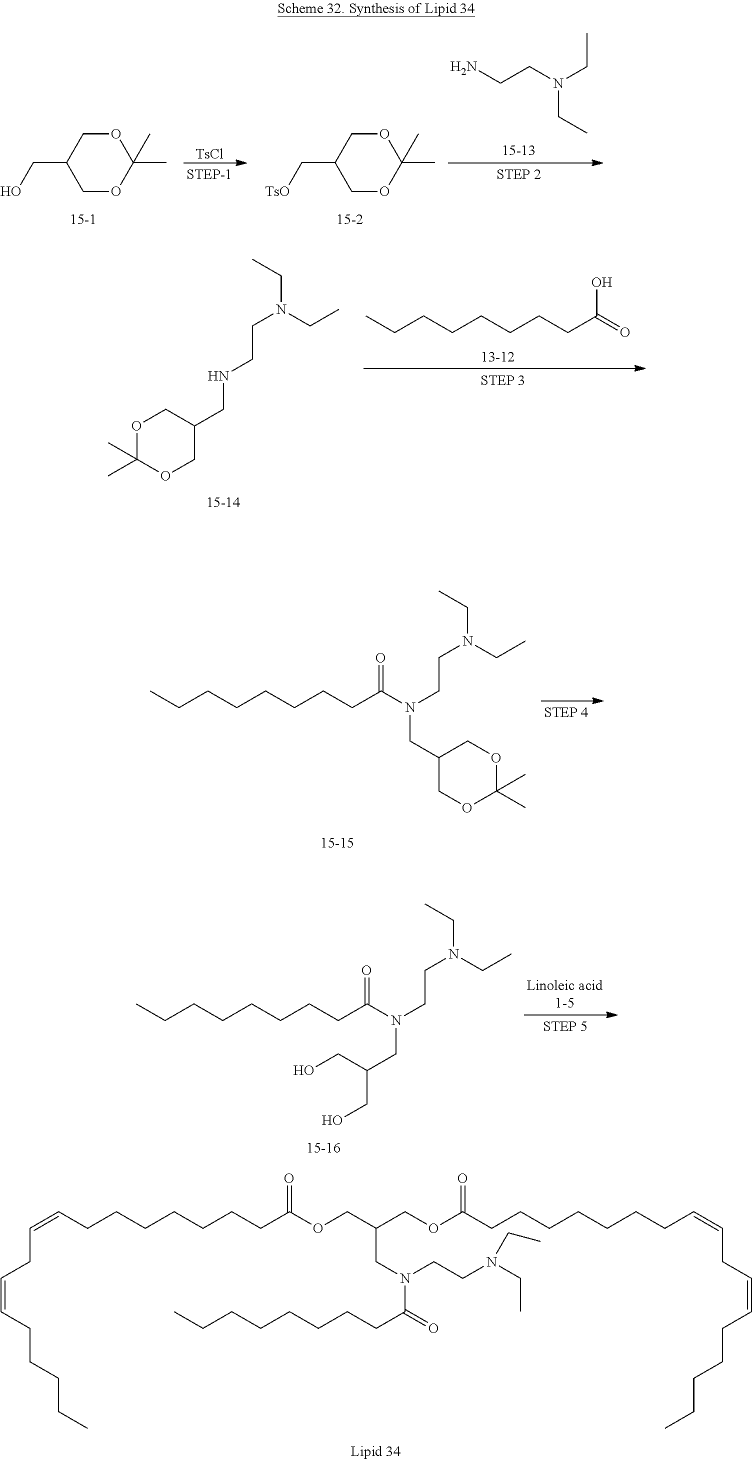

### Synthesis of Lipid 35

Lipid 35 was synthesized as provided in scheme 35 below.

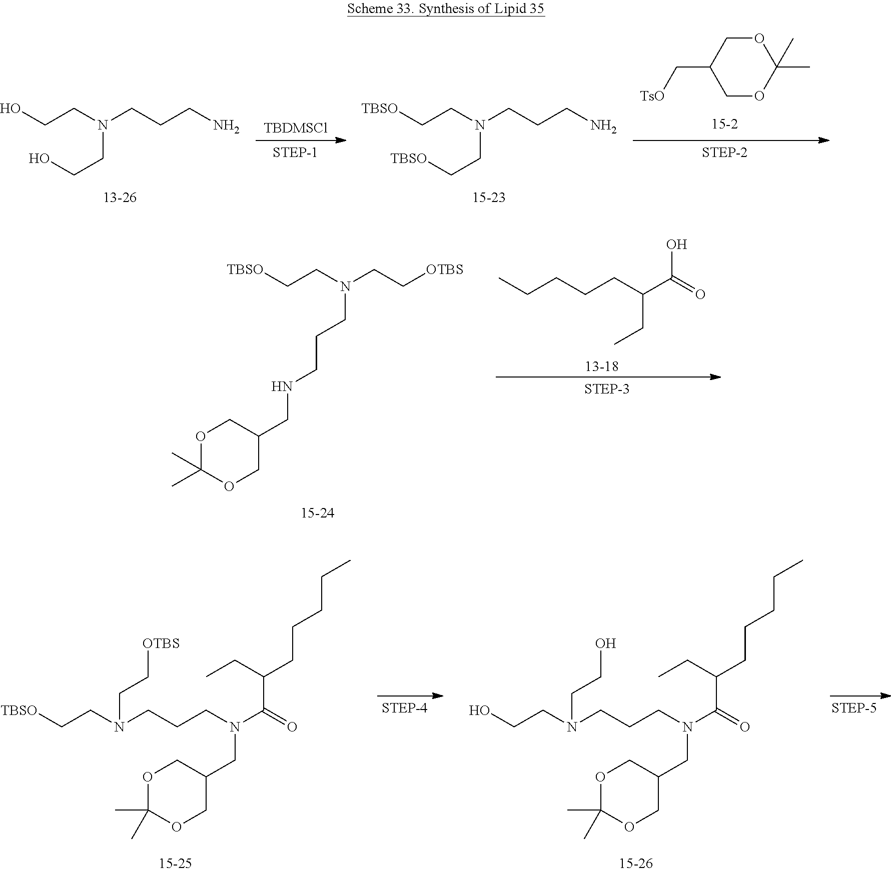

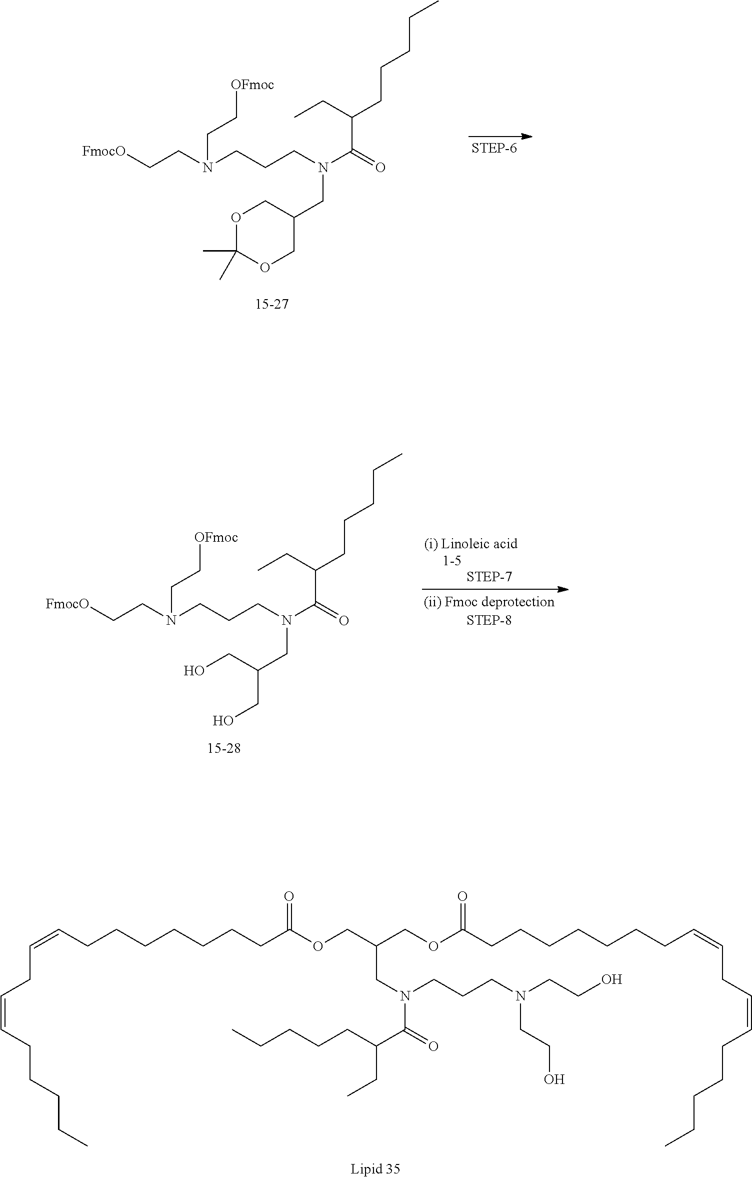

### Synthesis of Lipid 36

Lipid 36 was synthesized as provided in scheme 36 below.

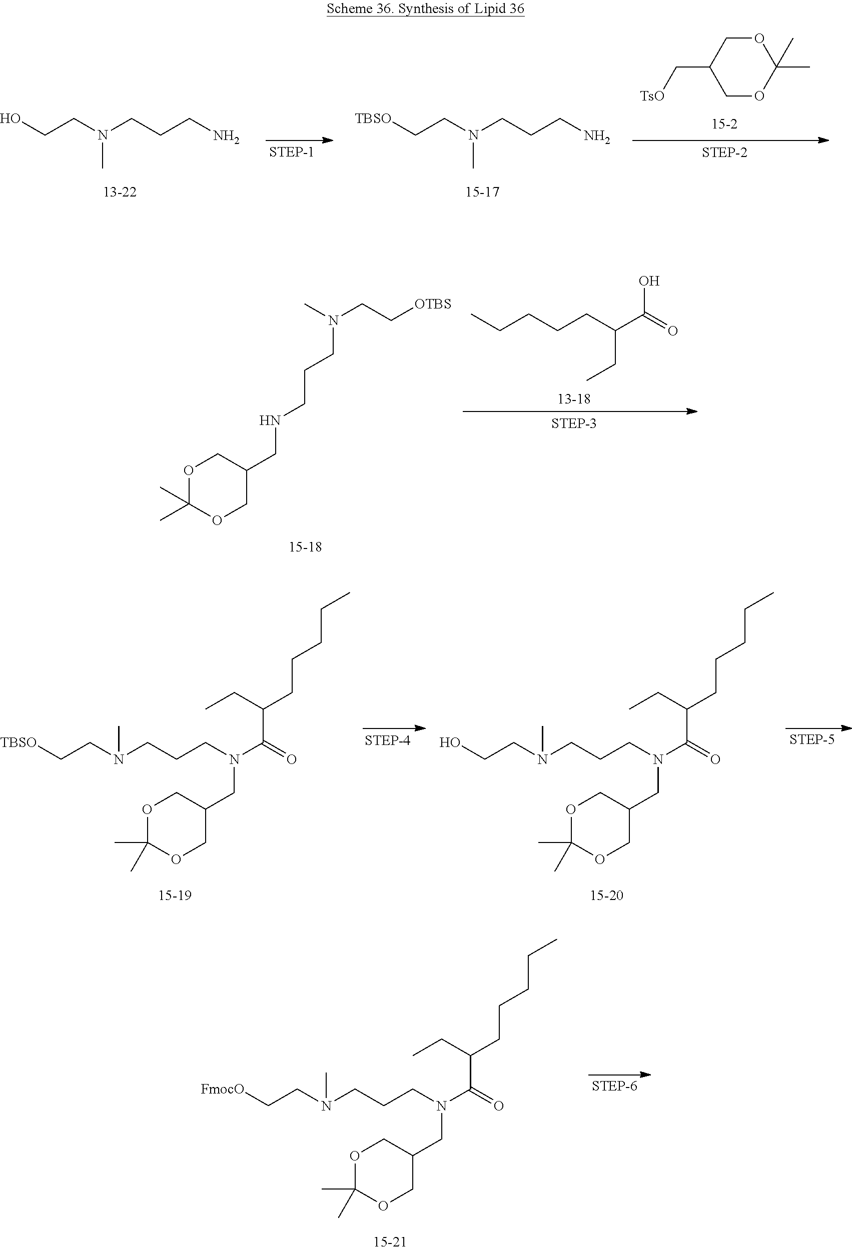

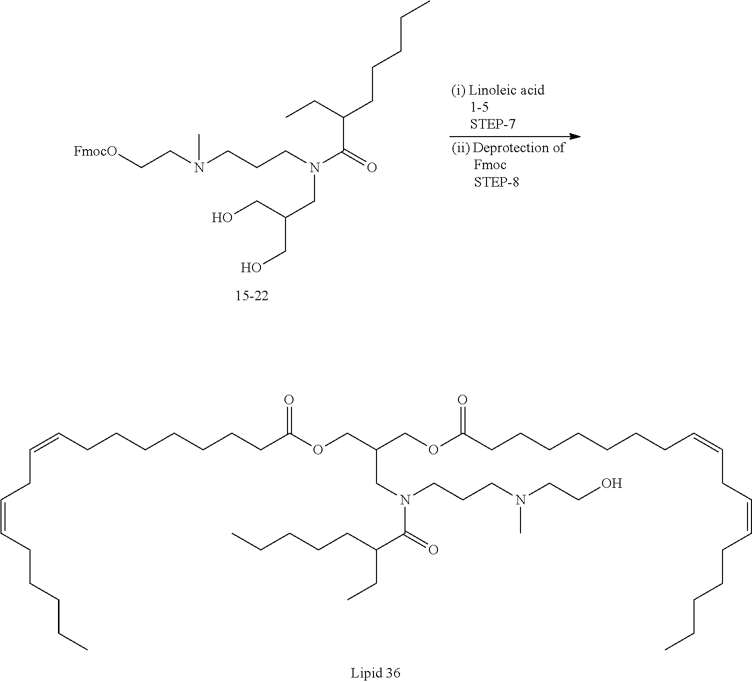

### Example 2: Preparation of LNPs By Microfluidic Mixing Using Exemplary Ionizable Lipids

Exemplary LNPs were produced using cationic Lipid 9 and cationic Lipid 15 as synthesized in Example 1.

LNPs encapsulating an mRNA payload were prepared by mixing an aqueous mRNA solution and an ethanolic lipid blend solution (containing ionizable lipid, DSPC, DPG-PEG and Cholesterol at lipid ratios shown in Table 5) using an in-line microfluidic mixing process. The mRNA (eGFP encoding mRNA, TriLink Biotechnologies, California, US) stock solution was diluted in pH 4 acetate buffer (yielding a 133 μg/mL solution of mRNA) in 21.7 mM pH 4 acetate buffer. The lipid components were dissolved in anhydrous ethanol at the relative ratios set forth in Table 5 below.

TABLE 5
Ratios of lipid components in LNPs.

| | | Ratio of Lipid to | | Theoretical |
|---|---|---|---|---|
| | | mRNA (nmol | Concentration in | LNP Lipid |
| | | lipid/100 μg | Lipid Solution | Composition |
| Lipid | Source | mRNA) | (mM) | (mol %) |
| Ionizable Cationic | — | 1,500 | 6 | 49.2 |
| Lipid | | | | |
| Cholesterol | Dishman | 1,200 | 4.8 | 39.4 |
| | Netherlands | | | |
| DSPC | Avanti Polar | 300 | 1.2 | 9.8 |
| | Lipids, Alabama, | | | |
| | U.S. | | | |
| DPG-PEG(2000) | NOF America, | 46 | 0.18 | 1.5 |
| | New York, U.S. | | | |
| DiIC18(5)-DS | Invitrogen, | 1.8 | 0.007 | 0.06 |
| | Massachusetts, US | | | |

The mRNA and lipid solutions were mixed using a NanoAssemblr Ignite microfluidic mixing device (part no. NIN0001) and NxGen mixing cartridge (part no. NIN0002) from Precision Nanosystems Inc. (British Columbia, CA). Briefly, the mRNA and lipid solutions were each loaded into separate polypropylene syringes. A mixing cartridge was inserted into the NanoAssemblr Ignite, and the syringes were directed mounted into the luer ports of the mixing cartridge. The two solutions were then mixed at a 3:1 v/v ratio of mRNA solution to lipid solution at a total flow rate of 9 mL/min using the NanoAssemblr Ignite. The resulting suspension was held at room temperature for a minimum of 5 minutes before proceeding to ethanol removal and buffer exchange.

Following mixing, ethanol removal and buffer exchange was performed on the resulting LNP suspension using a discontinuous diafiltration process. A centrifugal ultrafiltration device with 100,000 kDa MWCO regenerated cellulose membrane (Amicon Ultra-15, MilliporeSigma, Massachusetts, US) was sanitized with 70% ethanol solution and then washed twice with HBS exchange buffer (25 mM pH 7.4 HEPES buffer with 150 mM NaCl). The LNP suspension was then loaded into the device and centrifuged at 500 RCF until the volume was reduced by half volume. The suspension was then diluted with exchange buffer (25 mM pH 7.4 HEPES buffer) to bring the suspension back to the original volume. This process of two-fold concentration and two-fold dilution was repeated five additional times for a total of six discontinuous diafiltration steps. The LNP suspension was then exchanged into MBS (25 mM pH 6.5 MES buffer with 150 mL NaCl) by diluting ten-fold with MBS and centrifuging at 500 RCF until the volume was reduced by one tenth. This ten-fold dilution with MBS and ten-fold concentration step was repeated one more time. The retentate containing the LNPs in MBS was recovered from the centrifugal ultrafiltration device and stored at 4° C. until further use.

### Example 3: Characterization of LNPs

This Example describes the characterization of LNPs (e.g., LNPs comprising an ionizable cationic lipid, where the ionizable cationic lipid is KC3 or Lipid 15) produced according to methods described in Example 2.

Samples of the LNPs produced in Example 2 were characterized to determine the average hydrodynamic diameter, zeta potential, and mRNA content (total and dye-accessible mRNA). The hydrodynamic diameter was determined by dynamic light scattering (DLS) using a Zetasizer model ZEN3600 (Malvern Pananalytical, UK). The zeta potential was measured in 5 mM pH 5.5 MES buffer and 5 mM pH 7.4 HEPES buffer by laser Doppler electrophoresis using the Zetasizer.

RNA content of the nanoparticles is measured using Thermo Fisher Quant-iT RiboGreen RNA Assay Kit. Dye accessible RNA, which includes both un-encapsulated RNA and accessible RNA at the LNP surface, is measured by diluting the nanoparticles to approximately 1 μg/mL mRNA using HEPES buffered saline, and then adding Quant-iT reagent to the mixture. Total RNA content is measured by disrupting a nanoparticle suspension by dilution of the stock LNP batch (typically at ≥40 μg/mL RNA) in 0.5% Triton solution in HEPES buffered saline to obtain a 1 μg/mL RNA solution (final nominal concentration based on formulation input values) and subsequent heating at 60° C. for 30 minutes followed by addition of Quant-It reagent. RNA is quantified by measuring fluorescence at 485/535 nm, and concentration is determined relative to a contemporaneously run RNA standard curve.

### Example 4. Preparation of Conjugates to Enable Hematopoietic Stem Cell (HSC) Targeting

This Example describes a method for the production of lipid-HSC targeting group conjugates for incorporation into HSC-targeting LNPs (e.g., LNPs comprising an ionizable cationic lipid, where the ionizable cationic lipid is KC3 or Lipid 15).

Fabs and full-length antibodies that bind to HSC-specific targets (CD117, CD105, and CD34) were conjugated to DSPE-PEG(2k)-maleimide via covalent coupling between the maleimide group and a C-terminal cysteine in the heavy chain (HC). The protein (3-4 mg/mL), after buffer exchange into oxygen free, pH 7 phosphate buffer, was reduced in 0.2 mM TCEP in oxygen free pH 7 phosphate buffer for 1.5 hour at room temperature. The reduced protein was isolated using a 40 kDa SEC column to remove TCEP and buffer exchanged into fresh oxygen free pH 7 phosphate buffer.

The conjugation reaction was initiated by addition of a 10 mg/mL micellar suspension of DSPE-PEG-maleimide (Avanti Polar Lipids, Alabama, US) and 30 mg/mL DSPE-PEG-OCH₃ (Avanti Polar Lipids, Alabama, U.S.) (1:1 to 1:3 weight ratio is used depending on protein) in oxygen free pH 5.7 citrate buffer (1 mM Citrate). Protein solution was concentrated to—3-4 mg/mL using a 10 kDa Regenerated Cellulose Membrane and subsequently buffer exchanged in oxygen free pH 7 phosphate buffer using a 40 kDa Size Exclusion Column. The conjugation reaction was carried out using 2-4 mg/mL protein and a 3.5 molar excess of maleimide at 37° C. for 2 hours followed by incubation at room temperature for an additional −2-16 hours.

The production of the resulting conjugate was monitored by HPLC and the reaction quenched in 1.5 mM cysteine. The resulting conjugate (DSPE-PEG(2k)-anti-hSP34 Fab) was isolated using a 100 kDa Millipore Regenerated Cellulose membrane filtration using pH 7.4 HEPES buffer saline (25 mM HEPES, 150 mM NaCl) buffer and stored at 4° C. prior to use. After quenching, the final micelle composition consists of a mixture of DSPE-PEG-Fab, DSPE-PEG-maleimide (cysteine terminated), and DSPE-PEG-OCH₃. The ratio of the three components was DSPE-PEG-Fab: DSPE-PEG-maleimide (cysteine terminated). DSPE-PEG-OCH₃=1:2.45:3.45-10.35 (by mole)).

### Example 5: Preparation of LNPs Containing HSC Targeting Groups

This Example describes an exemplary method for the incorporation of an HSC targeting group lipid conjugate into a preformed LNP (e.g., an LNP comprising an ionizable cationic lipid, wherein the ionizable cationic lipid is KC3 or Lipid 15).

LNPs from Example 2 and HSC targeting group conjugates prepared using methods described in Example 4 were combined in an Eppendorf tube. The tubes were vortexed for 10 seconds at 2,500 rpm. The Eppendorf tubes were placed in the ThermoMixer at 60° C. at 300 rpm for 1 hour. Resulting targeted LNP suspension was subsequently stored at 4° C. until use or alternatively stored frozen after reconstitution into sucrose medium at final sucrose concentration of 9.6 wt. % by dilution using the appropriate volume of a 50 wt. % sucrose stock solution (in HEPES buffer saline; 25 mM HEPES, 150 mM NaCl)) and stored frozen at −80° C.

### Example 6: Preparation of LNPs By Microfluidic In-line Mixing and Tangential Flow Filtration Using an Exemplary Ionizable Lipid

This example describes preparation of LNPs (e.g., LNPs comprising an ionizable cationic lipid, wherein the ionizable cationic lipid is KC3 or Lipid 15) using scalable unit operations, namely in-line microfluidic mixing followed by tangential flow filtration (TFF) for ethanol removal and buffer exchange.

Using the mixing process in Example 2, an LNP mixture was produced totaling 12 mL at an RNA concentration of 300 μg/mL. Ethanol removal and buffer exchange was subsequently performed using tangential flow filtration (TFF).

Following mixing, ethanol removal and buffer exchange were performed on the resulting LNP suspension using a hollow fiber TFF module (Repligen, US P/N C02-E300-05-N). Briefly, the TFF module was rinsed with DI water and pumped dry before use. LNPs were then added to the reservoir, and the exchange buffer (25 mM pH 7.4 HEPES buffer with 150 mM NaCl) was used as the diafiltration buffer. The TFF module was primed, and diafiltrations (DVs) were then initiated by ramping up the peristaltic pump to target flow rate and adjusting Retentate valve until target transmembrane pressure (TMP) is reached. A flow rate of 35 mL/min and a TMP of 3.5 psi were the target operating parameters for the system once diafiltration was initiated. Throughout the diafiltration process, the TMP was kept constant by adjusting the retentate valve. Permeate flow rate was monitored and did not decrease significantly over time. Six diafiltrations were performed, with samples set aside at the end of each diafiltration to later track the buffer exchange process. Final ethanol content was <0.1%, as measured by refractive index measurements on DV samples, and pH measurements confirmed the buffer exchange into the exchange buffer. Upon the completion of six diafiltrations, the pump was stopped, and a concentration of the resulting LNP suspension was subsequently performed.

The concentration of the LNP suspension was performed using the same TFF module that was used during the buffer exchange process. TMP and flow rate (post pump ramp up) during the buffer exchange process were maintained and the suspension was allowed to concentrate by stopping the addition of diafiltration buffer retentate reservoir. The resulting LNP suspension was collected and filtered with a 0.2 μm syringe filter. The suspension was sampled for analytical purposes and then stored at 4° C. until further use.

Using the LNP characterization process in Example 3, LNP batch was characterized to determine the average hydrodynamic diameter and mRNA content (total and dye-accessible). The microfluidic mixing process with ethanol removal and buffer exchange by TFF results in sub-100 nm particles exhibiting narrow polydispersity and good mRNA encapsulation (<20% dye accessible RNA).

### Example 7: Method for Determination of the LNP Apparent pKa Using the Toluidinyl-Naphthalene Sulfonate (TNS) Fluorescent Probe

This example describes the fluorescent dye-based method used for measurement of the apparent pKₐ of the lipid nanoparticles (e.g., LNPs comprising an ionizable cationic lipid, wherein the ionizable cationic lipid is KC3 or Lipid 15). Apparent pKa determines the nanoparticle surface charge under physiological pH conditions, typically a pKa value in the endosomal pH range (6-7.4) results in LNPs that are neutral or slightly charged at plasma or the extracellular space (pH 7.4) and become strongly positive under acidic endosomal environments. This positive surface charge drives fusion of the LNP surface with negatively charged endosomal membranes resulting in destabilization and rupture of the endosomal compartment and LNP escape into the cytosolic compartment, a critical step in cytosolic delivery of mRNA and protein expression via engagement of the cells ribosomal machinery.

The apparent pKa of LNPs is determined by 6-(p-Toluidino)-2-naphthalenesulfonic acid (TNS) fluorescence measurement in aqueous buffers covering a range of pH values (pH-4-pH 10). TNS dye is non-fluorescent when free in solution, but fluoresces strongly when associated with a positively charged lipid nanoparticle. At pH values below the pKa of the nanoparticle, positive LNP surface charge results in dye recruitment at the particle interface resulting in TNS fluorescence. At pH values above the LNP pKa, the LNP surface charge is neutralized and TNS dye dissociates away from the particle interface resulting in loss of fluorescence signal. The apparent pKₐ of the LNP is reported as the pH at which the fluorescence is at 50% of its maximum, as determined using a four-point logistic curve fit.

### Example 8: General Formulation and Physiochemical Characterization Methodology for mRNA Encapsulating LNPs Based on Lipids 1-8, 9-15 and 31-34

Lipid nanoparticles (LNPs) (e.g., LNPs comprising an ionizable cationic lipid, wherein the ionizable cationic lipid is KC3 or Lipid 15) bearing a nucleic acid were formulated by a microfluidic mixing process using lipid and solvent compositions described in Example 2 and 6 above and buffer exchanged into pH 7.4 HEPES buffer saline (resulting in ethanol removal and pH adjustment) using either centrifugal ultrafiltration membrane filter devices or a tangential flow filtration (TFF) process; and characterized by Dynamic Light Scattering (DLS) for hydrodynamic size (diameter, nm), polydispersity (PDI) and charge at pH 5.5 and pH 7.4 (Zeta Potential, mV). The mRNA encapsulation efficiency (percent dye accessible RNA) and total mRNA content (ug/mL RNA in LNP suspension) were determined using methods described in Example 3. The formulated LNPs were subsequently buffer exchanged into pH 6.5 MES buffer saline and the size distribution was re-characterized by DLS prior to mixing with the desired quantity of targeting antibody conjugate (see Example 5) and incubated at 37° C. for 4 hours to facilitate antibody insertion (using process described in Example 5) resulting in final antibody targeted LNPs. The obtained targeted LNPs were sterile filtered and characterized by DLS (size (nm) and PDI) using methods described in Example 3.

### Example 9: Preparation of LNPs By Vortex Mixing Using Exemplary Ionizable Lipids

This examples provides an additional exemplary method for producing LNPs using exemplary ionizable cationic lipids (e.g., those synthesized in Example 1 or commercially available cationic lipids, such as KC3 or Lipid 15).

LNPs were created with an encapsulated mRNA payload and lipid blend by vortex mixing an aqueous mRNA solution and an ethanolic lipid solution. The mRNA (a 9:1 w/w mix of mRNA encoding eGFP and eGFP mRNA labeled with Cy5, TriLink Biotechnologies, California, US) was mixed with pH 4 acetate buffer to provide a final aqueous mRNA solution containing 133 μg/mL mRNA and 21.7 mM acetate buffer. The lipid components were dissolved in anhydrous ethanol at the relative ratios.

Briefly, the mRNA solution (375 μL) was transferred into a conical bottom centrifuge tube, and the lipid solution (125 μL) was rapidly added into the tube containing the mRNA solution (3.1 v/v ratio of mRNA solution to lipid solution). The tube containing the mixture was immediately capped and vortexed for 15 s at 2,500 rpm, followed by incubation at room temperature for not less than 5 min before proceeding to ethanol removal and buffer exchange.

Following mixing, ethanol removal and buffer exchange was performed on the resulting LNP suspension using a Sephadex G-25 resin packed SEC column (PD MiniTrap G-25, Cytiva, Massachusetts, U.S.), by gravity flow. Briefly, the SEC column was rinsed five times with 2.5 mL of exchange buffer (25 mM pH 7.4 HEPES buffer with 150 mM NaCl) before then loading 425 μL of LNP suspension. Once the LNP suspension fully moved into the resin bed, a 75 μL stacker volume of exchange buffer was applied to the column to achieve the specified target load volume of the column and maximize recovery, according to manufacturer specifications. After the stacker fully moved into the resin bed, the SEC column was transferred to a new centrifuge tube, and the LNP suspension was eluted by adding 1.0 mL of exchange buffer to the column. Eluate containing the LNPs in the exchange buffer was recovered and stored at 4° C. until further use.

### Example 10: Construction and Screening of Fab-Conjugated LNPs for HSC Transfection

In this Example, lipid nanoparticles (LNPs) (e.g., LNPs comprising an ionizable cationic lipid, wherein the ionizable cationic lipid is KC3 or Lipid 15) were formulated, conjugated with 23 different hemopoietic stem cell (HSC)-targeting Fabs and full-length antibodies, and screened for transfection of HSCs. Three of the LNP-Fab conjugates successfully transfected primary HSCs.

### HSC Thaw and Growth Methods

HSC media was made using SFEM II media from StemCell™ Technologies as the base media. The SFEM II media was supplemented with the CD34+ expansion supplement to make the final HSC media formulation. Cryovials of 10 million primary human HSCs (isolated from a leukopak of G-CSF and Plerixaflor mobilized patients) were thawed using HSC media. After thawing, 1 mL of media was added dropwise to the vial and the entire volume was transferred to a 15 mL conical tube. 8 mL of additional media was added to the cell suspension and the total number of cells was counted using an NC-202™ automated cell counter. The cells were spun down and resuspended at a concentration of 1 million cells per 1 mL of media. The cells were cultured in the appropriate flask for 3 days. On day 3 of culture, the cells were again counted on the NC-202™. Fresh HSC media was added to the cell culture to return the concentration of HSCs to 1 million cells per 1 mL of media. On day 4 of culture, the HSCs were collected for transfection using the LNPs.

### In Vitro LNP Treatment Methods

On the day of LNP treatment, the HSCs were collected and resuspended in fresh HSC media at a concentration of 75,000 cells per 100 μL. 30,000 cells in 40 μL is seeded into individual wells of a round bottom 96 well plate. The LNPs are added to the cells at the indicated dose. After adding the LNPs, HSC media was added to the wells to bring the total volume of the cultures to 100 μL.

On the day of LNP treatment, the HSCs were also stained for CD34 and CD117 (CD34 is a ubiquitous HSC marker and CD117 is a marker of long-term HSCs) to determine the purity of the culture after HSC expansion. After staining, the cells were analyzed by flow cytometry and CD34+Cd117+ cells were quantified.

### LNP Formulation and Conjugation

LNPs were formulated as described in Example 2 using commercially available DLin-KC3-DMA (KC3) ionizable cationic lipid, with the exception that the LNPs were left in HBS by omitting the exchange into MBS described in Example 2. KC3 LNPs were first formulated with GFP mRNA to identify an antibody that could successfully transfect HSCs with mRNA. GFP mRNA was procured from TriLink BioSciences and modified with N-1-methyl pseudo uridine. The resulting LNPs were characterized as described in Example 3, and the results are given in Table 6 below.

TABLE 6
LNP characterization results.

| | DLS Z- | | Zeta | Zeta | |
|---|---|---|---|---|---|
| | Avg. | | Potential | Potential | Dye- |
| | Diameter | DLS | at pH 5.5 | at pH 7.4 | Accessible |
| Lot Number | (nm) | PDI | (mV) | (mV) | mRNA (%) |
| EXP22002243- | 87 | 0.12 | 19.3 | 3.8 | 9.9 |
| N01H | | | | | |

Using the methods described in Examples 4 and 5, KC3 LNPs encapsulating GFP mRNA were conjugated with 23 Fabs and commercially-obtained full-length antibody combinations over 3 Fab/antibody densities that target specific cell-surface markers of HSCs, including CD34, CD105 and CD117 (Table 7). For conjugation with full-length antibodies, the LNPs were fused with a streptavidin moiety, and the conjugated full-size antibodies were tagged with biotin. Specifically, lysine groups on the streptavidin were reacted with Traut's reagent to covalently attach thiol groups. The thiolated streptavidin was then conjugated to DSPE-PEG(2k)-maleimide via covalent coupling between the maleimide group and the thiol groups attached to the streptavidin. The streptavidin-fused lipid was then reacted with the biotinylated antibody. Finally, the lipid-streptavidin-antibody conjugate was inserted into the LNP by incubation at 60° C. for 1 hour. The LNPs also incorporated a fluorescent lipid dye,′,1′-Dioctadecyl-3,′,3′,3′-Tetramethylindocarbocyanine-5,5′-Disulfonic Acid (DiIC(18)5-DS). The GFP mRNA was used to measure transfection, as the mRNA must enter the cell and escape the endosome to be transcribed into protein, allowing for fluorescence. The DiIC(18)5-DS was used as a measure of LNP targeting because the cells will have DiI fluorescence so long as the LNPs are capable of binding to the HSCs. Therefore, GFP expression provided a measure of LNP transfection while DiI positive events were representative of Fabs that target H—SCs. HSCs were treated with the LNPs at a constant RNA concentration of 1 pig/mL. The HSCs were incubated with the LNPs for 24 and 72 hours, after which the GFP fluorescence and DiI fluorescence were measured using flow cytometry.

TABLE 7
HSC-Targeting antibodies tested in screen.

| | | Fab |
|---|---|---|
| Modality | Conjugate | Density |
| Fab | anti-HuCD105 Ab3 Fab bDS | 5 |
| Fab | anti-HuCD105 Ab3 Fab bDS | 15 |
| Fab | anti-HuCD105 muRH105 VH2/VL2 Fab bDS | 5 |
| Fab | anti-HuCD105 muRH105 VH2/VL2 Fab bDS | 15 |
| Fab | Mixture of anti-HuCD105 Ab3 Fab bDS and anti- | 5 |
| | HuCD105 muRH105 VH2/VL2 Fab bDS | |
| Fab | Mixture of anti-HuCD105 Ab3 Fab bDS and anti- | 15 |
| | HuCD105 muRH105 VH2/VL2 Fab bDS | |
| Fab | anti-HuCD117 Ab2 Fab bDS | 5 |
| Fab | anti-HuCD117 Ab2 Fab bDS | 15 |
| Fab | anti-HuCD117 Ab2 Fab bDS | 45 |
| Fab | anti-HuCD117 Ab55 Fab bDS | 5 |
| Fab | anti-HuCD117 Ab55 Fab bDS | 15 |
| Fab | anti-HuCD117 Ab55 Fab bDS | 45 |
| Fab | anti-HuCD117 Ab1 Fab bDS | 5 |
| Fab | anti-HuCD117 Ab1 Fab bDS | 15 |
| Fab | anti-HuCD117 Ab1 Fab bDS | 45 |
| Fab | anti-HuCD117 CK6 Fab bDS | 5 |
| Fab | anti-HuCD117 CK6 Fab bDS | 15 |
| Fab | anti-HuCD117 CK6 Fab bDS | 45 |
| Fab | anti-HuCD117 hSR-1 Fab bDS | 5 |
| Fab | anti-HuCD117 hSR-1 Fab bDS | 15 |
| Fab | anti-HuCD117 hSR-1 Fab bDS | 45 |
| Fab | anti-HuCD117 6LUN1 Fab bDS | 5 |
| Fab | anti-HuCD117 6LUN1 Fab bDS | 15 |
| Fab | anti-HuCD117 6LUN1 Fab bDS | 45 |
| Ig | Biotin anti-HuCD34 (Clone 581) | 5 |
| Ig | Biotin anti-HuCD34 (Clone 581) | 15 |
| Ig | Biotin anti-HuCD34 (Clone 581) | 45 |
| Ig | Biotin anti-HuCD34 (Clone QBEND/10) | 5 |
| Ig | Biotin anti-HuCD34 (Clone QBEND/10) | 15 |
| Ig | Biotin anti-HuCD34 (Clone QBEND/10) | 45 |
| Ig | Biotin anti-HuCD34 (Clone 4H11) | 5 |
| Ig | Biotin anti-HuCD34 (Clone 4H11) | 15 |
| Ig | Biotin anti-HuCD34 (Clone 4H11) | 45 |
| Ig | Biotin anti-HuCD105 (Clone 43A3) | 5 |
| Ig | Biotin anti-HuCD105 (Clone 43A3) | 15 |
| Ig | Biotin anti-HuCD105 (Clone 43A3) | 45 |
| Ig | Biotin anti-HuCD105 (Clone 166707) | 5 |
| Ig | Biotin anti-HuCD105 (Clone 166707) | 15 |
| Ig | Biotin anti-HuCD105 (Clone 166707) | 45 |
| Ig | Biotin anti-HuCD105 (Clone MEM-229) | 5 |
| Ig | Biotin anti-HuCD105 (Clone MEM-229) | 15 |
| Ig | Biotin anti-HuCD105 (Clone MEM-229) | 45 |
| Ig | Biotin anti-HuCD117 (Clone 104D2) | 5 |
| Ig | Biotin anti-HuCD117 (Clone 104D2) | 15 |
| Ig | Biotin anti-HuCD117 (Clone 104D2) | 45 |
| Ig | Biotin anti-HuCD117 (Clone A3C6E2) | 5 |
| Ig | Biotin anti-HuCD117 (Clone A3C6E2) | 15 |
| Ig | Biotin anti-HuCD117 (Clone A3C6E2) | 45 |
| Ig | Biotin anti-HuCD117 (Clone OTI3F9) | 5 |
| Ig | Biotin anti-HuCD117 (Clone OTI3F9) | 15 |
| Ig | Biotin anti-HuCD117 (Clone OTI3F9) | 45 |
| Ig | Biotin anti-HuCD117 (Clone BA7.3C.9) | 5 |
| Ig | Biotin anti-HuCD117 (Clone BA7.3C.9) | 15 |
| Ig | Biotin anti-HuCD117 (Clone BA7.3C.9) | 45 |
| Ig | Biotin anti-HuCD117 (Clone B-K15) | 5 |
| Ig | Biotin anti-HuCD117 (Clone B-K15) | 15 |
| Ig | Biotin anti-HuCD117 (Clone B-K15) | 45 |

### Results

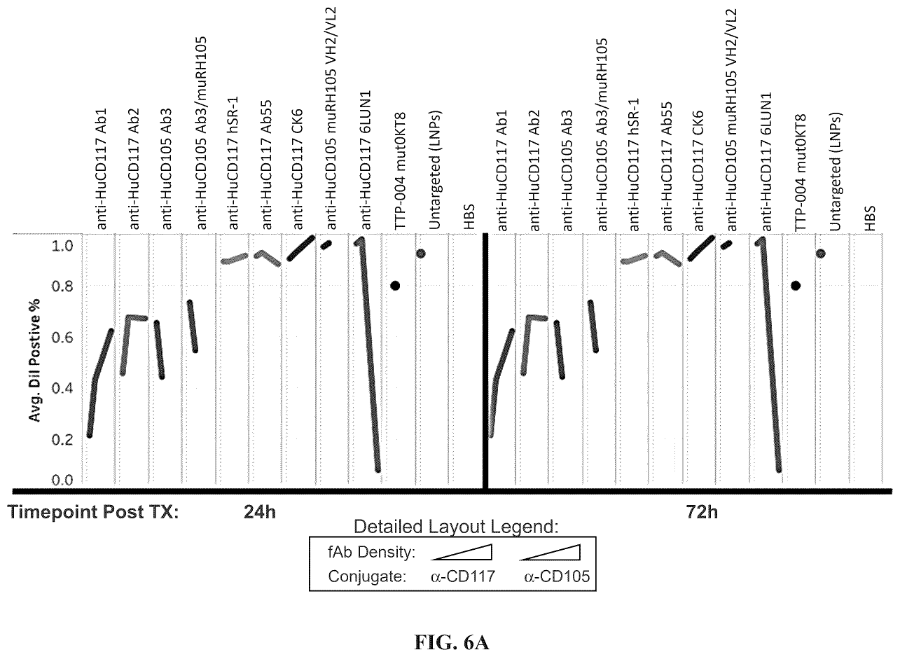

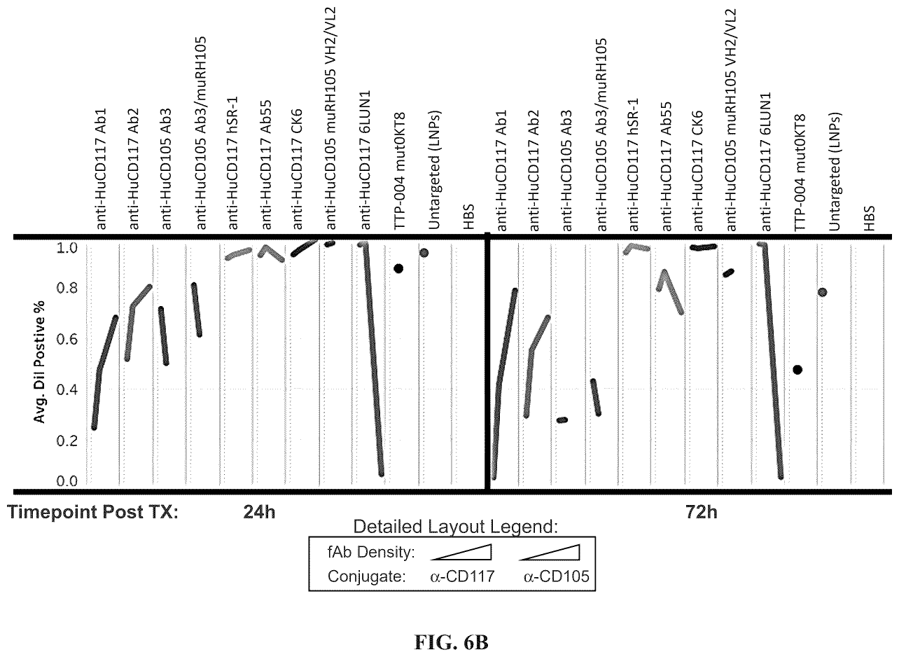

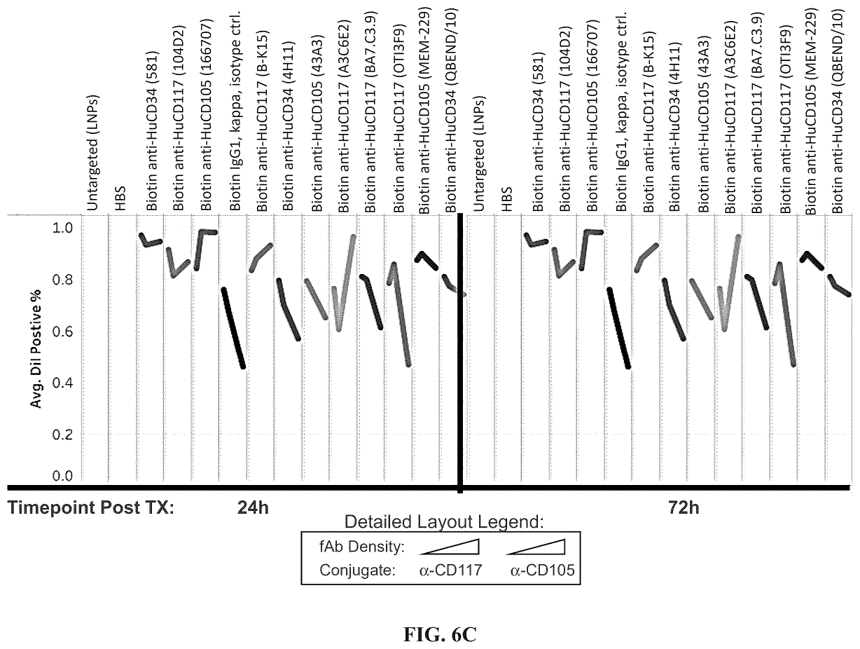

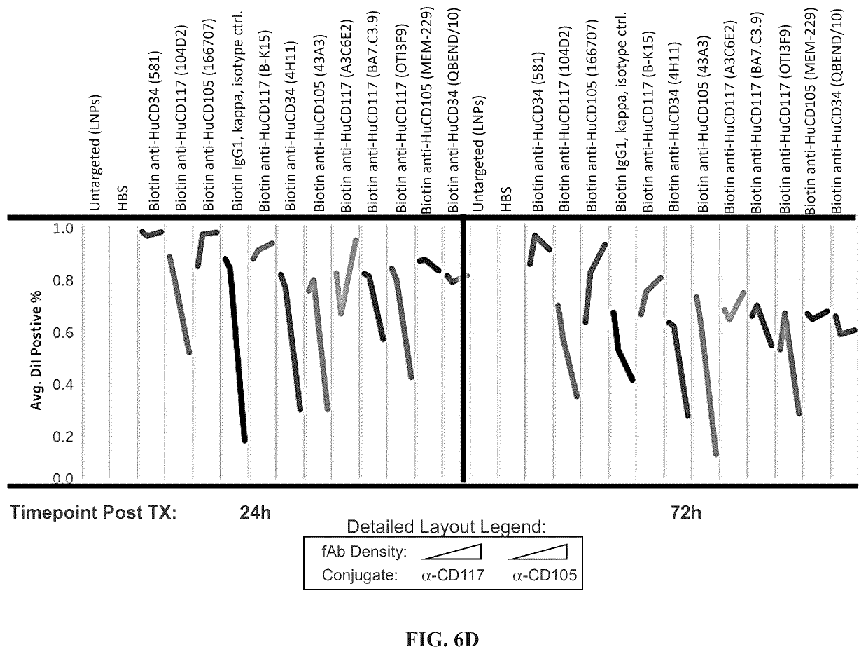

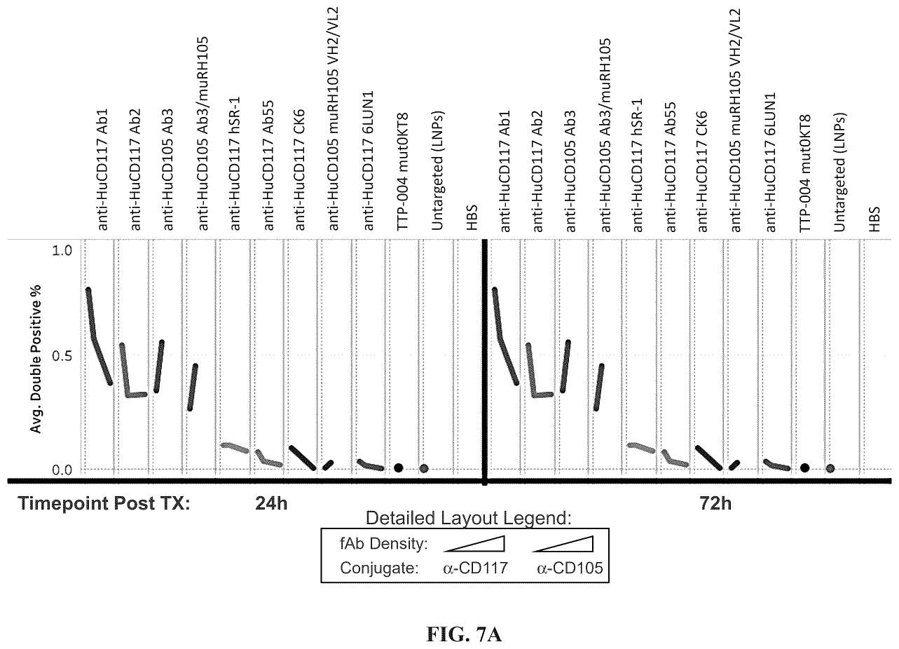

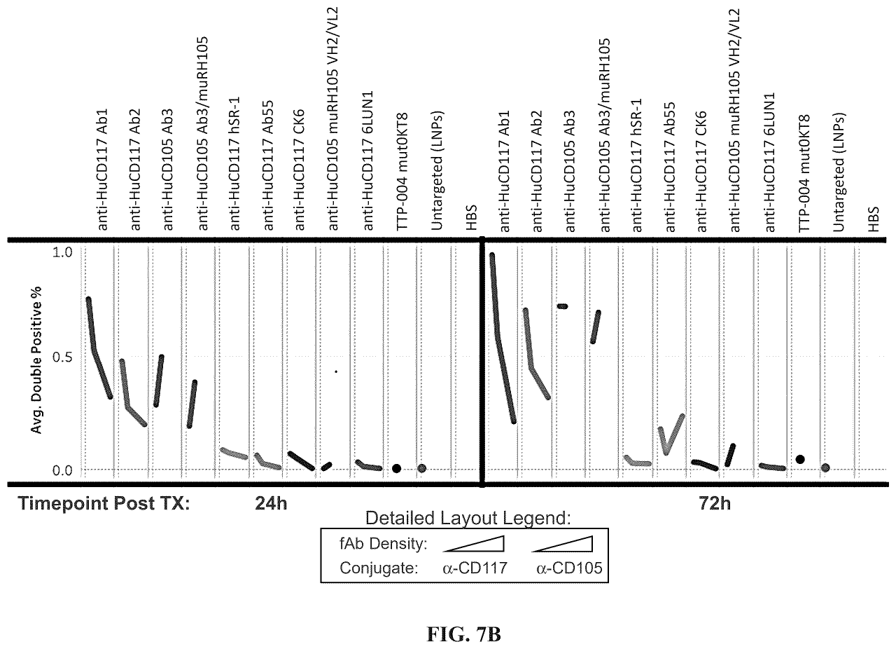

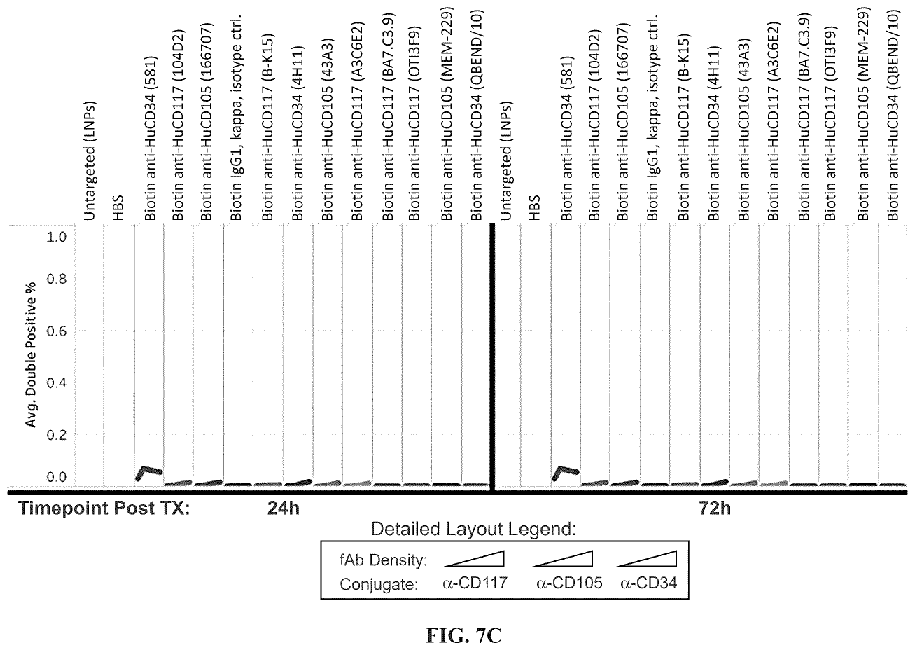

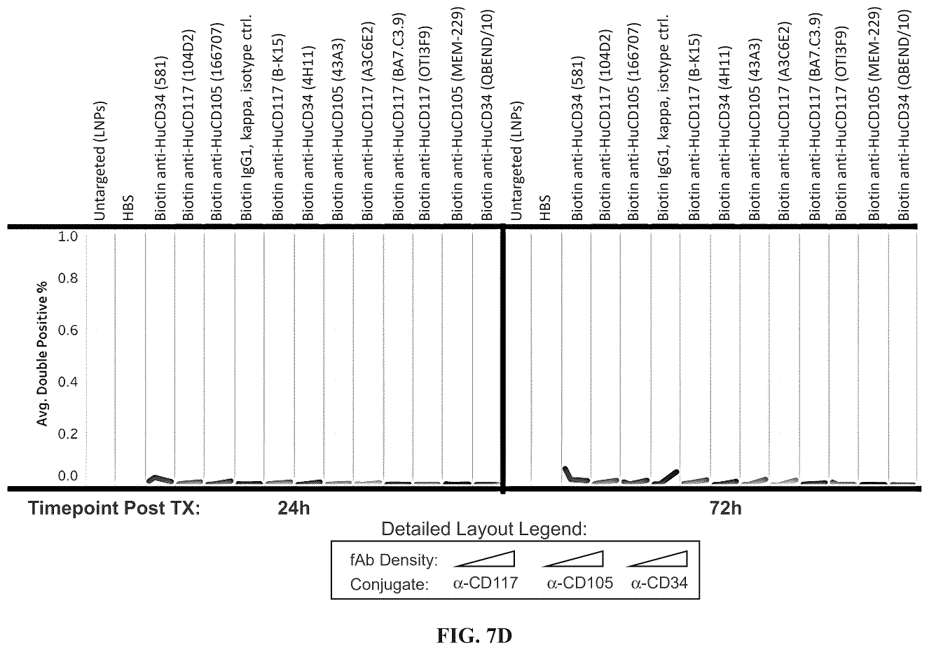

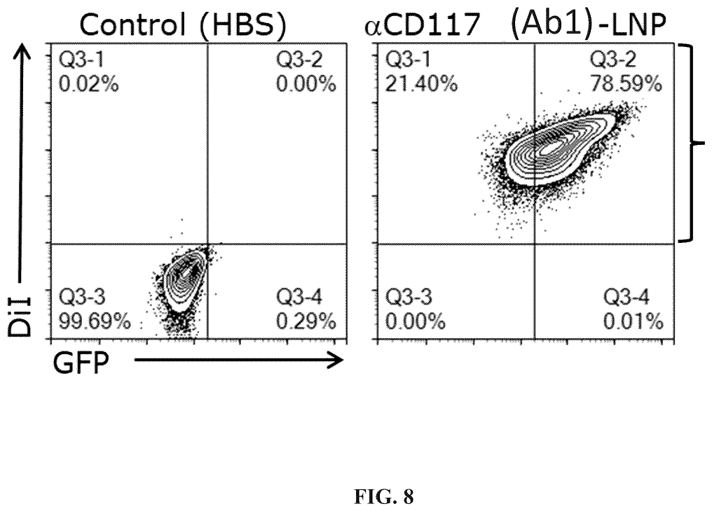

Multiple Fab-LNP and antibody-LNP combinations targeted HSCs (FIGS. 6A-6D), as indicated by positive DiI fluorescence. However, only three Fab-LNPs, i) anti-HuCD117 Ab1 Fab bDS, ii) anti-HuCD117 Ab2 Fab bDS, and iii) anti-HuCD105 Ab3 Fab bDS, successfully transfected primary HSCs with mRNA, as shown by GFP and DiI fluorescence (FIGS. 7A-7D). A single anti-CD117 clone had the highest transfection efficiency (Ab1) (FIG. 8).

### Example 11: Genetic Modification of HSCs Using LNP-Fab Conjugate

In this Example, HSC-targeting LNP-Fab conjugates encapsulating a CRISPR-Cas editing system were used to genetically modify the CD45 gene of HSCs. The induction of double-strand breaks in the CD45 genes and knockout of CD45 expression were observed.

LNP formulations (e.g., LNPs comprising an ionizable cationic lipid, wherein the ionizable cationic lipid is KC3 or Lipid 15) conjugated with or without Ab1 were used to encapsulate Cas9 mRNA with a gRNA specific for CD45 (a surrogate HSC cell surface marker that can be reliably measured by flow cytometry). Cas9 mRNA was obtained from TriLink Biosciences, and gRNA was obtained from Integrated DNA Technologies (IDT). LNPs were formulated and conjugated to Ab1 using the methods described in Examples 2 and 4-5. The ratio of Cas9 mRNA to gRNA within the LNPs was 1:1. To optimize the gRNA for LNP-based CRISPR editing, a chemical modification pattern incorporating phosphorothioate bonds and 2′-O-methyl substitutions was used with the encapsulated Cas9 mRNA. Primary human HSCs were treated with these LNPs at a dose range of 100 to 800 ng of total RNA for 7 days using the methods described in Example 1.

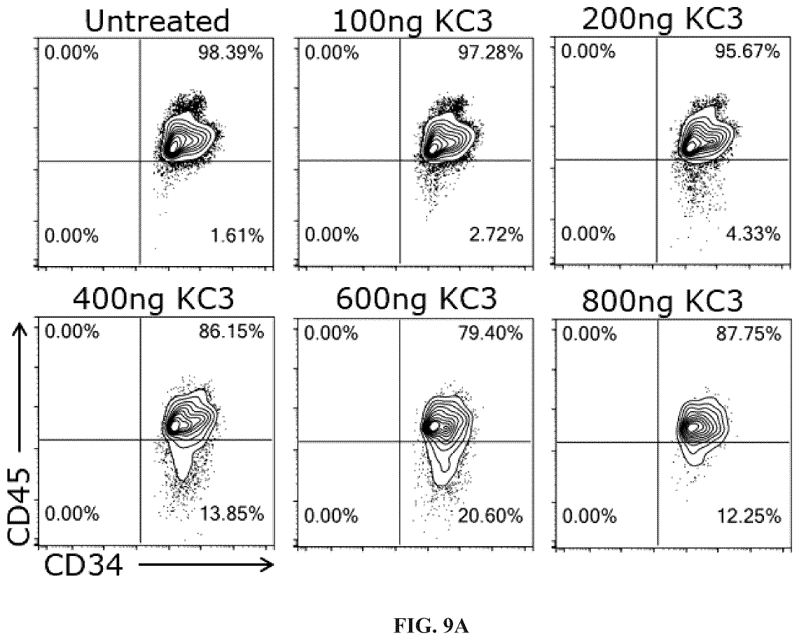

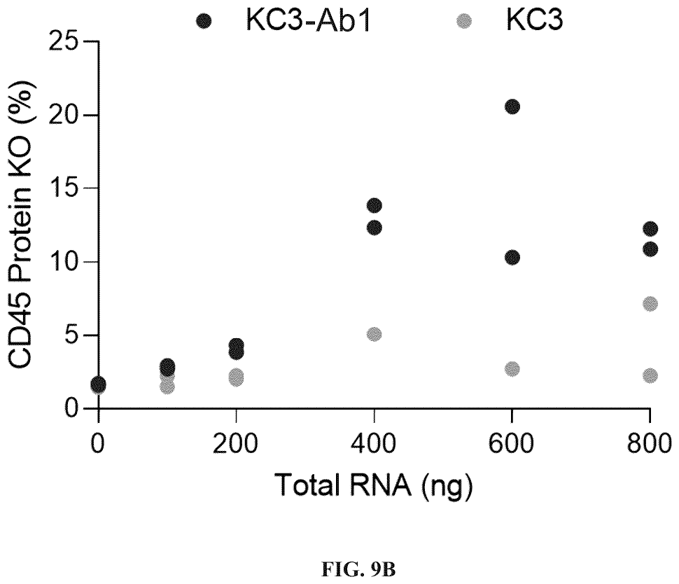

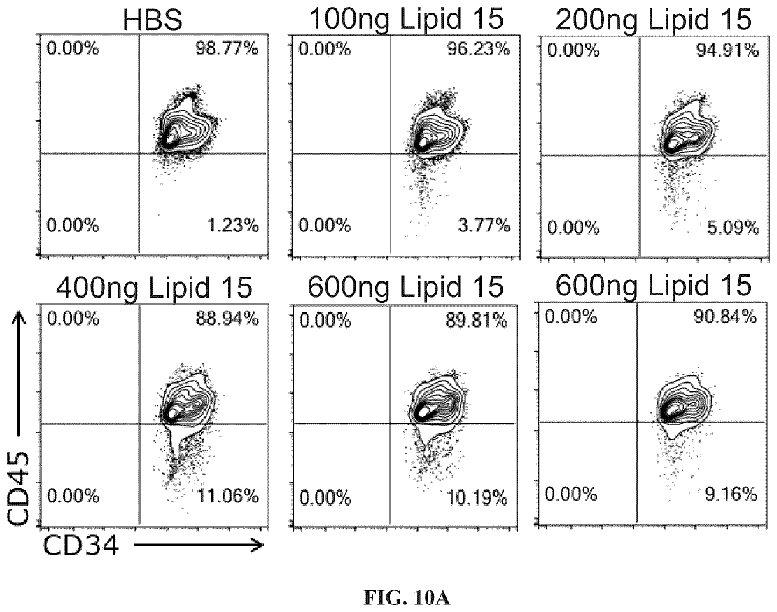

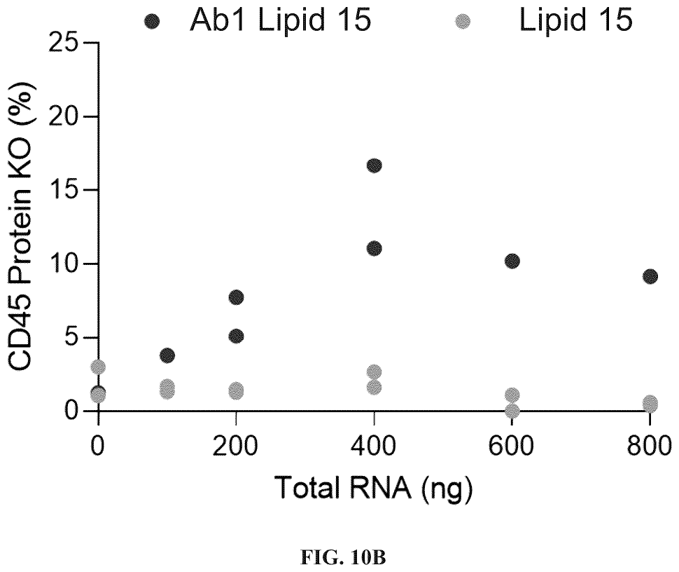

On day 7 post transfection, the HSCs were stained with fluorescent antibodies against CD45, CD34, and CD117. The fluorescence of each protein was quantified using flow cytometry. CD34 and CD117 were used to define HSC populations. CD45 fluorescence was used to define knockout of CD45. Through incorporation of the above strategies to enable LNP targeting and CRISPR-mRNA editing, CD45 protein was reduced in primary human HSCs seven days after targeted LNP dosing in vitro. Results for KC3 are shown in FIGS. 9A-9B, and results for Lipid 15 are shown in FIGS. 10A-10B.

Additionally, on Day 7 post transfection, the HSCs were collected for next-generation-sequencing (NGS) to quantify the indel rate at the genomic locus targeted by Cas9 and the CD45 gRNA using targeted amplicon sequencing. For amplicon sequencing, primers were designed to span a 300 base pair region surrounding the on-target cut site of the CD45 gRNA used. Following amplification, the 300 base pair sequences were quantified using an Illumina MiSeq. The CRISPR-LNP treatment produced significant indels at the target locus, further corroborating the reduction in CD45 protein that we observed (FIG. 11).

### Example 12: Disruption of the BCL11A Erythroid Enhancer in HSCs

LNPs (e.g., LNPs comprising an ionizable cationic lipid, wherein the ionizable cationic lipid is KC3 or Lipid 15) encapsulating a gRNA against BCL11a and Cas9 mRNA are formulated and conjugated with Ab1 and mutAb1 using the methods described in Examples 2 and 4-5. The LNPs are used to treat primary human HSCs in vitro. MutAb1 is a non-targeting Fab derived from Ab1 with mutations in alanine in each of the CDR loops of the light chain, and heavy chain of the antibody, as shown in Table 8 below.

TABLE 8
Ab1 and mutAb1 CDR sequences.

| Antibody | Ab1 | mutAb1 |
|---|---|---|
| CDR-H1 | FTFSNYAMS | FAAANYAMS |
| | (SEQ ID NO: 1) | (SEQ ID NO: 28) |
| CDR-H2 | AISGSGGSTYYADSVKG | AISGAAASTYYADSVKG |
| | (SEQ ID NO: 2) | (SEQ ID NO: 29) |
| CDR-H3 | AKGPPTYHTNYYYMDV | AKGPPTYAAAYYYMDV |
| | (SEQ ID NO: 3) | (SEQ ID NO: 30) |
| CDR-L1 | RASQGISSWLA | RASQAAASWLA |
| | (SEQ ID NO: 4) | (SEQ ID NO: 31) |
| CDR-L2 | AASSLOS | AAASLOS |
| | (SEQ ID NO: 5) | (SEQ ID NO: 32) |
| CDR-L3 | QQTNSFPYT | QQTAAAPYT |
| | (SEQ ID NO: 6) | (SEQ ID NO: 33) |

The HSCs are collected at 3 and 7 days after treatment, where they were then assayed for gene editing using targeted amplicon sequencing.

### Example 13: Further In Vitro Targeting and Genetic Modification of HSCs

### 13.1: Screening Optimization of Fab Conjugate Density on LNP For HSC Transfection

In a further experiment to characterize the Ab-conjugated LNPs, lipid nanoparticles (LNPs) were formulated with mCherry mRNA using Lipid 15 and exchanged directly into MBS using the methods described in Example 2. mCherry mRNA was produced by in vitro transcription using methods described in U.S. Pat. No. 10,143,758 (Example 7), modified with N-1-methyl pseudo uridine. Conjugates of Fabs Ab1, Ab2, and MutAb1 were prepared as in Example 4. Fab conjugates were then inserted into the formulated LNPs at 10 different densities listed in Table 9 below, using methods described in Examples 4-5, with the modification that the insertion was performed at 37° C. for 4 hours.

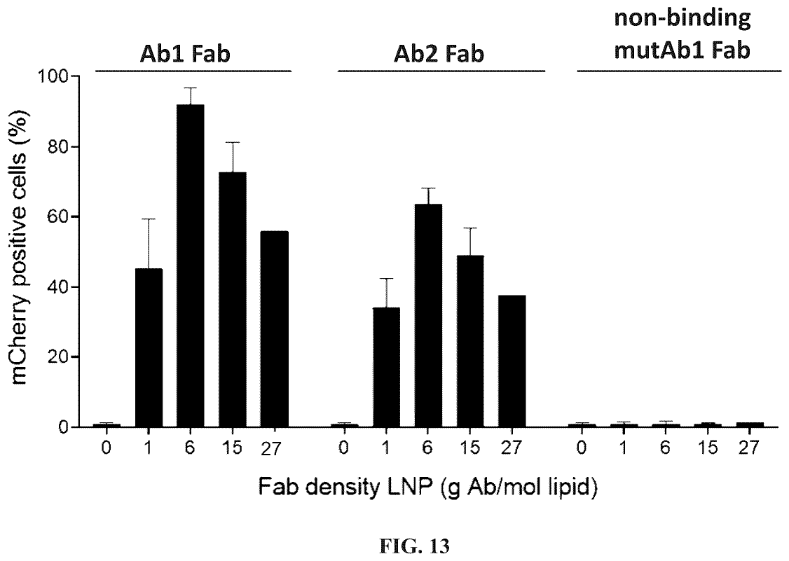

The LNPs were screened for levels of mCherry transfection in HSCs using method as described in Example 11. Specifically, HSCs were treated with a 100 ug mRNA/well dose with 30,000 cells per well and media was added to the wells to bring the total volume to 100 uL. HSCs were then incubated with LNPs for 6 hours, after which the culture media containing LNPs was replaced with fresh media. After 24 hours, the HSCs were stained with fluorescent antibodies against CD34 and CD117. Cells were gated for CD34+ and CD117+ staining, and the percent of CD34+CD117+mCherry+ cells was determined. The optimal range of Fab density was found to be about 3-9 g of Ab/mol of lipid to maximize HSC targeting and cell transfection. The results for a selection of Ab densities are shown in FIG. 13, which highlights the optimal range of Fab density (3-9 g of Ab/mol of lipid).

TABLE 9
HSC-Targeting antibody densities tested in the optimization.

| | | Fab Density (g Fab/ |
|---|---|---|
| Modality | Conjugate | mol lipid) |
| Parent LNP | None | 0 |
| Fab | anti-HuCD1117 Ab1 Fab bDS | 1 |
| Fab | anti-HuCD1117 Ab1 Fab bDS | 3 |
| Fab | anti-HuCD1117 Ab1 Fab bDS | 6 |
| Fab | anti-HuCD1117 Ab1 Fab bDS | 9 |
| Fab | anti-HuCD1117 Ab1 Fab bDS | 12 |
| Fab | anti-HuCD1117 Ab1 Fab bDS | 15 |
| Fab | anti-HuCD1117 Ab1 Fab bDS | 18 |
| Fab | anti-HuCD1117 Ab1 Fab bDS | 21 |
| Fab | anti-HuCD1117 Ab1 Fab bDS | 27 |
| Fab | anti-HuCD1117 Ab2 Fab bDS | 1 |
| Fab | anti-HuCD1117 Ab2 Fab bDS | 3 |
| Fab | anti-HuCD1117 Ab2 Fab bDS | 6 |
| Fab | anti-HuCD1117 Ab2 Fab bDS | 9 |
| Fab | anti-HuCD1117 Ab2 Fab bDS | 12 |
| Fab | anti-HuCD1117 Ab2 Fab bDS | 15 |
| Fab | anti-HuCD1117 Ab2 Fab bDS | 18 |
| Fab | anti-HuCD1117 Ab2 Fab bDS | 21 |
| Fab | anti-HuCD1117 Ab2 Fab bDS | 27 |
| Fab | anti-HuCD1117 MutAb1 Fab bDS | 1 |
| Fab | anti-HuCD1117 MutAb1 Fab bDS | 3 |
| Fab | anti-HuCD1117 MutAb1 Fab bDS | 6 |
| Fab | anti-HuCD1117 MutAb1 Fab bDS | 9 |
| Fab | anti-HuCD1117 MutAb1 Fab bDS | 12 |
| Fab | anti-HuCD1117 MutAb1 Fab bDS | 15 |
| Fab | anti-HuCD1117 MutAb1 Fab bDS | 18 |
| Fab | anti-HuCD1117 MutAb1 Fab bDS | 21 |
| Fab | anti-HuCD1117 MutAb1 Fab bDS | 27 |

### 13.2: In Vitro Editing or Primary HSCs at the B2M Locus

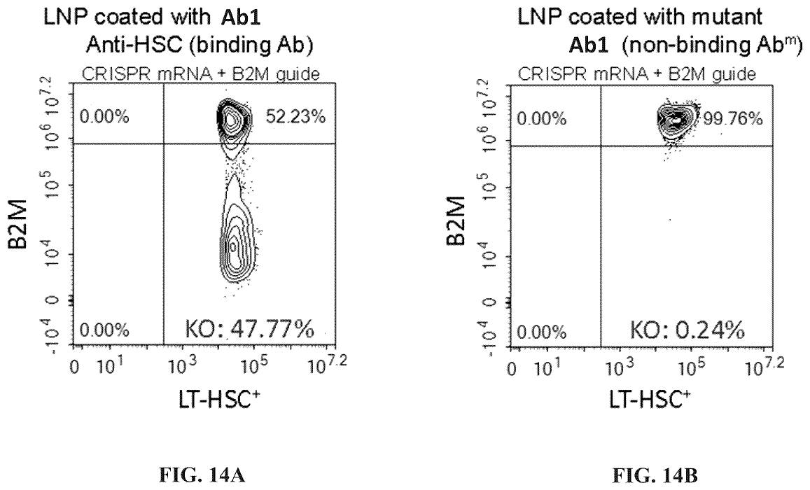

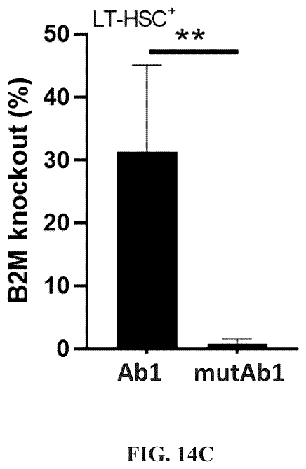

Next, primary HSCs were cultured in vitro and treated with LNPs formulated with Lipid 15 and encapsulating a Cas nuclease mRNA and a gRNA specific for the beta-2-microglobulin locus (B32M). The LNPs were coated with either Ab1 or non-binding mutAb1. Gene editing effect was quantified for 1B2M protein knockout using flow cytometry targeting 1B2M protein expression. As shown in FIGS. 14A-14C, the B32M locus was successfully edited in HSCs in vitro using the LNPs conjugated with Ab1, while the LNPs conjugated with mutAb1 did not result in editing.

### Example 14: Iv Vivo Genetic Modification of HSCs

### 14.1: In Vivo Transfection or Long-Term Hematopoietic Stem Cells Using Targeted LNP

LNPs (e.g., LNPs comprising an ionizable cationic lipid, wherein the ionizable cationic lipid is KC3 or Lipid 15) are conjugated with Ab1 and mutAb1 using the methods described in Examples 1 and 2. NSG mice are engrafted with primary human HSCs to establish a murine model whereby targeting human HSCs with targeted LNPs in vivo can be observed. In an initial experiment, LNPs encapsulating mCherry or eGFP mRNA are formulated. The LNPs are then intravenously injected into the mice via the tail vein. At 24 and 48 hours, the mice are euthanized and the bone marrow of the mice is collected and analyzed by flow cytometry to assay either mCherry or eGFP fluorescence in the HSC population.

14.1.1: In Vivo Transfection of Long-Term Hematopoietic Stems Cells Using Targeted LNP Carrying mCherry mRNA

### Materials and Methods

Mice. The analysis of human CD34⁺ hematopoietic stem cell-engrafted NSG™ mice was by routine methods. Experiments were conducted with mice over 12 weeks post-transplantation. LNPs were administered at 1 mg/kg (total nucleotide measured from mCherry mRNA) for all conditions via tail vein injection as a bolus. Mice were treated with targeted LNP coated with Ab1 Fab, de-targeted LNP coated with the non-binding mutAb1 Fab or by the parental LNP without Fab (uncoated/naked). Mice were euthanized 24 h post-treatment and virous tissues were collected to be analyzed. Treated Hu-CD34⁺-NSG™ mice were controlled by untreated Hu-CD34⁺-NSG™ mice.

Flow cytometry Analysis. Bone marrow cells were obtained by flushing tibias and femurs of euthanized mice with cold PBS containing 5% fetal bovine serum (FBS) and 2 mM EDTA (FACS Buffer). Cells were harvested in cold FACS Buffer, stained with monoclonal antibodies against human CD45 (clone 2D1, Cat #368542 from Biolegend™), mouse CD45, human CD34 (clone 561, Cat #343614 from Biolegend™) and human CD117 (clone 104D2, Cat #313206 from Biolegend™) for 20 min at room temperature, then analyzed by flow cytometry on a NovoCyte Penteon (Agilent). Both anti-mouse and anti-human CD45 antibodies were used to identify the human cells from the bone marrow to clearly differentiate the human and mouse HSCs in the mixed populations of bone marrow cells. Cells were analyzed based on mCherry positivity.

ELISA. ~30 mg of frozen liver tissues from the mice were homogenized in 300 μL RIPA buffer+1×HALT proteinase inhibitor cocktail (PI) (cat #78441) using a Tissuelyser (Qiagen) in cold room (Program P1: 30 frequency, 5 min, 4° C.) and then spun down at 12000 rpm at 4° C. for 5 min. mCherry ELISA (Cat #ab221829 from Abcam) was performed based on manufacturer recommendations. Product absorbances were measured using a SpectraMax Plus plate reader at 450 nm (Molecular Devices, San Jose, CA.

Statistics. Statistical analyses were performed using GraphPad Prism 9.0 software. A two-tailed Student's 1-test was used for individual comparisons if they were normally distributed

### Results

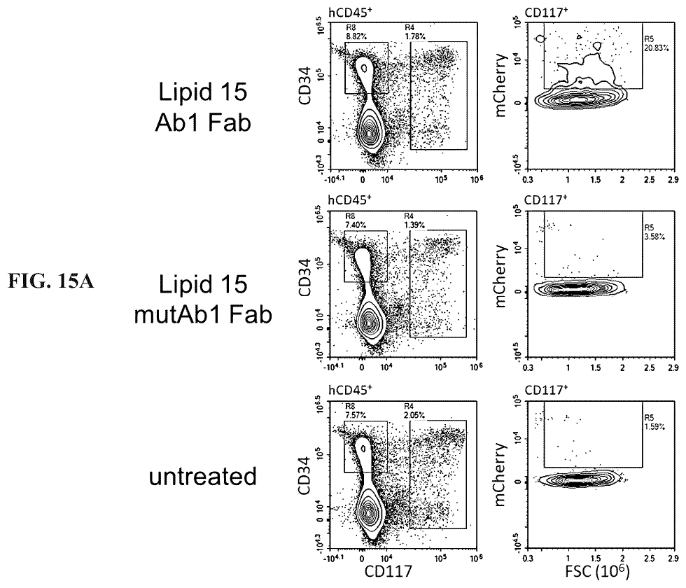

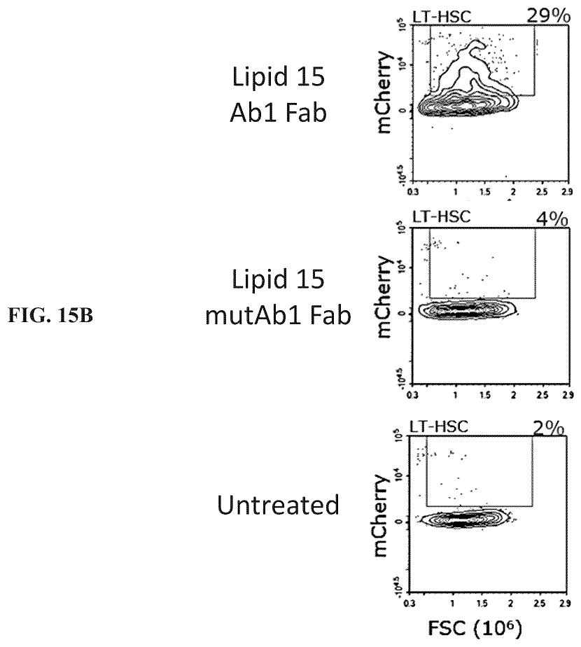

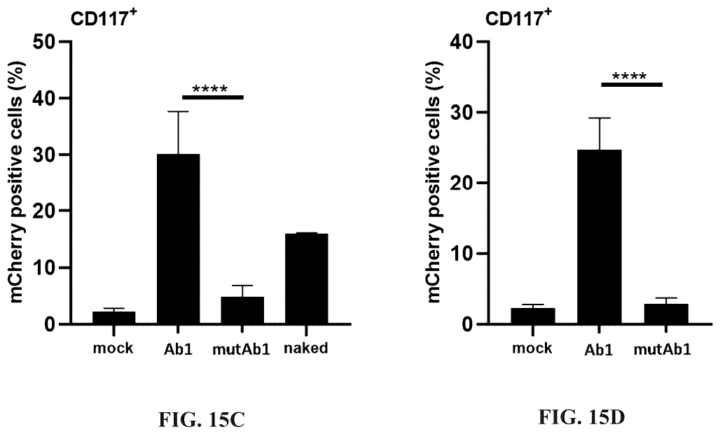

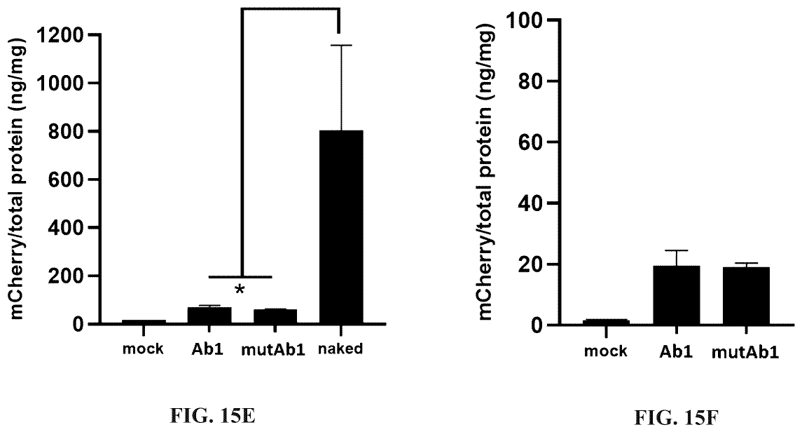

The results of this analysis indicated that engineered antibody-targeted LNPs can recognize, bind, and transfect LT-HSCs (CD117′) to deliver mRNA into the cytosol (FIGS. 15A-15B). In addition, the transfection efficiency was evaluated as 30% with Lipid 15 LNP coated with the Ab1 Fab, 5% with LNP coated with the untargeted mutAb1 Fab, and 16% with uncoated LNP from multiple experiments (FIGS. 15A-15C). Similar transfection efficiencies, 25% and 3% were measured with KC3 LNPs coated with Ab1 Fab and mutAb1 Fab respectively (FIG. 15D). Moreover, Fab coated Lipid 15 LNP have a lower tropism to liver cells than naked Lipid 15 LNP as 91% less mCherry signal was measured by ELISA after protein extraction from 2 different liver lobes of multiple treated animals (FIG. 15E). Finally, we measured 3 time less signal from liver cells treated by KC3 vs Lipid 15 LNPs (FIGS. 15E-15F).

14.1.2: Further In Vivo Transfection of Long-Term Hematopoietic Stem Cells Using Targeted LNP Carrying mCherry mRNA

In vivo transfection of human HSCs in human CD34+ HSC-engrafted mice (n=12) was further evaluated in a second experiment. Female Hu-CD34+ NSG™ mice (Jax laboratory; Hu-CD34+; NSG™ mice, NOD.Cg-Prkdcscid Il2rgtm1Wjl, Stock No. 005557) were treated with Lipid 15 LNPs coated with Ab1 to transfect human HSCs in the bone marrow environment. The LNPs were prepared with about 50% Lipid 15, about 40% cholesterol, about 10% DSPC, and about 1.5% PEG (DPG-PEG comprising PEG at a molecular weight of 2,000 Da). Lipid 15 LNPs were thawed at room temperature, mixed smoothly by inversion, and then were diluted in saline solution before dosing at 1.0 mg/kg by intravenous injection (IV) as a single bolus injection. 24 hours after treatment, whole bone marrow, liver, spleen, lung, and ovary tissues were collected and analyzed by flow cytometry, ELISA, or IHC, using mCherry reporter.

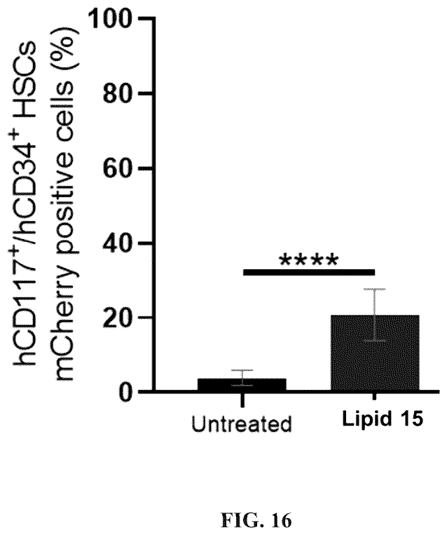

Bone marrow cells collected, from tibia and femur, were resuspended as a single cell suspension in DPBS complemented with 10% of fetal bovine serum (FBS). Cells were stained with hCD45, clone 2D1 (Biolegend™); hCD117, clone 104D2 (from Biolegend™); hCD34, clone 561 (Biolegend™) and analyzed by flow cytometry to assess transfection efficiency. As shown in FIG. 16, an average of about 20% of CD34+/CD117+ human HSCs were positive for mCherry fluorescence, demonstrating in vivo transfection of human LT-HSCs by the Lipid 15 LNPs coated with Ab1.

Liver, spleen and lung tissues were homogenized for protein extraction (using RIPA buffer complemented with 1×HALT™ proteinase inhibitor cocktail [Thermo Fisher cat #78441]) and analyzed by ELISA (ab221829 mCherry SimpleStep ELISA® Kit, from Abcam). Ovary tissues were embedded, sectioned, and analyzed by immunohistochemistry (IHC) using M11217 Ab (from Abcam). IHC tissues were analyzed and graded blindly by a certified pathologist. The results of the off-target tissue analysis are shown in Table 10.

TABLE 10
Detection of mCherry protein in off-target tissues.

| Tissue | mCherry Protein | | Detection Method |
|---|---|---|---|
| Liver | 54 | ng/mg | ELISA |
| Spleen | 260 | ng/mg | |
| Lung | 3.5 | ng/mg | |

| | | |
|---|---|---|
| Ovary | No signal | IHC |

14.2: In Vivo Editing of HSCs Using LNPs Encapsulating gRNA and Cas Nuclease mRNA

Next, LNPs encapsulating gRNA (e.g., gRNA targeting the BCL11a erythroid enhancer) and mRNA encoding a Cas nuclease (e.g., Cas9) are formulated and injected into NSG mice engrafted with primary human HSCs. At 7 days post-injection the mice are euthanized and the bone marrow collected. gDNA is isolated from the bone marrow cells and NGS-amplicon sequencing is performed on the samples to calculate in vivo gene editing.

### 14.3: In Vivo Transfection of Long-Term Hematopoietic Stem Cells Using Targeted LNP in Non-Human Primates (NHPs)

LNPs encapsulating mCherry mRNA were formulated and used to treat non-human primates (NHPs), specifically Mauritius origin cynomolgus macaques.

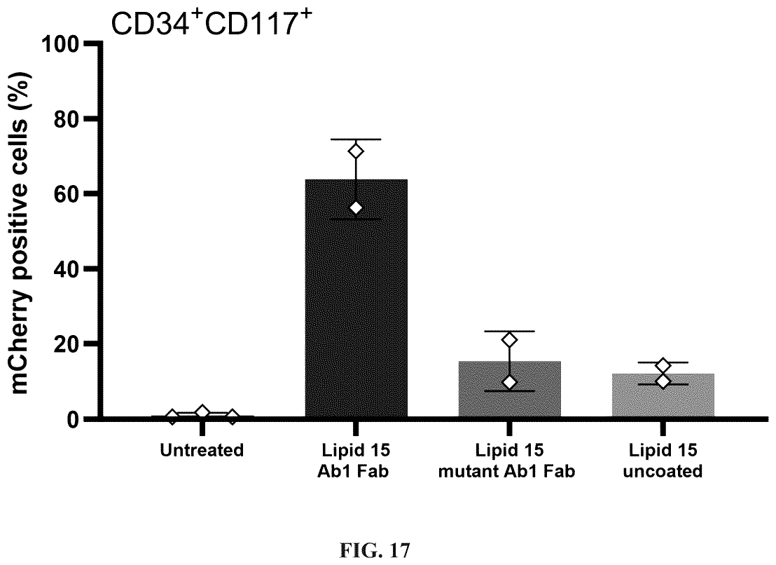

First, the ability of human HSC-targeting LNPs to target cynomolgus macaque HSCs was validated in vitro. Lipid 15 LNPs coated with Ab1 and encapsulating mCherry mRNA were used to treat HSCs isolated from cynomolgus macaques. Cynomolgus macaque HSC media was made using IMDM media from ThermoFisher™ Technologies as the base media. The IMDM media was supplemented with 10% FBS (Gibco™), rhSCF 100 ng/mL (PeproTech™), thrombopoietin 100 ng/mL (PeproTech™), rhuFlt3-L100 ng/mL (PeproTech™), interleukin-3 100 ng/mL (PeproTech™), interleukin-6 100 ng/mL (PeproTech™), G-CSF 100 ng/mL (PeproTech™) to make the final HSC media formulation. Bone marrow cells were resuspended as a single cell suspension and mixed in red blood cell (RBC) lysis buffer (00-4333-57 from Invitrogen) to eliminate erythrocytes. The cells were spun down and resuspended at a concentration of 1 million cells per 1 mL of media. The cells were cultured in the appropriate flask for 3 days. On day 3 of culture, the cells were again counted on the NC-202™. Fresh HSC media was added to the cell culture to return the concentration of HSCs to 1 million cells per 1 mL of media. On day 8 of culture, the HSCs were collected for transfection using the LNPs and analyzed the following day. The samples were analyzed using flow cytometry for mCherry expression to measure LT-HSC transfection. Cynomolgus macaque LT-HSCs were identified using the CD34 and CD117 markers. As shown in FIG. 17, about 65% of cynomolgus macaque HSCs were transfected with mCherry mRNA in cynomolgus macaques treated with Lipid 15 LNPs coated with Ab1, confirming that Lipid 15 LNPs coated with Ab1 can target cynomolgus macaque LT-HSCs.

Next, in vivo transfection of LT-HSCs in NPHs was evaluated. Male and female Mauritius origin cynomolgus macaques were infused with different dosing regimens of Lipid 15 LNPs or KC3 LNPs, each coated with Ab1 and encapsulating mCherry mRNA to transfect HSCs in the bone marrow environment. The LNPs were prepared with about 40% cholesterol, about 10% DSPC, about 1.5% PEG (DPG-PEG comprising PEG at a molecular weight of 2,000 Da) and about 50% Lipid 15 (for Lipid 15 LNPs) or about 50% KC3 lipid (for KC3 LNPs). NHPs were pretreated with 2 mg/kg of diphenhydramine, intramuscularly, 30 min prior to the LNP infusion. Lipid 15 LNPs and KC3 LNPs were thawed at room temperature, mixed smoothly by inversion, and then were diluted in saline solution before dosing at by intravenous injection (IV) as a single slow infusion (via pump at 5 mL/Kg) over a 1 hour duration. Lipid 15 LNPs were dosed at 0.5 mg/kg and 0.2 mg/kg, and KC3 LNPs were dosed at 1.0 mg/kg, 0.5 mg/kg, and 0.2 mg/kg. 24 hours after the NHPs were injected with the LNPs, whole bone marrow samples were collected 24 h post treatment by aspiration from the iliac crest. The design of the study is shown in Table 11.

TABLE 11
Design of study evaluating in vivo transfection of
LT-HSCs in Mauritius origin cynomolgus macaques.

| | No. of | | Dose | |
|---|---|---|---|---|
| Study | macaques | mRNA/LNP Treatment | (mg/kg) | Route |
| 23-01A | 2 | mCherry mRNA Lipid | 0.5 | IV |
| | | 15 Ab1 | | |
| | 2 | mCherry mRNA Lipid | 0.2 | IV |
| | | 15 Ab1 | | |
| | 1 | untreated | NA | NA |
| 23-01B | 1 | mCherry mRNA KC3 | 1.0 | IV |
| | | Ab1 | | |
| | 1 | mCherry mRNA KC3 | 0.5 | IV |
| | | Ab1 | | |
| | 2 | mCherry mRNA KC3 | 0.2 | IV |
| | | Ab1 | | |
| | 1 | untreated | NA | NA |

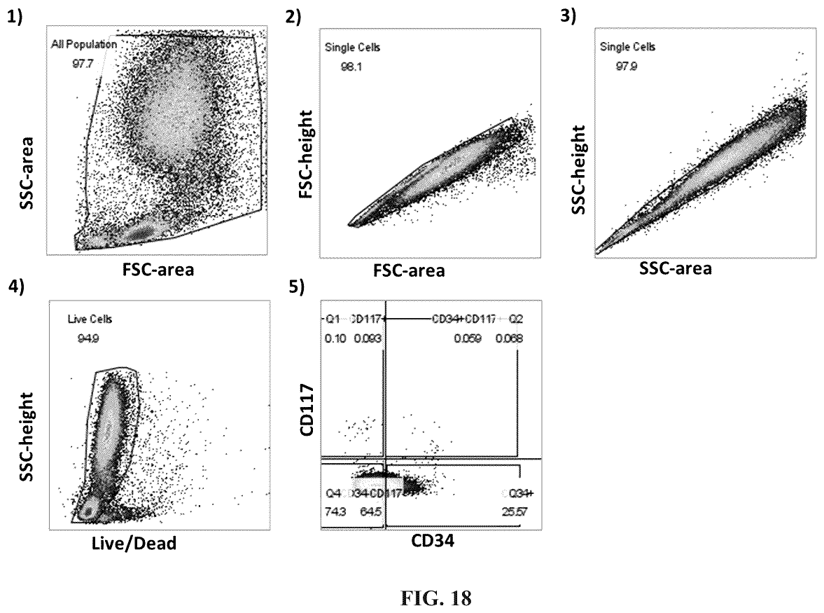

Flow cytometry was used to assay the bone marrow aspirates for NHP HSCs that have mCherry fluorescence. Bone marrow cells were resuspended as a single cell suspension and mixed in red blood cell (RBC) lysis buffer (00-4333-57 from Invitrogen) to eliminate erythrocytes. Cells were washed, resuspended in Dulbecco's phosphate-buffered saline (DPBS) complemented with 10% of fetal bovine serum (FBS), stained with anti-CD34 (clone 561, Cat #343614 from Biolegend™) and anti-CD117 (clone 104D2, Cat #313206 from Biolegend) antibodies, and analyzed by flow cytometry to assess transfection efficiency. An exemplary flow cytometry results illustrating the flow cytometry gating strategy to identify cynomolgus macaque LT-HSCs is shown in FIG. 18.

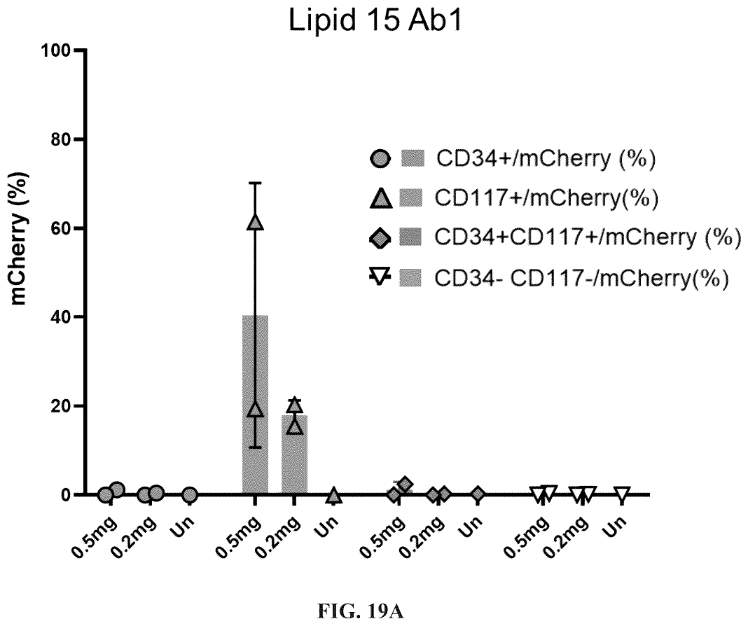

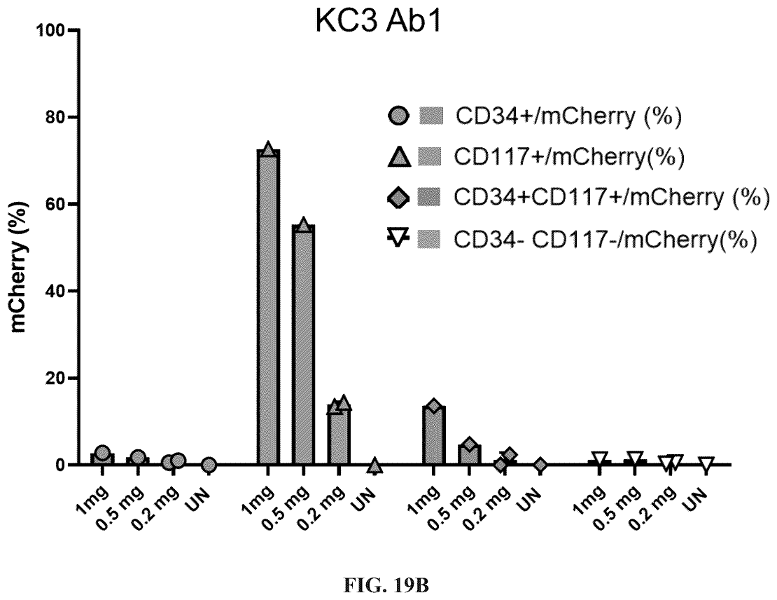

FIGS. 19A-19B show the average percentage of HSCs that are mCherry positive from cynomolgus macaques treated with Lipid 15 LNPs (FIG. 19A) or KC3 LNPs (FIG. 19B) coated with the Ab1 Fab encapsulating mCherry mRNA. Positive HSCs were transfected in vivo in a dose-dependent manner by both LNPs tested. This confirms that the human targeted HSC LNPs can efficiently transfect cynomolgus macaque LT-HSCs in vivo.

### Example 15: Preparation of Fab′ Conjugates to Enable In Vivo Hematopoietic Stem Cell (HSC) Targeting

This Example describes a method for the production of lipid-HSC targeting group conjugates for incorporation into HSC-targeting LNPs (e.g., LNPs comprising an ionizable cationic lipid, where the ionizable cationic lipid is KC3 or Lipid 15).

Fabs that bind to HSC-specific targets (CD117, CD105) were conjugated to DSPE-PEG(2k)-maleimide via covalent coupling between the maleimide group and a C-terminal cysteine in the heavy chain (HC), following initial reduction of the mixture of Fab′ and (Fab′)₂. The protein was reconstituted with molecular biology grade water at 10 mg/mL in phosphate buffered saline (10 mM phosphate, 140 mM NaCl pH 7.4) and further diluted to 5 mg/mL in reduction buffer containing final concentration of 50 mM phosphate, 10 mM citrate, 75 mM NaCl, 5 mM EDTA pH 6.0 with 20 mM L-cysteine reducing agent and incubated for 1 hr at 25° C. with agitation under an Argon atmosphere. The reduced protein was immediately buffer exchanged to 99.9% into conjugation buffer 5 mM citrate, 140 mM NaCl, 1 mM EDTA pH 6.0 with a 10 kDa molecular-weight cutoff regenerated cellulose membrane in 24-well polypropylene filtration plate at room temperature using automated ultrafiltration/diafiltration buffer exchange (Unchained Labs, California, U.S.) equipped with HEPA air filtration system. The free sulfhydryl content after reduction and buffer exchange was measured to be <1.1 per Fab molecule after reduction and buffer exchange using Ell'an's Reagent (′,5′-dithio-bis-[2-nitrobenzoic acid]) according to manufacturer's protocol (Thermo Fisher Scientific Peirce Biotechnology, Illinois, U.S.).

As soon as possible within 1 hr after buffer exchange the conjugation reaction was initiated by addition of a micellar suspension with 12 mg/mL DSPE-PEG-OCH3 (NOF America, New York, U.S.) and 8 mg/mL DSPE-PEG-maleimide (NOF America, New York, U.S.) in molecular biology grade water. The conjugation reaction was carried out with a final concentration of 3.8 mg/mL Fab and an 8.25 molar excess of maleimide at for 4 hr at 25° C. with agitation under Argon atmosphere. The production of the resulting conjugate was monitored by HPLC and SDS-PAGE. The reaction was quenched in 1.0 mM L-cysteine at room temperature for 10 min and stored at 4° C. for 12-16 hr. The resulting crude conjugate reaction containing DSPE-PEG(2k)-anti-hCD117 Fab was simultaneously purified from free Fab and buffer exchanged to 99.9% into 10 mM citrate, 10% (w/v) sucrose pH 7.0 with a 100 kDa molecular-weight cutoff regenerated cellulose membrane in 24-well polypropylene filtration plate at room temperature using automated ultrafiltration/diafiltration buffer exchange (Unchained Labs, California, U.S.) equipped with HEPA air filtration system. Purity of the final conjugate from was assessed by HPLC and by SDS-PAGE. After quenching, the final micelle composition consists of a mixture of DSPE-PEG-Fab, DSPE-PEG-maleimide (cysteine terminated), and DSPE-PEG-OCH3.

What is claimed is:

1. A lipid nanoparticle (LNP) for targeted delivery of one or more nucleic acids into a hematopoietic stem cell (HSC), wherein the LNP comprises:
(a) a lipid-antibody conjugate comprising the compound of Formula (I):
[Lipid]-[optional linker]-[antibody] (I),
wherein the antibody binds to CD105 and/or CD117;
(b) an ionizable cationic lipid comprising:
i. a compound of Formula (II′): 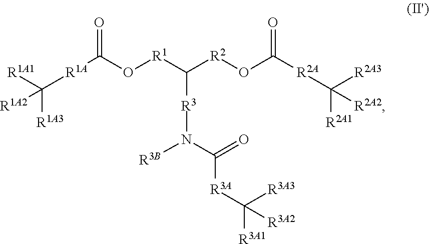
or a salt thereof, wherein:
R¹, R², and R³ are each independently a bond or C₁₋₃ alkylene;
R^{1A}, R^{2A}, and R^{3A} are each independently a bond or C₁₋₁₀ alkylene;
R^{1A1}, R^{1A2}, R^{1A3}, R^{2A1}, R^{2A2}, R^{2A3}, R^{3A1}, R^{3A}, and R^{3A3} are each independently H, C₁₋₂₀ alkyl, C₁₋₂₀ alkenyl, —(CH₂)₀₋₁₀C(O)OR^{a1}, or —(CH₂)₀₋₁₀OC(O)R^{a2};
R^{a1} and R^{a2} are each independently C₁₋₂₀ alkyl or C₁₋₂₀ alkenyl;
R^{3B} is 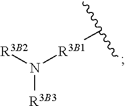
R^{3B1} is C₁₋₆ alkylene; and
R^{3B2} and R^{3B3} are each independently H or C₁₋₆ alkyl optionally substituted with one or more substituents each independently selected from the group consisting of —OH and —O—(C₁₋₆ alkyl); or
ii. a compound having the following structure: 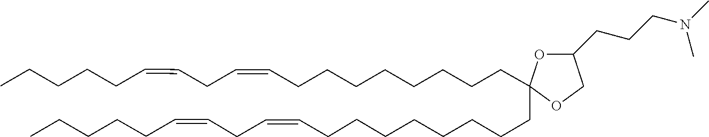 [SMILES: CCCCC/C=C\C/C=C\CCCCCCCCC1(CCCCCCCC/C=C\C/C=C\CCCCC)OCC(CCCN(C)C)O1]
or a salt thereof; and
(c) one or more nucleic acids disposed in the LNP.

2. The LNP of claim 1, wherein the LNP comprises two lipid-antibody conjugates comprising the compound of Formula (I), wherein the first lipid-antibody conjugate and the second lipid-antibody conjugate are the same or different.

3. The LNP of claim 1 or claim 2, wherein the antibody comprises an antibody heavy chain variable region (VH) and an antibody light chain variable region (VL), wherein:
(a) the antibody binds to CD117, and wherein
(i) the VH comprises a CDR-H1 comprising the amino acid sequence of FTFSNYAMS (SEQ ID NO: 1), a CDR-H2 comprising the amino acid sequence of AISGSGGSTYYADSVKG (SEQ ID NO: 2), and a CDR-H3 comprising the amino acid sequence of AKGPPTYHTNYYYMDV (SEQ ID NO: 3), and
(ii) the VL comprises CDR-L1 comprising the amino acid sequence of RASQGISSWLA (SEQ ID NO: 4), a CDR-L2 comprising the amino acid sequence of AASSLQS (SEQ ID NO: 5), and a CDR-L3 comprising the amino acid sequence of QQTNSFPYT (SEQ ID NO: 6);
(b) the antibody binds to CD117, and wherein
(i) the VH comprises a CDR-H1 comprising the amino acid sequence of FTFSDADMD (SEQ ID NO: 10), a CDR-H2 comprising the amino acid sequence of RTRNKAGSYTTEYAASVKG (SEQ ID NO: 11), and a CDR-H3 comprising the amino acid sequence of AREPKYWIDFDL (SEQ ID NO: 12), and
(ii) the VL comprises CDR-L1 comprising the amino acid sequence of RASQSISSYLN (SEQ ID NO: 13), a CDR-L2 comprising the amino acid sequence of AASSLQS (SEQ ID NO: 14), and a CDR-L3 comprising the amino acid sequence of QQSYIAPYT (SEQ ID NO: 15); or
(c) the antibody binds to CD105, and wherein
(i) the VH comprises a CDR-H1 comprising the amino acid sequence of DAWMD (SEQ ID NO: 19), a CDR-H2 comprising the amino acid sequence of EIRSKASNHATYYAESVKG (SEQ ID NO: 20), and a CDR-H3 comprising the amino acid sequence of WRRFFDS (SEQ ID NO. 21), and
(ii) the VL comprises CDR-L1 comprising the amino acid sequence of RASSSVSYMH (SEQ ID NO: 22), a CDR-L2 comprising the amino acid sequence of ATSNLAS (SEQ ID NO: 23), and a CDR-L3 comprising the amino acid sequence of QQWSSNPLT (SEQ ID NO: 24).

4. The LNP of any one of claims 1-3, wherein the antibody binds to CD117 and comprises an antibody heavy chain variable region (VH) and an antibody light chain variable region (VL), wherein
(a) the VH comprises a CDR-H1 comprising the amino acid sequence of FTFSNYAMS (SEQ ID NO: 1), a CDR-H2 comprising the amino acid sequence of AISGSGGSTYYADSVKG (SEQ ID NO: 2), and a CDR-H3 comprising the amino acid sequence of AKGPPTYHTNYYYMDV (SEQ ID NO: 3), and
(b) the VL comprises CDR-L1 comprising the amino acid sequence of RASQGISSWLA (SEQ ID NO: 4), a CDR-L2 comprising the amino acid sequence of AASSLQS (SEQ ID NO: 5), and a CDR-L3 comprising the amino acid sequence of QQTNSFPYT (SEQ ID NO: 6).

5. The LNP of any one of claims 1-3, wherein the antibody binds to CD117 and comprises an antibody heavy chain variable region (VH) and an antibody light chain variable region (VL), wherein
(a) the VH comprises a CDR-H1 comprising the amino acid sequence of FTFSDADMD (SEQ ID NO: 10), a CDR-H2 comprising the amino acid sequence of RTRNKAGSYTTEYAASVKG (SEQ ID NO: 11), and a CDR-H3 comprising the amino acid sequence of AREPKYWIDFDL (SEQ ID NO: 12), and
(b) the VL comprises CDR-L1 comprising the amino acid sequence of RASQSISSYLN (SEQ ID NO: 13), a CDR-L2 comprising the amino acid sequence of AASSLQS (SEQ ID NO: 14), and a CDR-L3 comprising the amino acid sequence of QQSYIAPYT (SEQ ID NO: 15).

6. The LNP of any one of claims 1-3, wherein the antibody binds to CD105 and comprises an antibody heavy chain variable region (VH) and an antibody light chain variable region (VL), wherein
(a) the VH comprises a CDR-H1 comprising the amino acid sequence of DAWMD (SEQ ID NO: 19), a CDR-H2 comprising the amino acid sequence of EIRSKASNHATYYAESVKG (SEQ ID NO: 20), and a CDR-H3 comprising the amino acid sequence of WRRFFDS (SEQ ID NO: 21), and
(b) the VL comprises CDR-L1 comprising the amino acid sequence of RASSSVSYMH (SEQ ID NO: 22), a CDR-L2 comprising the amino acid sequence of ATSNLAS (SEQ ID NO: 23), and a CDR-L3 comprising the amino acid sequence of QQWSSNPLT (SEQ ID NO: 24).

7. The LNP of any one of claims 1-6, wherein the antibody comprises an antibody heavy chain variable region (VH) and an antibody light chain variable region (VL), wherein
(a) the antibody binds to CD117, and wherein the VH comprises the amino acid sequence of SEQ ID NO: 7 and the VL comprises the amino acid sequence of SEQ ID NO: 8;
(b) the antibody binds to CD117, and wherein the VH comprises the amino acid sequence of SEQ ID NO: 16 and the VL comprises the amino acid sequence of SEQ ID NO: 17; or
(c) the antibody binds to CD105, and wherein the VH comprises the amino acid sequence of SEQ ID NO: 25 and the VL comprises the amino acid sequence of SEQ ID NO: 26.

8. The LNP of any one of claims 1-3, wherein the antibody binds to CD117 and comprises an antibody heavy chain variable region (VH) and an antibody light chain variable region (VL), wherein the VH comprises the amino acid sequence of SEQ ID NO: 7 and the VL comprises the amino acid sequence of SEQ ID NO: 8.

9. The LNP of any one of claims 1-3, wherein the antibody binds to CD117 and comprises an antibody heavy chain variable region (VH) and an antibody light chain variable region (VL), wherein the VH comprises the amino acid sequence of SEQ ID NO: 16 and the VL comprises the amino acid sequence of SEQ ID NO: 17.

10. The LNP of any one of claims 1-3, wherein the antibody binds to CD105 and comprises an antibody heavy chain variable region (VH) and an antibody light chain variable region (VL), wherein the VH comprises the amino acid sequence of SEQ ID NO: 25 and the VL comprises the amino acid sequence of SEQ ID NO: 26.

11. The LNP of any one of claims 1-10, wherein the antibody comprises a Fab, F(ab′)₂, Fab′-SH, Fv, scFv fragment, or immunoglobulin single variable domain.

12. The LNP of any one of claims 1-11, wherein the antibody comprises a Fab.

13. The LNP of claim 12, wherein the Fab comprises a heavy chain domain and a light chain domain, and wherein
(a) the heavy chain and light chain domains of the Fab comprise the amino acid sequences set forth in SEQ ID NOs: 9 and 38, or have at least 85, 90, 95, 96, 97, 98, 99, or 99.5% sequence identity with the amino acid sequences set forth in SEQ ID NOs: 9 and 38;
(b) the heavy chain and light chain domains of the Fab comprise the amino acid sequences set forth in SEQ ID NOs: 18 and 39, or have at least 85, 90, 95, 96, 97, 98, 99, or 99.5% sequence identity with the amino acid sequences set forth in SEQ ID NOs: 18 and 39; or
(c) the heavy chain and light chain domains of the Fab comprise the amino acid sequences set forth in SEQ ID NOs: 27 and 40, or have at least 85, 90, 95, 96, 97, 98, 99, or 99.5% sequence identity with the amino acid sequences set forth in SEQ ID NOs: 27 and 40.

14. The LNP of claim 12, wherein the Fab comprises a heavy chain domain and a light chain domain, and wherein the heavy chain and light chain domains of the Fab comprise the amino acid sequences set forth in SEQ ID NOs: 9 and 38, or have at least 85, 90, 95, 96, 97, 98, 99, or 99.5% sequence identity with the amino acid sequences set forth in SEQ ID NOs: 9 and 38.

15. The LNP of claim 12, wherein the Fab comprises a heavy chain domain and a light chain domain, and wherein the heavy chain and light chain domains of the Fab comprise the amino acid sequences set forth in SEQ ID NOs: 18 and 39, or have at least 85, 90, 95, 96, 97, 98, 99, or 99.5% sequence identity with the amino acid sequences set forth in SEQ ID NOs: 18 and 39.

16. The LNP of claim 12, wherein the Fab comprises a heavy chain domain and a light chain domain, and wherein the heavy chain and light chain domains of the Fab comprise the amino acid sequences set forth in SEQ ID NOs: 27 and 40, or have at least 85, 90, 95, 96, 97, 98, 99, or 99.5% sequence identity with the amino acid sequences set forth in SEQ ID NOs: 27 and 40.

17. The LNP of any one of claims 12-16, wherein the Fab comprises a heavy chain domain and a light chain domain and wherein the heavy chain domain, the light chain domain, or both comprise a cysteine residue at the C-terminus.

18. The LNP of any one of claims 1-17, wherein the antibody comprises an Fc domain.

19. The LNP of any one of claims 11-18, wherein the Fab lacks the native interchain disulfide bond at the C-terminus.

20. The LNP of any one of claims 11-19, wherein the Fab is engineered to replace one or both cysteines on the native constant light chain and the native constant heavy chain that form the native interchain disulfide with a non-cysteine amino acid, therefor to remove the native interchain disulfide bond in the Fab.

21. The LNP of any one of claims 11-20, wherein the Fab comprises a heavy chain fragment that comprises C233S substitution, and a light chain fragment that comprises C214S substitution, numbering according to Kabat.

22. The LNP of any one of claims 11-21, wherein the Fab has a non-natural interchain disulfide bond.

23. The LNP of any one of claims 11-22, wherein the Fab has an engineered, buried interchain disulfide bond.

24. The LNP of any one of claims 11-23, wherein the Fab comprises F174C substitution in the heavy chain fragment, and S176C substitution in the light chain fragment, numbering according to Kabat.

25. The LNP of any one of claims 11-24, wherein the Fab comprises a cysteine at the C-terminus of the heavy or light chain fragment.

26. The LNP of any one of claims 11-25, wherein the Fab further comprises one or more amino acids between the heavy chain fragment of the Fab and the C-terminal cysteine.

27. The LNP of any one of claims 1-26, wherein the antibody comprises an immunoglobulin single variable (ISV) domain.

28. The LNP of claim 27, wherein the ISV domain is a Nanobody® ISV domain.

29. The LNP of claim 27 or 28, wherein the immunoglobulin single variable domain comprises a cysteine at the C-terminus.

30. The LNP of any one of claims 27-29, wherein the antibody comprises two or more VHH domains.

31. The LNP of any one of claims 27-30, wherein the immunoglobulin single variable domain comprises a VHH domain and further comprises a spacer comprising one or more amino acids between the VHH domain and the C-terminal cysteine.

32. The LNP of any one of claims 27-31, wherein the two or more VHH domains are linked by an amino acid spacer.

33. The LNP of any one of claims 27-32, wherein the antibody comprises a first VHH domain linked to an antibody CH1 domain and a second VHH domain linked to an antibody light chain constant domain

34. The LNP of any one of claims 27-33, wherein the antibody CH1 domain and the antibody light chain constant domain are linked by one or more disulfide bonds.

35. The LNP of any one of claims 27-34, wherein the CH1 domain comprises F174C and C233S substitutions, and the light chain constant domain comprises S176C and C214S substitutions, numbering according to Kabat.

36. The LNP of any one of claims 1-35, wherein the antibody comprises an amino acid spacer or linker with the amino acid sequence AAA or with an amino acid sequence set forth in any one of SEQ ID NOs: 45-60.

37. The LNP of any one of claims 1-36, wherein the antibody comprises a bispecific antibody.

38. The LNP of any one of claims 1-37, wherein the one or more nucleic acids is DNA or RNA.

39. The LNP of claim 38, wherein the RNA is an mRNA.

40. The LNP of any one of claims 1-39, wherein the one or more nucleic acids comprise an mRNA encoding a site-directed nuclease, a chemical base editor, a prime editor, or an epigenome editor.

41. The LNP of any one of claims 1-40, wherein the one or more nucleic acids comprise an mRNA encoding a site-directed nuclease.

42. The LNP of claim 41, wherein the site-directed nuclease is a CRISPR-associated (Cas) nuclease, a zinc finger nuclease (ZFN), a transcription activator-like effector nuclease (TALEN), or a megaTAL.

43. The LNP of claim 41 or claim 42, wherein the site-directed nuclease is a Cas nuclease, ZFN, TALEN, or megaTAL comprising an amino acid sequence that confers binding to a target nucleotide sequence.

44. The LNP of any one of claims 1-43, wherein the one or more nucleic acids comprise
(a) an mRNA encoding a CRISPR-associated (Cas) nuclease or a chemical base editor; and
(b) a guide RNA (gRNA) comprising a nucleotide sequence that confers binding to a target nucleotide sequence.

45. The LNP of any one of claims 1-43, wherein the one or more nucleic acids comprise
(a) an mRNA encoding a prime editor; and
(b) a prime editing guide RNA (pegRNA) comprising a nucleotide sequence that confers binding to a target nucleotide sequence.

46. The LNP of claim 44 or claim 45, wherein the Cas nuclease is a Type II or a Type V Cas enzyme, or a variant thereof.

47. The LNP of any one of claims 44-46, wherein the Cas nuclease is a Cas9 enzyme, a Cas12 enzyme, a CasX enzyme, a Cas14 enzyme, or a variant thereof.

48. The LNP of any one of claims 44-47, wherein the gRNA or pegRNA comprises a sequence having at least 80% identity to at least 15 consecutive nucleotides of the target nucleotide sequence.

49. The LNP of any one of claims 1-48, wherein the one or more nucleic acids further comprise a donor template nucleic acid comprising a sequence having at least 80% identity to at least 15 consecutive nucleotides of the target nucleotide sequence.

50. The LNP of any one of claims 1-49, wherein the target nucleotide sequence comprises at least 15 consecutive nucleotides and is located within
a) a coding region of a gene;
b) an intronic region associated with a gene;
c) an exon region associated with a gene;
d) a 5′ untranslated region associated with a gene; or
e) a 3′ untranslated region associated with a gene;
wherein the gene is selected from the group consisting of HBB, HBG1, HBG2, HBA1, HBA2, HBD, BCL11A, BACH2, KLF1, LRF, ADA, DCLREIC, IL21G, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, 11)S, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C.

51. The LNP of any one of claims 1-50, wherein the target nucleotide sequence is within a regulatory region, optionally an enhancer region or a repressor region, of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C.

52. The LNP of any one of claims 1-51 wherein the target nucleotide sequence is within the BCL11A erythroid enhancer.

53. The LNP of any one of claims 1-52, wherein the ionizable cationic lipid comprises a compound of Formula (II′).

54. The LNP of claim 53, wherein R^{3B2} and R^{3B3} are each independently H or C₁₋₆ alkyl, optionally substituted with one or more substituents each independently selected from the group consisting of —OH and —O—(C₁₋₆ alkyl).

55. The LNP of claim 53 or claim 54, wherein R^{3B2} and R^{3B3} are each independently methyl or ethyl, each optionally substituted with one or more —OH.

56. The LNP of claim 55, wherein R^{3B2} and R^{3B3} are each unsubstituted methyl.

57. The LNP of claim 55, wherein 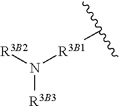
is

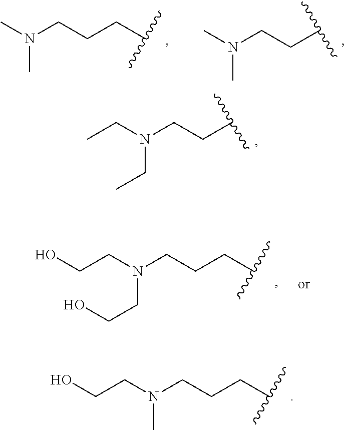

58. The LNP of claim 57, wherein 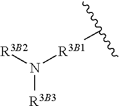
is

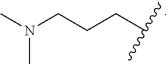 [SMILES: *CCCN(C)C]

59. The LNP of any one of claims 5-58, wherein R¹, R², and R³ are each independently a bond or methylene.

60. The LNP of any one of claims 53-59, wherein R¹ and R² are each methylene and R³ is a bond.

61. The LNP of any one of claims 53-60, wherein the ionizable cationic lipid is a compound of Formula (IIb):

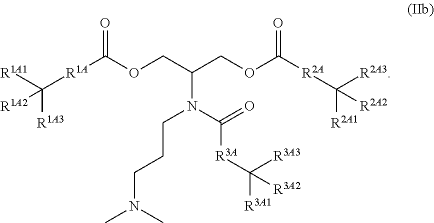

62. The LNP of any one of claims 53-61, wherein R^{1A}, R^{2A}, and R^{3A} are each independently a bond or —(CH₂)₁₋₁₀—.

63. The LNP of any one of claims 53-62, wherein R^{1A} and R^{2A} are each independently a bond, —CH₂—, —(CH₂)₂—, —(CH₂)₃—, —(CH₂)₄—, —(CH₂)₅—, —(CH₂)₆—, —(CH₂)₇—, or —(CH₂)₈—.

64. The LNP of any one of claims 53-63, wherein R^{1A} and R^{2A} are each independently a bond, —(CH₂)₂—, —(CH₂)₄—, —(CH₂)₆—, —(CH₂)₇—, or —(CH₂)₈—.

65. The LNP of any one of claims 53-64, wherein R^{3A} is a bond, —CH₂—, —(CH₂)₂—, or —(CH₂)₇—.

66. The LNP of any one of claims 53-65, wherein R^{1A1}, R^{1A2}, R^{1A3}, R^{2A1}, R^{2A2}, and R^{2A3} are each independently H, C₁₋₁₅ alkyl, —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), —(CH₂)₀₋₄OC(O)CH(C₁₋₁₀ alkyl)(C₁₋₁₅ alkyl), —(CH₂)₀₋₄C(O)OCH₂(C₁₋₁₅ alkyl), or —(CH₂)₀₋₄OC(O)CH₂(C₁₋₁₅ alkyl).

67. The LNP of any one of claims 53-66, wherein R^{1A1} and R^{2A1} are each independently —CH═CH—(C₁₋₁₅ alkyl), —CH═CH—CH₂—CH═CH—(C₁₋₁₀ alkyl), —(CH₂)₀₋₄C(O)OCH(C₁₋₁₀ alkyl)(C₁₋₁₅ alkyl), or —(CH₂)₀₋₄OC(O)CH(C₁₋₁₀ alkyl)(C₁₋₁₅ alkyl); and R^{1A2}, R^{1A3}, R^{2A2}, and R^{2A3} are each H.

68. The LNP of any one of claims 53-67, wherein R^{1A1} and R^{2A1} are each

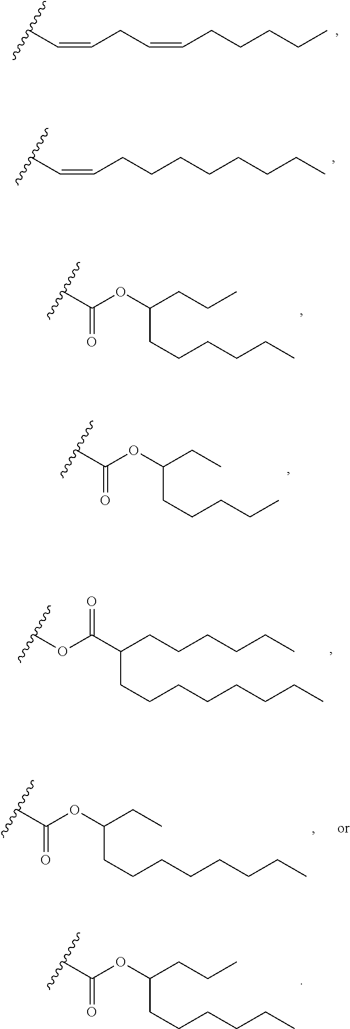

69. The LNP of any one of claims 53-68, wherein R^{1A1} and R^{2A1} are each

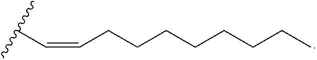 [SMILES: */C=C\CCCCCCCC]

70. The LNP of any one of claims 53-69, wherein R^{3A1}, R^{3A2}, and R^{3A3} are each independently H, C₁₋₁₅ alkyl, —(CH₂)₀₋₄C(O)OCH(C₁₋₁₅ alkyl)(C₁₋₁₀ alkyl), —(CH₂)₀₋₄OC(O)CH(C₁₋₅ alkyl)(C₁₋₁₀ alkyl), —(CH₂)₀₋₄C(O)OCH₂(C₁₋₁₀ alkyl), or —(CH₂)₀₋₄OC(O)CH₂(C₁₋₁₀ alkyl).

71. The LNP of any one of claims 53-70, wherein R^{3A1} and R^{3A2} are each independently C₁₋₁₅ alkyl; and R^{3A3} is H.

72. The LNP of any one of claims 53-71, wherein R^{3A1} and R^{3A2} are each independently ethyl, propyl,

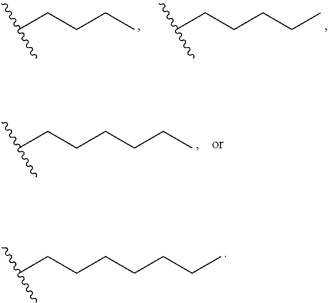

73. The LNP of any one of claims 53-72, wherein R^{3A1} is propyl and R^{3A2} is

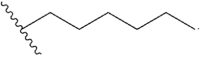 [SMILES: *CCCCCC]

74. The LNP of any one of claims 53-73, wherein the ionizable cationic lipid is

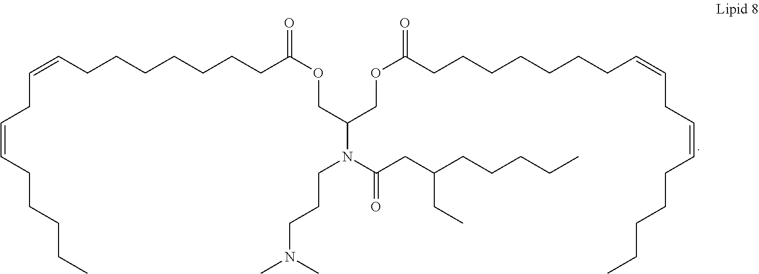 [SMILES: CCCCC/C=C\C/C=C\CCCCCCCC(=O)OCC(COC(=O)CCCCCCC/C=C\C/C=C\CCCCC)N(CCCN(C)C)C(=O)CC(CC)CCCCC]

75. The LNP of any one of claims 53-73, wherein the ionizable cationic lipid is

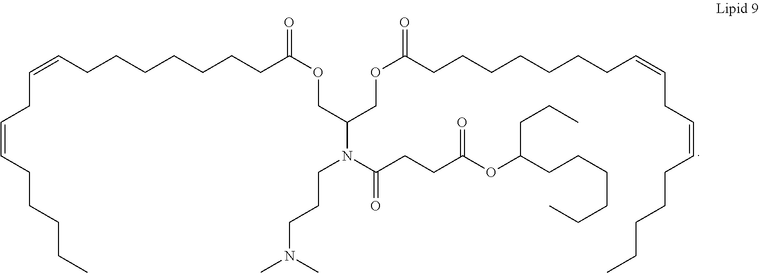 [SMILES: CCCCC/C=C\C/C=C\CCCCCCCC(=O)OCC(COC(=O)CCCCCCC/C=C\C/C=C\CCCCC)N(CCCN(C)C)C(=O)CCC(=O)OC(CCC)CCCCCC]

76. The LNP of any one of claims 53-73, wherein the ionizable cationic lipid is

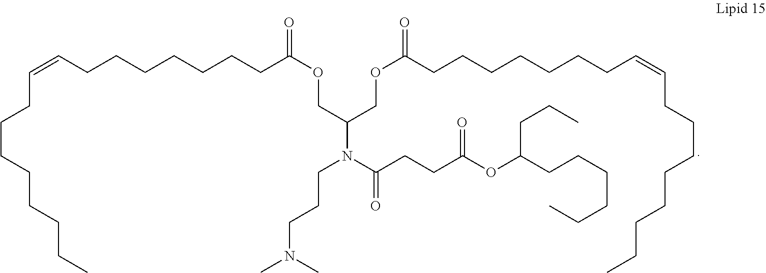 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

77. The LNP of any one of claims 53-73, wherein the ionizable cationic lipid comprises the compound having the structure

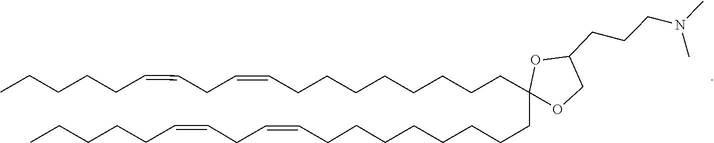 [SMILES: CCCCC/C=C\C/C=C\CCCCCCCCC1(CCCCCCCC/C=C\C/C=C\CCCCC)OCC(CCCN(C)C)O1]

78. The LNP of any one of claims 1-77, wherein the antibody is covalently coupled to a lipid in the LNP via a polyethylene glycol (PEG) containing linker.

79. The LNP of claim 78, wherein the lipid covalently coupled to the antibody via a PEG containing linker is distearoylglycerol (DSG), distearoylphosphatidylethanolamine (DSPE), dimyrstoyl-phosphatidylethanolamine (DMPE), distearoyl-glycero-phosphoglycerol (DSPG), dimyristoyl-glycerol (DMG), dipalmitoylphosphatidylethanolamine (DPPE), dipalmitoyl-glycerol (DPG), or ceramide.

80. The LNP of claim 78 and claim 79, wherein the PEG is PEG 2000 or PEG 3400.

81. The LNP of any one of claims 1-80, wherein the lipid antibody conjugate comprises [DSPE]-[PEG(2000)-maleimide]-[anti-hCD117 Fab], wherein the [DSPE]-[PEG(2000)-maleimide] is conjugated to the anti-hCD117 Fab via covalent coupling between the maleimide group and a C-terminal cysteine in the heavy chain (HC) of the anti-hCD117 Fab.

82. The LNP of any one of claims 1-80, wherein the lipid antibody conjugate comprises [DSPE]-[PEG(2000)-maleimide]-[anti-hCD105 Fab], wherein the [DSPE]-[PEG(2000)-maleimide] is conjugated to the anti-hCD105 Fab via covalent coupling between the maleimide group and a C-terminal cysteine in the heavy chain (HC) of the anti-hCD105 Fab.

83. The LNP of claim 81 or 82, wherein the ionizable cationic lipid is

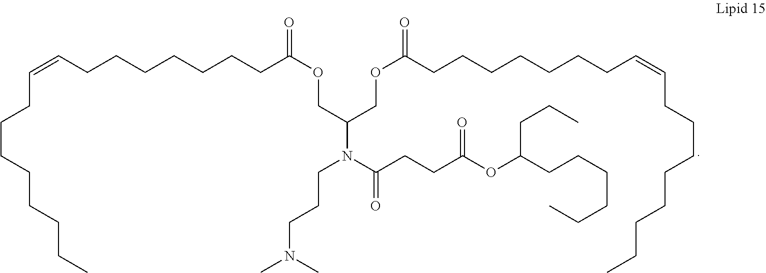 [SMILES: CCCCCCCC/C=C\CCCCCCCC(=O)OCC(COC(=O)CCCCCCC/C=C\CCCCCCCC)N(CCCN(C)C)C(=O)CCC(=O)OC(CCC)CCCCCC]

84. The LNP of any one of claims 81-83, wherein the LNP further comprises DSPE-PEG(2000)-maleimide (cysteine terminated) and DSPE-PEG-OCH₃.

85. The LNP of any one of claims 1-84, wherein the lipid-antibody conjugate is present in the LNP in a range of 0.001 to 0.5 mole percent.

86. The LNP of any one of claims 1-85, wherein the lipid-antibody conjugate is present in the LNP in a range of 0.002-0.2 mole percent.

87. The LNP of any one of claims 1-86, wherein the LNP further comprises one or more of a structural lipid, a neutral phospholipid, and a free PEG-lipid.

88. The LNP of claim 87, wherein the structural lipid is a sterol.

89. The LNP of claim 88, wherein the sterol is cholesterol.

90. The LNP of any one of claims 1-89, wherein the ionizable cationic lipid is present in the LNP in a range of 30-70 mole percent.

91. The LNP of any one of claims 1-90, wherein the ionizable cationic lipid is present in the LNP in a range of 40-60 mole percent.

92. The LNP of any one of claims 88-91, wherein the sterol is present in the LNP in a range of 20-70 mole percent.

93. The LNP of any one of claims 88-92, wherein the sterol is present in the LNP in a range of 30-50 mole percent.

94. The LNP of claim 87, wherein the neutral phospholipid is selected from the group consisting of phosphatidylcholine, phosphatidylethanolamine, distearoyl-snglycero-3-phosphoethanolamine (DSPE), 1,2-distearoyl-sn-glycero-3-phosphocholine (DSPC), 1,2-dioleoyl-sn-glycero-3-phosphoethanolamine (DOPE), 1,2-dioleoyl-sn-glycero-3-phosphocholine (DOPC), and sphingomyelin.

95. The LNP of claim 87 or claim 94, wherein the neutral phospholipid is present in the LNP in a range of 5-15 mole percent.

96. The LNP of claim 87, wherein the free PEG-lipid is selected from the group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols.

97. The LNP of claim 87 or claim 96, wherein the free PEG-lipid comprises PEG-dioleoylgylcerol (PEG-DOG), PEG-dimyristoyl-glycerol (PEG-DMG), PEG-dipalmitoylglycerol (PEG-DPG), PEG-dilinoleoyl-glycero-phosphatidyl ethanolamine (PEG-DLPE), PEG-dimyrstoyl-phosphatidylethanolamine (PEG-DMPE), PEG-dipalmitoylphosphatidylethanolamine (PEG-DPPE), PEG-distearoylglycerol (PEG-DSG), N-palmitoyl-sphingosine-1-{succinyl[methoxy(polyethylene glycol)](PEG-ceramide), PEG-distearoyl-glycero-phosphoglycerol (PEG-DSPG), PEG-dioleoyl-glycerophosphoethanolamine (PEG-DOPE), 2-[(polyethylene glycol)-2000]—N,Nditetradecylacetamide, PEG-distearoyl-phosphatidylethanolamine (PEG-DSPE), or a derivative thereof.

98. The LNP of any one of claims 87, 96, and 97, wherein the free PEG-lipid comprises a diacylphosphatidylethanolamine comprising Dipalmitoyl (C16) chain or Distearoyl (C18) chain, and optionally the free PEG-lipid comprises PEG-DPG and PEG-DMG.

99. The LNP of any one of claims 87 and 96-98, wherein the free PEG-lipid is present in the LNP in a range of 1-4 mole percent.

100. The LNP of any one of claims 87 and 96-99, wherein the free PEG-lipid comprises the same or a different lipid as the lipid in the lipid-antibody conjugate.

101. The LNP of any one of claims 87 and 96-100, wherein the free PEG-lipid comprises a PEG having a molecular weight of at least 2000 daltons.

102. The LNP of claim 92, wherein the PEG has a molecular weight of about 3000 to 5000 daltons.

103. The LNP of any one of claims 1-102, wherein the LNP has a mean diameter in the range of 50-200 nm.

104. The LNP of any one of claims 1-103, where the LNP has a mean diameter of about 100 nm.

105. The LNP of any one of claims 1-104, wherein the LNP has a polydispersity index in a range from 0.05 to 1.

106. The LNP of any one of claims 1-105, wherein the LNP has a zeta potential of from about +10 mV to about +30 mV at pH 5.

107. The LNP of any one of claims 1-105, wherein the LNP has a zeta potential of from about −30 mV to about +5 mV at pH 7.4.

108. The LNP of any of claims 87-107, wherein the LNP comprises:
(a) the ionizable cationic lipid;
(b) the lipid-antibody conjugate comprising the compound of the following formula:
[Lipid]-[optional linker]-[antibody],
wherein the antibody binds to CD105 and/or CD117;
(c) a sterol or other structural lipid;
(d) a neutral phospholipid;
(e) a free Polyethylene glycol (PEG) lipid; and
(f) the one or more nucleic acids.

109. A method of targeting the delivery of a nucleic acid to a hematopoietic stem cell (HSC), optionally ex vivo or in vivo in a subject, the method comprising administering to the subject the LNP of any one of claims 1-108, wherein the LNP comprises the nucleic acid.

110. The method of claim 109, wherein the method further comprises administering to the subject an HSC mobilization agent, and wherein the LNP is administered to the subject intravenously.

111. The method of claim 110, wherein the HSC mobilization agent is administered to the subject before, during, or before and during administration of the LNP.

112. The method of claim 110 or claim 111, wherein the HSC mobilization agent comprises plerixafor, granulocyte colony stimulating factor (G-CSF), granulocyte-macrophage colony stimulating factor (GM-CSF), or any combination thereof.

113. The method of any one of claims 110-112, wherein the HSC mobilization agent comprises plerixafor and G-CSF.

114. A method of genetically modifying a hematopoietic stem cell (HSC), optionally ex vivo or in vivo in a subject, the method comprising administering to the subject the LNP of any one of claims 1-108, wherein the one or more nucleic acids disposed in the LNP comprise an mRNA encoding a site-directed nuclease, a chemical base editor, a prime editor, or an epigenome editor.

115. A method of treating a disease in a subject in need thereof, the method comprising administering to the subject the LNP of any one of claims 1-108, wherein the one or more nucleic acids disposed in the LNP comprise a sequence, optionally an mRNA, encoding a site-directed nuclease, a chemical base editor, a prime editor, or an epigenome editor.

116. The method of claim 114 or claim 115, wherein the method further comprises administering to the subject an HSC mobilization agent, and wherein the LNP is administered to the subject intravenously.

117. The method of claim 116, wherein the HSC mobilization agent is administered to the subject before, during, or before and during administration of the LNP.

118. The method of claim 116 or claim 117, wherein the HSC mobilization agent comprises plerixafor, granulocyte colony stimulating factor (G-CSF), granulocyte-macrophage colony stimulating factor (GM-CSF), or any combination thereof.

119. The method of any one of claims 116-118, wherein the HSC mobilization agent comprises plerixafor and G-CSF.

120. The method of any one of claims 115-119, wherein the disease is a blood disease.

121. The method of any one of claims 115-119, wherein the disease is a hemoglobinopathy, a primary immune deficiency (PID), a congenital cytopenia, a hemophilia, a thrombophilia, an inborn error of metabolism, a neuropathy, or a viral disease.

122. The method of claim 121, wherein the disease is an α-hemoglobinopathy or a β-hemoglobinopathy.

123. The method of claim 121 or claim 122, wherein the β-hemoglobinopathy is β-thalassemia or sickle cell disease.

124. The method of any one of claims 121-123, wherein administration of the LNPs results in one or more of:
(a) insertion of an HBB transgene, or a fragment thereof, into at least one HSC of the subject;
(b) increased expression of β-globin in the subject,
(c) an increased amount of α₂β₂ adult hemoglobin (HbA) in the subject;
(d) insertion of an HBG1 transgene, or a fragment thereof, into at least one HSC of the subject;
(e) insertion of an HBG2 transgene, or a fragment thereof, into at least one HSC of the subject;
(f) increased expression of γ-globin in the subject;
(g) an increased amount of α₂γ₂ fetal hemoglobin (HbF) in the subject;
(h) disruption of the HBA1 gene, the HBA2 gene, or a combination thereof in at least one HSC of the subject;
(i) decreased expression of α-globin in the subject; and
(j) a decreased amount of α₄ α-globin heterotetramers the subject.

125. The method of claim 121, wherein the disease is a PID.

126. The method of claim 125, wherein the PID is a severe combined immunodeficiency (SCID), Wiskott-Aldrich syndrome, chronic granulomatous disease, immunodysregulation polyendocrinopathy enteropathay X-linked (IPEX), a hyper IgM syndrome, or X-linked agammaglobulinemia.

127. The method of claim 126, wherein the PLD is a SCID.

128. The method of claim 127, wherein the SCID is Artemis-SCID (ART-SCID), recombination activating gene SCID (RAG-SCID), X-linked SCID (X-SCID), adenosine deaminase-deficient SCID, interleukin 7 receptor deficiency SCID, or JAK3 SCID.

129. The method of claim 128, wherein the SCID is ART-SCID, and wherein administration of the LNP results in insertion of a DCLREIC transgene, or a fragment thereof, into at least one HSC of the subject; increased expression of functional Artemis protein in the subject; or a combination thereof.

130. The method of claim 128, wherein the SCID is RAG-SCID, and wherein administration of the LNP results in insertion of a RAG1 transgene or a RAG2 trangene, or a fragment thereof, into at least one HSC of the subject; increased expression of functional RAG1 protein or RAG2 protein in the subject; or a combination thereof.

131. The method of claim 128, wherein the SCID is X-SCID, and wherein administration of the LNP results in insertion of an IL2RG transgene, or a fragment thereof, in at least one HSC of the subject; increased expression of functional IL2RG protein in the subject; or a combination thereof.

132. The method of claim 125 or claim 126, wherein the PID is Wiskott-Aldrich syndrome.

133. The method of claim 132, wherein the PID is Wiskott-Aldrich syndrome, and wherein administration of the LNP results in insertion of a WAS transgene, or a fragment thereof, into at least one HSC of the subject; increased expression of functional WASP protein expression in the subject; or a combination thereof.

134. The method of claim 125 or claim 126, wherein the PID is chronic granulomatous disease.

135. The method of any one of claims 125, 126 and 134, wherein the PID is X-linked chronic granulomatous disease.

136. The method of any one of claims 125, 126, and 134, wherein the PID is chronic granulomatous disease, and wherein administration of the LNP results in one or more of:
(a) insertion of a CYBA transgene, a CYBB transgene, an NCF1 transgene, NCF2 transgene, or an NCF4 transgene, or a fragment thereof, into at least one HSC of the subject;
(b) introduction of a point 676C>T pointe mutation in the CYBB gene of at least one HSC in the subject;
(c) increased expression of functional CYBA protein, CYBB protein, NCF1 protein, NCF2 protein, or NCF4 protein in the subject; and
(d) an increased amount of functional NADPH oxidase enzyme complex in the subject.

137. The method of any one of claims 125 and 126, wherein the PID is IPEX.

138. The method of any one of claims 125, 126 and 137, wherein the PID is IPEX, and wherein administration of the LNP results in insertion of an FOXP3 transgene, or a fragment thereof, into at least one HSC of the subject; increased expression of functional FOXP3 protein in the subject; or a combination thereof.

139. The method of any one of claims 125 and 126, wherein the PID is hyper IgM syndrome.

140. The method of any one of claims 125, 126 and 139, wherein the PID is hyper IgM syndrome, and wherein administration of the LNP results in one or more of:
(a) insertion of a AICDA transgene, a (UNG transgene, an CD40 transgene, or a CD40LG transgene, or a fragment thereof, into at least one HSC of the subject;
(b) increased expression of functional AICDA protein, UNG protein, CD40 protein, or CD40LG protein in the subject;
(c) a decreased amount of IgM antibodies in the subject; and
(d) an increased amount of IgG, IgA, or IgE antibodies in the subject.

141. The method of claim 121, wherein the disease is a congenital cytopenia.

142. The method of claim 141, wherein the congenital cytopenia is Fanconia anemia, Shwachman-Diamond syndrome, Blackfan-Diamond anemia, dyskeratosis congenita, congenital amegakaryocytic thrombocytopenia, or reticular dysgenesis.

143. The method of claim 141 or claim 142, wherein the congenital cytopenia is Fanconia anemia, and wherein administration of the LNP results in insertion of one or more FANC genes, or a fragment thereof, into at least one HSC in the subject; increased expression of one or more functional FANC proteins in the subject; or a combination thereof.

144. The method of any one of claims 141-143, wherein the congenital cytopenia is Fanconia anemia, and wherein administration of the LNP insertion of a FANCA transgene, or a fragment thereof, into at least one HSC of the subject; increased expression of functional FANCA in the subject; or a combination thereof.

145. The method of claim 121, wherein the disease is a hemophilia.

146. The method of claim 145, wherein the hemophilia is hemophilia A, hemophilia B, or hemophilia C.

147. The method of claim 121, wherein the disease is a hemophilia, and wherein administration of the LNP results in:
(a) insertion of a F8 transgene, a F9 transgene, or an F11, or a fragment thereof, into at least one HSC of the subject;
(b) increased expression of functional factor VIII protein, factor IX protein, or factor XI protein in the subject; and
(c) increased blood clotting in the subject.

148. The method of claim 121, wherein the disease is a thrombophilia.

149. The method of claim 148, wherein the thrombophilia is amegakaryocytic thrombocytopenia or factor X deficiency.

150. The method of claim 121, wherein the disease is a thrombophilia, and wherein administration of the LNP results in one or more of:
(a) insertion of a F5 transgene, a F2 transgene, a transgene encoding antithrombin III, a transgene encoding protein C, or a transgene encoding protein S, or a fragment thereof, into at least one HSC of the subject;
(b) increased expression of functional factor V protein, factor II protein, antithrombin III protein, protein C, or protein S in the subject; and
(c) reduced blood clotting in the subject.

151. The method of claim 121, wherein the disease is an inborn error of metabolism.

152. The method of claim 151, wherein the inborn error of metabolism is phenylketoneuria (PKU), medium-chain acyl-CoA dehydrogenase (MCAD) deficiency, a lysosomal storage disease, a glycogen storage disorder, a peroxisomal disorder, Fabry disease, Gaucher disease, Hurler syndrome, Hunter syndrome, Wolman disease, or pyruvate kinase deficiency.

153. The method of claim 152, wherein the peroxisomal disorder is X-linked adrenoleukodystrophy.

154. The method of claim 152, wherein the lysosomal storage disease is metachromatic leukodystrophy, mucopolysaccharidosis I, or mucopolysaccharidosis II.

155. The method of claim 121, wherein the disease is a neuropathy.

156. The method of claim 155, wherein the neuropathy is Friedrich's ataxia.

157. The method of claim 121, wherein the disease is a viral disease.

158. The method of claim 157, wherein the viral disease is HIV/AIDS.

159. The method of claim 157 or claim 158, wherein the viral disease is HIV/AIDS, and wherein administration of the LNP prevents infection by HIV, progression of HIV/AIDS, or a combination thereof.

160. The method of any one of claims 109-159, wherein the one or more nucleic acids disposed in the LNP comprise an mRNA encoding a site-directed nuclease.

161. The method of claim 160, wherein the site-directed nuclease is a CRISPR-associated (Cas) nuclease, a zinc finger nuclease (ZFN), a transcription activator-like effector nuclease (TALEN), or a megaTAL.

162. The method of claim 160 or claim 161, wherein the site-directed nuclease is a Cas nuclease, ZFN, TALEN, or megaTAL comprising an amino acid sequence that confers binding to a target nucleotide sequence.

163. The method of any one of claims 109-162, wherein the one or more nucleic acids disposed in the LNP comprise:
(a) an mRNA encoding a CRISPR-associated (Cas) nuclease or a chemical base editor; and
(b) a guide RNA (gRNA) comprising a nucleotide sequence that confers binding to a target nucleotide sequence.

164. The method of any one of claims 109-163, wherein the one or more nucleic acids disposed in the LNP comprise:
(a) an mRNA encoding a prime editor; and
(b) a prime editing guide RNA (pegRNA) comprising a nucleotide sequence that confers binding to a target nucleotide sequence.

165. The method of any one of claims 161-164, wherein the Cas nuclease is a Type 11 or a Type V Cas enzyme, or a variant thereof.

166. The method of any one of claims 161-165, wherein the Cas nuclease is a Cas9 enzyme, a Cas12 enzyme, a CasX enzyme, or a Cas 14 enzyme, or a variant thereof.

167. The method of any one of claims 109-166, wherein the gRNA or pegRNA comprises a sequence having at least 80% identity to at least 15 consecutive nucleotides of the target nucleotide sequence.

168. The method of any one of claims 109-167, wherein the one or more nucleic acids disposed in the LNP further comprise a donor template nucleic acid comprising a sequence having at least 80% identity to at least 15 consecutive nucleotides of the target nucleotide sequence.

169. The method of any one of claims 162-168, wherein the target nucleotide sequence comprises at least 15 consecutive nucleotides and is located within
a) a coding region of a gene;
b) an intronic region associated with a gene;
c) an exon region associated with a gene;
d) a 5′ untranslated region associated with a gene; or
e) a 3′ untranslated region associated with a gene;
wherein the gene is selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, W4S, F8, F9, F11, F10, PKLR, RPS19, CYBA, CYBB, NCF1, NCF1B, NCF1C, NCF2, NCF4, ELANE, ABCD1, ARSA, FXN, GBA, IDS, IDUA, TCIRG, AICDA, UNG, CD40, CD40LG, FOXP3, IL4, IL10, IL13, IL7R, PRF1, FANCA, FANCB, FANCC, FANCC, FANCD1/BRACA2, FANCD2, MPL, CCR5, CXCR4, F5, F2, antithrombin III, and protein C.

170. The method of any one of claims 162-169, wherein the target nucleotide sequence is within a regulatory region, optionally an enhancer region or a repressor region, of a gene selected from the group consisting of HBB, HBG1, HBG2, HBA1, HBA2, HBD, BCL11A, BACH2, KLF1, LRF, ADA, DCLREIC, IL2RG, RAG1, RAG2, JAK3, BTK, WAS, F8, F9, F11, F10, PKLR, RPS19, CYBA, CYBB, NCF1, NCF1B, NCF1C, NCF2, NCF4, ELANE, ABCD1, ARSA, FM, GBA, IDS, IDUA, TCIRG, AICDA, UNG, CD40, CD40LG, FOXP3, IL4, IL10, IL13, IL7R, PRF1, FANCA, FANCB, FANCC, FANCD1/BRACA2, FANCD2, MPL, CCR5, CXCR4, F5, F2, antithrombin III, and protein C.

171. The method of any one of claims 162-170, wherein the target nucleotide sequence is within the BCL11A erythroid enhancer.

172. The method of any one of claims 109-171, wherein the subject is a human.


SEQUENCE LISTING (62 sequences)

SEQ ID NO: 1 - protein, 9 aa, synthetic construct
FTFSNYAMS

SEQ ID NO: 2 - protein, 17 aa, synthetic construct
AISGSGGSTYYADSVKG

SEQ ID NO: 3 - protein, 16 aa, synthetic construct
AKGPPTYHTNYYYMDV

SEQ ID NO: 4 - protein, 11 aa, synthetic construct
RASQGISSWLA

SEQ ID NO: 5 - protein, 7 aa, synthetic construct
AASSLQS

SEQ ID NO: 6 - protein, 9 aa, synthetic construct
QQTNSFPYT

SEQ ID NO: 7 - protein, 123 aa, synthetic construct
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSS

SEQ ID NO: 8 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIK

SEQ ID NO: 9 - protein, 240 aa, synthetic construct
EVQLLESGGGLVQPGGSLRLSCAASGFTFSNYAMSWVRQAPGKGLEWVSAISGSGGSTYYADSVKGRFTISRDNSKNTLYLQMNSLRAEDTAVYYCAKGPPTYHTNYYYMDVWGKGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 10 - protein, 9 aa, synthetic construct
FTFSDADMD

SEQ ID NO: 11 - protein, 19 aa, synthetic construct
RTRNKAGSYTTEYAASVKG

SEQ ID NO: 12 - protein, 12 aa, synthetic construct
AREPKYWIDFDL

SEQ ID NO: 13 - protein, 11 aa, synthetic construct
RASQSISSYLN

SEQ ID NO: 14 - protein, 7 aa, synthetic construct
AASSLQS

SEQ ID NO: 15 - protein, 9 aa, synthetic construct
QQSYIAPYT

SEQ ID NO: 16 - protein, 121 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSS

SEQ ID NO: 17 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIK

SEQ ID NO: 18 - protein, 238 aa, synthetic construct
EVQLVESGGGLVQPGGSLRLSCAASGFTFSDADMDWVRQAPGKGLEWVGRTRNKAGSYTTEYAASVKGRFTISRDDSKNSLYLQMNSLKTEDTAVYYCAREPKYWIDFDLWGRGTLVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 19 - protein, 5 aa, synthetic construct
DAWMD

SEQ ID NO: 20 - protein, 19 aa, synthetic construct
EIRSKASNHATYYAESVKG

SEQ ID NO: 21 - protein, 7 aa, synthetic construct
WRRFFDS

SEQ ID NO: 22 - protein, 10 aa, synthetic construct
RASSSVSYMH

SEQ ID NO: 23 - protein, 7 aa, synthetic construct
ATSNLAS

SEQ ID NO: 24 - protein, 9 aa, synthetic construct
QQWSSNPLT

SEQ ID NO: 25 - protein, 118 aa, synthetic construct
EVKLEESGGGLVQPGGSMKLSCAASGFTFSDAWMDWVRQSPEKGLEWVAEIRSKASNHATYYAESVKGRFTISRDDSKSSVYLQMNSLRAEDTGIYYCTRWRRFFDSWGQGTTLTVSS

SEQ ID NO: 26 - protein, 106 aa, synthetic construct
QIVLSQSPAILSASPGEKVTMTCRASSSVSYMHWYQQKPGSSPKPWIYATSNLASGVPVRFSGSGSGTSYSLTISRVEAEDAATYYCQQWSSNPLTFGAGTKLELK

SEQ ID NO: 27 - protein, 235 aa, synthetic construct
EVKLEESGGGLVQPGGSMKLSCAASGFTFSDAWMDWVRQSPEKGLEWVAEIRSKASNHATYYAESVKGRFTISRDDSKSSVYLQMNSLRAEDTGIYYCTRWRRFFDSWGQGTTLTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 28 - protein, 9 aa, synthetic construct
FAAANYAMS

SEQ ID NO: 29 - protein, 17 aa, synthetic construct
AISGAAASTYYADSVKG

SEQ ID NO: 30 - protein, 16 aa, synthetic construct
AKGPPTYAAAYYYMDV

SEQ ID NO: 31 - protein, 11 aa, synthetic construct
RASQAAASWLA

SEQ ID NO: 32 - protein, 7 aa, synthetic construct
AAASLQS

SEQ ID NO: 33 - protein, 9 aa, synthetic construct
QQTAAAPYT

SEQ ID NO: 34 - protein, 123 aa, synthetic construct
EVQLLESGGGLVQPGGSLRLSCAASGFAAANYAMSWVRQAPGKGLEWVSAISGAAASTYYADSVKGRFTISRDNSKNTLYLQMNSLRAEDTAVYYCAKGPPTYAAAYYYMDVWGKGTTVTVSS

SEQ ID NO: 35 - protein, 107 aa, synthetic construct
DIQMTQSPSSVSASVGDRVTITCRASQAAASWLAWYQQKPGKAPKLLIYAAASLQSGVPSRFSGSGSGTDFTLTISSLQPEDFATYYCQQTAAAPYTFGGGTKVEIK

SEQ ID NO: 36 - protein, 240 aa, synthetic construct
EVQLLESGGGLVQPGGSLRLSCAASGFAAANYAMSWVRQAPGKGLEWVSAISGAAASTYYADSVKGRFTISRDNSKNTLYLQMNSLRAEDTAVYYCAKGPPTYAAAYYYMDVWGKGTTVTVSSASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKVEPKSSDKTHTCGGHHHHHH

SEQ ID NO: 37 - protein, 214 aa, synthetic construct
DIQMTQSPSSVSASVGDRVTITCRASQAAASWLAWYQQKPGKAPKLLIYAAASLQSGVPSRFSGSGSGTDFTLTISSLQPEDFATYYCQQTAAAPYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 38 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQTNSFPYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 39 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYIAPYTFGGGTKVEIKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 40 - protein, 213 aa, synthetic construct
QIVLSQSPAILSASPGEKVTMTCRASSSVSYMHWYQQKPGSSPKPWIYATSNLASGVPVRFSGSGSGTSYSLTISRVEAEDAATYYCQQWSSNPLTFGAGTKLELKRTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 41 - protein, 98 aa, synthetic construct
ASTKGPSVFPLAPSSKSTSGGTAALGCLVKDYFPEPVTVSWNSGALTSGVHTCPAVLQSSGLYSLSSVVTVPSSSLGTQTYICNVNHKPSNTKVDKKV

SEQ ID NO: 42 - protein, 19 aa, synthetic construct
EPKSSDKTHTCGGHHHHHH

SEQ ID NO: 43 - protein, 107 aa, synthetic construct
RTVAAPSVFIFPPSDEQLKSGTASVVCLLNNFYPREAKVQWKVDNALQSGNSQESVTEQDSKDSTYSLCSTLTLSKADYEKHKVYACEVTHQGLSSPVTKSFNRGES

SEQ ID NO: 44 - skipped

SEQ ID NO: 45 - protein, 5 aa, synthetic construct
GGGGS

SEQ ID NO: 46 - protein, 7 aa, synthetic construct
SGGSGGS

SEQ ID NO: 47 - protein, 8 aa, synthetic construct
GGGGSGGS

SEQ ID NO: 48 - protein, 9 aa, synthetic construct
GGGGSGGGS

SEQ ID NO: 49 - protein, 10 aa, synthetic construct
GGGGSGGGGS

SEQ ID NO: 50 - protein, 15 aa, synthetic construct
GGGGSGGGGSGGGGS

SEQ ID NO: 51 - protein, 18 aa, synthetic construct
GGGGSGGGGSGGGGSGGS

SEQ ID NO: 52 - protein, 20 aa, synthetic construct
GGGGSGGGGSGGGGSGGGGS

SEQ ID NO: 53 - protein, 25 aa, synthetic construct
GGGGSGGGGSGGGGSGGGGSGGGGS

SEQ ID NO: 54 - protein, 30 aa, synthetic construct
GGGGSGGGGSGGGGSGGGGSGGGGSGGGGS

SEQ ID NO: 55 - protein, 35 aa, synthetic construct
GGGGSGGGGSGGGGSGGGGSGGGGSGGGGSGGGGS

SEQ ID NO: 56 - protein, 40 aa, synthetic construct
GGGGSGGGGSGGGGSGGGGSGGGGSGGGGSGGGGSGGGGS

SEQ ID NO: 57 - protein, 15 aa, synthetic construct
EPKSCDKTHTCPPCP

SEQ ID NO: 58 - protein, 24 aa, synthetic construct
GGGGSGGGSEPKSCDKTHTCPPCP

SEQ ID NO: 59 - protein, 12 aa, synthetic construct
EPKTPKPQPAAA

SEQ ID NO: 60 - protein, 62 aa, synthetic construct
ELKTPLGDTTHTCPRCPEPKSCDTPPPCPRCPEPKSCDTPPPCPRCPEPKSCDTPPPCPRCP

SEQ ID NO: 61 - protein, 9 aa, synthetic construct
LAGLIDADG

SEQ ID NO: 62 - DNA, 20 bp, synthetic construct
  source 1..20: mol_type = other DNA
gtgataaaagcaactgttag